(19) United States
(12) Patent Application Publication
(10) Pub. No.: US 2020/0061175 A1
(43) Pub. Date: Feb. 27, 2020

(54) IMMUNOGENS OBTAINED FROM PLASMODIUM YOELII USING QUANTITATIVE SEQUENCELINKAGE GROUP SELECTION METHOD

(71) Applicant: King Abdullah University of Science and Technology, Thuwal (SA)

(72) Inventors: Arnab Pain, Thuwal (SA); Richard Culleton, Nagasaki (JP); Christopher J.R. Illingworth, Cambridgeshire (GB)

(21) Appl. No.: 16/612,686

(22) PCT Filed: May 11, 2018
(86) PCT No.: PCT/IB2018/053270

§ 371 Date: Nov. 11, 2019

Related U.S. Application Data
(60) Provisional application No. 62/504,798, filed on May 11, 2017.
(60) Provisional application No. 62/619,586, filed on Jan. 19, 2018.

(51) Int. Cl. A61K 39/015; C07K 14/445; A61P 33/02

(52) U.S. Cl. CPC A61K 39/015; A61P 33/02; C07K 14/445

(57) ABSTRACT

Provided herein are immunogenic compositions against Plasmodium, comprising an immunogenic polypeptide. Also provided are methods of immunizing a subject against Plasmodium, methods of eliciting an immune response in a subject against Plasmodium, and methods of identifying parasite genes driving medically important selectable phenotypes.

### BACKGROUND

Malaria parasite strains are genotypically polymorphic, leading to a diversity of phenotypic characteristics that impact on disease severity. Discovering the genetic basis for such phenotypic traits can inform the design of new drugs and vaccines. Both association mapping and linkage analyses approaches have been adopted to understand the genetic mechanisms behind various phenotypes of malaria parasites and with the application of whole genome sequencing (WGS), the resolution of these methodologies has been dramatically improved, allowing the discovery of selective sweeps as they arise in the field. However, both approaches suffer from drawbacks when working with malaria parasites: linkage mapping requires the cloning of individual recombinant offspring, a process that is both laborious and time-consuming, and association studies require the collection of a large number of individual parasites (usually in the thousands) from diverse geographical origins and over periods of several months or years to produce enough resolution for the detection of selective sweeps.

Linkage Group Selection (LGS), like linkage mapping, relies on the generation of genetic crosses, but bypasses the need for extracting and phenotyping individual recombinant clones. Instead, it relies on quantitative molecular markers to measure allele frequencies in the recombinant progeny and identify loci under selection. This approach bears similarity to Bulked Segregant Analysis (BSA), a technique developed to study disease resistance in plants. In BSA, individuals from a population are segregated based upon their phenotype (e.g. disease resistance), following which the frequencies of genetic markers in each population are analysed, identifying loci at which different alleles are found for the differently phenotypes populations. Segregating individuals by phenotype, while relatively straight forward for large organisms such as plants, is not feasible for unicellular pathogens such as malaria parasites. Instead, in LGS, the segregating population is grown both in the presence or absence of a selection pressure (e.g. drug treatment, immune pressure, etc.). Selection removes susceptible individuals in the selected “pool”, while leaving both susceptible and resistant individuals in the unselected “pool”. In its original implementation, LGS was successfully applied in studying strain-specific immunity (SSI), drug resistance and growth rate in malaria and SSI in Eimeria tenella. LGS is essentially identical to the extreme QTL approach (xQTL) that was independently developed by yeast researchers based on BSA.

In both the original implementations of BSA and LGS a limiting factor is the availability of molecular markers differentiating the two populations. One step in increasing the number of molecular markers was through the use of array hybridisation that allowed the identification of thousands of SNPs as molecular markers in Arabidopsis thaliana. BSA (still using pre-selected pools) was also combined with tiling microarray hybridisation and used probe intensities to detect a gene underlying xylose utilisation in yeast. The xQTL method increased the power and rapidity of the approach by making use of available yeast microarray data as well as Next Generation Sequencing (NGS) of DNA hybridised to microarray probes to identify a large number of markers across the genome, this time comparing selected and unselected populations, rather then generating pools based on phenotype. In the absence of microarray databases, an alternative approach was to use NGS short reads to identify genome-wide SNPs between two parents and then use these SNPs as molecular markers to identify target genes in the selected progeny population compared against the unselected population, as done to study chloroquine resistance in malaria.

Identifying the genetic determinants of phenotypes that impact disease severity is of fundamental importance for the design of new interventions against malaria. Here we use a novel approach to study two important properties of the parasite; the rate at which parasites grow within a single host, and the means by which parasites are affected by the host immune system.

### BRIEF DESCRIPTION OF THE FIGURES

Having thus described the invention in general terms, reference will now be made to the accompanying drawings, which are not necessarily drawn to scale.

FIG. 1 shows a schematic representation of the multi-crossing LGS approach. The process starts with the identification of distinct selectable phenotypes in cloned strains of the pathogen population (in this case malaria parasites) and their sequencing, usually from the vertebrate blood stage. A genetic cross between two cloned strains is subsequently produced, in this case inside the mosquito vector. The cross progeny is then grown with and without selection pressure(s). Selection pressure will remove those progeny individuals carrying allele(s) associated with sensitivity to the selection pressure(s), while allowing progeny individuals with the resistant allele(s) to survive. DNA is then extracted from the whole, uncloned progeny for sequencing. SNPs distinguishing both parents are used to measure allele frequencies in the selected and unselected progenies. A mathematical model is then applied to identify and define loci under selection. Regions in these loci are then analyzed in detail to identify potential target polymorphisms underlying the phenotype(s) under investigation. Targeted capillary sequencing can be employed to verify or further characterize polymorphisms. Finally, where applicable, allele replacement experiments can be carried out to confirm the effect of target polymorphisms.

FIG. 2 shows pure strain growth rates. (A) Growth rate of Plasmodium yoelii strains 17X1.1pp and CU in CBA mice inoculated with 1×10⁶ iRBCs on Day 0. Error bars indicate the standard error of the mean for three mice per group. (B) The relative proportions of CU and 17X1.1pp were measured by Q-RT-PCR targeting the polymorphic msp1 locus at Day 4 post-inoculation with a mixed inoculum containing approximately equal proportions of both strains in naïve mice and mice immunized with one of the two strains. Error bars show the standard error of the mean of five mice per group. *p<0.05, Wilcoxon rank sum test, W=25, p=0.0119, n=5; ** p<0.01, Wilcoxon rank sum test, W=25, p=0.0075, n=5.

FIGS. 3A and 3B shows genome-wide sequencing data. FIG. 3A shows genome-wide Plasmodium yoelii CU allele frequency of two independent genetic crosses grown in (a,b) naïve mice, (c,d) 17X1.1pp immunized mice and (e,f) CU-immunized mice. Light gray dots represent observed allele frequencies. Dark gray dots represent allele frequencies retained after filtering. Dark blue lines represent a smoothed approximation of the underlying allele frequency; a region of uncertainty in this frequency, of size three standard deviations, is shown in light blue. A conservative confidence interval describing the position of an allele evolving under selection is shown via a red bar. Allele frequencies are shown in log scale. FIG. 3B shows evolutionary models fitted to allele frequency data. Filtered allele frequencies are shown as gray dots, while the model fit is shown as a red line. Dark blue and light blue vertical bars show combined and conservative confidence intervals for the location of the selected allele as reported in FIG. 9. Numbers in parentheses equate figures with locations in FIG. 3A. A black vertical line shows the position of a gene of interest.

FIGS. 4A, 4B, and 4C shows EBL Amino acid sequence alignment of various malaria species and Plasmodium yoelii strains, and predicted protein structure consequences of the C351Y polymorphism. FIG. 4A shows EBL orthologous and paralogous sequences from a variety of malaria species and P. yoelii strains were aligned using ClustalW. Only the amino acids surrounding position 351 are shown. The cysteine in position 351 in P. yoelii is highly conserved across strains and species, with only strain 17X1.1pp bearing a C to Y substitution. PchAS: Plasmodium chabaudi AS strain; PbANKA: Plasmodium berghei ANKA strain; Py17X/17X1.1pp/CU/YM: P. yoelii 17X, 17X1.1pp,CU,YM strains; Pk-DBLα/β/γ: Plasmodium knowlesi Duffy Binding Ligand alpha/beta/gamma (H strain); PvDBP: Plasmodium vivax Duffy Binding Protein (Sal-I strain); PcynB_DBP1/2: Plasmodium cynomolgi Duffy Binding Proteins 1/2 (B strain); Pf3D7_EBA140/175/181: Plasmodium falciparum Erythrocyte Binding Antigens 140/175/181 (3D7 strain). FIG. 4B shows energy minimized homology model of the wild type P. yoelii (Py17XWT) Erythrocyte Binding Ligand (EBL). Inset depicts the disulfide bond between C351 and C420. (The protein is represented in cyan and the disulfide bonds are in yellow). FIG. 4C shows energy minimized homology model of the mutant (C351Y) P. yoelii (Py17X1.1pp) Erythrocyte Binding Ligand (EBL). Inset depicts the lack of a disulfide bond between Y351 (substituted C351) and C420. (The protein is represented in cyan and the disulfide bonds are in yellow and Tyr351 [mutated] is represented in magenta).

FIG. 5 shows localization of EBL. The C351Y polymorphism does not affect EBL subcellular localization in Plasmodium yoelii. (A) P. yoelii schizonts of wild type and transgenic parasite lines were incubated with fluorescent mouse anti-EBL serum, fluorescent rabbit anti-AMA1 serum, and DAPI nuclear staining. Colors indicate the localization of the Pyebl (green) and AMA-1 (red) proteins, as well as nuclear DNA (blue). 17XL: fast growing 17X clone previously shown to traffic EBL to the dense granules, not the micronemes, 17X1.1pp: 17x1.1pp strain, CU: CU strain, 17X1.1-351Y>C: 17X1.1pp strain transfected with the CU allele for Pyebl, CU-351C>Y: CU strain transfected with the 17X1.1pp allele of Pyebl. (B) The distance of EBL from AMA1 measured for five parasite strains and for 5-9 schizonts per strain; stars indicate p<0.01 using a Mann-Whitney U test. This indicates a shift in the location of Pyebl occurring in 17XL, but not in any other parasite lines.

FIG. 6 shows site directed mutagenesis of pyebl AA position 351 reverses the phenotypes of parasites with slow and intermediate growth rates. (A) Growth rate of P. yoelii strains 17X1.1pp, CU and of the CU-strains transfected with either CU (CU-EBL-351C>C) or 17X1.1 (CU-EBL-351C>Y) Pyebl gene in CBA mice inoculated with 1×106 iRBCs on Day 0. (B) Growth rate of P. yoelii strains 17X1.1pp, CU and of the 17X1.1pp-strains transfected with either 17X1.1 (17X1.1pp-EBL-351Y>Y) or CU (17X1.1pp-EBL-351Y>C) Pyebl gene alleles in CBA mice inoculated with 1×106 iRBCs on Day 0. Transfection with the 17X1.1pp (EBL-351Y) allele produces a significantly increased growth rate in the CU strain (CU-EBL-351C>C vs CU-EBL-351C>Y: p<0.01, Two-way ANOVA with Tukey post-test correction) that is not significantly different from 17X1.1pp growth rate following transfection with its native allele (17X1.1pp-EBL-351Y>Y vs. CU-EBL-351C>Y: p>0.05, Two-way ANOVA with Tukey post-test correction). Conversely, transfection with the CU (EBA-351C) allele significantly reduces growth (17X1.1pp-EBL-351Y>Y vs 17X1.1pp-EBL-351Y>C: p<0.01, Two-way ANOVA with Tukey post-test correction) and produces a phenotype that is not significantly different from CU transfected with its own allele (CU EBL-351C>C vs 17X1.1pp-EBL-351Y>C: p>0.05, Two-way ANOVA with Tukey post-test correction).

FIG. 7 shows sudden changes in allele frequency identified using a jump-diffusion model. Details are given for loci at which a sudden jump in frequency was inferred with probability at least 1%. The latter value is the inferred probability that the change in allele frequency at a given locus arose from a jump to a random position between 0 and 1, as opposed to arising from a small change to the frequency at the previous locus. Data are shown for the naive and 17-X immunized experiments; no jumps of this significance were inferred for the CU-immunized experiment.

FIG. 8 shows identification of candidate regions by non-neutrality score and SD model selected allele location. The non-neutrality score for region in replica r is denoted Sᵣ. The optimal driver location in the same region is given by i*ᵣ. Where a chromosome is divided into parts, by potential jump alleles, the resulting genomic regions are denoted by their chromosome number, a subscript indicating which part of the genome was under consideration. Identified candidate regions were defined as those at which selection was identified at positions within 200 kb in both replicates, and are here highlighted in bold type.

FIG. 9 shows confidence intervals for driver locations as determined by mathematical modeling.

FIG. 10 shows parasitaemias after immune challenges. (A) The course of infection of 1:1 mixtures of blood stage Plasmodium yoelii yoelii 17x1.1 and CU parasites in mock-immunised (red line), 17x1.1 (green line) and CU (purple line) immunised mice through time. Error bars indicate standard errors of the mean of 6 mice per group. (B) The course of infection of uncloned recombinant progeny of a cross between Plasmodium yoelii yoelii 17x1.1 and CU parasites in mock-immunised (red line), 17x1.1 (green line) and CU (purple line) immunised mice through time. (C-E) The course of infection of 1:1 mixtures of blood stage Plasmodium yoelii yoelii 17x1.1 and CU parasites in mock-immunised (blue lines), 17x1.1 (red lines) and CU (green lines) immunised mice through time in BALB/c (C), CBA/n (D) and C57/BL6 (E) mice. Error bars indicate standard errors of the mean of 3 mice per group.

FIG. 11 shows intracellular localization of EBL in parasite strains CU, 17XL, 17X1.1pp and in transfected parasites CU(CY) and 17X1.1pp(YC). (A) Antibody-mediated staining of EBL (green), AMA1 (red) and DAPI staining of DNA (blue) inside the parasite cell in strain 17XL. (B) Intensity of fluorescent staining related to location in strain 17XL, Y-axis indicates fluorescence intensity, X-axis indicates distance along the merozoite starting from the posterior terminal end. (C) Comparisons of the distances of EBL from DNA and AMA1 from DNA in the 5 parasite strains. The distance of EBL or AMA1 from DNA measured across 5 parasite strains and between 5-9 merozoites for each strain; stars indicate p<0.05 using a Wilcoxon signed-rank test.

FIG. 12 shows expression of Pyebl alleles in both wild type (WT) and transfected strains. mRNA from the parental WT strains CU and 17X1.1pp, as well the CU strain transfected with the 17X1.1pp allele (CU C351Y) and the 17XNL strain (which also carries a C at position 351) was sequenced by strand-specific RNA sequencing. Reads were visualized on the genome using the Artemis software. (A) Each strain displays the expected allele at position 351 (highlighted in red) of the Pyebl gene. (B) The pyebl gene is expressed in all samples, including the transfected CU strain (CU C351Y).

FIG. 13 shows selected alleles identified by the SDR model. The identified alleles are substantially closer than those identified with the more basic SD model (t indicates that the identified selected alleles were under selection for alleles from different parents).

FIG. 14 shows Bayesian Information Criterion (BIC) values for varying models for candidate regions of the genome, within each replica, calculated under different models. BIC scores are given for the maximum likelihood candidate allele, i* found within each region, in each replica. Optimal BIC scores for each genomic region within each replica, are given in bold text. In the first part of chromosome VIII, and the second part of chromosome XIII, a candidate allele could only be identified in only one of the two replicas.

FIG. 15 shows inferred recombination rates from driver models. Recombination rates were inferred close to selected loci within each cross population. A step-wise model of recombination was applied. Recombination rates are described as number of events per base per generation.

FIG. 16 shows list of genes contained within the mathematically defined Confidence Intervals (725,528-813,866 bp) of the locus under selection on Chromosome 7. The figure shows gene ID and location for P. yoelii, protein description, number of Transmembrane domains, presence of a signal peptide, P. falciparum orthologous gene and non-synonymous to synonymous SNP ratio in P. falciparum.

FIG. 17 shows list of genes contained within the mathematically defined Confidence Intervals (1,229,582-1,363,920 bp) of the locus under selection on Chromosome 8. The figure shows gene ID and location for P. yoelii, protein description, number of Transmembrane domains, presence of a signal peptide, P. falciparum orthologous gene and non-synonymous to synonymous SNP ratio in P. falciparum.

FIG. 18 shows list of genes contained within the mathematically defined Confidence Intervals (1,436,717-1,528,275 bp) of the locus under selection on Chromosome 13. The figure shows gene ID and location for P. yoelii, protein description, number of Transmembrane domains, presence of a signal peptide, P. falciparum orthologous gene and non-synonymous to synonymous SNP ratio in P. falciparum.

FIG. 19 shows PCR primers used to generate constructs for transfection experiments.

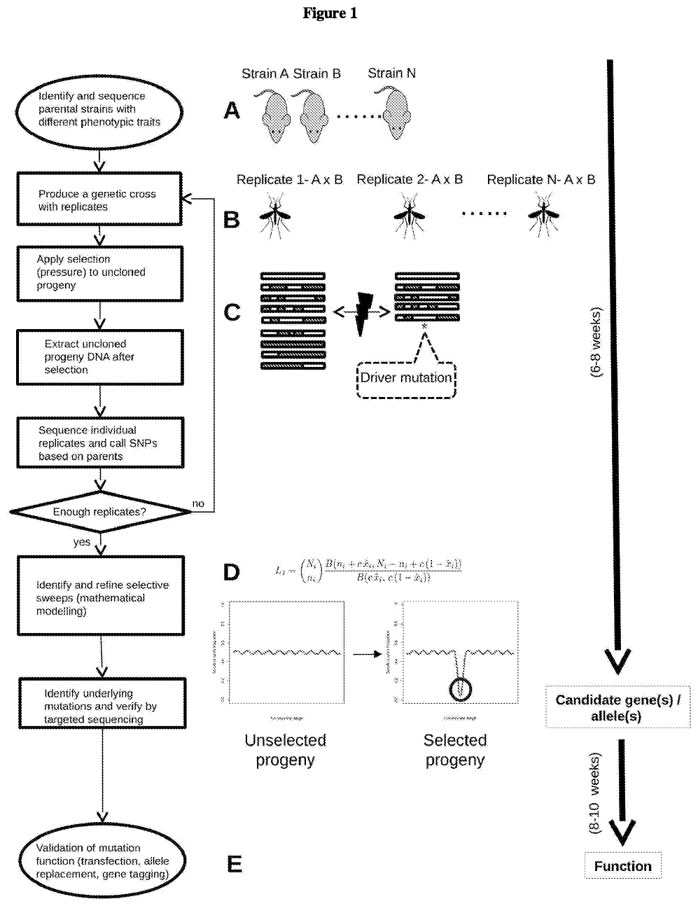

### DEFINITIONS

The terms “protein,” “polypeptide,” and “peptide,” used interchangeably herein, refer to polymeric forms of amino acids of any length, including coded and non-coded amino acids and chemically or biochemically modified or derivatized amino acids. The terms include polymers that have been modified, such as polypeptides having modified peptide backbones.

Proteins are said to have an “N-terminus” and a “C-terminus.” The term “N-terminus” relates to the start of a protein or polypeptide, terminated by an amino acid with a free amine group (—NH2). The term “C-terminus” relates to the end of an amino acid chain (protein or polypeptide), terminated by a free carboxyl group (—COOH).

The terms “nucleic acid” and “polynucleotide,” used interchangeably herein, refer to polymeric forms of nucleotides of any length, including ribonucleotides, deoxyribonucleotides, or analogs or modified versions thereof. They include single-, double-, and multi-stranded DNA or RNA, genomic DNA, cDNA, DNA-RNA hybrids, and polymers comprising purine bases, pyrimidine bases, or other natural, chemically modified, biochemically modified, non-natural, or derivatized nucleotide bases.

Nucleic acids are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. An end of an oligonucleotide is referred to as the “5′ end” if its 5′ phosphate is not linked to the 3′ oxygen of a mononucleotide pentose ring. An end of an oligonucleotide is referred to as the “3′ end” if its 3′ oxygen is not linked to a 5′ phosphate of another mononucleotide pentose ring. A nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements.

“Codon optimization” refers to a process of modifying a nucleic acid sequence for enhanced expression in particular host cells by replacing at least one codon of the native sequence with a codon that is more frequently or most frequently used in the genes of the host cell while maintaining the native amino acid sequence. For example, a polynucleotide encoding a fusion polypeptide can be modified to substitute codons having a higher frequency of usage in a given host cell as compared to the naturally occurring nucleic acid sequence. Codon usage tables are readily available, for example, at the “Codon Usage Database.” The optimal codons utilized by L. monocytogenes for each amino acid are shown US 2007/0207170, herein incorporated by reference in its entirety for all purposes. These tables can be adapted in a number of ways. See Nakamura et al. (2000) Nucleic Acids Research 28:292, herein incorporated by reference in its entirety for all purposes. Computer algorithms for codon optimization of a particular sequence for expression in a particular host are also available (see, e.g., Gene Forge).

“Sequence identity” or “identity” in the context of two polynucleotides or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically, this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

“Percentage of sequence identity” refers to the value determined by comparing two optimally aligned sequences (greatest number of perfectly matched residues) over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. Unless otherwise specified (e.g., the shorter sequence includes a linked heterologous sequence), the comparison window is the full length of the shorter of the two sequences being compared.

Unless otherwise stated, sequence identity/similarity values refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. “Equivalent program” includes any sequence comparison program that, for any two sequences in question, generates an alignment having identical nucleotide or amino acid residue matches and an identical percent sequence identity when compared to the corresponding alignment generated by GAP Version 10.

The term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine, or leucine for another non-polar residue. Likewise, examples of conservative substitutions include the substitution of one polar (hydrophilic) residue for another such as between arginine and lysine, between glutamine and asparagine, or between glycine and serine. Additionally, the substitution of a basic residue such as lysine, arginine, or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, or methionine for a polar (hydrophilic) residue such as cysteine, glutamine, glutamic acid or lysine and/or a polar residue for a non-polar residue. Typical amino acid categorizations are summarized below.

| | | | | | |
|---|---|---|---|---|---|
| Alanine | Ala | A | Nonpolar | Neutral | 1.8 |
| Arginine | Arg | R | Polar | Positive | −4.5 |
| Asparagine | Asn | N | Polar | Neutral | −3.5 |
| Aspartic acid | Asp | D | Polar | Negative | −3.5 |
| Cysteine | Cys | C | Nonpolar | Neutral | 2.5 |
| Glutamic acid | Glu | E | Polar | Negative | −3.5 |
| Glutamine | Gln | Q | Polar | Neutral | −3.5 |
| Glycine | Gly | G | Nonpolar | Neutral | −0.4 |
| Histidine | His | H | Polar | Positive | −3.2 |
| Isoleucine | Ile | I | Nonpolar | Neutral | 4.5 |
| Leucine | Leu | L | Nonpolar | Neutral | 3.8 |
| Lysine | Lys | K | Polar | Positive | −3.9 |
| Methionine | Met | M | Nonpolar | Neutral | 1.9 |
| Phenylalanine | Phe | F | Nonpolar | Neutral | 2.8 |
| Proline | Pro | P | Nonpolar | Neutral | −1.6 |
| Serine | Ser | S | Polar | Neutral | −0.8 |
| Threonine | Thr | T | Polar | Neutral | −0.7 |
| Tryptophan | Trp | W | Nonpolar | Neutral | −0.9 |
| Tyrosine | Tyr | Y | Polar | Neutral | −1.3 |
| Valine | Val | V | Nonpolar | Neutral | 4.2 |

A “homologous” sequence (e.g., nucleic acid sequence) refers to a sequence that is either identical or substantially similar to a known reference sequence, such that it is, for example, at least 50%, at least 55%, at least 60%, at least 65%, at least 70%, at least 75%, at least 80%, at least 85%, at least 90%, at least 95%, at least 96%, at least 97%, at least 98%, at least 99%, or 100% identical to the known reference sequence.

The term “fragment” when referring to a protein means a protein that is shorter or has fewer amino acids than the full length protein. The term “fragment” when referring to a nucleic acid means a nucleic acid that is shorter or has fewer nucleotides than the full length nucleic acid. A fragment can be, for example, an N-terminal fragment (i.e., removal of a portion of the C-terminal end of the protein), a C-terminal fragment (i.e., removal of a portion of the N-terminal end of the protein), or an internal fragment. A fragment can also be, for example, a functional fragment or an immunogenic fragment.

The terms “immunogenicity” or “immunogenic” refer to the innate ability of a molecule (e.g., a protein, a nucleic acid, an antigen, or an organism) to elicit an immune response in a subject when administered to the subject. Immunogenicity can be measured, for example, by a greater number of antibodies to the molecule, a greater diversity of antibodies to the molecule, a greater number of T-cells specific for the molecule, a greater cytotoxic or helper T-cell response to the molecule, and the like.

The term “antigen” is used herein to refer to a substance that, when placed in contact with a subject or organism (e.g., when present in or when detected by the subject or organism), results in a detectable immune response from the subject or organism. An antigen may be, for example, a lipid, a protein, a carbohydrate, a nucleic acid, or combinations and variations thereof. For example, an “antigenic peptide” refers to a peptide that leads to the mounting of an immune response in a subject or organism when present in or detected by the subject or organism. For example, such an “antigenic peptide” may encompass proteins that are loaded onto and presented on MHC class I and/or class II molecules on a host cell's surface and can be recognized or detected by an immune cell of the host, thereby leading to the mounting of an immune response against the protein. Such an immune response may also extend to other cells within the host, such as diseased cells (e.g., tumor or cancer cells) that express the same protein.

The term “in vitro” refers to artificial environments and to processes or reactions that occur within an artificial environment (e.g., a test tube).

The term “in vivo” refers to natural environments (e.g., a cell or organism or body) and to processes or reactions that occur within a natural environment.

Compositions or methods “comprising” or “including” one or more recited elements may include other elements not specifically recited. For example, a composition that “comprises” or “includes” a protein may contain the protein alone or in combination with other ingredients.

Designation of a range of values includes all integers within or defining the range, and all subranges defined by integers within the range.

Unless otherwise apparent from the context, the term “about” encompasses values within a standard margin of error of measurement (e.g., SEM) of a stated value or variations ±0.5%, 1%, 5%, or 10% from a specified value.

The singular forms of the articles “a,” “an,” and “the” include plural references unless the context clearly dictates otherwise. For example, the term “an antigen” or “at least one antigen” can include a plurality of antigens, including mixtures thereof.

Statistically significant means p<0.05.

### DETAILED DESCRIPTION

Various embodiments of the inventions now will be described more fully hereinafter with reference to the attached Appendices A-C, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. The term “or” is used herein in both the alternative and conjunctive sense, unless otherwise indicated. The terms “illustrative” and “exemplary” are used to be examples with no indication of quality level.

### I. Exemplary Embodiments

Details regarding various embodiments are described in connection with the attached Appendices A-C, which are herein incorporated by reference. By way of background, identifying the genetic determinants of phenotypes that impact on disease severity is of fundamental importance for the design of new interventions against malaria. Presented herein is a novel, rapid, genome-wide approach (termed quantitative-seq Linkage Group Selection, qSeq-LGS) capable of identifying multiple genetic drivers of medically relevant phenotypes within malaria parasites via a single experiment at single gene or allele resolution. In a proof of principle study disclosed herein, a previously undescribed single nucleotide polymorphism in the binding domain of the erythrocyte binding like protein (EBL) conferred a dramatic change in red blood cell invasion in mutant rodent malaria parasites Plasmodium yoelii. In the same experiment, merozoite surface protein 1 (MSP1) and other polymorphic antigen genes were implicated as major drivers of strain-specific immunity. Using allelic replacement, functional validation of the mutation in the EBL gene controlling the growth rate in the blood stages of the parasites was provided. Using this new approach which combines genetics, genomics and mathematical modelling, the inventors identified several new genes as malaria vaccine candidates. In some embodiments, the presently disclosed subject matter provides new potential vaccine candidates for human malaria parasites.

Also provided are immunogenic compositions, pharmaceutical compositions, or vaccines comprising an immunogenic polypeptide as disclosed herein, a nucleic acid encoding an immunogenic polypeptide as disclosed herein. In one embodiment, immunogenic polypeptide is encoded by a nucleic acid sequence with at least 70%, 75%, 80%, 85%, 90%, 95%, 96%, 97%, 98% or 99% sequence identity to SEQ ID NOs: 7, 8, 9, 10, 11, 12, or a fragment thereof. In one embodiment, immunogenic polypeptide is encoded by a nucleic acid sequence with at least 70%, 75%, 80%, 85%, 90%, 95%, 96%, 97%, 98% or 99% sequence identity to SEQ ID NOs: 19, 20, 21, 22, 23, 24, or a fragment thereof. In one embodiment, immunogenic polypeptide is encoded by a nucleic acid sequence with at least 70%, 75%, 80%, 85%, 90%, 95%, 96%, 97%, 98% or 99% sequence identity to SEQ ID NOs: 31, 32, 33, 34, 35, 36, or a fragment thereof. In one embodiment, immunogenic polypeptide is encoded by a nucleic acid sequence with at least 70%, 75%, 80%, 85%, 90%, 95%, 96%, 97%, 98% or 99% sequence identity to SEQ ID NOs: 43, 44, 45, 46, 47, 48, or a fragment thereof.

The term “immunogenic composition” refers to any composition containing an antigen that elicits an immune response against the antigen in a subject upon exposure to the composition. The immune response elicited by an immunogenic composition can be to a particular antigen or to a particular epitope on the antigen.

An immunogenic composition can additionally comprise an adjuvant (e.g., two or more adjuvants), a cytokine, a chemokine, or combination thereof. Optionally, an immunogenic composition can additionally comprises antigen presenting cells (APCs), which can be autologous or can be allogeneic to the subject.

The term adjuvant includes compounds or mixtures that enhance the immune response to an antigen. For example, an adjuvant can be a non-specific stimulator of an immune response or substances that allow generation of a depot in a subject which when combined with an immunogenic composition disclosed herein provides for an even more enhanced and/or prolonged immune response. An adjuvant can favor, for example, a predominantly Th1-mediated immune response, a Th1-type immune response, or a Th1-mediated immune response. Likewise, an adjuvant can favor a cell-mediated immune response over an antibody-mediated response. Alternatively, an adjuvant can favor an antibody-mediated response. Some adjuvants can enhance the immune response by slowly releasing the antigen, while other adjuvants can mediate their effects by any of the following mechanisms: increasing cellular infiltration, inflammation, and trafficking to the injection site, particularly for antigen-presenting cells (APC); promoting the activation state of APCs by upregulating costimulatory signals or major histocompatibility complex (MHC) expression; enhancing antigen presentation; or inducing cytokine release for indirect effect.

Examples of adjuvants include saponin QS21, CpG oligonucleotides, unmethylated CpG-containing oligonucleotides, MPL, TLR agonists, TLR4 agonists, TLR9 agonists, Resiquimod®, imiquimod, cytokines or nucleic acids encoding the same, chemokines or nucleic acids encoding same, IL-12 or a nucleic acid encoding the same, IL-6 or a nucleic acid encoding the same, and lipopolysaccharides. Another example of a suitable adjuvant is Montanide ISA 51. Montanide ISA 51 contains a natural metabolizable oil and a refined emulsifier. Other examples of a suitable adjuvant include granulocyte/macrophage colony-stimulating factor (GM-CSF) or a nucleic acid encoding the same and keyhole limpet hemocyanin (KLH) proteins or nucleic acids encoding the same. The GM-CSF can be, for example, a human protein grown in a yeast (S. cerevisiae) vector. GM-CSF promotes clonal expansion and differentiation of hematopoietic progenitor cells, antigen presenting cells (APCs), dendritic cells, and T cells.

Yet other examples of adjuvants include growth factors or nucleic acids encoding the same, cell populations, Freund's incomplete adjuvant, aluminum phosphate, aluminum hydroxide, BCG (bacille Calmette-Guerin), alum, interleukins or nucleic acids encoding the same, quill glycosides, monophosphoryl lipid A, liposomes, bacterial mitogens, bacterial toxins, or any other type of known adjuvant (see, e.g., Fundamental Immunology, 5th ed. (August 2003): William E. Paul (Editor); Lippincott Williams & Wilkins Publishers; Chapter 43: Vaccines, GJV Nossal, which is herein incorporated by reference in its entirety for all purposes).

An immunogenic composition can further comprise one or more immunomodulatory molecules. Examples include interferon gamma, a cytokine, a chemokine, and a T cell stimulant.

An immunogenic composition can be in the form of a vaccine or pharmaceutical composition. The terms “vaccine” and “pharmaceutical composition” are interchangeable and refer to an immunogenic composition in a pharmaceutically acceptable carrier for in vivo administration to a subject. A vaccine may be, for example, a peptide vaccine (e.g., comprising a recombinant fusion polypeptide as disclosed herein), a DNA vaccine (e.g., comprising a nucleic acid encoding a recombinant fusion polypeptide as disclosed herein), or a vaccine contained within and delivered by a cell (e.g., a attenuated bacterial cell). A vaccine may prevent a subject from contracting or developing a disease or condition and/or a vaccine may be therapeutic to a subject having a disease or condition. Methods for preparing peptide vaccines are well known and are described, for example, in EP 1408048, US 2007/0154953, and Ogasawara et al. (1992) Proc. Natl Acad Sci USA 89:8995-8999, each of which is herein incorporated by reference in its entirety for all purposes. Optionally, peptide evolution techniques can be used to create an antigen with higher immunogenicity. Techniques for peptide evolution are well known and are described, for example, in U.S. Pat. No. 6,773,900, herein incorporated by reference in its entirety for all purposes.

A “pharmaceutically acceptable carrier” refers to a vehicle for containing an immunogenic composition that can be introduced into a subject without significant adverse effects and without having deleterious effects on the immunogenic composition. That is, “pharmaceutically acceptable” refers to any formulation which is safe, and provides the appropriate delivery for the desired route of administration of an effective amount of at least one immunogenic composition for use in the methods disclosed herein. Pharmaceutically acceptable carriers or vehicles or excipients are well known. Descriptions of suitable pharmaceutically acceptable carriers, and factors involved in their selection, are found in a variety of readily available sources such as, for example, Remington's Pharmaceutical Sciences, 18th ed., 1990, herein incorporated by reference in its entirety for all purposes. Such carriers can be suitable for any route of administration (e.g., parenteral, enteral (e.g., oral), or topical application). Such pharmaceutical compositions can be buffered, for example, wherein the pH is maintained at a particular desired value, ranging from pH 4.0 to pH 9.0, in accordance with the stability of the immunogenic compositions and route of administration.

Suitable pharmaceutically acceptable carriers include, for example, sterile water, salt solutions such as saline, glucose, buffered solutions such as phosphate buffered solutions or bicarbonate buffered solutions, alcohols, gum arabic, vegetable oils, benzyl alcohols, polyethylene glycols, gelatine, carbohydrates (e.g., lactose, amylose or starch), magnesium stearate, talc, silicic acid, viscous paraffin, white paraffin, glycerol, alginates, hyaluronic acid, collagen, perfume oil, fatty acid monoglycerides and diglycerides, pentaerythritol fatty acid esters, hydroxy methylcellulose, polyvinyl pyrrolidone, and the like. Pharmaceutical compositions or vaccines may also include auxiliary agents including, for example, diluents, stabilizers (e.g., sugars and amino acids), preservatives, wetting agents, emulsifiers, pH buffering agents, viscosity enhancing additives, lubricants, salts for influencing osmotic pressure, buffers, vitamins, coloring, flavoring, aromatic substances, and the like which do not deleteriously react with the immunogenic composition.

For liquid formulations, for example, pharmaceutically acceptable carriers may be aqueous or non-aqueous solutions, suspensions, emulsions, or oils. Non-aqueous solvents include, for example, propylene glycol, polyethylene glycol, and injectable organic esters such as ethyl oleate. Aqueous carriers include, for example, water, alcoholic/aqueous solutions, emulsions or suspensions, including saline and buffered media. Examples of oils include those of petroleum, animal, vegetable, or synthetic origin, such as peanut oil, soybean oil, mineral oil, olive oil, sunflower oil, and fish-liver oil. Solid carriers/diluents include, for example, a gum, a starch (e.g., corn starch, pregeletanized starch), a sugar (e.g., lactose, mannitol, sucrose, or dextrose), a cellulosic material (e.g., microcrystalline cellulose), an acrylate (e.g., polymethylacrylate), calcium carbonate, magnesium oxide, talc, or mixtures thereof.

Optionally, sustained or directed release pharmaceutical compositions or vaccines can be formulated. This can be accomplished, for example, through use of liposomes or compositions wherein the active compound is protected with differentially degradable coatings (e.g., by microencapsulation, multiple coatings, and so forth). Such compositions may be formulated for immediate or slow release. It is also possible to freeze-dry the compositions and use the lyophilisates obtained (e.g., for the preparation of products for injection).

### II. Listing of Embodiments

The subject matter disclosed herein includes, but is not limited to, the following embodiments.

1. An immunogenic composition against Plasmodium comprising all or part of the nucleotide sequence PY17X_0721800 found in genomic location Py17X-07-v2: 799,281-800,081 (+) on chromosome 7 of Plasmodium yoelii or an ortholog thereof in Plasmodium falciparum or a polypeptide encoded by all or part of the nucleotide sequence PY17X_0721800 or an ortholog thereof in Plasmodium falciparum.

2. An immunogenic composition against Plasmodium comprising an immunogenic polypeptide, wherein the immunogenic polypeptide is encoded by a nucleic acid sequence with at least 75% sequence identity to a sequence selected from the group consisting of: SEQ ID NOs: 7, 8, 9, 10, 11, 12, or a fragment thereof, optionally wherein the immunogenic polypeptide is encoded by a nucleic acid sequence with at least 80% sequence identity to a sequence selected from the group consisting of: SEQ ID NOs: 7, 8, 9, 10, 11, 12, or a fragment thereof.

3. The immunogenic composition of embodiment 2, wherein the immunogenic polypeptide is encoded by a nucleic acid sequence with at least 90% sequence identity to a sequence selected from the group consisting of: SEQ ID NOs: 7, 8, 9, 10, 11, 12, or a fragment thereof.

4. An immunogenic composition against Plasmodium comprising all or part of the nucleotide sequence PY17X_0720100 found in genomic location Py17X-07-v2: 727,812-742,672 (+) on chromosome 7 of Plasmodium yoelii or an ortholog thereof in Plasmodium falciparum or a polypeptide encoded by all or part of the nucleotide sequence PY17X_0720100 or an ortholog thereof in Plasmodium falciparum.

5. An immunogenic composition against Plasmodium comprising an immunogenic polypeptide, wherein the immunogenic polypeptide is encoded by a nucleic acid sequence with at least 75% sequence identity to a sequence selected from the group consisting of: SEQ ID NOs: 19, 20, 21, 22, 23, 24, or a fragment thereof, optionally wherein the immunogenic polypeptide is encoded by a nucleic acid sequence with at least 80% sequence identity to a sequence selected from the group consisting of: SEQ ID NOs: 19, 20, 21, 22, 23, 24, or a fragment thereof.

6. The immunogenic composition of embodiment 5, wherein the immunogenic polypeptide is encoded by a nucleic acid sequence with at least 90% sequence identity to a sequence selected from the group consisting of: SEQ ID NOs: 19, 20, 21, 22, 23, 24, or a fragment thereof.

7. An immunogenic composition against Plasmodium comprising all or part of the nucleotide sequence PY17X_0721500 found in genomic location Py17X-07-v2: 784,994-791,991 (+) on chromosome 7 of Plasmodium yoelii or an ortholog thereof in Plasmodium falciparum or a polypeptide encoded by all or part of the nucleotide sequence PY17X_0721500 or an ortholog thereof in Plasmodium falciparum.

8. An immunogenic composition against Plasmodium comprising an immunogenic polypeptide, wherein the immunogenic polypeptide is encoded by a nucleic acid sequence with at least 75% sequence identity to a sequence selected from the group consisting of: SEQ ID Nos: 31, 32, 33, 34, 35, 36, or a fragment thereof, optionally wherein the immunogenic polypeptide is encoded by a nucleic acid sequence with at least 80% sequence identity to a sequence selected from the group consisting of: SEQ ID Nos: 31, 32, 33, 34, 35, 36, or a fragment thereof.

9. The immunogenic composition of embodiment 8, wherein the immunogenic polypeptide is encoded by a nucleic acid sequence with at least 90% sequence identity to a sequence selected from the group consisting of: SEQ ID Nos: 31, 32, 33, 34, 35, 36, or a fragment thereof.

10. The immunogenic composition of any one of embodiments 1 to 9, wherein the immunogenic composition comprises an adjuvant, optionally wherein the adjuvant comprises a granulocyte/macrophage colony-stimulating factor (GM-CSF) protein, a nucleotide molecule encoding a GM-CSF protein, saponin QS21, monophosphoryl lipid A, or an unmethylated CpG-containing oligonucleotide.

11. The immunogenic composition of any one of embodiments 1 to 10, wherein the immunogenic composition is against Plasmodium falciparum.

12. An immunogenic composition for use in a method of immunizing a subject against Plasmodium, the method comprising the step of administering to the subject an immunogenic amount of the immunogenic composition of any one of embodiments 1 to 11, optionally wherein the Plasmodium is Plasmodium falciparum.

13. An immunogenic composition for use in a method of eliciting an immune response in a subject against Plasmodium, the method comprising the step of administering to the subject an immunogenic amount of the immunogenic composition of any one of embodiments 1 to 11, optionally wherein the Plasmodium is Plasmodium falciparum.

14. A method of identifying parasite genes driving medically important selectable phenotypes, comprising performing a quantitative-seq linkage group selection (qSeq-LGS) method as described herein.

15. A kit, comprising a container, wherein the container comprises at least one dose of an immunogenic composition against Plasmodium comprising an immunogenic polypeptide encoded by a nucleic acid sequence with at least 90% sequence identity to a sequence selected from the group consisting of: SEQ ID NOs: 7, 8, 9, 10, 11, 12, 19, 20, 21, 22, 23, 24, 31, 32, 33, 34, 35, 36, or a fragment thereof.

### III. Examples

### Materials and Methods

Parasites, Mice and Mosquitoes

Plasmodium yoelii CU (with slow growth rate phenotype) and 17X1.1pp (with intermediate growth rate phenotype) strains were maintained in CBA mice (SLC Inc., Shizuoka, Japan) housed at 23° C. and fed on maintenance diet with 0.05% para-aminobenzoic acid (PABA)-supplemented water to assist with parasite growth. Anopheles stephensi mosquitoes were housed in a temperature and humidity controlled insectary at 24° C. and 70% humidity, adult flies were maintained on 10% glucose solution supplemented with 0.05% PABA.

Testing Parasite Strains for Growth Rate and SSI

Plasmodium yoelii parasite strains were typed for growth rate in groups of mice following the intravenous inoculation of 1×10⁶ iRBCs of either CU, 17X1.1pp or transfected clones per mouse and measuring parasitaemia over 8-9 days. In order to verify the existence of SSI between the CU and 17X1.1pp strains, groups of five mice were inoculated intravenously with 1×10⁶ iRBCs of either CU or 17X1.1pp parasite strains. After four days, mice were treated with mefloquine (20 mg/kg/per day, orally) for four days to remove infections. Three weeks post immunization, mice were then challenged intravenously with 1×10⁶ iRBCs of a mixed infection of 17X1.1pp and CU parasites. A group of five naïve control mice was simultaneously infected with the same material. After four days of growth 10 μl of blood were sampled from each mouse and DNA extracted.

Strain proportions were then measured by Quantitative Real Time PCR using primers designed to amplify the msp1 gene. All measurements were plotted and standard errors calculated using the Graphpad Prism software (v6.01) (http://www.graphpad.com/scientific-software/prism/). Wilcoxon rank sum tests with continuity corrections were used to measure the SSI effect, and were performed in R. Linear mixed model analyses and likelihood ratio tests to test parasite strain differences in growth rate were performed on log-transformed parasitaemia by choosing parasitaemia and strain as fixed factors and mouse nested in strain as a random factor, as described previously. Pair-wise comparisons of samples for the transfection experiments were performed using multiple 2-way ANOVA tests and corrected with a Tukey's post-test in Graphpad Prism software (v6.01).

Preparation of Genetic Cross

Plasmodium yoelii CU and 17X1.1pp parasite clones were initially grown separately in donor mice. These parasite clones were then harvested from the donors, accurately mixed to produce an inoculum in a proportion of 1:1 and inoculated intravenously at 1×10⁶ infected red blood cells (iRBCs) per mouse into a group of CBA mice. Three days after inoculation, the presence of gametocytes of both sexes was confirmed microscopically and mice were anesthetized and placed on a mosquito cage containing ˜400 female A. stephensi mosquitoes six to eight days post emergence. Mosquitoes were then allowed to feed on the mice without interruption. Seven days after the blood meal, 10 female mosquitoes from this cage were dissected to examine for the presence of oocysts in mosquito midguts. Seventeen days after the initial blood meal, the mosquitoes were dissected, and the salivary glands (containing sporozoites) were removed. The glands were placed in 0.2-0.4 mL volumes of 1:1 foetal bovine serum/Ringer's solution (2.7 mM potassium chloride, 1.8 mM calcium chloride, 154 mM sodium chloride) and gently disrupted to release sporozoites. The suspensions were injected intravenously into groups of CBA mice in 0.1 mL aliquots to obtain blood stage P. yoelii CU17X1.1pp cross progeny. Three days after inoculation with sporozoites, blood stage P. yoelii CU17X1.1pp cross progeny parasitized-RBC (pRBC) were harvested.

Two independent genetic crosses between CU and 17X1.1pp were produced. In the first cross, 150 mosquitoes were allowed to feed on mice inoculated 3 days previously with a 50:50 mixture of the two parental strains. Seven days later, a sub sample of mosquitoes (n=25) were dissected for oocyst detection. In this case, 90% of mosquitoes were infected, with an average burden of 87 oocysts per mosquito. Given that 50% of the oocysts are expected to be the products of selfing (i.e. CU male gametes fertilizing CU female gametes, and 17X1.1pp male gametes fertilizing 17x1.1pp female gametes), and that the remaining 50% of oocysts resulting from cross-strain fertilization would each produce four recombinant progeny types, we estimate that this cross resulted in the inoculation of 15,660 recombinant progeny types to recipient mice on day 21 post-mosquito feed, when 100 mosquitoes were dissected and the sporozoites removed from the salivary glands for inoculation. For the second cross, which followed the same protocol, 60% of mosquitoes were infected with an average oocyst burden of 77 oocysts per mosquito, leading to an estimated 9240 recombinants in the cross inoculation.

Selection of Uncloned Cross Progeny for Linkage Group Selection Analysis.

For immune selection, mice immunized with blood stage parasites of either P. yoelii CU or 17X1.1pp through exposure and drug cure (as above) were inoculated intravenously with 1×10⁶ parasitized-RBC (pRBC) of the uncloned cross progeny, as described above. The resulting infections were followed by microscopic examination of thin blood smears stained with Giemsa's solution.

DNA and RNA Isolation

Parental strains and growth rate- or immune-selected recombinant parasites were grown in naïve mice. Parasite-infected blood was passed through a single CF11 cellulose column to deplete host leukocytes, and the genomic DNA (gDNA) was isolated from the saponin-lysed parasite pellet using DNAzol reagent (Invitrogen, Carlsbad, Calif., USA) according to the manufacturer's instructions. For RNA isolation, a schizont-enriched fraction was collected on a 50% Nycodenz solution (Sigma Aldrich) and total RNA was then isolated using TRIzol (Invitrogen).

Whole Genome Re-Sequencing and Mapping

Plasmodium yoelii genomic DNA was sequenced using paired end Illumina reads (100 bp), which are available at the European Nucleotide Archive (ENA: PRJEB15102).

The paired-end Illumina data were first quality-trimmed using Trimmomatic. Illumina sequencing adaptors were then removed from the sequences. Following that, trailing bases from both the 5′ and 3′ ends with less than Q20 were trimmed. Lastly, reads with an average base quality of less than Q20 within a window size of four bases were discarded. Only read pairs where both reads were retained after trimming were used for mapping with BWA version 0.6.1 using standard options onto the publicly available genome of P. yoelii 17X strain (May 2013 release; ftp://ftp.sanger.ac.uk/pub/pathogens/Plasmodium/yoelii17X/version_2/May_2013/). The SAM alignment files were converted to BAM using Samtools. Duplicated reads were marked and removed using Picard (http://picard.sourceforge.net).

SNP Calling

The Python script used to determine SNP functions as a wrapper for SAMtools mpileup and SNP calls based on mapping quality and Phred base quality scores. In this experiment the values were set at 30 for mapping quality and 20 for base quality. Also, since the P. yoelii genome is haploid and the parental strains are clonal, only SNPs where the proportion of the major non-reference allele was more than 80% were retained, to exclude possible sequencing errors or genuine but uninformative SNPs. The script produces a tab-delimited, human readable table that shows the total number of reads for each of the four possible nucleotides at each SNP. SNPS were called on both parental strains. CU SNPs were then filtered against the 17X1.1pp SNPs to remove any shared SNP calls. The remaining CU SNPs were then used as reference positions to measure the number of reads for each nucleotide in the genetic crosses produced in this study through another Python script. This script produced a final table consisting of read counts for each nucleotide of the original CU SNPs in every sample.

Mathematical Methods for the Identification of Loci Under Growth Rate and Immune Selection

SNP frequencies were processed to filter potential misalignment events. We note that, during the cross, a set of individual recombinant genomes are generated. Considering the individual genome g, we define the function a_{g}(i) as being equal to 1 if the genome has the CU allele at locus i, and equal to 0 if the genome has the 17X1.1pp allele at this locus. In any subsequent population of N individuals, the allele frequency q(i) at locus i can then be expressed as

$$q ( i ) = \frac{1}{N} {\sum }_{g} {n}_{g} {a}_{g} ( i )$$

To filter the allele frequencies, we note that each function a_{g}(i) changes only at recombination points in the genome g. As such, q(i) should change relatively smoothly with respect to i. Using an adapted version of code developed for the inference of subclones in populations, we therefore modeled the reported frequencies q(i) as being (beta-binomially distributed) emissions from an underlying diffusion process (denoted by x(i)) along each chromosome, plus uniformly distributed errors, using a hidden Markov model to infer the variance of the diffusion process, the emission parameters, and an error rate. A likelihood ratio test was then applied to identify reported frequencies that were inconsistent with having been emitted from the inferred frequency x(i) at locus i relative to having been emitted from an inferred global frequency distribution fitted using the Mathematica package via Gaussian kernel estimation to the complete set of values x(i); this test filters out reported frequencies potentially arising from elsewhere in the genome.

Next, the above logic was extended to filter out clonal growth. In the event that a specific genome g is highly beneficial, this genome may grow rapidly in the population, such that n_{g} becomes large. Under such circumstances the allele frequency q(i) gains a step-like quality, mirroring the pattern of a_{g}(i). Such steps may potentially mimic selection valleys, confounding any analysis. As such, a jump-diffusion variant of the above hidden Markov model was applied, in which the allele frequency can change either through a diffusion process or via sudden jumps in allele frequency, modeled as random emissions from a uniform distribution on the interval [0,1]. For each interval (i,I+1) the probability that a jump in allele frequency had occurred was estimated. Where potential jumps were identified, the allele frequency data were split, such that analyses of the allele frequencies did not span sets of alleles containing such jumps. The resulting segments of genome were then analyzed under the assumption that they were free of allele frequency change due to clonal behavior.

Inference of the presence of selected alleles was performed using a series of methods. In the absence of selection in a chromosome, the allele frequency is likely to remain relatively constant across each chromosome. A ‘non-neutrality’ likelihood ratio test was applied to each contiguous section of genome, calculating the likelihood difference between a model of constant frequency x(i) and the variable frequency function x(i) inferred using the jump-diffusion model. Next, an inference was made of the position of the allele potentially under selection in each region. Under the assumptions that selection acts for an allele at locus i, and that the rate of recombination is constant within a region of the genome, previous work on the evolution of cross populations can be extended to show that the allele frequencies within that region of the genome at the time of sequencing are given by

$$x ( i ) = x + {Δ}_{x}$$

$$x ( j ) = [ X + \frac{1}{2} ( 1 - X ) \text{(} 1 + {e}^{- ρ {Δ}_{ij}} ] x + [ \frac{1}{2} X ( 1 - {e}^{- ρ {Δ}_{ij}} ) ] ( 1 - x ) + {Δ}_{x}$$

for each locus j not equal to i, where X is the CU allele frequency at the time of the cross, ρ is the local recombination rate, Δᵢⱼ is the distance between the loci i and j, x is an allele frequency, and Δₓ describes the effect of selection acting upon alleles in other regions of the genome. A likelihood-based inference was used to identify the locus at which selection was most likely to act. In regions for which the ‘non-neutrality’ test produced a positive result for data from both replica crosses, and for which both the inferred locus under selection, and the direction of selection acting at that locus were consistent between replicas, an inference of selection was made.

For regions in which an inference of selection was made, an extended version of the above model was applied, in which the assumption of locally constant recombination rate was relaxed. Successive models, including an increasing number of step-wise changes in the recombination rate, were applied, using the Bayesian Information Criterion for model selection. A model of selection at two loci within a region of the genome was also examined. Given an inference of selection, a likelihood-based model was used to derive confidence intervals for the position of the locus under selection.

Filtering of Allele Frequency Data: Diffusion Model

Allele frequency data were filtered using a likelihood ratio in an effort to remove sites where alleles had been mapped to the wrong genomic location. Given the structure of the genetic cross, the allele frequency is expected to change incrementally with small changes in genetic location. We therefore generated a smoothed representation of the underlying allele frequencies. For each genetic locus i, with read depth Nᵢ, we denote the read count of CU alleles by nᵢ, and the true underlying CU allele frequency by xᵢ. We then suppose that, with some probability 1−r, nᵢ was drawn from a beta-binomial distribution Beta(Nᵢ, α, β), where α=cxᵢ, and β=c(1−x_i), for some unknown parameter c, while with probability r, nᵢ resulted from a mapping error, being drawn from the uniform distribution U(0, Nᵢ). We further supposed that changes in the true allele frequencies between nearby loci are small, being represented by a diffusion process:

xᵢ₊₁=xᵢ+N(0,s\sqrt{Δ_{i,i+1}}),

in which the difference between subsequent allele frequencies is normally distributed with zero mean and standard deviation proportional to s times the square root of the distance between the segregating sites (reflecting boundaries were used to keep xᵢ. within the interval [0,1]). Given this model, a forward-backward algorithm was used to identify maximum likelihood values for r, c, and s. Our algorithm gave a posterior distribution for each of the \hat{x}ₗ; calculated the mean of this distribution to obtain approximations for each locus.

A likelihood ratio test was then applied to exclude frequencies of alleles that were likely to have been mapped to the wrong location in the genome. Expressed in terms of the above parameters, the likelihood L₁ that an allele frequency belonged to the genomic region with which it had been associated was estimated as

$${L}_{1} = ( \begin{matrix}{N}_{i} \\ {n}_{i}\end{matrix} ) \frac{B ( {n}_{i} + c {\hat{x}}_{i} , {N}_{i} - {n}_{i} + c ( 1 - {\hat{x}}_{i} ) )}{B ( c {\hat{x}}_{i} , c ( 1 - {\hat{x}}_{i} ) )}$$

where B(a,b) is the beta function. In contrast to this, a mismapped read could arise from anywhere in the genome. Using the Mathematica software package, a smooth kernel distribution was fitted to the set {\hat{x}ᵢ}, of all observed frequencies genome-wide, obtaining the probability density function P for this distribution. The likelihood L₂ was then calculated as

L₂= [symbol] (\hat{x}ᵢ)

Data from loci for which the log ratio log(L₁/L₂)<−10 in at least one of the replicates were excluded from further analysis in all datasets.

Particular care was taken with alleles mapped to regions at the ends of chromosomes. Firstly, small sets of isolated allele frequencies, occurring at the ends of chromosomes, were excluded from the analysis. Loci within each chromosome were partitioned into subsets, separated by gaps of at least 20 kb in which no SNPs were observed. Subsets of fewer than 10 isolated loci at the ends of chromosomes were removed from the data.

Jump Diffusion Analysis

From visual inspection of the data, occasional apparent discontinuities were seen, at which the observed allele frequency changed substantially between adjacent SNPs. These jumps could occur either from the growth of a clone, or clones, with near-identical genomes, in the experimental population, or alternatively through some gross misalignment of data, whereby regions some distance apart in the genome were placed together.

The location of significant jumps in the allele frequency was inferred by modeling the observed data as being generated by a jump-diffusion process, fitting a set of frequencies xᵢ to the observations which change either smoothly, according to a diffusion model as described above, or through sudden changes to different, arbitrary frequencies. Specifically, xᵢ was modeled as changing via the equations

xᵢ₊₁=xᵢ+N(0,s\sqrt{Δ_{i,i+1}})

with probability k

(1−p)^{Δ}^{_{i,i+1}} and xᵢ₊₁˜ [symbol] (0,1)

with probability

1−(1−p)^{Δ}^{_{i,i+1}},

where the value p represents the probability per base of a jump in allele frequency. Parameters were inferred as above, with the addition of the value p. The beta-binomial coefficient c was fixed as the value inferred for each dataset from the previous calculation. Due to the earlier filtering steps, applied above, the inferred error rate r was less than 10⁻¹⁰ for each set of allele frequencies, so was removed from the model. For each locus i the posterior probability pᵢ that a jump occurred at i was calculated.

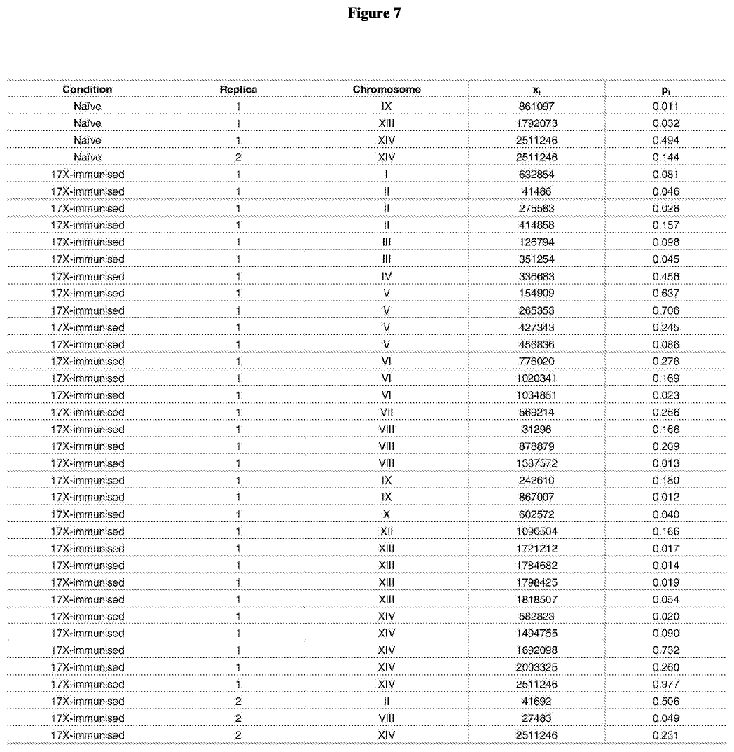

Loci with posterior jump probabilities greater than 1% are listed in FIG. 7. Three of these loci, towards the ends of chromosomes, were conserved between replicates, being seen in both of the 17X-immunised datasets, a jump in chromosome XIV being observed in both naïve replicates as well. Such consistency in the location of jumps between replica experiments is highly improbable if they occur independently; we supposed these jumps to result from misalignment errors, or errors in the genome reference sequence. Alleles further towards the end of each chromosome than these jumps were removed from consideration in all datasets.

Other loci at which jumps were inferred were only seen in the first replicate experiment, primarily in the 17X-immunised data, but also in the naïve dataset. This result is consistent with the existence of clonal growth in the first replica experiment, some of it occurring before the separation of parasite populations into naïve and selected groups. The reduced number of jumps in the naïve and CU-immunised cases may be explained by a difficulty in inference; due to pervading selection for 17X alleles, the mean allele frequency in these two populations is generally close to 0, reducing the magnitude of observed jumps in frequency.

In order to fit models of continuous allele frequency change to the observed frequency data, chromosomes were subdivided into smaller regions at the location of potential jumps, such that the frequencies within each region under analysis changed in a continuous manner.

Likelihood Models

Regions of the genome containing alleles under selection were identified using a likelihood-based modeling framework. Given a model M describing allele frequencies after selection, the model parameters were optimised to identify the maximum likelihood fit between the model, and the observed frequencies in a genomic region, using the noise model learnt in the diffusion model above:

$$log {L}^{M} = {\sum }_{i} log ( \begin{matrix}{N}_{i} \\ {n}_{i}\end{matrix} ) \frac{B ( {n}_{i} + c {x}_{i} , {N}_{i} - {n}_{i} + c ( 1 - {x}_{i} ) )}{B ( c {x}_{i} , c ( 1 - {x}_{i} ) )}$$

In order to distinguish between likelihoods generated from models with differing numbers of parameters, the Bayesian Information Criterion (BIC) was used. For a given model fit to the data, the BIC value is given by

BIC=−2L+k log(n)

where k is the number of model parameters, and n is the number of loci to which the model was fitted. In any comparison between models, the model giving the lowest BIC value was selected.

A variety of models were applied, modeling changes in the allele frequency over time between the beginning of the experiment and the time of sequencing. A neutral model assumed that no alleles were under selection. A single driver model (SD) assumed that a single allele, or “driver” within the region was under selection. These standard models assumed a locally-constant recombination rate; extensions of the single-driver model allowed for one (SDR), two (SD2R), or three (SD3R) changes in recombination rate within the local region. Further comparison was made to the jump-diffusion (J-D) model described above, in which a smooth line was fitted directly to the allele frequencies; the jump-diffusion model is by its definition a very good fit to the data.

Identification of Non-Neutral Regions of the Genome

Non-neutral regions of the genome were identified according to two characteristics. Firstly, we note that, if no alleles in a given region of the genome are under selection, the allele frequencies in this region may still change during the experiment, due to selection acting upon pure genotypes during the cross, but will do so in a uniform way, plus noise. However, if a single allele is under selection, this will result in local variation in the observed allele frequencies, according to the pattern of a selective sweep. As such, regions of the genome were tested for deviation from neutrality; comparing the log likelihoods generated by the neutral and J-D models. The “non-neutrality score” S for a region of the genome g taken from replica r, was defined as

$${S}_{r , g} = \frac{{L}_{r , g}^{J D} - {L}_{r , g}^{neutral}}{{n}_{g}}$$

where division of the likelihood difference by n_{g}, the number of loci in the region g, normalises the score per locus.

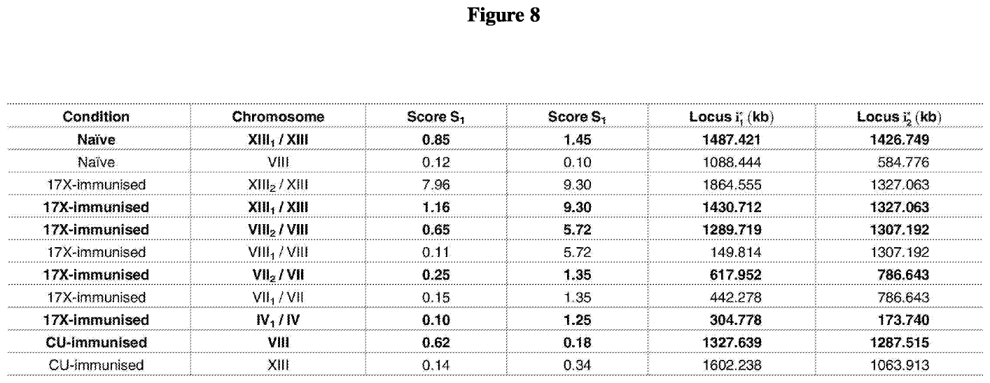

In order to identify candidate alleles under selection, the sum of the non-neutrality scores from both replicas, S_{1,g}+S_{2,g}, was calculated for each region of the genome, ranking the results by this score, and retaining regions for which both S_{1,g} and S_{2,g} were greater than 0.1 (FIG. 8). Next, the SD model was fitted to the allele frequency data, identifying a putative locus under selection. Regions for which the driver alleles identified within both replicas were within 200 kb, and for which the direction of selection was consistent between the two replicas, were retained for further investigation. On this basis, six regions of the genome were retained.

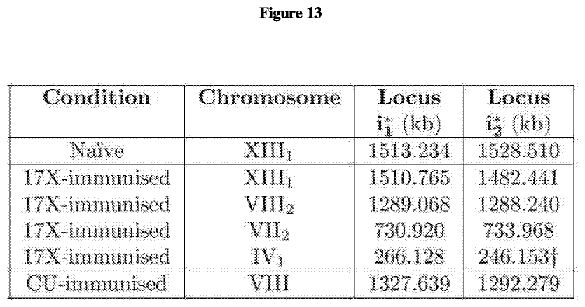

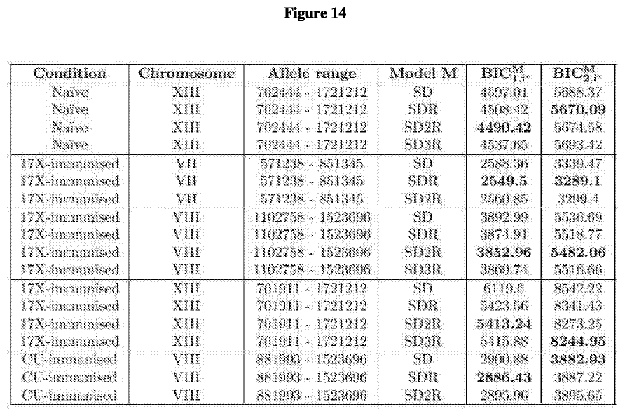

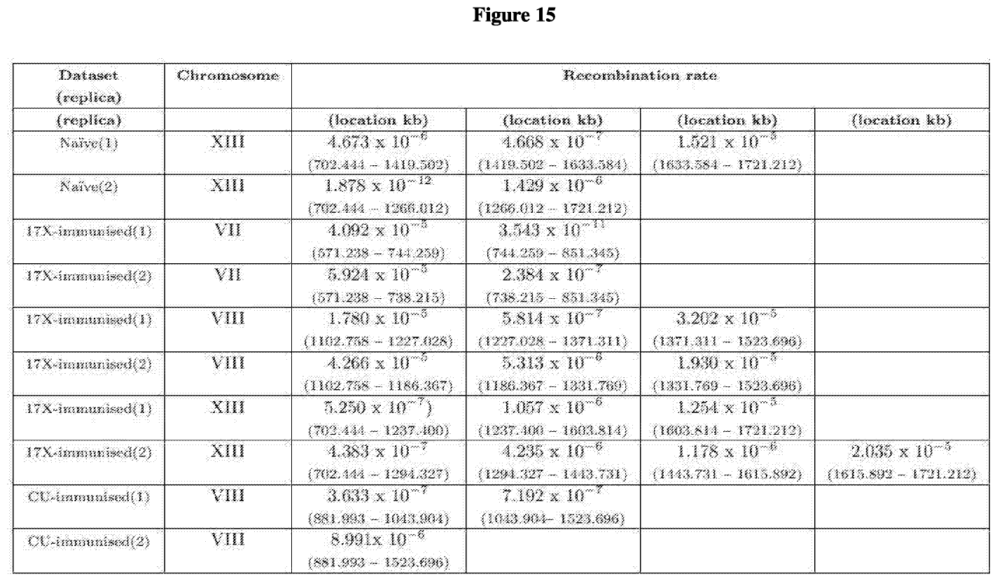

Retained regions were analysed using successively more complex models of recombination, allowing for increasing numbers of changes in the recombination rate, and performing model selection using BIC. Under this approach, the distance between candidate alleles in the two replicas narrowed, from a mean of 87 kb to just over 17 kb (FIG. 13). The candidate region in chromosome IV, however, was identified as a false positive of the previous method, the SDR model suggesting selection for alleles from different parents in the two replica datasets; this region was excluded from further analysis. Increasingly complex models of recombination change were fitted to the data using BIC for model selection. Calculated BIC values are shown in FIG. 14, with local inferences of recombination rate given in FIG. 15.

Confidence Intervals for Allele Locations

Confidence intervals for the location of each inferred selected were found by calculating likelihoods for models in which the location of the selected allele was fixed. Regions of the genome for which the calculated model likelihood was consistently within 3 log likelihood units of the maximum log likelihood were derived, corresponding roughly to a 99% confidence interval.

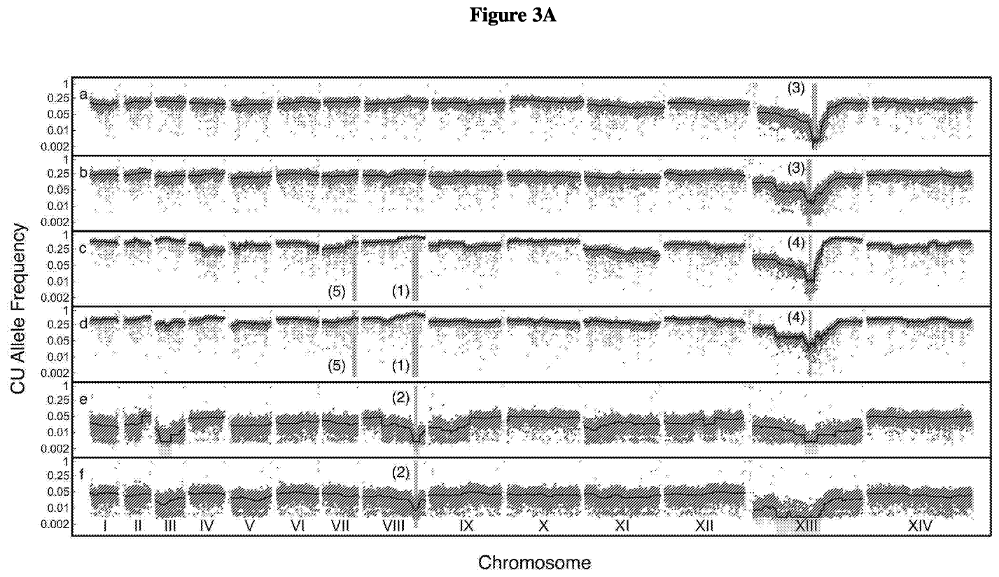

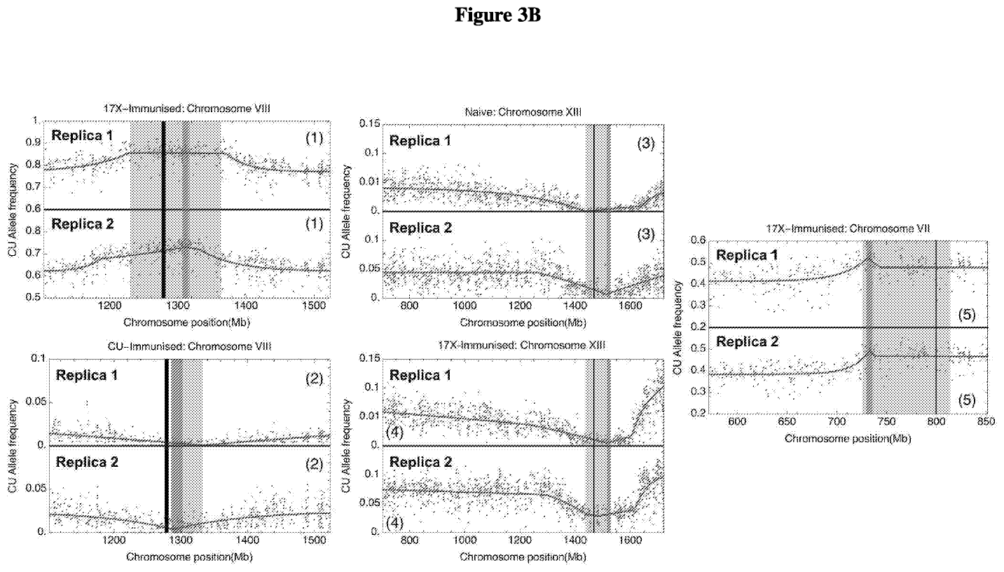

A first confidence interval was generated in this manner by forcing the location of the selected allele to be consistent between the two replicates, and calculating the sum of the model log likelihoods for the two replicates. Allowing for the potential effects of biological noise in the data, a second, more conservative interval was also generated, representing the span of alleles for which the likelihood calculated in either replicate was within 3 log likelihood units of the maximum; this second interval becomes large when data in either one of the two experiments is ambiguous about the allele location. Confidence intervals are illustrated in FIG. 3 of the main text.

Mathematical Models of Allele Frequency Change

For convenience, we denote the 17X allele at any locus as 1, and the CU allele as 0. Thus, at a given locus i we denote the frequency of the 17X allele, as xᵢ¹, and the frequency of the CU allele as xᵢ⁰. Given a set of two loci, i and j, we denote the frequency of individuals with allele a at locus i and allele b at locus j as xᵢⱼ^{ab}, where a and b are either 0 or 1.

We assume that, before the cross occurs, changes in the frequency of the CU and 17X malaria types may occur due to selection upon one type or another. At the time of the cross, we assume that the frequency of 17X types is equal to some value, X, where 0≤X≤1. Following the cross, the population comprises a fraction X² of pure 17X individuals, (1−X)² pure CU individuals, and 2X(1−X) individuals which have undergone crossing. Subsequent selection can change both the fraction of pure types in the population, and the composition of the crossed individuals.

Neutral Model

The neutral model assumes that a given region of the genome does not contain an allele under selection. Under this model, over the course of time, allele frequencies in the region can change, but only due to selection upon pure types acting at alleles elsewhere in the genome. In consequence, the allele frequencies are expected to remain uniform across the region. We describe the allele frequencies as

xᵢ¹=x∀i,

learning the value of the frequency parameter x.

Single Driver Model

Given a region of the genome, we suppose that the allele 1 at locus i is under selection, with strength σ (which may be positive or negative).

We denote the time of the cross as t_{c}. Following the cross, the selected allele is modeled as changing frequency deterministically according to the equation

$${x}_{i}^{1} ( t ) = \frac{{Xe}^{σ ( t - {t}_{c} )}}{1 - X + {Xe}^{σ ( t - {t}_{c} )}}$$

We denote the frequency of this allele at the time of observation as xᵢ¹(tₒ).

Between t_{c} and tₒ, the frequency of an allele j≠i, while not itself under selection, will change via linkage disequilibrium with the allele at i, as described by the equation

$${x}_{j}^{1} ( t ) = {x}_{i}^{1} ( t ) \frac{{x}_{ij}^{11} ( {t}_{c} )}{{x}_{i}^{1} ( {t}_{c} )} + {x}_{i}^{0} ( t ) \frac{{x}_{ij}^{01} ( {t}_{c} )}{{x}_{i}^{0} ( {t}_{c} )}$$

To calculate the haplotype frequencies, xᵢⱼ¹¹(t_{c}) and xᵢⱼ⁰¹(t_{c}), we consider separately the pure and crossed genotypes. The pure genotypes contribute a frequency X² towards the frequency xᵢⱼ¹¹(t_{c}), but make no contribution to the frequency xᵢⱼ⁰¹(t_{c}). Considering allele frequencies among the crossed fraction of the population, we denote by \hat{x}ᵢ¹ the frequency of the allele 1 at the locus i within the crossed individuals alone. Following the cross, we have that

\tilde{x}ᵢⱼ¹¹(t_{c})=\tilde{x}ᵢ¹(t_{c})\tilde{x}ⱼ¹(t_{c})+D′ᵢⱼe^{-ρΔij},

where ρ is the rate of recombination per site per generation, Δᵢⱼ is the sequence length between the loci i and j, and D′ᵢⱼ is the linkage disequilibrium between alleles at i and j before the cross. Assuming that no selection took place during the crossing procedure, we have

\tilde{x}ᵢ¹(t_{c})=\tilde{x}ⱼ¹(t_{c})=0.5,

Furthermore, the mating process involves equal numbers of pure types, so that D′ᵢⱼ=0.25. We thus have the result

\tilde{x}ᵢⱼ¹¹(t_{c})=¼(1+e^{-ρΔij}),

and, combining the cross and pure types,

xᵢⱼ¹¹(t_{c})=X²+½X(1−X)(1+e^{-ρΔij}).

In a similar manner, we obtain the result

\tilde{x}ᵢⱼ⁰¹(t_{c})=\tilde{x}ᵢ⁰(t₁)\tilde{x}ⱼ¹(t_{c})−D′ᵢⱼe^{-ρΔij}=¼(1−e^{-ρΔij})

so that

xᵢⱼ⁰¹(t_{c})=½X(1−X)(1−e^{-ρΔij}).

Combining these terms, and remembering that xᵢ¹(t_{c})=X, while xᵢ⁰(t_{c})=1−X, we derive the equation

xⱼ¹(t)=[X+½(1−X)(1+e^{-ρΔij})]xᵢ¹(t)+[½X(1−e^{-ρΔij})]xᵢ⁰(t)

We add to this one other term, e, denoting the effect of selection acting upon loci in other chromosomes upon the frequencies of the pure genotypes, obtaining the final model

xᵢ¹(tₒ)=x+e

xⱼ¹(tₒ)=[X+½(1−X)(1+e^{-ρΔij})]x+[½X(1−e^{-ρΔij})](1−x)+e

where x is equivalent to xᵢ¹(tₒ) in the model above. To specify the model, it is sufficient to learn the parameters i, X, ρ and e, where i denotes a locus in the given genomic region, 0≤X≤1, −X²≤e≤(1−X)², X²≤×<(1−X)², and 0≤x+e≤1.

Single-Driver with Variable Recombination Rate

The models above assume that the rate of recombination during the cross is constant within each chromosome. However, where the rate of recombination is variable, such an assumption can lead to incorrect placement of the locus under selection. We therefore developed a hierarchy of SD models, allowing for variable recombination rate. In the k^{th} such model, we learnt k recombination rates ρ₁, . . . , ρₖ, and k−1 loci, i_{ρ1}, . . . , i_{ρk-1}, such that, where i_{ρ0} and i_{ρk} are defined as the first and last loci in the genomic region, the recombination rate between locus i_{ρj} and i_{ρj+1} was equal to i_{ρ1}. Mathematically, such a model is identical to the SD model described above, except that the term ρΔᵢⱼ, describing the breakage in linkage disequilibrium between loci i and j, is replaced by the sum

$${\sum }_{k = 1}^{K} {ρ}_{{n}_{k}}$$

where ρₙₖ is the recombination rate between the alleles nₖ and nₖ₊₁, n₁=i and n_{K}=j. We denote the SD model with one change of recombination rate as the SDR model, the SD model with two changes of recombination rate as the SD2R model, and so forth.

Information on Genes in Identified Loci Under Selection

For each combined conservative interval of relevant loci under selection, genes were listed based on the annotation available in version 6.2 of PlasmoDB and verified against the current annotation (release 26). For each gene, information on predicted transmembrane domains, signal peptides and P. falciparum orthologues. For the P. falciparum orthologues, the NS/S SNP ratios were obtained from PlasmoDB, based on the count of synonymous and non-synonymous SNPs found in 202 individual strains collected from 6 data sets stored on the website. More details on the data sets can be found at the following link: https://goo.gl/lUwKn1.

Plasmid Construction to Modify P. yoelii Ebl Gene Locus

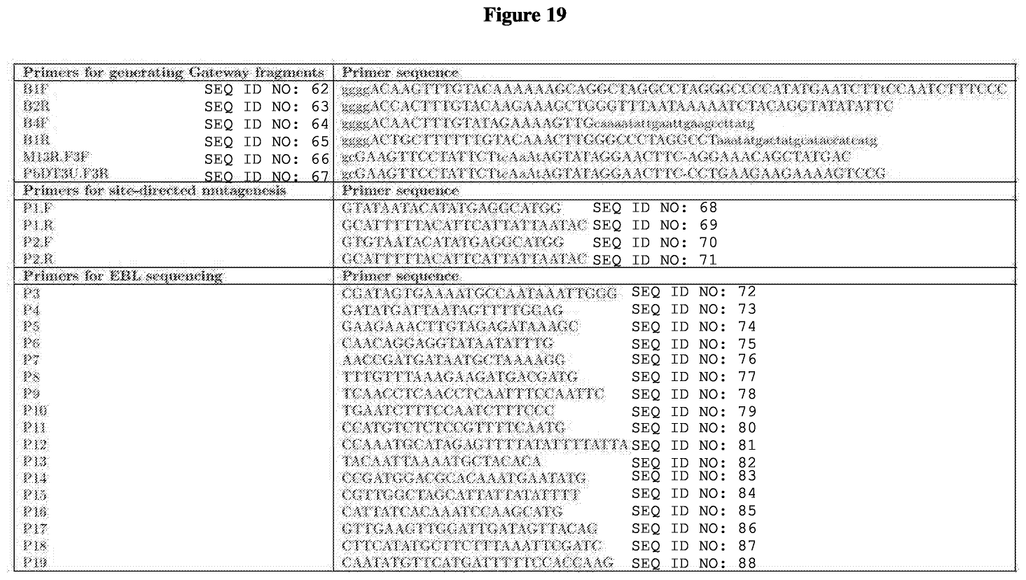

All primer sequences are given in FIG. 19. Plasmids were constructed using MultiSite Gateway cloning system (Invitrogen).

PCR Amplification and Sequencing of the Pyebl Gene

The Pyebl gene was PCR-amplified from gDNA using KOD Plus Neo DNA polymerase (Toyobo, Japan) with specific primers designed based on the ebl sequence in PlasmoDB (PY17X_1337400). Pyebl sequences of CU and 17X1.1pp strains were determined by direct sequencing using an ABI PRISM 310 genetic analyzer (Applied Biosystems) from PCR-amplified products. Sequences were aligned using online sequence alignment software Clustal Omega (https://www.ebi.ac.uk/Tools/msa/clustalo/) provided by EMBL-EBI.

Plasmid Construction to Modify the Pyebl Locus

attB-flanked ebl gene products, attB12-PyCU-EBL.ORF and attB12-Py17X1.1pp-EBLORF, were generated by PCR-amplifying both P. yoelii CU and P. yoelii 17X1.1pp ebl gene with yEBL-ORF.B1F and yEBL-ORF.B2R primers. attB-flanked ebl-3U (attB41-PyCU-EBL-3U and attB41-Py17X1.1pp-EBL-3U) was similarly generated by PCR-amplifying P. yoelii gDNA with yEBL-3U.B4F and yEBL-3U.B1R primers. attB12-PyCU-EBL.ORF and attB12-Py17X1.1pp-EBLORF were then subjected to a separate BP recombination with pDONR 221 (Invitrogen) to yield entry plasmids, pENT12-PyCU-EBL.ORF and pENT12-Py17X1.1pp-EBLORF, respectively. attB41-PyCU-EBL-3U and attB41-Py17X1.1pp-EBL-3U fragments were also subjected to independent BP recombination with pDONR P4-P1R (Invitrogen) to generate pENT41-PyCU-EBL-3U and pENT41-Py17X1.1pp-EBL-3U, respectively.

All BP reactions were performed using the BP Clonase II enzyme mix (Invitrogen) according to the manufacturer's instructions. To change P. yoelii CU ebl gene nucleotide 1052G to 1052A (351Cys to 351Tyr), pENT12-PyCU-EBL.ORF entry clone was modified using KOD-Plus-Mutagenesis Kit (TOYOBO) with primers P1.F and P1.R to yield pENT12-PyCU-EBL.ORF-C351Y. pENT12-Py17X1.1pp-EBL.ORF was also modified from 1052A to 1052G (351Tyr to 351Cys) using primers P2.F and P1.R to yield pENT12-Py17X1.1pp-EBLORF-Y351C. pHDEF1-mh that contains a pyrimethamine resistant gene selection cassette (a gift from Hernando del Portillo) was digested with SmaI and ApaI to remove PfHRP2 3′ UTR DNA fragment, cohesive end was blunted, and a DNA fragment containing ccdB-R43 cassette and P. berghei DHFR-TS 3′ UTR that was amplified from pCHD43(II) with primers M13R.F3F and PbDT3U.F3R was ligated to generate pDST43-HDEF-F3. pENT12-PyCU-EBL.ORF-C351Y and pENT12-Py17X1.1pp-EBLORF-Y351C entry plasmids were each separately subjected to LR recombination reaction (Invitrogen) with a destination vector pDST43-HDEF-F3, pENT41-PyCU-EBL-3U or pENT41-Py17X1.1pp-EBL-3U and a linker pENT23-3Ty1 vector to yield replacement constructs pREP-PyCU-EBL-C351Y and pREP-Py17X1.1pp-EBL-Y351C, respectively. Control constructs pREP-PyCU-EBL-C351C and pREP-Py17X1.1pp-EBL-Y351Y were also prepared in a similar manner. These LR reactions were performed using the LR Clonase II Plus enzyme mix (Invitrogen) according to the manufacturer's instructions.

Phenotype Analysis

To assess the course of infection of wild type and transgenic parasite lines, 1×10⁶ pRBCs were injected intravenously into five 8-week old female CBA mice for each parasite line. Since the 17X1.1p and CU-recipient strains were transfected on separate occasions, the transgenic lines were tested separately. Thin blood smears were made daily, stained with Giemsa's solution, and parasitaemias were examined microscopically.

RNA-seq

Whole blood from mice infected with P. yoelii on day 5 post-infection were host WBC depleted and saponin lysed to obtain the parasite pellet. Total RNA was extracted using TRIzol reagent. Strand-specific RNA sequencing was performed from total RNA using TruSeq Stranded mRNA Sample Prep Kit LT according to manufacturer's instructions. Libraries were sequenced on an Illumina HiSeq 2000 with paired-end 100 bp read chemistry and are publicly available at the European Nucleotide Archive (ENA: PRJEB15102). RNA-seq reads were mapped onto P. yoelii 17X version 2 from GeneDB (http://www.genedb.org) using TopHat 2.0.13 and visualized using Artemis genome visualization tool.

Indirect Immunofluorescence Assay

Schizont-rich whole blood was obtained from P. yoelii infected mouse tail and prepared air-dried thin smears on glass slides. The smears were fixed in 4% paraformaldehyde containing 0.0075% glutaraldehyde (Nacalai Tesque) in PBS at room temperature (RT) for 15 min, rinsed with 50 mM glycine (Wako) in PBS. Samples were permeabilized with 0.1% Triton X-100 (Calbiochem) in PBS for 10 min, then blocked with 3% BSA (Sigma) in PBS at RT for 30 min. Next, samples were immunostained with primary antibodies using mouse anti-PyEBL (final 1:500) and Rabbit anti-PyAMA1 (a gift from Takafumi Tsuboi, final concentration 1:500) at 37° C. for 1 h. This was followed by 3 washes with PBS then incubation with Alexa Fluor 488 goat anti-mouse and Alexa Fluor 594 goat anti-rabbit antibodies (Invitrogen; final 1:1000) in 3% BSA in PBS at 37° C. for 30 min. Parasite nuclei were stained with 4′, 6-diamidino-2-phenylindole (DAPI; Invitrogen, final 0.2 μg/mL). Stained parasites were mounted with Prolong Gold antifade reagent (Invitrogen). Slides were visualized using a fluorescence microscope (Axio imager Z2; Carl Zeiss) with 100× oil objective lens (NA 1.4, Carl Zeiss). Images were captured using a CCD camera (AxioCam MRm; Carl Zeiss) and imaged using AxioVision software (Carl Zeiss). Mann-Whitney U tests were performed using Graphpad Prism software (v6.01).

Structural Modeling of PyEBL Protein in Wild-Type and Mutant Parasites

Since the atomic structures of EBL protein of P. yoelii Wild Type: (Py17X-WT) and its mutant P. yoelii (C351Y): (Py17X1.1pp) are not known, homology models were generated. The homology models were generated using P. vivax Duffy Binding Protein (PvDBP) atomic structure (PDB ID: 3RRC), with the Swiss-Model server (https://swissmodel.expasy.org). The homology models showed maximum amino acid sequence homology of 32% with Py17X-WT EBL, compared to another homologous protein P. falciparum Erythrocyte Binding Antigen 140 (PfEBA-140/BAEBL) (PDB ID: 4GF2) that had 26% sequence homology. These models were then subsequently stabilized by minimizing their energies for at least 10 times each, to attain reasonably well equilibrated structures using the YASARA server (www.yasara.org).

The prediction of disulfide bonds in our homology models were performed using DISULFIND (http://disulfind.dsi.unifi.it). Our analysis showed high probability of disulfide bond formation by this Cys351 residue. Confirming that C351 is a potential residue for forming a disulfide bond, the energy minimized stable homology models were subjected to Disulfide bond visualization to check whether the Cys351 is involved in any disulfide bond formation with any other Cys and what is the effect of the C351Y substitution.

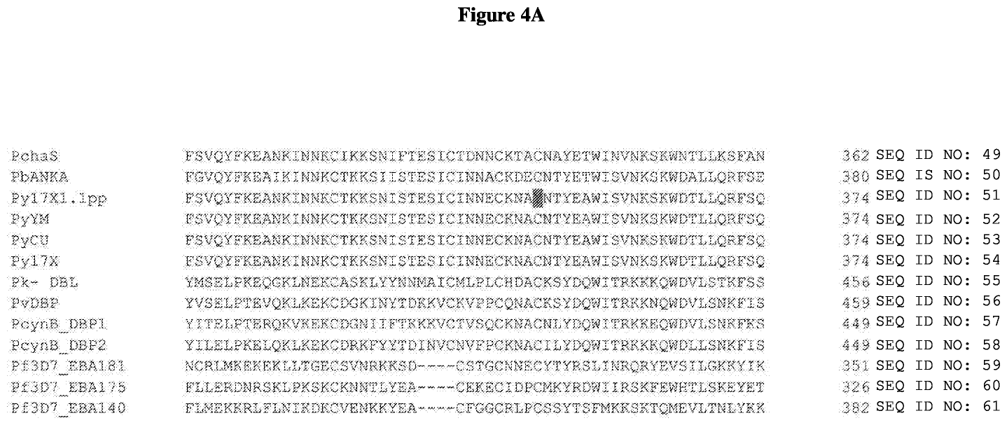

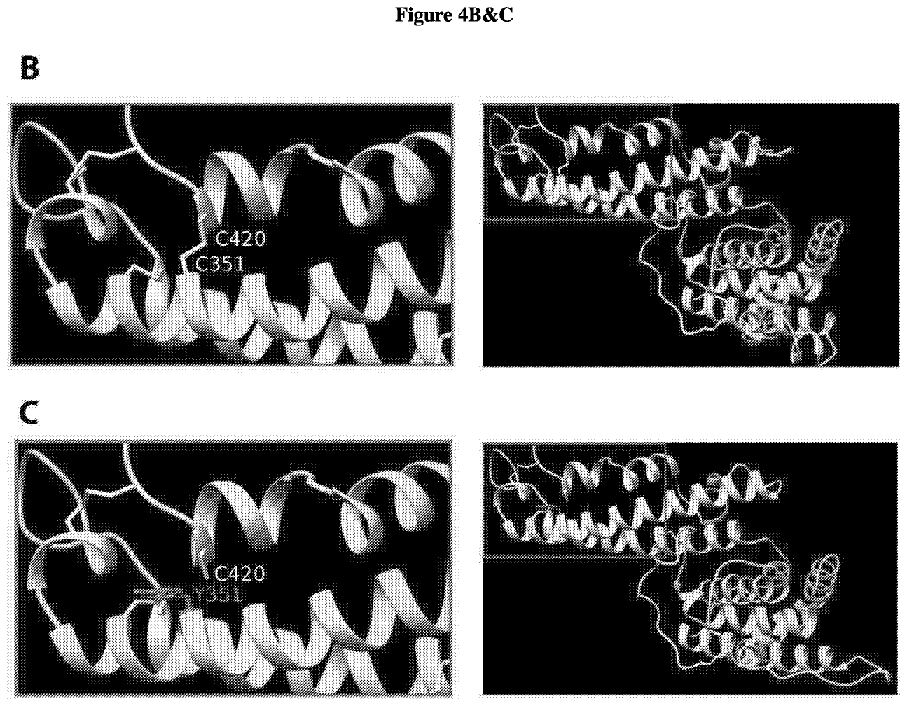

The homology models along with their disulfide bonds were visualized (FIG. 4B and FIG. 4C) and the images were obtained using the “Disulfide by Design 2.0” server (http://cptweb.cpt.wayne.edu).

### Example 1: Identifying Candidate Genes

The development of LGS has facilitated functional genomic analysis of malaria parasites over the past decade. In particular, it has simplified and accelerated the detection of loci underlying selectable phenotypes such as drug resistance, SSI and growth rate. Here we present a radically modified LGS approach that utilizes deep, quantitative WGS of parasite progenies and the respective parental populations, multiple crossing and mathematical modeling to identify loci under selection at ultra-high resolution. This enables the accurate definition of loci under selection and the identification of multiple genes driving selectable phenotypes within a very short space of time. This modified approach allows the simultaneous detection of genes or alleles underlying multiple phenotypes, including those with a multigenic basis.

Applying this modified LGS approach to study SSI and growth rate in P. yoelii, we identified three loci under selection that contained three strong candidate genes controlling both phenotypes. Two loci were implicated in SSI; the first time LGS has identified multigenic drivers of phenotypic differences in malaria parasites in a single experimental set-up. The strong locus under selection in Chr VIII, associated with the gene encoding MSP1, is consistent with existing knowledge of malaria immunity. The Chr VII locus, which includes the orthologue of Pf34 as well as other potential unannotated antigens, underscores the power for hypothesis generation and gene detection of the LGS approach using multiple crosses.

Our approach also provided a genetic rationale for the difference in growth rate of the parental clones CU and 17X1.1pp. Phenotypically, this occurs due to the ability of 17X1.1pp to invade both reticulocytes and normocytes, while CU is restricted to reticulocytes. Previously, differences in growth rates between strains of P. yoelii have been linked to a polymorphism in Region 6 of the Pyebl gene that alters its trafficking so that the protein locates in the dense granules rather than the micronemes. In the case of 17x1.1pp however, direct sequencing of the Pyebl gene revealed a previously unknown SNP in region 2, the predicted receptor-binding region of the protein, with no polymorphism in region 6. Consistent with this, the EBL protein of 17X1.1pp was shown to be located in the micronemes, indicating that protein trafficking was unaffected by the region 2 substitution. Allelic replacement of the parasite strains with the alternative allele resulted in a switching of the growth rate to that of the other clone, thus confirming the role of the substitution.

Region 2 of the Pyebl orthologues of P. falciparum and Plasmodium vivax are known to interact with receptors on the red blood cell (RBC) surface. Furthermore, the substitution falls within the central portion of the region, which has been previously described as being the principal site of receptor recognition in P. vivax. Wild-type strains of P. yoelii (such as CU) preferentially invade reticulocytes but not mature RBCs, whereas highly virulent strains are known to invade a broader repertoire of RBCs. Further structural and functional studies are required to elucidate how the polymorphism described here enables mutant parasites to invade a larger repertoire of erythrocytes than wild type parasites. We show that the cysteine residue at position 351 in EBL forms a disulphide bond with a cysteine at position 420, and that this is abolished following the C351Y substitution, altering the tertiary structure of the binding region. This leads to the possibility that such an alteration of the shape of the binding domain may enable the ligand to bind to a larger repertoire of receptors.

### Characterization of Strain Specific Immunity and Growth Rate Phenotypic Differences Between CU and 17X1.1pp

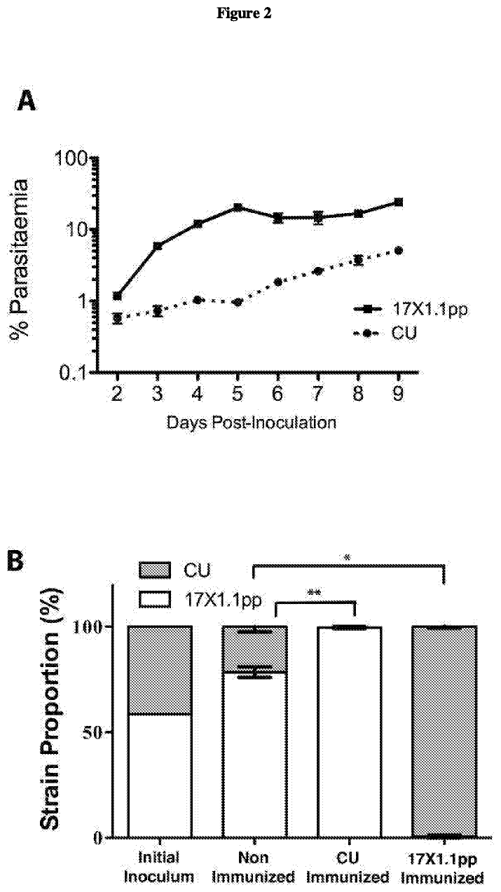

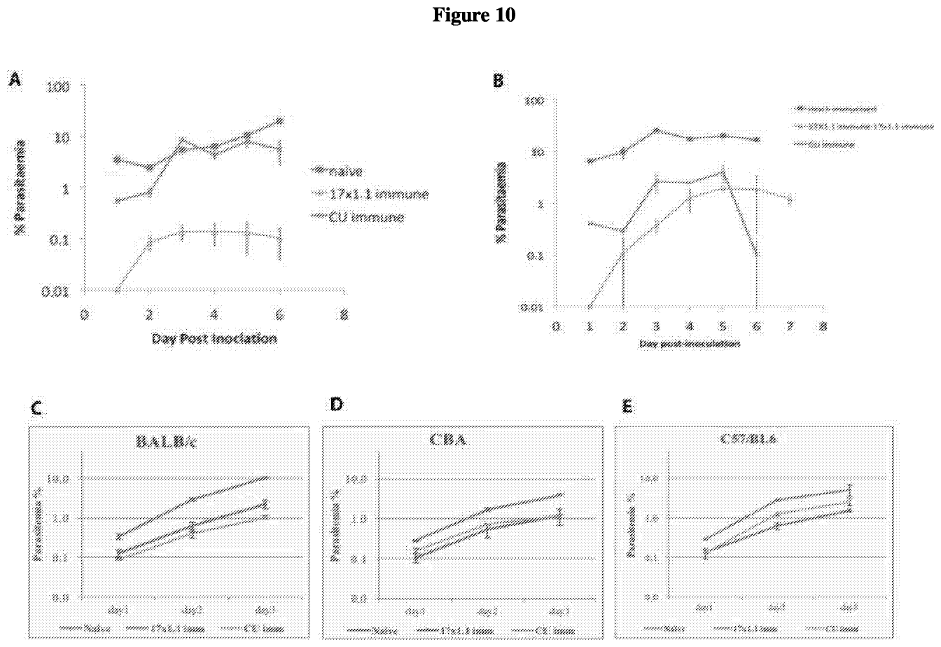

The difference in blood-stage parasite growth rate between the two clones was followed in vivo for nine days in CBA mice. A likelihood ratio test using general linear mixed models indicated a more pronounced growth rate for 17X1.1pp compared to CU clone by time interaction term, L=88.60, df=21, p<0.0001, FIG. 2A). To verify that the two malaria clones could also be used to generate protective SSI, groups of mice were immunized with 17X1.1pp, CU or mock immunized, prior to challenge with a mixture of the two clones (FIG. 10). The relative proportions of the two clones were measured on day four of the infection by real time quantitative PCR (Q-RT-PCR) targeting the polymorphic msp1 locus. A strong, statistically significant SSI was induced by both parasite strains in CBA mice (FIG. 2B).

### Identification of High-Confidence SNPs

Two kinds of selection pressure were applied in this study: growth rate driven selection and SSI. Two independent genetic crosses between 17X1.1pp and CU were produced, and both these crosses were subjected to immune selection (in which the progeny were grown in mice made immune to either of the two parental clones), and grown in non-immune mice. Progeny were harvested from mice four days after challenge, at which point strain-specific immune selection in the immunized mice, and selection of faster growing parasites in the non-immune mice had occurred. Using deep sequencing by Illumina technology, a total of 29,053 high confidence genome-wide SNPs that distinguish the parental strains were produced by read mapping with custom-made Python scripts. SNP frequencies from these loci from each population were filtered using a likelihood ratio test to remove sites where alleles had been erroneously mapped to the wrong genome location.

Identification of Clonality within the Data

A hidden Markov model was applied to the data to identify allele frequency changes (FIG. 7) that were likely to have arisen from the clonal growth of individuals within the cross population or possible incorrect assembly of the reference genome, as discussed above). In a genetic cross population, an especially high fitness clone generated by random recombination events can grow to substantial frequency, this being manifested as sudden jumps in allele frequency occurring at the recombination points in this individual. Jumps of this type were primarily identified in the 17X-immunized population, where the increased virulence of the 17X strain had less of an effect in driving alleles to high frequency, and in the first replica experiment; the data in the first experiment seemed to have been more affected by clonal growth in the population. The consistency of identified jumps between treatment conditions reflects the common origin of the differently treated populations; the jump at the end of chromosome XIV inferred in both replicas may be artefactual.

### Identification of Loci Under Selection

Based upon an analytical evolutionary model describing patterns of allele frequencies following selection, a maximum likelihood approach was used to define confidence intervals for the positions of alleles under selection in each of the genetic cross populations. In the absence of selection acting for a variant in a region of the genome, the allele frequencies in that region are expected to be locally constant. In common with a previous approach to identifying selected alleles, a search was therefore made for regions of the genome in which allele frequencies varied substantially according to their position in the genome. Next, wherever deviations of this form were consistently identified in both replica experiments a model of selection was applied to the data, inferring for each set of replica data the position in that region of the genome that was most likely to be under selection; this model was based upon expected changes in allele frequency under a constant local rate of recombination and is described further in the Methods section. Regions of the genome in which this inference of selection produced consistent results across replica datasets were then identified (FIG. 8). Of a total of 11 genomic regions suggesting evidence of non-neutrality, six showed sufficient evidence of consistent selection.

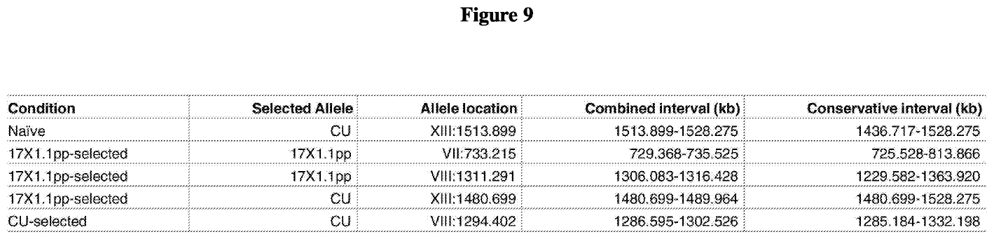

For each of these regions of the genome, a more sophisticated evolutionary model, accounting for variation in the local recombination rate, was then applied to the data, refining the position of the putatively selected allele. At this point, a putative selected allele in chromosome IV was removed from consideration, leaving five cases of potential alleles under selection in three regions of the genomes; confidence intervals for the positions of the selected loci are given in FIG. 9. Optimal positions of variant loci derived from each replicate are detailed in FIG. 13; results of the variable recombination rate model are shown in FIG. 14, with inferred recombination rates in FIG. 15.

Of the final three putative loci, two were detected under multiple experimental conditions (FIG. 3). When considering the combined largest intervals, a selective sweep was inferred at position 1,436-1,529 kb on Chromosome (Chr) XIII in replicate crosses grown in both non-immunized mice and 17X1.1pp-immunized mice, resulting from selection against CU-specific alleles at the target locus. A second sweep was inferred at position 1,229-1,364 kb on Chr VIII, detected in the parasite crosses grown in both CU and 17X1.1pp immunized mice, though not in the non-immunized mice. Here, selection pressure acted against different alleles according to the strain against which mice were immunized. The third sweep was detected at a locus between positions 725-814 kb on Chr VII. This event was only detected in mice replicates immunized with the 17X1.1pp strain, albeit that a consistent change in allele frequencies was also observed between replicas grown under these conditions (FIG. 3B). The remaining loci (on Chrs VIII and XIII) were not consistently detected between replicates (FIG. 13) and were thus considered to be non-significant.

Potential Target Genes within the Three Main Loci Under Selection

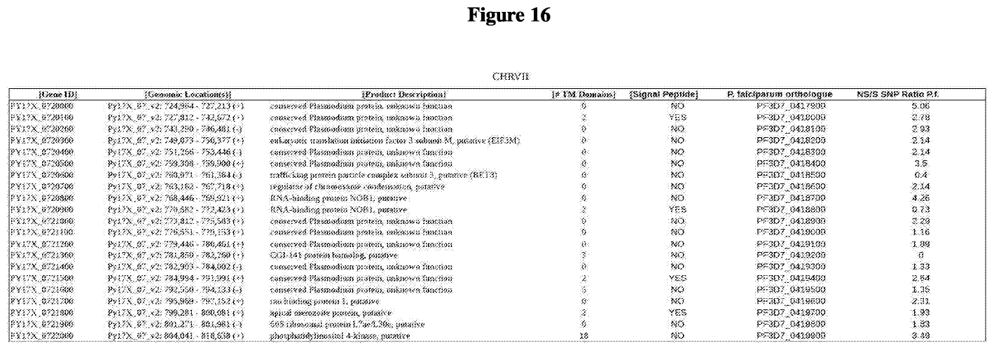

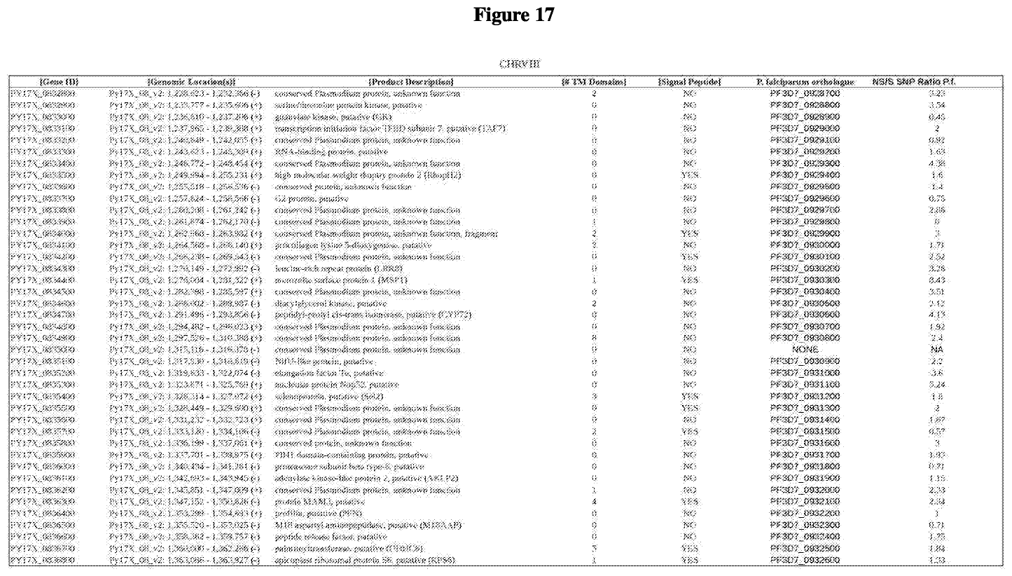

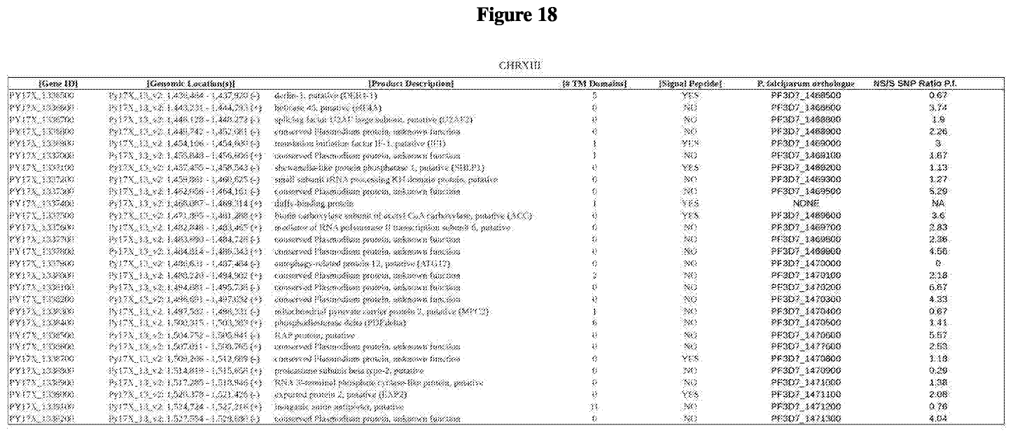

All the genes in the combined conservative intervals of the three main loci under selection are listed in FIGS. 16-18, along with annotation pertaining to function, structure, orthology with P. falciparum genes and Non-synonymous/Synonomous SNP (NS/S) ratio in the P. falciparum orthologue, which is calculated by the PlasmoDB website (6.2) based on SNP data from 202 individual strains. These include both laboratory strains and field isolates obtained from six collections (see Methods for more details). The locus associated with SSI on Chr VIII contains 41 genes. We considered the presence of either transmembrane (TM) domains or a signal peptides as necessary features of potential antigen-encoding genes. Only 16 genes met these criteria. Functional annotation indicated 10 likely candidates among these; eight genes described as “conserved Plasmodium proteins”, and two encoding RhopH2 and merozoite surface protein 1 (MSP1). Of these genes, the P. falciparum orthologue of msp1 had the highest NS/S SNP ratio (8.43). MSP1 is a well characterized major antigen of malaria parasites that has formed the basis of several vaccine studies and has been previously linked to SSI in Plasmodium chabaudi.

The locus under selection on Chr VII consists of 21 genes. Only seven contained TM domains and/or a signal peptide motif. Based on functional annotation, four of these could be potential targets for SSI. One of these genes, PY17X_0721800, encodes an apical membrane protein orthologous to Pf34 in P. falciparum. This protein has recently been described as a surface antigen that can elicit an immune response. Three conserved proteins of unknown function (PY17X_0720100, PY17X_0721500 and PY17X_0721600) also displayed potential signatures as target antigens. PY17X_0721800, PY17X_0720100, PY17X_0721500 were selected as candidate genes based on their predicted immunogenicity.

The growth rate associated selected locus on Chr XIII contains 29 genes. In this case, the presence of TM domains or signal peptide motifs were not considered informative criteria. Only eight genes contained NS SNPs between the parental strains 17X1.1pp and CU according to the WGS data. Among these was a duffy binding protein, Pyebl. Pyebl, is a gene that has been previously implicated in growth rate differences between strains of P. yoelii. A single NS SNP was predicted from the WGS data in this gene. Due to the very high likelihood of its involvement based on previous work, this gene was considered for further analysis.

### Characterization of EBL as the Major Driver of Growth Rate Differences Through Allelic Replacement

Examining the Pyebl gene, Sanger capillary sequencing re-confirmed the existence in 17X1.1pp of an amino acid substitution (Cys>Tyr) at position 351 within region 2 of the encoded protein. When aligned against other P. yoelii strains and other Plasmodium species, this cysteine residue is highly conserved, and the substitution observed in 17X1.1pp was novel (FIG. 4A). Crucially, no other polymorphisms were detected in the coding sequence of the gene, including in region 6, the location of the SNP previously implicated in parasite virulence in other strains of P. yoelii. Structural modeling of the EBL protein in both wild-type and 17x1.1pp (C351Y) mutants predicted the abolition of a a disulphide bond between C351 and C420 in the mutant parasites that alters the tertiary structure of the receptor binding region of the ligand in these parasites (FIGS. 4B and 4C).

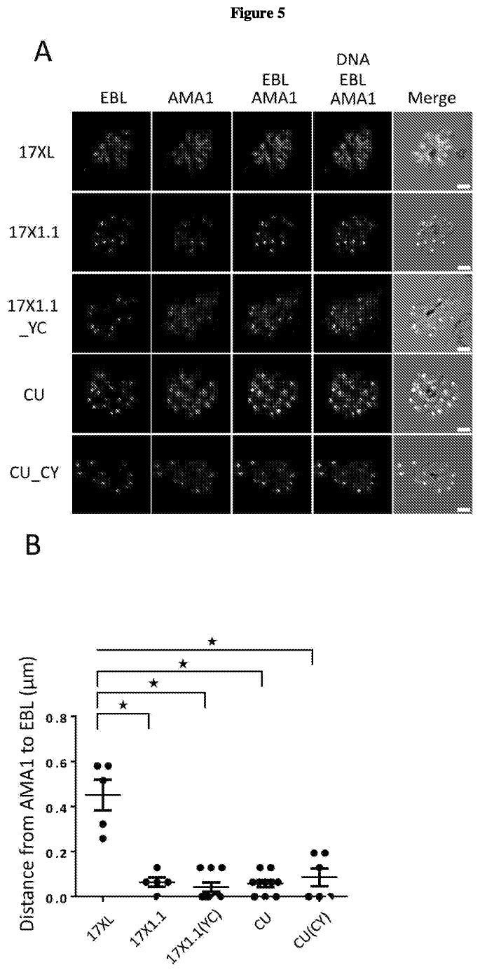

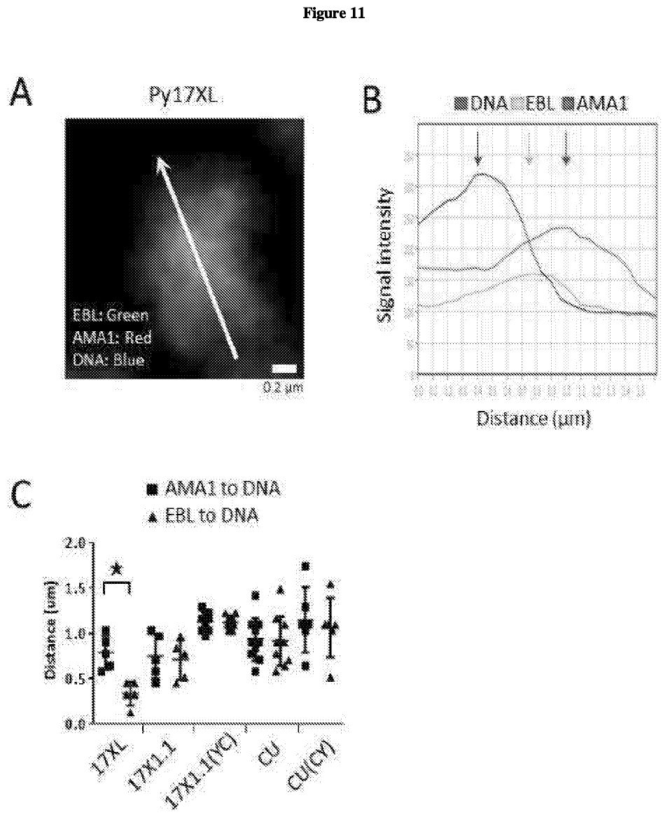

The functional role of this polymorphism was verified by experimental means. In order to study the functional consequences of the polymorphism, the Pyebl alleles of slow growing CU and faster growing 17X1.1pp clones were replaced with the alternative allele (i.e. CU-EBL-351C>Y and 7x1.1pp-EBL-351Y>C), as well as with the homologous allele (i.e. CU-EBL-351C>C and 17x1.1pp-EBL-351Y>Y). The latter served as a control for the actual allelic swap, as the insertion of the plasmid for allelic substitution could potentially affect parasite fitness independently of the allele being inserted. To establish whether the C351Y substitution affected EBL localization, as was shown for the previously described region 6 mutation, Immunoflurescence Analysis (IFA) was performed. This revealed that, unlike the known mutation in region 6, the EBL proteins of 17X1.1pp and CU were both found to be located in the micronemes (FIGS. 5 and 11).

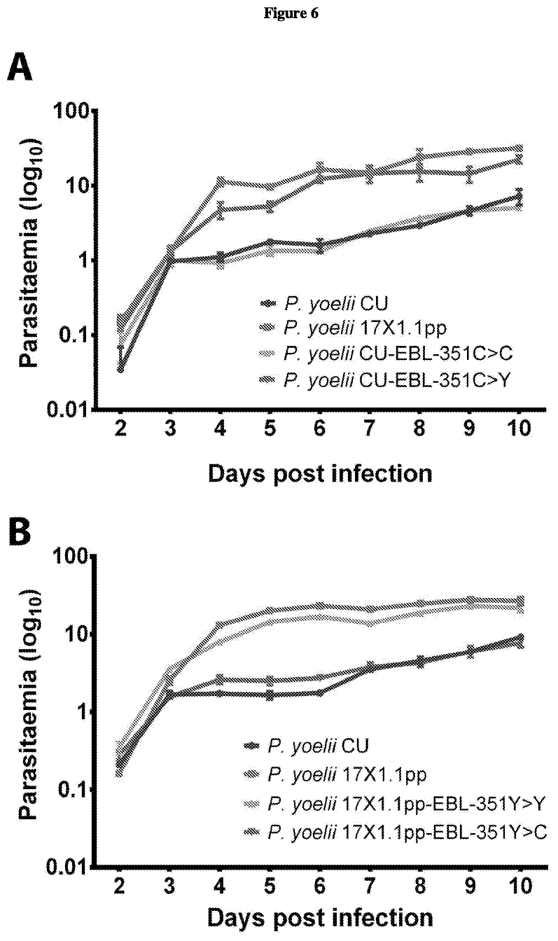

Transgenic clones were grown in mice for 10 days alongside wild-type clones. Pair-wise comparisons between transgenic clones with the parental allele against transgenic clones with the alternative allele (that is CU-EBL-351C>C vs CU-EBL-351C>Y and 17x1.1pp-EBL-351Y>Y vs 17x1.1pp-EBL-351Y>C) showed that allele substitution could switch growth phenotypes in both strains (FIGS. 6A and 6B). This confirmed the role of the C351Y mutation as underlying the observed growth rate difference.

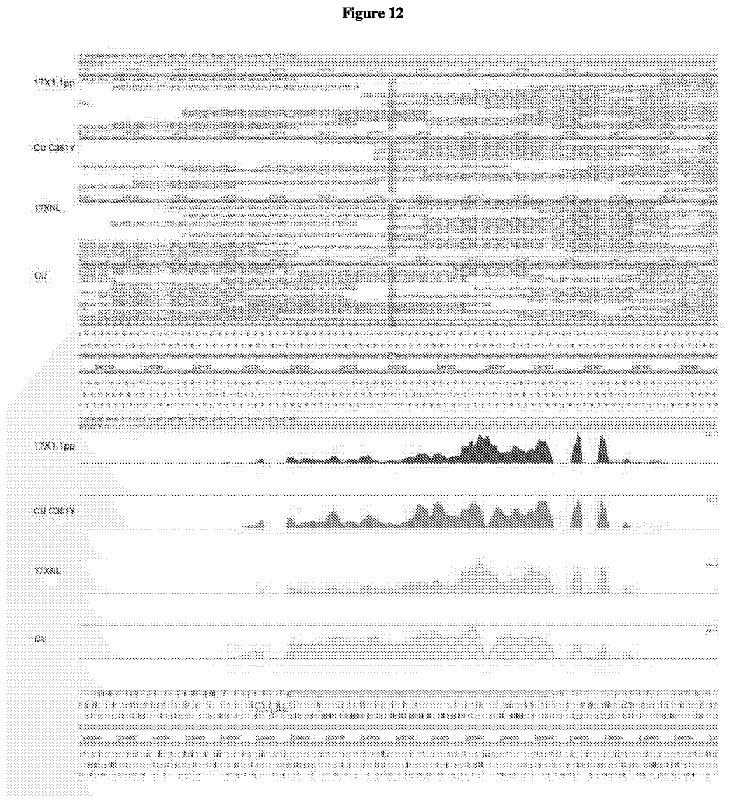

RNA-seq analysis revealed that transfected EBL gene alleles were expressed normally, (FIG. 12), thus indicating a structural effect of the polymorphism on parasite fitness, rather than an alteration in protein expression.

LGS with multiple crosses offers a powerful and rapid methodology for identifying genes or non-coding regions controlling important phenotypes in malaria parasites and, potentially, in other apicomplexan parasites. Through bypassing the need to clone and type hundreds of individual progeny, and by harnessing the power of genetics, genomics and mathematical modeling, genes can be linked to phenotypes with high precision in a matter of a few months, rather than years. Here we have demonstrated the ability of LGS to identify multiple genetic polymorphisms underlying two independent phenotypic differences between a pair of malaria parasite strains; growth rate and SSI. This methodology has the potential power to identify the genetic components controlling a broad range of selectable phenotypes, and can be applied to studies of drug resistance, transmissibility, virulence, host preference, etc., in a range of apicomplexan parasites that are amenable to genetic crossing.

The applicability of the approach to human malaria species has been recently demonstrated: the original LGS approach was successfully applied to study P. falciparum immune evasion in mosquitoes in vivo, while we recently tested its applicability in vitro to detect loci under selection following antifolate drug treatment and in vitro growth rate competition. With the advent of humanized mice that are able to support the complete malaria life cycle, the generation of new genetic crosses between strains of human malaria has become more feasible, as recently demonstrated. With the ability to maintain these crosses without the need of simian hosts, application of a broader range of selection pressures (excluding, for now, selection mediated by the presence of a complete immune response) is now more feasible in vivo, thus extending the application of the LGS approach to medically relevant malaria species.

The qSEQ-LGS method described herein enables us to quickly and more precisely identify antigens or drug/vaccine targets within the malaria parasite's genome that would be effective drug or vaccine targets.

### Example 2: In Vivo Experimentation with Candidate Genes

Healthy mice are administered an immunogenic composition comprising an immunogenic polypeptide encoded by the nucleic acid sequence PY17X_0721800, PY17X_0720100, PY17X_0721500, or a fragment thereof (treatment groups).

Both treatment group and control mice will then be exposed to a malaria parasite.

It is expected that mice in the treatment group will have protective immunity against the subsequent malaria parasite challenge while control mice who did not receive the immunization will have a higher rate of malaria parasite infection.

Treatment group mice will then be rechallenged at with the malaria parasite at several time points to test the lasting effects of the protective immunity.

### Brief Description of the Sequences

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. The amino acid sequences follow the standard convention of beginning at the amino terminus of the sequence and proceeding forward (i.e., from left to right in each line) to the carboxy terminus.

PY17X 070800 Protein
>PvivaxP01|PVP01_0529100.1: pep
(SEQ ID NO: 1)
MMNVFFCLSCFLVIKTLFQSCPAKCNELNVSGKGNMLYDYLFTPEVKAPKDDGG
KEQDTLAGNKQMSYKTSEVQIKHHEEVHTGGMENGDHRKELKEHMDQLKMLH
KSLKSKDRNTYGQVKPSGGESVHSNDGALGDTHTGGDQTSEESLAKYVQNEIKN
NEKLNKEKKSYDEINLLEDNSKKLQNDIHTWLQSVKNITEKTSKLKDIKTQLLNNI
ASLNETLTEEIENINEIKKLQKEQNEIFSENWLYFLPSTSDSLASEGRDGSFQVLNY
LEKFSRRDAPQVSSEPASGELQNGGLPNGGLPNGGLPNGGFPNGGLPNGGPPNDG
NVRKAHSHTDDGAKSSDSRSFCSVLVLLAIAFLLS
>Pknowlesi|PKNH_0512400.1: pep
(SEQ ID NO: 2)
MMNVFVCLSFILVISIFLQSNPAKCNELNISEKGKMLYDYFFTPKVRGPKDGGTDQ
DVHTGDKHLSYTTSEAQIRHHEQVHIGGMKNNDHKKELKEHMDQLKMLHKSLK
DKDKNAHDPELKVSGGESVHNTDGTLGDTHANGEETSDDSLAKYVQEEIRKNEK
LNNEKKSYDEINILEDNSKKLQNDIHTWLQSVKNISEKTNKLKDIKTQLLNNIASL
NETLTEEIENIKEIKKLQMEQNQIFSENWLYFLPSTFDNLASHGQDGNFQVLNYLE
KFNRRDGLHVSGEKANLEQPSGTLANEGNVRKQNAHTDGGVKSSDFHSFCNVIV
LLAVVTFLLS
>Pmalariae|PmUG01_05037200.1: pep
(SEQ ID NO: 3)
MYVRTAVCCIRLHVTDNTFSVPMNFQKCKVFFLFSHLFLFLDFPLFNIIMNCGFSFF
CILIINIILSINLIVECNKLMLNDKDKMTVDAILSKEVKENKGFNIQQVEKGNAISHK
KLEMNINHHNVNDNDLKDHMDKLRKLHKDLKNSSSSSSNSNNSSTNSSNSSSKA
YSKGDILKVEDQNSSNKTDKNNHSTNGKGEKDNNDDSLRKYILQEINNNRKLDTE
KKSYEEINLLEENSKKLQNDIHTWLKSVNDIEEKSKKLKDIKSQLLNNIASLNQTL
TEEIENINDIKKLQKEQNDIFSENWLYFFPSTPDYLIEEKNKSNYNILNYLESFSKKN
KQQSYESNIRKQQKPHHYDNTNSSDIFHFLSYFVLLMFFFFFS
>Povale|PocGH01_05030900.1: pep
(SEQ ID NO: 4)
MILRFSFLCVLVINVLSTNLFVLCNKLKINGNGKSVNESFFPDGTKNESHMNKNIM
KKGREREIPRKETQANIGHHNFHENELKKNIEMLKQMQGDLKNGKTPLHKKAIN
GADKGGHIGEWDREDELRKYVMEEIRNNSKLDREKKSSEEIKLLEDKSIQLQNDI
HTWLQSIHNIEEKSTKLKDIKKELLHNITSLNETLVEEIENINDIKKLQKEQNEIFAE
NWLYFFPSISDNVSEDGSDSNHNILNYLEMYNKKDKEKQKESEMERGVWKEHLR
ENATMKQYGESSKSAGMGCVVNHILFLLGVIFLF
>Pfalciparum|PF3D7_0419700.1: pep
(SEQ ID NO: 5)
MYGTFLKGSIFYLCIFFPFFSCECNNIKLNDKENINFESYFNKRTNEENVLNKNVSK
EMGDTFVAHKAIELNINHHHVNNDKEFNNNNNNKHQPYYHNEHDKKFSESLKA
HMDHLKILNNDLKQHIDKKERNEIYENNDLKKYIIKEIQNNKYLNKEKKSSEDIQI
LEEHSKKLQKEIHEWLESVNNIEEKSNILKNIKSQLLNNIASLNHTLSEEIKNINDIK
ELQKQQNDLFSENWLYFLPSSSDYLLNEKKKNLYDNQDNSMKDDINNNDKYNIF
NYLQNVQDKDNQYEVMKQDNNNIHSGSSTHNHLLLTCIIFLLILLIL
>Pyoelii|PY17X_0721800.1: pep
(SEQ ID NO: 6)
MNYNFLFFSILIINIFSTHLISKCNKLKLSSKRKSNQDALIPNEGNYMNNNIIDGEKN
NKENINEEIIKQDGKIDNGENYIEIKLDYNKNDRINNEKINTNINHIDNVQNEYDNI
KEMEKMTKSYEDMSILEENSKKLQNDIHSWIKSVHSIEEKTNTLKNIKNDLLNNIT
SLNKTLLEEIENINEIKKLQNEQNEIFSENLLYFFPSMPEKLQENVEKDYNILNYLEI
YNKKDNLKKVDTNISSTCMFSFFSYLILFSATVFFFL
PY17X 070800 DNA
>PvivaxP01|PVP01_0529100.1: pep
(SEQ ID NO: 7)
Atgatgaatgttttcttctgcctctcgtgctttttggtaataaaaaccctcttccagagctgcccagccaaatgcaacgaattaaatgta
agtggcaaggggaatatgctgtacgattatttgttcacccctgaggtgaaggccccaaaggatgatggggggaaggagcaggac
accctggcagggaacaagcaaatgtcctacaaaacatcagaggtacaaattaagcaccacgaagaagtgcacacaggagggat
ggaaaatggtgatcataggaaggagctcaaggagcatatggaccagctaaaaatgctacataaaagtttaaaaagcaaagatcga
aatacatacggtcaggtaaaaccaagtggaggcgaaagtgtacacagtaatgatggcgcactgggggatacccatactggggg
ggaccaaacgagtgaagagagtctggccaaatatgtgcaaaacgaaataaaaaacaacgagaaattaaataaagaaaaaaaaa
gctacgacgaaattaacctactggaggataattcaaaaaaattacaaaatgatattcacacctggctccagtctgtaaaaaatataac
ggaaaagacgagcaaattgaaggacattaaaacgcagctgctgaacaacatcgcctcgttaaatgaaaccctgacggaagaaat
tgaaaatataaacgaaattaaaaagttgcaaaaggaacagaatgaaatattttccgaaaattggctctacttcctcccctccacgtcg
gacagcttggccagcgagggacgcgacggcagctttcaagtcctcaactacttggagaagtttagcaggagggacgctccgca
agttagtagtgagcccgcgagtggggaactccaaaatggggggcttcctaatgggggccttccaaatgggggtcttccaaatgg
gggtttcccaaacgggggtctcccaaacgggggtcccccaaacgatgggaacgttcgtaaggcgcactctcacaccgacgacg
gggcgaagtcgtccgactcgcgcagcttctgcagcgtgctcgtcctgctcgcgatcgcctttctgctcagc
>Pknowlesi|PKNH_0512400.1: pep
(SEQ ID NO: 8)
Atgatgaatgttttcgtttgtctctcgttcattctggttataagcatattcctccagagcaacccggccaaatgcaacgaattaaatata
agtgaaaaggggaagatgttgtacgattatttctttacccctaaggtaaggggcccaaaggatgggggtacggaccaggatgtcc
acacaggggataagcacctgtcgtacacaacatcggaagcacaaattaggcaccacgaacaagtgcacataggaggaatgaaa
aataatgaccacaaaaaggaactgaaagagcatatggatcagctaaaaatgctacataagagtttaaaagacaaggacaaaaatg
cacacgatcctgagttgaaagtaagtggaggcgaaagtgtgcacaatactgatggcacactgggggatacccatgctaatggag
aggaaacgagtgatgacagcctggcgaaatatgtgcaagaagaaataaggaaaaacgagaaattaaataatgaaaaaaagagc
tatgacgaaattaacatactcgaggataattcgaagaaattacaaaatgatattcatacctggcttcagtcagtaaaaaacatatcgg
aaaagacaaacaaattgaaggacattaagacgcagcttctgaacaacatcgcctcattaaatgagacacttacggaagaaattgaa
aacataaaagaaataaaaaagttacaaatggagcaaaatcaaatattttcggaaaattggctctactttttgccttccacatttgacaac
cttgctagccacgggcaagacggcaattttcaagtactgaactatttggagaagtttaacaggagggatggtcttcacgtgagtggt
gagaaggcgaatctggaacaaccaagcggaacacttgcaaatgagggaaacgttcgtaagcagaatgcgcacaccgacggag
gggtgaagtcgtccgactttcacagcttctgcaacgtgatcgttctactggcagtggttacctttctgctcagctag
>Pmalariae|PmUG01_05037200.1: pep
(SEQ ID NO: 9)
Atgtatgtacgcactgctgtatgttgtattcgtcttcacgtaacggacaatacgtttagtgtccctatgaattttcagaaatgtaaagtttt
ctttttattttcccatctttttctttttttagattttcccctttttaatattataatgaattgcggtttttcttttttttgtattttaataataaat
ataattctttcaataaatttaatcgttgaatgtaataaattaatgttaaatgacaaagacaaaatgacagtggatgctattctttcaaaagaggtgaa
agagaacaaaggattcaacatacaacaagtagaaaaaggaaacgccatttcgcacaaaaaacttgaaatgaacattaaccatcac
aatgtgaatgacaatgatttgaaggatcatatggacaagctaaggaaattacacaaagatttaaaaaatagtagtagtagtagtagta
atagtaataatagtagtactaatagtagtaatagtagtagtaaagcatattcaaaaggggatatactaaaagttgaagatcagaatagt
agtaataagacagataagaataatcatagtacaaatggaaaaggggagaaggataataacgatgatagcttaagaaaatacatttt
acaagaaataaataacaacagaaaattagatacagagaaaaaaagctatgaagaaataaatttgttagaagaaaattcaaaaaaac
tacaaaatgatattcatacttggttaaagtcagtaaatgacatagaagaaaagtccaaaaaactaaaagatattaaaagtcaactatta
aataatattgcatctttaaatcaaactttaacagaagaaatagaaaatataaatgatattaaaaagttacaaaaagaacaaaatgacatt
ttttctgaaaattggttatactttttcccctcaacacctgactatttaatagaagaaaaaaataaatcgaattataatattttaaattatttag
aatcctttagtaaaaaaaataaacaacagagttacgaaagtaatataaggaaacaacaaaaacctcatcattatgataatactaattct
agtgatattttccactttttaagctatttcgtcctcttaatgtttttttttttttttagttaa
>Povale|PocGH01_05030900.1: pep
(SEQ ID NO: 10)
Atgattttgagattttcctttttgtgcgttttggtaattaacgtactttcaaccaatttgttcgtcctatgcaacaaattgaaaataaatgga
aacgggaaatcagtaaatgaatcgttcttcccagatgggacaaaaaacgaaagtcatatgaataaaaatattatgaagaaaggacg
agaaagagaaatcccgagaaaggaaacacaggcaaacattggtcatcacaattttcatgaaaacgagctgaagaaaaatatcga
gatgttaaagcaaatgcaaggggatttaaaaaacgggaaaactccattacacaaaaaagcaatcaatggagcagacaaggggg
ggcacattggtgaatgggatagagaagacgaactgagaaaatatgttatggaagaaatacgtaataatagcaaattggacagaga
aaaaaaaagttctgaggaaattaagttattagaggataaatcaatacaactacaaaacgatatacatacatggttacaatcaattcata
atatagaggaaaagtctactaaattgaaagacataaaaaaggaattgttacataacataacctcattaaatgagacattagtagaaga
gatagaaaatattaatgatattaaaaagctacaaaaggagcaaaatgaaatatttgcagaaaattggctctattttttcccctctatatc
ggataacgtatcagaagatgggtcggacagtaaccataacattcttaactacctagaaatgtacaataaaaaggacaaagaaaaac
agaaagagtcagaaatggaaagaggcgtgtggaaagaacatcttcgtgaaaacgccactatgaagcaatatggcgaaagttcaa
aatcagcaggtatgggttgcgttgtaaaccatatccttttccttttaggagttatattcctcttttga
>Pfalciparum|PF3D7_0419700.1: pep
(SEQ ID NO: 11)
Atgtatggtacatttttgaaggggtccattttttacctgtgtatatttttcccatttttttcgtgtgagtgtaataatataaaattaaacgataa
agagaatataaattttgagagttattttaataaaaggacaaatgaagaaaatgtattaaataaaaacgtatcgaaagaaatgggggat
acgtttgttgcacataaggctatagaattaaacattaatcatcaccacgttaataatgataaagaatttaataataataataataataaac
atcagccttattatcataatgagcatgataagaaattttctgaaagtttaaaagcacatatggatcaccttaagatattaaataatgattta
aaacaacatatagataaaaaagagagaaatgaaatatatgaaaataatgatttaaaaaaatatataataaaagagatacaaaataata
aatatttaaataaagaaaagaaaagcagtgaagatattcaaatattagaagagcattcaaaaaaattacaaaaagaaattcatgaatg
gttagaatctgttaataatattgaagagaaatcaaatattttaaaaaatatcaaaagtcaattattaaataatatagcttctttaaatcatac
gctctcagaagaaataaaaaatattaacgatataaaagaattacaaaaacaacaaaatgatttattttctgaaaattggttatattttcttc
catcctcatcagattatctcttaaacgaaaaaaaaaaaaatttatatgataatcaagataatagtatgaaggatgatataaataataatg
acaaatataatatttttaattatttacaaaacgttcaagataaggataaccaatatgaagttatgaaacaagacaataataatatacatag
tggttcctctactcataatcatctattattaacttgtataatttttttgttaatacttttaattttataa
>Pyoelii|PY17X_0721800.1: pep
(SEQ ID NO: 12)
Atgaattataattttttatttttttcgattttaataataaatatattttcaacacatttaataagcaaatgtaacaaactgaaattaagttcaaa
aagaaaaagtaaccaggatgctcttattccaaatgagggaaattatatgaacaataatataattgatggtgaaaaaaataataagga
aaatataaatgaagaaataattaaacaggatggaaaaatagacaatggagaaaattatatagaaattaaattagattataataaaaac
gatagaataaataatgagaaaataaatacaaatattaatcatattgataatgtacaaaatgaatatgataatattaaagaaatggaaaa
aatgacaaaaagctatgaagatatgagtattttagaagaaaattcaaaaaaattacaaaatgatatacattcatggataaaatctgtac
atagtattgaagaaaagacaaatacattaaaaaatataaaaaatgatttattaaataatattacatcattaaataaaacattattagaaga
aattgaaaatattaatgaaattaaaaaacttcaaaatgaacaaaatgaaatattttctgaaaatttgttgtattttttcccatcaatgcctga
aaaattacaagaaaatgtagaaaaagattataatattttaaattatttagaaatttacaataaaaaagataatttaaaaaaagttgataca
aatatatcatctacttgtatgtttagtttttttagttatttaattttgttttcagcaacagttttcttttttttataa
PY17X 070100 Protein
>PvivaxP01|PVP01_0527400.1: pep
(SEQ ID NO: 13)
MPGETQNTFDLVDVEPKFLEFHYEGADSVEAFLENKKKVIKRKGLKIKNICTKTQ
NIKICECDSKCLKIKGLKKKKLAPGTSEQILVEFSFADVNFKESKRGKQEDILEVVN
NVEATSYVTIQSEYTTLSIPIYIKKSIPVFAYDDVINFGVCNANRTYHFALRVKNVG
TRRGAFTLSLGDSPGGNADECEDGKRAESAQSGENSVKQHRRNCHSEDMLIRLD
QSSLELDVGETKTINITVSSKVEGKMHREYIVVSNQHSYFVEKQRNINVLAIFTNS
HTSFLFENGKTNLLNLHCLYYGSRKKYQGKIKNENNYGIFCTPRVDAVHVFYSKE
ELQTFAAARGLDLGRICRDVFLEEEERLSEGEKKEREKLFAITKKRLKGEEHIGVT
FCRGEGYPVERLSYQEVTFEIQSKSDAGLLHRLSRDTAYLLNFPVVVELSLRVGRS
RREASQVGITTKQAHRSTSQVCSSAKQEHRSTSQVCSSAKLPHSSSKLPPLSTKLPH
SSTEVGDSHPCDEEVKLWAAFCLTFPCVLPSTYLLSYGSIAPNEGKTVILELTNRN
KFLKVSYTLSKIPYLTLSKKEGVIPPQGKALLSLKLQCDSLKMVEEYMYIYFCNNL
FFFVLLVRGNITSANALRKGTSSILLDPKGDSLKRNTPPQGDSKQDRDQVYEQILH
NLELEKVSRNYYQLDGKLDKYKYNERFINFLKKNKKKYNDVLKLMYEERKKSES
VRSGTQSQGSPDKSGKIIKGGTDKLSKIMTDKFIYVNDPPVDRISNTCIEKGVYERE
RKKYIQMKTTNWGVKNGEPSQGGEEQPYLLQFDMLDEEELKRVQFVRVIQYGNI
FLGKEYTRKFAVFNRNKHCSVRVALTHSENVTTEGDNPLVIHRDSHKMGIIKLNV
KRFSTAEEPHHDAVSDGDFRSQTGVSTPLFEAVNLPTGGGTSTPEGNTPTALCADP
CNDFFLLKTFQEKISLTVNDSHEREVTLGATLVFLNILVHPHTLHFRFHDEDVGMF
CERHLVLYNPFNVPLRVGMACNRACVQLDAEISVPPNSHKIVPVKFVATESATSV
AEAIRLYLDGSRLYRSLTCKWIANKCSYRVTPAELNFENVLLNKTYVKEFFLINTG
DSPVVFRCSYKPDCVNLLSKHNYVKKNEKVKISVLLNLKECKKMKDKVVLAVR
GAPQLLLPLSAKGTPSNVLISGGLRIRQRRGELKCYEMKIANRGPCEEAILLDTSPV
GFLNIQMGHNNERTFSESTGKESKDASKRENVLTVERTFNQEYDDLLTDECAKYQ
YNNFLRLHNLVSSCYQNKGQILFNEKRERKNIYRVVVPSKCFLPLYIQCYSSRALT
KEITLHSLLHCNRAAYECKEESITVDIEEGHLDVSPPFLLFTDLLPQKADEAYITYFS
RERRKRLTIRNVLSQPVQWAVRLDEEKRRELYTVRGAHRGGEQIAERRGEAGKS
RRVGSDIKGSAIGGSAKRGEAPETCVTQGSAIGGSAKRGGATKSPESDEPAEERQY
QVHPSRERGILQPNEETTVEIKLSGFKQCGRYVDVLDISSSPVGPPHGIDSEEAQEQ
REAAEGETHFALYMLIRCDVPKLQFDVTYINITHNKLNECISFSFGIYSHGYHYVR
VDSHLKNPHAECFSISLHFIDGNEINERVKKLRVQLKCKASRWLTFKSSIVLSVMN
HHQYALPLFVSIDEGVDFLLGRASGEGSGETSGETSGEISGETSGVTSGETSGEISG
ETTKGATLERSLHQHLNTTDLPFDKQNELLSFFCKNVEESKGGSTNGDIYKGLKIG
KIKNVYCHEEEGVYPLSEIVGDYLFVFFQKMLLGKSSFPQVIESTNDLFLFFVDLLF
DLFSATYPNRNLQNAIAEVKRHRAVVVKDMERQDLLDERLAKHVKALWKCLQE
VGTGRLRLAVHHPATLYATSSPRYVSCHFITPLLFTPLQECLPVDHLLYENLLPAD
LYLPHILANVPDHFHVEEKDEEDYGDSAGHLSMRDVESFLLNGNKKVFYFERIFK
THVRLFSYSWVTIFLEILNRKFFGRINISSLVTLRGIKLYSIENKLNENIKMCKISYLV
ILDTQDREVNKHVSFLSEKKGRSRRKDVTTLTNLQVVSGGTATLRTGGQSINTRN
GNPTDGDKGTPQRREKYSPNYVKKRTAKDKPCCAINYFNFYENNFIDLKKIANKI
SGFCSPGGEEAAQGEAGAPIAQRTPSRPFKKEPTKETAKQTSPNAPNGIVHFQSAE
CILFEWLYFHYNNVHYAKVEDKRYVFREARGGGDEELSHTNGAMKGGEPEGAR
RRGSPSGSTNCAPSGNPRFAPSGNPSFTPSGNPSFTTSGNPSFTPSGNPRFAPSGNPS
FTTSGNPSFTPSPVGKNPKMSRINFQVEIKRKKKEAEKKKKVKGIDELKDLVMVL
YTIISHIPYYLFFKNKIKVKCKSKRDYNTNVEILLIMLSEMKLSSLISRGVLAQFNHL
HIYLFLASLYFILPSYLPGYYLILDDFPAYEVDLGLITDEVVNGRKKWKQQPGLRQ
PPTKGKQQTGLKQPPGKSPLPPQSKANPHTDGGGEKTGCGDGHSLANERIITVYN
LNSHKCKYEVFLIGCHKYQLDRNHLCIDPLKAEQIKLKIDRGYDEQALSYSYHTSI
KLSSFKKKRDKGDMCDGEEPHLEDSCSVSSCSSEECQCPSDGADKVDAGTSGESR
PTPGEDYSILVLRAEGNHLQDEQHSEKYICIKLVERDEKGKIELTSPGASIRGKQNE
GKNVKANLHNGVPRSEVNKMRTANSSQLNIMLNDSEVKCFNLRGKVYERKCLEF
NLCNNTGKEVEVRSYLYHVYGKDLKEEKRRGNLEADVVSSWGNSQCAVCSLVS
ANLRRHLSGTPTHFSCFHLSGAPPHFCCLQNERKKIQVQFCPFAEGSYSCFLLLNV
NDKKSNYVVSACKVNCFFNVKNVKEEEVIKMRSQTFSFSYQLEVCPLNREFLHCV
KFILCNLNHLEESHFLAYVRGYLKGIQHGGDNFYILCDNEDKVTIERGVATFGVLD
AEKYVEKAAHSEGANVEKLHQMVKRDVNYMCLLQIAEQTSGVFEYNCALVRQR
GAPRGRNHLSYEELRNLHIKEQQEWFDPFYKVYKIVANVNDAGNTQSLSITFKTS
AFLMAKKTIPLYNYTNGVVTYRRVLSDVIDENNNVVDYQIFSCPEYVEIGKDVEF
AQYEVSCYSIIGLTCSCCITLYNVNDEEDRIKINVQANVMKPYPKDTIHLKLLNRM
KKTAQVEIYNELNSHCEFKVFSDLPILFGGKKIKMLPKERKAYTFFVTSAHIGEYM
GCIIFKFHKLLRSGGAEEKTRDASFPFANYFFWYKLNITVELNKPLKILFLETNVGE
EVTKEIVLKNNGTQDEDYFLLAYMNEYIERTQVQVPRNDIYVYSIRYAPKMPNCF
EGASSGGAPLGGSAALGENHPTVRGDLQNVYFPQNGDSEWCGPREEALRGKPST
DYPNEEAPSRTPSAEVPPNHGAAPKRKPFPPNEKKIFEELISYCKKYIQVDIPYRPQ
NIGFFFIYNKNEGINYYILMLLSRLRSELEVGRFSTCLSKSCLLHLHFHFNDEDKRY
VHLLQEKGDPRAGNYHYEIGEGVKHPQGGGNALKGENFQQGTPHTGRAYQTYD
DEWGEEIHRGDAKNGKDAPICKDNPCSGSGSDQEKHELKCIYLSNRKNFFTVNHE
DVGRIVLKYRPTVGTSQECYAIVRSNLHGDFLYRISGTYTVKRRIKEKLVFYNSCC
LYSFNVNLFNPFDCKVRVKCRFKGGGPSQACSGRGGVGGAEEGAEEGTDERDTE
RNCLTSMQTERVLLNNERNYLKMMNKENFKIKMRRHFTIMMTYFCKEVYSRRA
VLVVMKPLRRGDATREDVPPMITYEYDFVFNNLLRSGRLPGALRCVPIAGEGKRA
GEKGNRGEERSQKGEDPGVSTSCNFHSGVDALRIGSRVGGDFTRRSEGVEAENNL
PEVADGEDNPPEVSDGEAHPSEQSPSSHTSRSPSAASERSHFTLNASELGTTDVLEG
ELKSEDADAAEFEKHPPGDKALWRPNDYNVEEDVRRERVLPCAAGGAAGRAAIS
ATDSNEVVHYDGKVFIESMCKAKTCVEVHIDKAKRRAGATPPQGGGQLEGEKQE
GKQQAGKQQAGKQQAGQNSALHRNVQRGKYNYKIFLQNVKNVRKGGGSGGGG
EGGGECGRETPGMDVKTNALLFDVSEELLNDYLYVHLVEDTAARLVLSLELLAL
LPFHCTFEVLVSRTGEANEADEAGEANEADEANEVVGESSKGEPPRRRATLERNR
INVEAFNCMNVSRQYLNITVDENLCAEAKLNIFYNHTSDAYFDAYVVRHNQLNSS
SHLKDEIVNFDVKPKCGILKHGSYNTFFITRANRNGVVSFNNSFLFLIKTERNLFSY
IVFSHYRPAPRDALATKEHNKIKEILNENKKRFSVLFSSFKQEKKESSIFNQKNQIII
EDISKSTNEIRNG
>Pknowlesi|PKNH_0510400.1: pep
(SEQ ID NO: 14)
MSREVQNKFDLVDVEPKFLEFLYECTDNVETFLENKKNVIKRKRLKIKNICTKTQ
NIKICEPDSKYLKIKGIKNKKLAPGTSEQIFIEFSFADVNFKTSKFVQTDNILDVVNN
VESTSYVTIRSEYTTLNIPIYIKKSIPVFAYDDVINFGVCNANRAYQFALRVKNVGT
RRGTFTLSLDDSPRECSDECADDQQAECAQSRKNSGKFHLRKYRSEDMMIQLDQS
SLDLDVGETKIINISISSMAEGKMHKEYRVISNQHSYFVEKQRKITIFAIFTSSHTSFL
FNNEKTNLVNLHCLYYGSRKKYQGKIKNENNYAIFCIPQVDAVNVFYSKEDLEAY
AVAKGLDLSRICPDVYLQEEEHLSEGEKKEKEKLFSITKKRLKGEEHIGITFSRVQG
YPIEKLSYQEISFEVYSKCDTDLLHRLSRDTSYLLNFPVVVELSLRVGRSHRETGQ
GDSATKQVHSTTKQVHSGTKHIHSATKQNHSATEQVHSATLEGAHTLDEEVKICL
AFCLTFPCILPSTYLLNYDTITPNEGKTLIIELKNRNKFMKLNYTLSKIPYLTLSKSE
GVISPQGKVVLSLKLQCDSVKMMDDYMYLYFCNNLYFIVLLVRGNIISSNALRKG
SSSILLNLKAEPFRRNETHGKIKQNHDEVYEQILKNLELERVDRNFYQLDGKLDKY
KYNERFINFLKKNKKKYNDVLKLMYEKRKKGEESIQNASPSAASPDTSEQMVKG
KDRLSKILKDKFIYVNDPPVERISNTYINKGVYEQERKKYIQMKTKNLKIKNEERA
QGGEEEEPYLMQFDMLDEEELNKVEFVRMIQYGYIFLGADYTRKFVVFNRNKHC
NVKVALTHGDNITTDGEKILLVGRDSHKMRNVKLNIKCVSREEEVHQDDKNCND
ICLNSGKIGSQTGVSTSFFGEDIITGSCADLHNDFFLLKMFEEKISLMINDSHKREVT
LGATLVFLNVLVNPTTVYFRFHDEDVEMVCERHLVLYNPFNVPLNVGMVCNQA
HIALDSEILVPPNSHKIVPLNFVATESATSVSETIRLYLDGSRLYKSLKCKFIANKCS
YRVTPTELNFENILLNRNYVKEFFLINTGDSPVVFRCNYKPDCVSLLSKYNYVKKN
EKVKISVLLNLKEGKKIKDKIVLTVRGAPQLILPLNVKGTPSNVLICEGIHIQQRRG
ELKCYEVKILNRGSCDETILVDTSSVDFLNVQMGNKKETTFSKCISKEYKDANKM
EDVVTVQRIYSEEYDDLLTDECAKYHYNNFLKLHNSISVLYQNKGQILLKDRGEV
KSMYRVVVPSKCFLPLCIQCYSSKAIRKEIAFQDLLHHNRVTYDITEELIKIDIEEGH
LDVAPSFLHFTDLIPHQADEAYISYFSKEKTKRITIRNVFSQPVQWAIRLDEEKRREI
HVVTNVHRGVPEFTERKGGMGKSKRMDSVKSRGSSKSHQTDEPIWERQYQVYPS
RHRGILQPNEETTVDITLSGFKLCGRYVDVLDISSSIFSDQNAGCIDGGNDASNDAS
NDARNDARNDVSNHASDDARNDASDMLSNLVGEAEPAQKEEAEREVRFSLYMLI
CCDIPKLQFDVTYINIIHNKLNDYCSFPFDIYNHGYNYVRVDYHFKNLHAECFSISL
DFINGNEINEQVKKLRMSLKCKATKSLKFKSHIVFTVMDYHQYTIPLFVNIDEGID
FLLEKAATSSSVMSSIEETGEVIDEVVGEVIGEVVGEVIGEVVGEVIGEVVGEVIGE
VVGEVVGEGIPKQMHHMDDYLPPENSPKGASKGTYPAGKPTEGIIMDSSLQQHM
NTTDIQFDKQNELFSFFCKNMEEVKKGSTNGNNIYKGLKINNIRNVYYNEEEEGV
YPLREIIGDYLFVFIQRIMLRKSYFPQVIESTNDLFLFFLDLLLDLFSSIYPNRNLQNII
VELKKRPTVVVKDMKREDLLDEMFTKNVQTLWKSLKEEEYIPLDHIIYENLLPAD
LYLFHVLDKVPDYFHVEEKHYEDNTENLSIKNFESFLLNGNKKVFYFEKIFKTHVR
LFSYSWVTIFLEVLNRKMFGAINMSSLTNLRGIKLYSIENKLNENIKMYKISYLVIL
DTEDMEVNKHGSFLNGKKGRSRRKEASTLTKLHVNRGGTGTRNVAKSSLNTRNG
NLIYRDKSTSRGQDKYAYNYVKKKTTKDKPSCTINYFNFYENNFIDLKKIVNKISR
FCYSGGVEELPNGKQETVQGKNELESSKGNTNIEGDDIPTGGETNTPFKKKSTKED
VKESTIKYSKDTTKDAPNGIVHFKSAECILFEWLYFHYNNVYHAKVEDKRYIFKE
ARRKSDDELSYTKGNSNETQERGKPEGSRRKGNPSGSPSFVPSDSHHVSASGELVP
SQVHENTKMSTSNFQVEIKRKKKEVQKKKKAKSIHELKDLVMILYTIISHIPYYLFF
KKKIKLNCKSKRDYSQNIDVLLIMLSEMNLSNLISRQVIVQFNYMHIYLFLASLYFI
LPKYLPCYYLVLDDLPAYKVDLSWINDEVVNSKKGGKSKPMGKAQQSSRGKQA
PQKKTNAPIDGENEKTSCGDDQTMADERIITVYNLNSHICKYEVFLIGCNKYQIDR
NYVSIDPLKSEQIKLKIDKGYDEKVLKYSNHTSIKLPYFKKKRHKGDMCDGEEPH
FEDNYSVSSCSSEDCQCPSDGADKVDAGIDEDSRVSQGENYTILVLRSESNRLQDE
KDSEKYICIKLVELGEKGKIELAPPGTNIRGEKNEGNNVNANVNNGGTRSEVNKV
RTTNSSHLNIMLNDSETKCFNLRGKVYENKCIELNLFNNTGKEVEVNSNLYHLYV
KNLKKEEKGKENFETDVVNKWGDDHLGGHNQCVVCSIVNANLRNHLSDTSTHF
SCVHLVDKNRFTCLQNERKKIQVQFCPFAEGSYFCFLLFNVNDKKTNYIVSACKV
NCSFNINNVKEEEIIKMNSKIFNFSYHLKVCPVNREFLKCVKFILCNLNHLDERHFL
SYLRGYLEDIHKGRKFFSVLCDNEDKISIEKGFATFGPLDCNKYMEKILYSEGVDI
DQLYELVKKETNYTCTLEIAEKTNGVFEYNCALVSHRNVLQGKSKLSYEQMRKL
HIKEQKEICDPFYKVYKIIASVNDGNSSQSLSITFKTSAFQMVKKSIPVYNYTNGVV
TYRRVYSDVIDENNKIVGYNIFSCPEYIEIGKEVESVNFEVSCYSIIGLTCSCCITLYN
VNNEEEQIKINVQANVMKPYPKDTIHLKLLNRTRKTAQIEIYNDLNFHCEFKVYSD
LPILFGVKKIEMMPKQKKTYTFFVTSAHIGEYMGCIIFKHYKLLRSGHGEEKKGDS
FFPFANYFFWYKLNITVELNKPLKILFLETNVGEEVKKDIVLKNNGNENENYFLLA
YMNEYIERRQVQIPKNDIYVYSIKYAPKIPNYFNEDGNGESASGENNDPTLQRDIQ
NFYYPHNGDSDWCGPPGGDEPAHGGKEPWELSPNQGATLKHKHKPFPPTEKKIFE
DLISYCNKYIQVDIAYRPHNIGFFFIYNKNEGINYYILMLLSRMKSHLDVRNFSTCL
SKSCFLHLHFQFNEEDERYVHLLEGMETPPSSNYHYQIVEGVKCPPGEKHILIEENF
RGAVDMCRAYEEYDDKWGEEMQKGNANNYMDAPIYKDNPYNGGARDQVKHE
LKCIYLSNRNNFHIVKHKDVNRVVLKYRPTVNTSQECYAIIRSNLHGDFLYRIHGK
YTVKRKIKEKLVLYNSCCLYNFNVNLFNPFNCKVRVKCRFKGGNATQLCNHMCG
AQGMKEKDIDEKYMKSFQKERVVLNKERYYLKILNKENFKIKMRKHFTIMMTYF
CKAVYSRRTVLIMMKPFRRDRTHVDGPPMIIYEYDFVFNNFLRSRRIQNVLQCIPA
VGEIKDEQGRGNCDQKQPSTECEDLGVNTSCYFHSSVDEPESGNQASGNFTMRHS
ERGDVEANLSEVSDVEAHQSEQSESIHTSRSSSVASERSHFTLNASELGVMDVAEG
DFKLEDADTIEIGKNHTCFTHPDDKALLSPNDYDVEHEIKHERVLPCASDNKEVV
HYDGKIFIESMCKTKTYVEVHIDKTKMGTWASCPQGMPQGMDQLEMQNNSLHR
NVEMGNYNYKIFLLNLKNVRKNMDTVDETVVGGTGGAIGMDVKMNTLLFDVN
EGLLNDYLYVHIVEDTEARLVLTLELLAFLPFHCSFDVFVSRRGGEGATHHGESG
KADQAAATNKAEATNQAAATGQSVEEAYNVVEEFCGGKPRHRCTTFEQNHISVE
ILNCMNVSRQYLNITVGDNLCGETRLNIFYNHTRDSYFDAYMVKHNQLNNSSHW
KDEILNFEVKPKCGILKHGSYNKFFITRTNKNGLLTFNNSFLLLIKTERNVFSYMVF
SHYRAAPSDALATEEHNKIKEILNENKKKFSVLFSSFRKEKKESSIFNQKNQIIIEDIS
KSTNEIRNA
>Pmalariae|PmUG01_05035500.1: pep
(SEQ ID NO: 15)
MNEEVTNKFDLIDVEPKSLEFFYDCAENVDVFVKNKNNIIEKKVLTIKNICTKTQN
IKIFESDSIYLKIKGLKKKKLAPGTSEKILVQFSFCNFDTKKCKYYSGNSNSNSGDN
NGNSKSNNNNKTGLLRKLNNIECTVYVKVQSEYSTLHIPIYIKKKIPLFVFNNIINF
GVCKNNMTYHFSLQVRNEGTKKGTFTICKENFIEEGNEQSRNNEIDGGKHALVRF
NGNIETKEKEDKIIKKEMSEKKNNLIIDFDKTSIDLDINQSEIINIKIINTKEEKIQRTY
KVLTQEHSYFCEKPKNIIIIAIFVNSQTSFIFDNVKTNQINLNYMYYGNNRKFQGRL
KNENNYPIFCKYEICKVNVFYSIQNFQKYADDNNLDVGLLMHDLNDKAENELSEE
EKREKEKLFTIAKKRLKAEDNITVKLDKEEGYPVQKLGYHEILFELQSKCDINFLE
KINRNTSYLLNFPAVLELNLHIRKVDEDKKKGNDDTPNRDGYTDMHSSTNRGDE
EIKLFFIFCLTFPCFASSSYFLNYETVTLNEGKTLIIELMNKNKFLKINYCISKIPYLT
LNKKEGCIVPLGKDIISLKLKCDTIKNIDEYMYIFFCNNLYFFVLLIYAQVKSVFAS
RHVSSTILINKKRTNYNGDVVDSKFSSSSTKKKNDEIYEQIIKNLELERVDRNLYLE
DGKLDKYKYNESFMNFLKDNKSKYNDILKYMYKKRKKRGKINNNNNNDNNND
NNNDNNNDNNNDNNNDDKVVHDKDYKREKRAEERINKIVKDLNIYLNDTGMGR
MDEKSKMSKTNRTYKTGKACKISSTCIQHAVYEERRKKYMNMKKKIFKEKDIEE
MSKKDADLFFHEILEQHQLNNVNFPKIIHFDNVLLNNNYQKQFVITNSNKDCSVKI
NFIHSDNIELNKDILILSKNSDKHVNINFKLLSHTNLINRYRNYQNDSIFKQSSEIFN
EQLNEKTSPQNEEIKCVLNEKGNNSTYDESNKIFSNSINDFFLEQTYKEMIQLKINN
AYTEFIIIKANLVYLNVLLVSDVLYFRFQGTDLGMIQENKLVMYNPFNTPICVRTT
CNEQLFKVDAEFTIPRNAYKTVPVKFIAPQNASNVEEFIQIYLDGSRLYKSVKCECF
VNKVFYKVNTTEINFEHILLNKNYEKDFYLTNTSDSLLSFECVYKPDCVSIISKYNF
VNKNEKSKITVCINLKESEKIKDKIIFRIRGSENLVIPITAKPSPSNVLLCNNVQVAQ
ESNEIKNYEINILNKGLCEESIILDLSELSFLNIYTNNKGQKKLMMSNNKEYKNADE
MIDSQMILSKISKLEHEDILTDECTKYYYNQFIKLYNFISTYYKHKGEIRFSEKGEK
GVPLPKMYRINIPPKSFASLYFQCYYNKCLKKEIAFNGLLRYNGKNDNSNNDNSN
NGNSYHTAYGMKEEEVKIEIRASCLEVEPYFLYFEDMLPSSSDKALITYFTKEKTKI
LKIRNASSQVVRWEIDICNEKEKQNYLIGIRGGDCRERVEKGTVHSTLEEEAQKKG
RKDIPKKGRSEWGHMGQNGEKTVQALKVERGTESKVGKRNERGEIGEKGGVGD
TGYIFDKEKTDNTGKQNYWQKYEIELGKKEGTLEPKGEVEIQVKLKNFTQSGRYV
DILSISYDPVEDHIERSSNEKEGGSKEIIISNYKMELKKINYCIFMLLSCKIPRLLFDV
TYINIIHNKLNEYITFPFHIYNSGYNYVRVNYYLNNLYEEYFSISLNFTNGNEINEKI
KKVDVCLKCMAIRNLKFKTYIIITVLDHDQYVIPLFVNIDENIDYLFDKLDQENEH
LHVNHSPSMPALHNMHIQCGKKNLSELEKIKDNPIGYRNNDQGIEDRDDGYGDT
QHNINHCSTYRDDRDKQSAKDMSHTRRSNKEKTNGGECVYNSEYESTSCTCSSV
CCDDNMLRCNCQERNSKFLSFYSKNAGQRDIRKSVCIYKELKIKHITNVYYNKEK
YLLNISEITKDYVFIFLQNILLSKYYAPSVIENTNDLFLFFINLLYELFLLYPNKNIQN
IVNIINDVKSYKNISMQDLEKNNFLQEKFLKNMKALLKYLEEENLLVYHIIYENLL
PLDLYLWYIFDKVPDYFYLKKGKGGEEEINKRKCIQIEDVENFLTNGNNKIFYLDR
IFKTHLKLFSYSWITILFELLNKKLFSCVNVTSLNKLRGIKVCNIENKLNENIKIYKI
NYLIVLNKQDKEPNKHTIFLSEKRKTSKRKENSSSTNITPKDNNPYEDKSAKVSSVI
NMGKNKNNYLSNFYMKNGRPTHHNSIIKKKKMNNGKNTYTINYFNFYENNFIDL
KTVVNKINNFTHCEEFEQGVEKERDISLEKHEQIINRDDKETTQRNIRNKTYSKGE
EQEKEEKDNVQFKRDTVVKEQKPCFHYFKSAEYILFEWLYFHYNNINFANVEYK
RYLFKEDNEKIKKQQQKQNDLIFPNVPMNKSKEEEETSSKCMENNDVPLILNRSN
FYVEKKKKKIMEKKDKITSICDLKDLVIIIYTIISHIPYYLFFKNKIKTKCISKKDYIY
NIDILLIILHEMKLDKLISRQVLIEFNYIHIFLFLASMYFILPSYLPSYYLVVDDFASY
TNCVGLIKEEMISNNKNKKKNTCNQNENEKAGIIKLNLTTPEERVITIYNLNNYKC
KYNVFLIGCNKYQIDRDHIVIDPLKSEEIKLKIDKNYDQTICKYNNNTSIQFSTFKN
RRKKKETIDETTTYFDESYSFSSCSSDDCKCNSDEAEKISVSNNGDDSLTPNENYAI
LVLRAENNYFPDDNDDEKCICIKLIEKDTHEKSDLQASNLKKEKNEWNDLNINVK
NSETRNNNISKVHLSNSSQLNVMLNNSEVKCLNLRGKVYENKCLEINLINNTGRN
VEINSNIYHLYLKDLKKEEKKKKIFEIDIINNLNISNDQIYKVCDVCTIINSNLRNHL
NRKENYFSCFYVDNTIPIIISRENEKKKKKIQLHFFPFMEGYYFCFILFYVKDVKTK
YLISMCKVNCFFYINNIKEEEIIKMNSQLYDFSFNIKLNPINKEFLKCVKYILCNLNK
MDEKFFLIYLKSYLQNVSKNRSSFYITCDRKDKMIIKKNFVSFENFDYNNYISHINT
PNLDIDRLYDIIKEGINYLCELSIKEESNGVFEYNCVLLMRKGEAEKQNKKSHKHT
QQKEGLKTEKDADCNMLSYEQIGHSHLREQIGNHEICYKLYKIIANVNNKKNNNI
NSIITFNTNAYLETKKTISIYNYTNSTITYKCTYSDTVDHNNNKINYKIFSSNEYVEI
PKDVETFNFEVTCYSIVEVSCSCFIIFTNIKDEEDQIKIKLQANIKKPYPKETIHIKLL
NRMKKTVQIELYNELQFHCEFKIYSDLFILFGDKTIEMLPKQRKVYTFCVRSAHIG
EFVGCLIFKFYRLIGAERGDLHRSNVIVSNISNVSSESRSNVSGESRSNVSGDDTRE
HLFYYFFWYKLNITIELNKPIKILFLETSVGDKVNKEIVLKNNGNESEDYFLLTYM
NEYIEKKQVKIQKNGTYVHNIKYMPKIPNYFHNIKEASDNFKLSTKRNKSFHYCSV
DQLDKEVEGTNPLQSGYNDFPSGKNRDCTYKGGKEMDLAKELHKFYFPPNEEKE
EEKKEVEAKWEEEKREEVNDDEDEAKSQGEDEGKGQNSHLAEEDKKETAKKCEI
RNRSRSRSRNRSRNRNRSRSRSRNRSRNRSRSRNRSRSRNRSRSRSRSRSRNRSRSR
NSSRSRSTSTSINVLRGVRSSSGKDPQRKSPSRDHSRRKNKSDMLRKKCKKNVER
DNKKIFEELINYCKRYINTNINYKNQNIGFFFIYNKNEGINYYILILLAKMRRELVID
NFSACLTKYCLLNLHINFNNENKRYVKDLNTNKGTCYSDNYSYEIVECVNKNGG
WEAESELAGKCKDTQPDVSVNKHVNRGKIYGDTYDSTISRNNQINIDDHKYDNA
NVEEIINSQIKCIYLSNKTNYSIFKHTDKNNVIIKYKPTVKTSQECYAIIRSNLYGDFI
YLIKGMYTVKRKIKEKIVMYNCCCLYHFNINLFNPLNCSIYVKCRFKREKSKNDEL
YDNFYLDNNKVGRKAEREECCFSSMEHEKILLNKERSYFKILSNEHFKIKKRTHFS
MLISYFCKEALSKHTLIVTLKPFGTDKASDGTLPPIIYKYDITFNNVVRQGIEAYSLT
GSGAQSTDELTARGDSSSGSSGRSRSGDGSSDRSSSSSPSRRNRHACESGGTCEKT
KSYSVTSQSSSNMLNGSELSKSDLFQRGLKKKYNSKANELVEEQEICLHACDEILV
EDKEEKDKYERNMSYVVDRKEEVVYCDGKIFIESISKKKTCLKINIDKRKIMERAD
YNNIINELENCKKEKSILHRNIEKDKCNYKIFLINLKNVRDATMDEQNKTNLHMN
KILFDIREQLLNDYLYVHIIEDTASALVLSLELFAHLPFHCSFFIMIKRVELGSREVG
SDEVERGEMNSEEEDNYVLVESLKSEKRKELTTIEKNYIFVEIFNYINLSKKYINVI
VDDNLCSETKLNIFYNHTNDSYFDAYIVSYNQQSNDPFADEIMNFDIKPKSGILKH
SSYNKFVITRSNKNNIITFNSSFLLLIKTEKHIFSYIVFARYTPASDLHTAFSLYFQQE
QNKVKEMLNENKKKFSAVFNTFKQEKKESSIFNKKGQIIIEDISKSTNEILNC
>POVALE|POCGH_.: PEP
(SEQ ID NO: 16)
MNEATNKFDLIDVEPKFIEFFYDNVEDVDTFVQSKNNVIEKKILKIKNACTKTQNV
NIFESESRYLKIKGVKKKKLAPGTSEKILVEFSFSNLDVKKYRFGKKKDILNILNNV
ECTVSLKIQSEYTTVHIPIYIKKKVPIFIFDNIINLGICKSNRTYHYSLKVKNGGSKRG
KFTISMDNIVEEEKTEQTHDYKRDEKQTKVHFNGDINKRKKEEIERKDVHTSKND
LFVQFDKTTVDLNINETATINITIKNKREEKIYREYKVYTKEHSYFSEKTKNVIIIAIF
TNSQTSFIFENEKRNQINLNYMYLGHKKKFQGQIKNENNYPVFCTHKVGKVNAFF
SMQEFEAYSEENGLDIDQLSRGEDDNNEGKFVGTFVTESPCSSTAYSFSYTQNTFT
SLTGSRDFLDTTVLWTSALLIYFLFYFFFLSSFPIHILADMRGVTQNEMCEEERKEK
EKLFAIAKKRLKQEENITVKLSKEDGYTVEKLSYHDVSVEVQSKCDVRFLEKVNR
NMSYLLNFPVVVELMLYVCSGESGVSGSGGIKCCHESCDNRNLLDEEIKLFLVFC
LTFPCVVSNTYFLNYETVSLNEGKTLIIEVTNRNKFLQVSYCFSKIPYLTLNRKEGC
IDPMCKEIITLKLKCDTIKKIDEYIYVFFCNNLYFFAMLMTAEVKSAYALRQASSL
HLEQGKGEKGLTNRGSGDRDLGNRQDDEIFEKIIKKLDLEKVDKNLYLVDGKLD
KYKYNENFIAFLKNNKKKYNDILKYMYEKRKGRRKDKTNMDMKEKEEECMEK
KKLKKKISKVINDIWVYINDTGLTKISTTCIQHSVYEERRKKYMHIKREHLEIKDTN
SASNECMNVPCDEVLEEHQLDKVIFPKNIHFNHVLLNISYKKEFTVHNSNTDCNIR
LDFTHSDNVKIDNDMLIVGRNSNKNINLELKISPFFKKVENMTNEKGTTTCLFPTP
SLQIERENNMLFNNPEIVMIPSVNDFFLQQIYTERIPLKLNNIYEQQITVKANLILLN
VLIKEQTLHFYFENSDVQMCCERQIILYNPFDFPVAVSVACNESLFQIDAHITIHPNS
CKMVPVKFLAPPRAGVLEDFIYIYLDGARLVQCMCFVNKCSYKTDITEINFENIILN
KHYTKDFYLTNTADYPLIFECSHKPECVYVQYKYSYVQKNERLKVTVSVNLKDG
KKVKDKIVFSVRGAENLVIPISAKAVPTHVLFMRGLHIKQESCEMMDYKMDILNK
GICEEKVILDLTELNFLSISISIREKKEKKTLTYNSKEYKHAEEKRDDFPILREIFHVE
YEDIITEESTKWYYNKFIGLYNFICNYCRSRGSIQFTERGEKSSQCLYMVNIPPKSF
VSMYINCYYDKTIKKKMKFNSLLLQNKIIYEKKEEEIKVEIDSTCLDISPAFIHFTNV
LPSNSHRSLISYFTKEKKKTLKIKNVSDYPIEWKIYVYTNEQKKVHLMRRTGGKA
RATATHEGNQTARGVFTGEKAAREIATKQITAKHITAKQITAKQITAKQIAEKQSV
AMGVSNRDIETKGDLVMGRDEVEEVAKAYELDVSKQSGLLKPEEGTEIEVKIKN
VLQGGRYVEILGISATFVDNEMGKKTDSNVGDTEIEVLKSTVRREEKTYCVYMLL
TCETPKLFFDVTYINIIHNKLNEWFTFPFHIYNSGYDYVRVKYHFNNLHEDYFFLSL
DFEKGGEINEKVKKINVSLKCKAYKNVKFKSNLSVSVLEHDQYNIPLFVNIDENID
YLLDKAGGVEEVSFQRGNKNGEKTQKGDIHVGRKMLDNIDEREDYRYDNPWKE
QLDDTTPFEGCNTESNISKQTKDTLLPFFCSNSVEKDEEEIVNIYKGLQIRNITNVY
YNKENFTHNLTAILNDYIFIFIGRIVLNKSYLPYVIENTNDLFLFFLNLLNDLFLLYP
NRSIQKLINGIKFYENIAMTNFEKNTLLHDSILQNVKGILKHLKEEKLLVDHILYEN
LLPVDVYMYYIFNKVPEHFYVKGKKEELGKNDNGDSNEYICNVDVNQFLLNGGE
GVFFFDRVFKTHLKLFSYSWITIFLEFLNKNMCSCVNVNSLSKLRGIKLCSIENEIN
ENIKMIKINYLIVLNKQDRELNKHTAFFDERKRRSRRSEGTSSGTICSGNGNASSSG
RGKGMQRMPVGVIGMEKGKKTMPIKEAKGDPRNGKNAHHGKRMKVGLIKNSS
AINYFNFYENNFIDLKKVVQKINSLNCEGGDAEGKKRDDQLTLEGSRKERDTNSP
KGSSPMEELREGRNNGILKREVEKEQTRNFHQIKGVECILFEWLYFHYNNVHYSD
VQNERYVFKESWNENEKGVADLGKLPSSRKVVNVAEVRREEVEEEVTPQHEVNE
VVNQHENSEGRPVKLNKSDFYMEMGKKKKRKHFSEKKDKITSVHELKDLVMIIY
TLISHIPYYLFFKNKIKVHCKSERDYINNVDILLVILNELKLNNLISRKVLIEFNYIH
MFIFLTSLYFILPNYLPSYYLLVDDSSSYKNDICAINEELIREKKTKNVRVQSENKKI
GIPKSVSKVEERVITIYNLNSYKCKYNVFLIGCNKYHVDKNFIHIDPLKSEEIKIKID
KNYEEAIFKYDYSTSIKFPPLKNGFPSDAPRFDDSEGTDASTASSHMGRSSSTSCLS
DESHEEDAIMGKKTKHRSFTKNENYAVIVLRSECYLPEERDSERYICIRLVEGSGE
EETVILANKGNLKDKKNEGSNLNVDMRVSGGRNDVSRVRPTNSSQINIMLSSSEV
KCFNLRGKVYERKNLEINLINNTEKRVEVNSNVYHLYIRDLKKEEKKKDNFEMD
MLTVDKGTHEMYNQCVVCSMINTNLRNYLNEHEHFFSCFYVENGKDLTIGKNER
RKIQLHFCPFVEGNYFGFLLFCVKDRRTKYLLSTCKVNCFFYINNVKEDEVIKMNT
LSYNFTYDMKLNPINKEFLKCVKFVLCTLNKMDKKYFMAYLKSYLKSVNQSRHN
FYIICDKYDKVAIKKSLFSFQNLDYARYVSGVDAGSSGDVDVDELCDAVKRDTNC
FCNLNVREESGGVYEYNCILLVKKNHEEKKKDISNFVTYEDVRNMSVREQNCIHE
MCYRLYKIIANVDSEKANKVFTIVFNTSAYLLSKKSIPIYNYTNEKLTYRCIHSEIID
QHNNKIDYKIFKNDEYVEIPKDIETFNFEVICYSIVEVVCSCIILMTNVENEEDQIKV
NVSANIKKPYPKETIHIKLLNRMKKTVQMELYNELDFHCEFKVYSDLPILFGDKKI
EMLPKQRKVYTFFVRSAHIGEFVGCLIFKFFNLVNMKGTHASTRYSSNHHDSDRP
NSTVDSFPFSDYFFWYKLNITVELNQPLKILLLETTVGDEISKEIVLKNNGREKEKY
FLLTYMHEFTERTQIEIPKNDIHVYNIKYAPKIPNYFDNLRDTMECYKLDSEQEKID
PFCGTTISNSLGREKERVSSTEGDAPSFTGGGFDIAQELSKFYFPPNEESEEEMLVES
AVTRESSSGRAVIGNVANGEEANGEEANGEEANGETVRGEKDEYAASRVKNLYL
RSETMKNGGEQPKKTASMGVKVRSMEKKEHPILRQNVKHKGKKNFPHNEKRLF
EELIQYCKRYINVSINYTNQNIGFLFIYNKKEGVNYYILILLAKLRDQLTISNFSACL
SKSCFIHLHFDFNSEEKRYVSMLSSKENAHSNEYFYQVGEYVRQRGGSDAGKDK
HKVKCIYLSNRTNYTIGGIADGGVDVDVGGLVCEREDEHTDEPGNHVVIRYKPTT
NSSKGCYALVRSDIYGDFLYFVKGVYTVKRKIKEKMIMYNSGCLYHFNVNLFNPF
NCKVCIKCRFKRRRDERGGGSHIISDSSEHEAEKRMKERSVHSVSVKKEKVLINNE
KKYFKILSRENFIVNKRRHFSLLMTYFCNKITSVQTLEIVLKPLQDITNRGSFQSIAY
QYKFVFKNTQKGSLIGEKHLVCDVPNDEVTYHNRFDENSLTDSSTGGEEPYPCNV
VKKVDYNHVVYTKLNEVFTTEEGEADREEEKHSWRMISTSKWEENMEMHTNEG
IASPGCGSTTTDSECTQDGDLVSTPVKKTPFGVCEDRKDVMEEQVEQENCGLSSK
EVVYYDGKLFIESICKTKTQMLIYINKKKMMEREDYDKFIDELENRKREKNMLHR
NVEKGKYNYKIFLINLKNLNSNWGGLNGKTCVKMNNILFDMNEELLNNYLYMHI
VKDTPTDLVLSLELLAYLPFHCSFCVVVKRVEGRGGAAMEEAEEVRETGEAGET
GEADGIDWEDGIYADAGERCVQVECLKREETKKFTIVEKNYISVEIFNYMNVSEK
YINVFVDRNLCSETKLNIFYRHTNDSYFEAYMVSYNQMSNYILTEEIKNFEIKPQS
GILKHGSHNMFLITRINKNNLVSFSNSFLLLIKTERNIFSYIIFSRYSPPRGLRTTREQ
NMLKEILNENKKKFSVVFNTFRQEKKESAIFNKKDQIIIEDISKSTSEMRNY
>Pfalciparum|PF3D7_0418000.1: pep
(SEQ ID NO: 17)
MNEISENNLNILDVSPRNVEFFYECLEDVDEFAQKKKENIIEKKSLKIKNICSKIQNI
EIFETEYKYLKIKGSKKKKLAPGTCEHICIEFSLYNNVDIKKYKNDKRKEDVLNIIN
NIERTIFLKIKTEYTSLSIPIYIKKKVAVFFYDNIINFGLCKQNMTYHYPMKVTNVG
KKKGTFTISSDMYQSMKEKGNHIFFQDEKGERKKKEFCMENNKGDHQNEDNHDI
KSNHNIKSNHNIKSNHNIKNDDNIKNDHHIKNNHHIKNNHNIKNNHNIKNNHNIK
NNHNIKNNMKEKNLLVDEKKDNTLITFDKTSITLDINESKIINIEIKNLKEEKVHRE
YNILTLENSYFVEKPRNIIIIAVFINSSTSFYYDNIKTKEINMNYIYYGNKKKIQGQIK
NENNYNISYQYKMKKVHAFYTLDSFKKYIQLNNLSAGSLEEELCEMEKLLGQEE
KEDKEKIMSMAKKMMNIEKNIHVKMNNDIVCNVKKLSYESVFFEIDSKHDLLFLE
KINKNKSYLLNIPIIIDITLFVNKSDDEKDKTNKITTGGISKMHNDNNMKIINKRKNI
NNNYDNNCGNNCDDNYDNNCDDNHDNNCDNNCDNNCDNNCDNNYDNNHDN
NHDNNHTNNQHNNNICCTNLLQLHKREEKDRDSSIHHASNCYEEEINLHFIFCLTL
PCLITNIYFLNYNKVTTEETKSMIIELLNKNKFLKINYSISKIPYITINKKEGKINSQE
KDIITITLKCNVIKKINEYMYIFFCNNLYFFVIFINGEVTQISNINDDNMLTIKNIKNI
YDNNMCDEKGITNNDNTYILNNKSLLKKKKEKRYTDDHNNNNNDHDDKIFERIL
KNVECNKVNRHLYEQDEQVDKYKYNEHFINYLKNNKKKYNDVLKYMYKKRHN
SKHNILLKDNIKNKESMIIKKNSSYNLKFSEDNNINKEYNIDHKRTNVLKDIYIYVN
EEYINKITNIYIPQHFYEEKRKQYINMKKKNNKQYLVGTMKKEKYLNQSLCLKKN
NKINNINIINNDDNFQNVQIIKQYLIYPQIIHFNYILLNKEFETFIHLHNTNSIHPINIYF
FFHSDNIKITYPEETKTTCHNKINDSNICTPYQSTKVTIKQNTQQKICVTLNLKNYNI
FNNQKNKKCSFSQSENMDNEEEQKICNHIISKRNIKNKEHHNYLDDTYKDEPYNI
YHHKTTPFFEIYIYYEYISIKTQDHYDIDKIKVQANLILLNLFINTNILYFSITNMDID
MFYHNYLVIYNPFNITIQMKLKYNEQVLKMKDHISIYPGEYKMVPVQFITSEATAC
VEEFIYVYLDDFRLYKSIKCKCFPSPCSYKINVNEINFDNVLLNRNYLKSFYITNTS
NFPLVFMCVYKPPYVHVLAKRNYANKNEKIKITILINVKEEIKIKDKLIFYVRQREN
LILPISVKLTQSNIVIVNCPDIIQESMERKRYNINILNKGMYEESVIINFNEIPFLNINIE
DEDTGNKLNYINYKNKEYKHSEEKKRNTFVNIYNLEYDNNIIIDEYIKYYYNEFIL
LYNNLCNNNYKIKGILHISSDQKCLQNCYKIIIPHNTFITLMIECYINKIYETDLYLY
QHVLLNLNNPFVKDEYYKDKLKINIKNEYLKFTPSYLYFENILLCSSEKYLITSFQK
TITKKIQIINVYHQNLKCTVKICPYNKEQTKTSQPVKVSSNNNNNNNNNNSIIPSDH
DMICTHSTWYENNHTKKKQHYIDISNVPDILKVNEESYINIKVDNIEKEGRYVEML
EICAESVNKKENEEKIKYSLYILINCKDVKLYFDVSYINIIHNKLNQYNYFSFHIYN
DGYNYVRANYLFNNIFKEHFFLDLQFLPNNQLNKKNKKIKVLLKYLSKINIQLKTF
ITITVMDHEKYDIPLFINIHEEIDYLMEKENKHKQNYTHCHNYTHGHNYTHGHNY
LHETDELYEKHNGNENSDDIKHPYPYDREIKSEGSTNKSHILSSHLSGDHKDEIPIIN
NNKKKNISYFTKEEEFALYNNMSIPYSLKYKQNKKMDVDENQNMLSFYRKNMEE
SSNIYTNIFKNLQIDNIRNPYYNTNEEGIINLKEIVHDYMFIFLQHIIINKNYFPQIIEN
TNDLYVFFLNILYDLSFIFNSNKNIQNIINELKAYEHMQNGSVDENEFIKNMKTLTE
LLNQEKLLINHIMYENLLPLHLYLHHIFNNIPEYFYIKKESDKKEKCKEINNLDNTI
LYDNKNDIDEEEYNKEKNKFLNISDIQSFLKNDNYKGDNNIFYLDNIFKTYVKLFI
YSWITIFLDIIFKKIFSFINITSLYNSRGTKLCTLENNIYENINIHKINYLIVLNIENKKI
NTYTYSCTNEKTQKCIVKKKCIDEKKKENANINVKELSFNNHKNNEDLNIQEKAY
FHFYQNNFIDIKEVLHKIGQSTDTDHSKKNQEIKETKEIKVTKEIKVTKEIKVTKNK
DSYHTNNLYNNTNNFKNTVKEKKQINKTLKKETHKILNQKKKKKNTNIDEDKKK
TTESSTYTEKYEHNKHLNIKVQTNINYYRKIEVILYEWLYFHYNNVYNTKVKKQK
FIFTQQKKDISKHNKLYLQYDQNKRNSEIEHTNHKEDYSMCDNVITSKMSHISNM
DNQYGNNITCDIPFFVEKKTNKKKKTKKKKKKKKKKNIFKKKKEFITSLYELNDF
VIIIYTIISHIPYFLFFKNKIKDPCKCQKDYLYNIDILLLILNTIKLNNLISRNVLIQFNY
VHIFIFLSSLYIILPNYIPNYYLILDDFSSYKNDVRVIKGNLQKNTRTNKLKQLHMN
NKKKNNNNKNTSDNHVHNNYNQEKQTKKNITCDEKINERIIYIYNSNKYMCSYDI
FLIGSNKYTIDKNHITIHSMKHEEITISIDPHYDDHIIKYDCNNEIYLKDINKIKYKKK
DKSSEHTNSENRFSDSSKNYSSLSSSLSSSLSSSLSSSLSSSLSSSLSSSLSSSLSSSLSS
SNSYIHSRCDSSNIPKNIDTNKTNSKHIKHIHSDKSNNYSILVLRESNYNLLDLKTA
EEKFICIKIIEGNNEDSEKSLLGRNKNNIKNKDNEYNSVYDKTKERQIKGDIHKNN
NSSDCGEMNIILNDSEVKYINFKGMVYEKKNIQINMINNFDRKVEVTTNVYHIYLN
NRKKEIKKKENFDKDIYNLKHTKVEENNIHNNNNNNNNNSSIPFNANYDMYYEC
DACTNINNHLKAYINKDDINFQCVSLDKEKNIYIGKDNGNNKINFHFSPFFQGTYY
CFLLFYVLCTKTKLLLSTCKINCVFYINYIKPEEVIKINYKSYNFTYDLILRPLNKQF
LKSIYYILCKLNNSSNNVFFSIYIKRYLDNIYMNKENFFILCDSIKKVKIKDKKISFN
NFNYTKYFKDDIINKNIDIDLLCDMIKRDVTYSCKLEINEEMNGVFEYNFYLLKGK
QYNVENCLFMIKDGGENYVNDQVSLYINGNKNVNNMFSPPKNNNVNSMNNNND
DDNDDNNDDNNDDNNNNNHNNNNHNNNNHNNNHNNNNNNHNNNYYYYNSC
DGDELVPLCKPKGPDNYHYCDVKLYKIICNVNDENNIRTININIKAGIVYKKYIPLY
NYTNNNITYKCAFSEIINDKNMIINYNIFTCDELVKLKKDMEIFNFEVTCFCPVGGI
KYNSFFILTNVLNEQDKIKIKIQGYISKPLPRENIQINVLNKIKKNIQIEIFNELDIPCE
FKIYSDLFILFGEKKKIEMMPKQKKLYTFFIKSAHIGEFIGCLIFKFYKYKDTNNEN
NASFFSDYFFWYRLNIVVQLNEPMKILYLETFIGQEITKDIIIKNNANQKEEYFLLL
YINEYIEKKQIQIEKNDIYIYKIIYLPYIPNYFYNVETSSCSHSKTLTGEENNNYSNEF
HESKQLFMDKGEYASDHFIHNEDEKNEKMKNYSLKNYIGDIINSQSNNHVQHNN
HIINSLHNFYYSKGEYNNMPQCLHKSKNKDTFTQEYEENNVNTSDLLFEHKQKKN
FQSKEKQIFEELINYCKNKYININDINYDNHNIGFFFIYNKKQGINYYILILLSKFKN
KLIINNFYTSLSKACYINIHFHFQHEQERQVKQLQEKDKQKKLHSFSYEIIEKMDK
MDKMEKIEKGENKNKNKNNIYSASYDICSNNDQPFSSSTHDVYKNHLKNSLLDSY
GQNHSSNIKCIYLSNKINYSILQNEDKSSNKIVIKYKPTMETSQDCYIIIRNNLYGDF
IYFMKGMYVVKKKIKEQMVLYNSSSLYRFAINLFNPFSCNVCVKCGLKENKITNK
NNKKIIKNIKYMKNIKNHNVHNNNTNTVNKKNKHKSSRFKGNQLLLNNKKNYFL
IINNENFIIKDKKDFPLLIISFCKYEPSHHKTLIVKLQPIYNKNMNNKQIPYIIYEYSL
VFNKTNQQKNQIKSNTLNHFVLSDLYKNESLTHEYQTTENEENENDVEECQVNIH
ESNENENDMCESGENENDMCESGENENNICESGENENNICETAENESISMYESGD
KYTTDSQKSDTANELSIGELRREYTDKMSMSQNGNDDKSKYDYSYDDDCYLING
NDIIYYDKKIFVESICKKKTCVRIYIDKKKMRESQNYDSLMDDLEICKKKKCMLHR
NVEKDEWNYKIYLIKLTNVYNNNNNNNNSDDIVNKRMITMNNILFEMNDEMLN
DYIYVNVIEDTEEGLLIDIELLAYLPFHCNFFLMIKKKMKEKINGDKKGINILSETC
NGKDINILPEYKDNNKYIINGVEKNCIFVEIFNYLRICSKYINIFVDKNLCSEKKLNI
YYKNNSDSYFNAYMINYNEYSTDDVMDEILNYDIKPSSGVLKHNKYNKFIIIRTNK
NGVVNMNNIFLLLIKTERKIFSYIIFSHYKHTRTICTMDEYNKIYDIMNENKKKFSE
VFNTFKEEKKESVVFNERNQIIIEDIQKSTNEIQNI
>Pyoelii|PY17X_0720100.1: pep
(SEQ ID NO: 18)
MGLDAEKNKFDLIDVEPKSIEFYYDRIESFEEFVEKKKKVIEKKVIKIKNICTKIQNI
KIFESESKYLKIKGIKKKKLAPGTYEKILVEFSFCDVNINKYKYEKIKNILDVINNIE
GTINVKVQSEYTTLYIPIYIKKKVPIFKFNNVFNLGLCKTNLIYDVKLEVKNVGNK
KGTLSICLDNINNEAAKNEAVNDDNKNSNTLSNSDNKHIIENNFFIYFDKNTICLNP
NESTIINIKIKNKNEEKIQKSYKIITQEHSYFSQSPKNLTMIAIFINSQVSFIYENMKT
NQIDLNYIYFGNSKRINGQIKNENQYPVICKLKKTQIKMFYDSKEFASHAVDRGVD
VRTLLRDLDNFPEFDVNDEEKKKSEENILDVGNNLKLEEKIKIKLEKQNDIYFIDK
QSCTDILLYIEIESCIDILNKIDKNYSYMLNYPVMAEITITANKCDAKCKPSTNRKK
AITDLRQFDKLRNGANTVNGANSVNGANSVNGANGESVECSEGCEEDLTFYVFF
CLTFPSIVSNIYFLNYDMVGQNEEKTLILELNNLNKFLKINYNFSKISHIHLNKKEG
VINPLSKDIIALNLKCNVIKNIDDYLYLFFCNKLYFFVFFVKAQIKKRISIGRSIVIKN
DKMIKADGKTKQINRNTIELEKITTSKKEDENSNEDIIYEQILKKLDLEKIDKNLYTL
DGKLDKYKYNEKFMSFLKENKKKYNDILKYMYNKRNEKKEKQGIKNPIPENDQN
EKINNIINDKFIYISDKQVIKNTNMFIHKSIYEEKRKKYIDMKKRKEIKIFNIKKNDD
KKEDIQLEYNDILEENQIKNLIFDKVIYFNYVLFNQVYTKIFNVHNKNDECNIKINF
THSSNLNLDKNLLFIKGNSKEMIKTNLLLNLPIIKTDVENCNKTININEIKYRNIFDN
EKTNFQILTDHTFNQKEQNNLSLHENENLSNNNKSYLFDHKYYTENITLKINDNYE
DMIVVKGNIILLNIVIKINTLYFYFKNSDISMFCENNLILYNPFNIPIPITLEFSKEIFEA
KNKLVINPSEYKTVAIKFIGTQNIERMDNFINLYLDNNRLYKRVKCIFDVNKCSCKI
NVNEIKFENIILNKKYFKHFYLTSTGDTPVVFNCVAKPDCVNIYYKYNYVNKNEK
VKIIVSVKLKENKQIKDKIILSIRGSDNIIIPIIVTKVYSSNLLFLNDIKIDQETNELKR
YEIKIVNKGGCEENVILNLSELNLSELNFLNIELNKKTENNRIIYNNKEYKNVRNM
KDDFMVLKKIFKTEYENIITNKCVKYYFNKLIDLYNFLHNFSKNKFKIIFVENKNN
DIKINEKNMYKIKISPKCFVSLYIQCYYNNIIKKEIKFNKILFENKCLHELSNEVININ
IKNSQLSISPNLLLFQNIIPYNSERAFITYYKKEGIQKIKLKNNSSYPIKLEILLYDKK
QKDSLLATSSVDNFVEMERKQIIKNNEESNEKIEINKTLPRAVPKKDSNLNLKKGN
IQENVGNKSNEHVEKMYEIELNKESGIIKANEEIEVEIKLKNCKDIGKFVDILEIKTN
VVKNEQTDKIFDEKKYCIYILSVIEIPKLYFDITYINIIYNKLNDTFIFPFKIYNYGYS
YVRINYKFENLYTDFFFLSLDFLQGNEIDIKTKKINVELKCKSIKNLKFKSEIIISILD
YDIYKIPVFVNIDENIDYFLEKVQYEKFDALTSQANNYSLDQYMCKENSRSNLSEII
DEVVWTDKQFNSGKAENGNVENGNFENGKAENGNVENGNFENGNNYNISNVSE
QNNELLSFYCKNICTENCNIFKKRCIYKNLKINNIRNVYYNKENEFIEINEIPKDYVF
VFLQNILLNKSCAPTFLENTNDLFLFFINLLINFFHIYPNRNIYNLLHEIKIYQNISMQ
DFEKNIFIHENILANIKQILKSLKEEKLLVNHIIYENLLPIDLYCCHILDKVPDNFYIK
NVKCKKDSFEIKDIYNFINNNDDKILYFDRILKTHLKIFSYSWITIFFELLSKEIFNCV
NIESLYKLRGIKLCNIENKIYENIKIQKINFLLILNKEDNELNKHTSMLENIRNKTKR
EKIYGDNNINGGNKKIKYSQINEEKKEKKEKKINNIKNGVIKIRKQPTTIKKMGTK
KKDLDLNYFNFYENNLIDLKDLVNKINNLNLCSGEGEGEKGMENKAVLEGCQNK
AVLEGCQNKAALEGCQNKAALEGCQNKAALEGCQNKAALEECQNKIASTNQTNI
SKYYKNIEYILFEWLYFHYNNVYYNKVKNEQYIFKNKENEYNKNESNLINDDTNS
KQSNYMKKNDDNSSGYINNEHSYKNFEKSNFYREEKKKKNIVRENKLKIINIYSL
KNLVIILYTIISHIPYFLFFKNKIKVECKNRKDYMHNIDILLIILNEIKLDNLITRQVLI
DFNYIHIFLFLNCLYFILPNYLPKYYLDVDDISSYKSDRVLIKEEIVKKKNTIKNKK
NIDLHKDKERKRNHINNNDKSVDQNERIILIYNLNDNKCKYTVFLIGCSKYEIDKE
YIEIDSHKSEEIKLKIDPNYDKNIFKYDYNINIKFPSFTKKINNIHKKDNLLSHENVN
NTIPYQKNTSNFGDYSSISSSISSSISSSSIDSFQSSLSYDGIQNVEENNYENNFYHNE
NYTILVLRKESNLFPGDNDEDKFICIKLVETNKSKLKSEIVRSNSNLLVEGKEWNK
LNSNLKFEEGKVNMKNGSGTNSSKINIMLKNSEIKCFNMRGKIYENKSVEINLINDI
SKKVNINMNMYHIYIKNLKREEIKKGKFDIDILNNKENGNYKNDINRNINEEKGY
KECDVCSLLNLNLKKNLNCDKNYFNCFYIENMKPFVSRENEKRTIKLHFCPFIEGY
YLCFLLFCAKDLKTKCVMSSCKLNCLFYINNIKEEEVIKIDSQLSKFSYQIKLIPINK
RFLKCIQFILCYLNKMNNKVFLSYIKNYLNYINKISDQFYIICDKVGKNGIKEKLPS
FQKYNYNDFINQINENNLFLDNFYDQIKEKNNYLYEFNINEENMGVYEYNIILMA
NNKKKYNEDINKSIQLSKYDELEVDENSIYNACNKLYKIIANINKKENNNIINVTFN
QNAYLLSKKCVSIYNYTNKNLIYKCSNSEVLDMNKNKIDYKIFNYDEYIEIDKDVE
SFNFEIRCYSVVEVECSCFIFLTNVENEQDVIKINVKANIVKPYPKDTIHLKILNKM
KKAVSIEIYNELDFHCEYKIYSDLPIIFGDQKIEMLPKQKKVYTFFVRSIHIGEFIGCII
FKCFRFLDVNKIKDSRDIIKNDSNITSKNFSLVDSFFWYKLKVTVDLNKPINVLTLE
SNVGDVLNKEIVLKNNGNKKEEYFLLSYMNEYIEKKKIEIPANDAYVYTIKYAPKI
PNYFDNILKHKNEEDTKDTNFELDEKKEKGSKYNKTKKSECMKRKDEIKQNENIK
FSDISNELSNFYFLQNEEEEIKNEGDCLQKNMTKKLNLEKNEKKIFEELIKYCEKYI
NVDIKYTSHNIGFFFIYNKNEGVSYYILILLARFRTQLDISNFSSLLSKSCLIHLNFEF
SNKNKRYVNKLETDQQKNTSYYYQIGEYIEENNINKNNSYNCIENYKLNNGFEFY
NSKIERGADKSSNIKEFDGDGNGNENDDGDGDENDDGDENDDGDENDDGDEND
DGDENDDGDDDDMSTEQIVRHNIKGIYLSNKINYSMINCDNKKSGNYILIKYKPT
VGTSQECYVIIRSDLFGDFIYFLKGGYIEEKKIKERIIIHNSFCLYNFNINLFNPFSSKI
YVKCIIETDRSKNYEYNNMNKINCLKLYKSEKSRNIENGKEKILLNTKYKYFKLLS
NEHFKIKKRKNFSIFSTYFCKYAKSIEKLIILLYPINNKKKEKKINQYIIYEYELFFNN
TKSDFFLETDRIIADKEEEEEKEEEEEKEEEEEKEEEEEKEEKEEKEEKEENNLDNIE
NVCTKQLNYISNEMSKSSSFSFNGLEKDGSAEDGLEKDGSAENGVEKDRSDEYSL
EETESESYSTIEVGSKRNNHILYLDKLSISDSSDVGIEECYEEGMEINNKFDKNIDGI
NNKFDKNIDGINKKEVVYSDGKIFIQSICKIKTSMKIFINKKKIGEMEKYYQTIKELE
KKKQEKNIMHRNILLEKSNYKIFFLNLNNLKNSDKIEKNCKDNYGNEVLNNILFN
MNENILNNFLYINIVEDKENYLVISLELLANIPFHCNFFIMIKKVEIRENNKNDELD
VRNDEIYQYENDDKILKVKSIKSEKIKEFITVEKNYIFLEIFNYINISKKCINIYIDNN
LYSETNLNIFYKNNNDSYFDAYIVGYNQLLDKNNSDEIHNYTIRPKQGILKYNHFN
KFIITRTNKINIMAFNSSFLLLIKTENNLFSYIISSHFTPSHELYTEAEKNVIRDILKEN
RTKFSIFFNEFKQEKRESTIFNKKDEIIIYDISESNEIQSG
PY17X 070100 DNA
>PvivaxP01|PVP01_0527400.1: pep
(SEQ ID NO: 19)
atgccgggggaaacgcaaaacacgttcgacctggtggacgtggagcccaaatttctggagttccactacgagggggcagacag
cgtggaagccttcctcgaaaacaaaaaaaaagtaataaaaaggaaggggctgaaaataaaaaacatttgcacgaagacgcaaaa
cattaagatatgcgaatgcgattccaagtgtctcaaaattaaggggttgaaaaagaagaagttggctcctgggacgagcgagcaa
attttggtcgagttttccatgcggacgtaaattttaaagagagcaaacgtgggaagcaagaagacatcctggaggtagttaacaac
gtagaggcaacatcctacgtgactatacagagcgaatataccacgctgagcattccaatctacataaagaagagcatccccgtgttt
gcctacgacgatgtgataaactttggagtttgcaacgccaatcggacgtaccttttagccttgagggtgaagaatgtgggtacgag
gaggggcgcctttacgctgtccctgggtgactcgccaggtggaaatgcagacgaatgcgaagatggtaaacgggcggagagtg
cacagagcggggaaaactcagtcaaacagcaccgccgaaactgccactctgaagacatgctgattcggctggaccagtcttccc
tcgagctggacgtgggagaaacgaagaccatcaacatcaccgtcagcagcaaagtggaggggaaaatgcacagggagtacat
agtcgtttcgaaccaacatagctacttcgtggagaagcagaggaacatcaacgtcttggccattttcaccaacagccatacgtcctt
cctcttcgaaaatggaaaaacgaatctcctaaatctccactgcttgtactatgggagtaggaaaaagtaccaggggaaaataaaaa
atgaaaacaactacggcatattttgcaccccccgagtggatgccgtccatgtgttttactccaaggaggagctccaaacgtttgcgg
cagccagagggttggacctgggccggatctgccgagatgtattccttgaggaggaagaacgcctaagcgaaggagaaaaaaa
ggaaagggagaaactattcgccatcacgaagaagaggctaaagggggaggagcacataggagtcactttctgcaggggggag
ggctacccagttgaaaggctgtcctatcaagaggtcacatttgagattcagtcgaagagtgacgccggcctcttgcacagactcag
cagggacacggcctacctgctcaacttccccgtcgtggtggagttgtctctgcgggtggggcgctcgcgccgcgaggccagcc
aggttggcatcactacaaagcaggcgcacagatcaaccagccaggtttgcagctctgcaaagcaggagcacagatcaaccagt
caggtttgcagctctgcaaaactgccgcatagctcatccaaactgccgcccctctcaacaaaactgccacacagctcaaccgaag
tgggggactcccacccctgcgacgaggaggttaaactgtgggcggcgttctgcctgaccttcccctgcgtcctccccagcaccta
cctcctaagctacggcagcatcgcccccaacgaggggaaaacggtcatcctcgagctaacaaacaggaataaatttctaaaggtt
agctacaccctttcgaagatcccctacttgacactaagcaaaaaggagggggtaatccccccccaggggaaggcactcttatcgc
taaagctccagtgtgacagcctcaaaatggtggaagaatacatgtacatatatttttgcaacaatctgtttttctttgtccttctggtgag
ggggaacatcacgtctgctaatgcactacggaaggggacatcctccattctgctcgacccaaaaggagactcactgaagaggaa
cactcccccacagggggactccaaacaggaccgagaccaggtatatgagcagatcctccacaatttagaactcgaaaaagtgag
caggaattactaccaactagatgggaagctagacaaatataagtacaacgaaaggtttatcaattttttaaaaaaaaataaaaaaaa
gtacaacgatgttttgaagctcatgtatgaggagaggaagaaaagcgagtcagtcagaagtgggacccaaagtcaaggctctcct
gacaaaagtgggaaaataataaagggggggacagataaactctccaaaattatgacggacaaatttatatacgtaaatgaccctcc
agtggacagaatctccaacacgtgcatcgagaagggggtctacgaacgagagaggaagaaatacatccaaatgaagaccacaa
attggggagtaaaaaatggggaaccttcccaaggaggggaggagcaaccctacttgttacaattcgacatgctagatgaggaag
aactgaagagagtgcaattcgtaagggtcattcagtatggtaatatctttctgggaaaggagtacacgagaaagtttgccgtctttaa
caggaacaagcactgcagtgtaagggtggccctcactcatagtgaaaacgtcacgactgagggggacaaccctttggtaattcat
agggactctcacaaaatgggaatcatcaaactgaacgttaagcggttttcaacagcggaagagcctcatcatgatgcggttagcga
tggggattttcgaagccaaacgggagtgtctacccccctttttgaggccgtcaatttgccaaccggtggaggcacttccacaccgg
agggaaacacacctacagctctgtgcgccgacccatgcaacgacttcttcctcctcaaaacgttccaagagaaaatttccctaaca
gttaacgactcacatgagagggaggtgactctcggggcgactctcgtctttcttaacatcctcgtccacccacacactctccactttc
gcttccacgatgaggacgtcgggatgttttgcgagcgccacctcgttttgtataaccccttcaacgtccctctgcgcgtggggatgg
cctgcaaccgggcgtgcgtccagttggacgcggagatctcggtaccccccaattcgcacaaaatagtccccgtcaagtttgtcgc
cacggaaagtgccaccagcgtggcggaggccattcggctgtacctggacgggagcaggctctacagaagcttaacctgcaagt
ggatcgccaacaagtgctcctacagagtgacccccgccgagctcaactttgaaaacgtccttctaaacaaaacttatgtgaaagag
tttttcctaatcaacacgggagactcccctgtggtgttccgatgcagctacaaacccgattgcgtgaatttactctcgaagcataacta
cgtgaagaagaatgagaaggtcaaaatatcggtgctgctcaatttgaaggaatgtaaaaaaatgaaggacaaggttgtgctggcc
gttaggggcgcgcctcagttgctcctccccctgagtgccaagggcaccccctccaacgtcctcatcagcggcgggctgcgcatc
cgccaaaggagaggagagctcaagtgctacgaaatgaaaatagcgaacagaggaccatgtgaggaagccatcctcctagaca
catcccctgtgggctttctaaacatccaaatgggccacaacaacgaaaggaccttctccgagagtaccggcaaagagtccaagg
atgccagtaaaagagaaaatgtcctaacggtggagaggaccttcaaccaagagtatgatgacctcctgacggatgaatgtgccaa
atatcaatacaacaattttttaaggcttcacaatttggtgtcctcctgctatcaaaataaagggcaaatcctttttaatgaaaagaggga
gcgaaaaaacatctacagagttgtggtgccatccaagtgcttcctccccttgtacattcaatgctactcctcgagggcgctcacgaa
agaaattacactacatagtttgctccactgtaatagagctgcatacgaatgtaaggaagaatcaatcaccgtagatattgaggaagg
gcacttggacgtgtctcccccttttctccttttcaccgatttgctcccgcagaaagctgacgaagcttacataacatatttttcgagaga
gaggaggaagagactgacaattagaaatgtgctgagtcaacctgtgcagtgggcagtccgcttggatgaggagaagcgcaggg
aactctacacggttaggggcgctcacaggggtggggagcaaattgctgagcggaggggagaggcggggaagtcgaggaga
gtggggagtgacataaaagggagcgccatagggggtagcgccaagcggggggaagcacccgaaacgtgtgtcacacaggg
gagcgccatagggggtagtgccaaacggggaggcgcaaccaaatcgcccgagtcggatgaacccgcggaggagcgccagt
accaagtgcaccccagcagggagagaggcattctacagccaaacgaagaaaccacggtggaaataaagctaagcggcttcaa
gcagtgcgggaggtacgtggacgtgctggacatctcttcctccccagtgggtcccccccacggtatcgacagcgaagaggcgc
aagagcaaagggaagcagcagaaggggagacacatttcgccctctacatgctcatacggtgcgacgtcccgaagctccagttc
gacgtgacctacataaacataactcacaacaagttgaatgagtgcatttcattctccttcggaatatacagccatgggtaccactacg
tgagggtcgattcgcatctgaagaacccccacgcagagtgcttttccatctccctccacttcatcgacggaaatgaaataaacgaac
gggtgaagaagctgcgcgtgcagctcaagtgcaaagcgtccaggtggctgacattcaaatccagcatcgtccttagcgtgatgaa
ccaccaccagtacgcccttccgctcttcgtgagcatcgacgagggggtggacttcctcctgggcagggccagcggtgagggca
gcggtgagacaagcggtgagacaagcggtgagatcagcggggagacaagcggtgtgaccagcggtgagacaagcggtgag
atcagcggtgagaccaccaaaggcgcaaccctagagagaagcctgcaccaacatttgaacacaacagacctcccatttgacaag
caaaacgaactcctctccttcttttgcaaaaatgtggaggaatcaaaagggggaagcaccaacggagatatatacaaaggactca
aaatagggaaaatcaaaaatgtgtattgccatgaggaagagggggtgtacccactgagcgaaatcgttggagattaccttttcgtgt
ttttccaaaaaatgctattaggtaaaagttccttcccccaggtgattgaaagcaccaacgatttgtttctcttcttcgtggacctcctgttc
gacctcttttccgccacctacccaaatagaaatctgcaaaatgctattgccgaggttaagaggcaccgagctgtcgtggtgaagga
catggagcgacaggacttgctggatgagaggttggcgaaacatgtgaaggccctctggaagtgcttgcaggaggtaggcacag
ggaggctccgtttggccgtacatcacccggctactttgtatgccacttcatcaccccgctacgtttcatgccacttcatcaccccgct
gcttttcacacctcttcaggaatgcctcccagtggaccacctcctttacgaaaacctgctgccagcggatctgtaccttccccacatt
ctggccaacgtgccggaccacttccacgttgaagagaaggacgaggaggactatggtgatagcgcgggtcacctctccatgcg
agacgtcgagtcatttttgctaaacggaaataaaaaagtattctactttgagcgaatttttaaaacgcacgtaaggctattcagctactc
ctgggttaccatcttcctggagatactaaataggaagttctttggccggatcaacatcagctcgttggtcactctacgtgggattaaac
tctacagcatagaaaacaagctaaacgaaaatataaaaatgtgtaaaattagctaccttgtaattttagatactcaagacagggaggt
gaacaagcacgtctcatttttaagtgaaaagaagggcaggtccaggagaaaggacgtcaccacgttgaccaacctgcaggttgtt
agcggcggtactgctacgctacgcacaggaggacaatctataaacactcgtaatggcaatcccactgatggggataaaggtacc
ccccagagaagagaaaaatattcgcccaattatgtgaagaaaaggacggccaaggacaaaccctgctgcgccatcaactacttc
aatttttacgaaaataatttcatcgatttgaagaaaattgccaacaaaattagcggcttctgctcccccgggggggaagaagcggcg
cagggggaagccggcgcgccgatcgcgcagcgcacgccgagcaggccttttaagaaagagcccactaaggagaccgctaag
caaacctccccaaacgcgccaaacggaattgtgcacttccaaagcgcggagtgcatcctgttcgagtggctctatttccactacaa
caatgtgcactacgcgaaggtggaggataagcggtacgtgttcagggaggcccgggggggaggcgacgaggagttgtcccac
acgaatggggcgatgaaggggggagagccggagggggcgcgcaggagaggaagccccagtggaagcaccaattgcgccc
caagtggcaatccgcgcttcgccccaagtggcaatccgagcttcaccccaagtggcaatccgagcttcaccacaagtggcaatc
cgagcttcaccccaagtggcaatccgcgcttcgccccaagtggcaatccgagcttcaccacaagtggcaatccgagcttcacccc
aagcccagttgggaaaaacccgaagatgagcagaatcaacttccaggtcgaaataaaaaggaagaaaaaggaggccgaaaaa
aagaaaaaagtgaaaggcatcgacgaattgaaggacctagtgatggtcctctacaccatcatttcgcacatcccctattacctattct
ttaagaacaaaattaaagtgaaatgcaagtcaaaaagggattacaacacaaacgtggaaattctcctcataatgctcagcgagatg
aagttgagcagcctaatcagtagaggggtgctcgcgcagtttaaccacctccacatctacctgttcttggcctctctgtatttcatcct
gccgagctatttgcccggctactacttgatcctggacgacttccccgcgtacgaggtggacttgggcttgataactgacgaggttgt
gaacggccgcaagaagtggaagcagcaaccgggattgaggcagccgccaaccaaggggaagcagcaaacgggattgaagc
agccgccaggtaagagcccactaccgccccaaagcaaagccaacccacacaccgatggggggggcgaaaaaaccggttgcg
gcgacggacactccctggcaaacgagcgaatcatcacggtgtacaatttaaatagccacaaatgcaaatatgaggtgtttctcattg
gctgccacaagtaccaactggatagaaaccacctctgcattgaccccctgaaggcggaacaaataaaattaaaaatagacaggg
ggtatgacgaacaggcgttgagctatagctaccacactagcattaagctttcatcatttaagaaaaaaagagacaaaggggacatg
tgcgacggggaggaaccccacctggaggacagctgctccgtttcgtcctgctcctcggaagagtgccagtgcccctccgacgg
ggccgacaaggtggacgcggggaccagcggcgagagtaggcccaccccaggggaagattactccattttggtgctgcgggc
ggaaggcaaccacctgcaggacgaacagcacagcgagaagtacatctgcatcaagctggtagaacgggacgaaaaggggaa
aatcgaattgacctccccaggagcgagcataagggggaagcaaaatgagggaaagaacgtaaaagctaatctacacaatggag
tacccaggagcgaggttaacaaaatgagaacggccaattcgtcgcagctaaatattatgctaaacgatagcgaagtaaagtgcttt
aatttgaggggcaaagtgtacgaaaggaaatgcctggaatttaatttgtgcaacaacacggggaaggaggtggaggtgcgttcgt
atttgtaccacgtgtatgggaaggatttgaaggaggagaagcgaaggggaaatttggaagcggacgtagtgagttcatggggga
acagccaatgcgcggtgtgctccttagttagtgccaacttgaggcgccacttgagcggcacgccgacccacttcagctgcttccac
ttgagcggcgcgccgccccacttctgctgcctccaaaatgaaaggaaaaaaatacaagtgcagttctgcccctttgcagaaggga
gttactcctgcttcctcctgctcaacgtgaatgacaaaaagagcaactacgtggtgtctgcatgcaaagtcaactgcttctttaacgta
aagaatgtgaaggaggaggaggtcataaagatgaggtcccaaactttcagcttctcataccagttggaagtttgccccctaaatag
ggagttcctccactgtgtgaagttcatcctgtgtaatttgaaccacctggaggagagccattttctggcctacgtaaggggctacctg
aagggtatacaacatgggggggacaatttttatatcctgtgtgataatgaagataaggtgaccatcgaaaggggggttgcaacgttt
ggagtgcttgacgccgagaagtacgtggagaaggccgcacattctgaaggcgcaaatgtagagaagctacaccagatggtgaa
gagggatgtcaattatatgtgcttacttcagatagctgagcagactagtggagtgttcgaatacaactgcgctctggtgaggcagag
gggtgccccccgaggtaggaaccacctatcctatgaggagctgcgcaatctgcacatcaaggagcagcaggaatggttcgacc
ctttctacaaagtgtacaaaattgtagccaacgtgaatgacgcagggaatacccaatcgttgtccatcacgttcaagacgagtgcctt
tctgatggccaaaaagaccattcctctgtacaactacacaaacggggtggtaacctacaggcgagtgctctcagatgtgatcgacg
aaaataacaacgtggtagattaccaaatttttagctgcccagaatatgtagaaataggaaaagacgtggaatttgcccaatacgaag
ttagctgttactctatcatcgggttgacatgctcctgctgcatcactctgtacaatgtgaatgatgaggaggatcggataaagataaac
gtgcaggcaaatgttatgaagccctacccgaaagataccatccacttgaagctactaaatagaatgaaaaaaactgcgcaggtgg
aaatctacaatgagctaaactcccactgtgaatttaaggtcttctcagatttgccaatcttatttggtgggaagaaaataaaaatgctcc
caaaagagaggaaggcctacaccttcttcgtcaccagtgcacacataggtgaatacatggggtgcatcatcttcaagtttcacaag
ctgctccgttcaggtggtgcagaagaaaaaacgagggacgcctccttcccctttgcgaactacttcttctggtacaaactcaacatc
accgtggaattgaacaaaccgctaaaaatcttatttttagaaacaaatgtaggagaggaagtcaccaaagaaattgtcttgaaaaat
aatggcactcaggatgaggactattttttgctcgcctacatgaatgagtacatcgagaggacgcaggtccaggtgcccaggaatga
catctacgtgtacagcattaggtacgctccgaaaatgcccaactgctttgagggggcgtccagcgggggggcccctctaggggg
gagtgccgctctaggggagaatcacccaaccgtccggggggacctccaaaatgtttacttcccgcagaatggggactccgagtg
gtgtggcccaagggaagaggccctccgtgggaagccctcaacggattaccccaatgaagaggccccttcccggacgccctcag
cggaagtgccccccaaccatggtgctgccccgaagcgcaagcccttcccccccaacgagaagaaaatattcgaagagctgatc
agctactgcaagaagtacatccaagtggacatcccctacaggccccagaacatagggttctttttcatctacaacaaaaatgaggg
aataaattactacattttgatgttgctctccaggctgaggagcgaattggaggtggggaggttctccacctgcctgtcaaagtcctgc
ctcctccatttgcacttccattttaatgacgaagataagcgatacgttcatctgctgcaagagaagggggacccccgtgctgggaatt
atcattacgaaattggggagggggtcaaacatccccaggggggaggaaatgctttaaagggagagaatttccaacaaggtaccc
cccacacaggtcgcgcttaccaaacgtatgatgatgaatggggggaggagatccacaggggggatgcaaaaaacgggaaaga
tgcccccatttgtaaggacaacccatgcagtgggagcggatcggaccaggagaaacacgaactgaagtgcatctacctaagcaa
cagaaagaacttcttcacagtaaaccatgaagatgtaggcagaattgttctaaagtacagaccgacggtgggtacatcccaagaat
gctacgccatcgttaggagcaacctccacggggacttcctctaccgcatcagcggaacgtacacggtgaagaggagaataaag
gaaaagctggttttctacaactcctgctgtttgtacagttttaatgtaaatctgttcaacccgttcgactgcaaggtgagggtaaagtgt
aggttcaagggggggggcccgagccaggcgtgcagcggaagaggtggcgtggggggagcggaagagggagcggaagag
ggaacagacgagagagacacagagaggaactgcctcacctcgatgcagacggagcgagttctgctcaacaacgaacggaatt
acctaaagatgatgaataaggaaaactttaaaataaaaatgaggaggcacttcaccattatgatgacgtacttttgcaaagaagtttat
tctaggagggcggtgctcgtggtgatgaagccgcttcgcaggggggatgccactcgtgaggacgtccccccgatgattacttac
gagtacgattttgtctttaacaatttgttgaggagtggcaggcttccgggtgcgctgcggtgcgtccccattgcaggtgagggtaag
agagcgggagaaaaggggaatcgcggcgaggagcgctcccaaaaaggagaagacccaggtgtaagcacatcatgcaacttc
cattctggtgtggacgcactgcggattggcagccgcgtgggtggagatttcacgaggcgttccgaaggggtcgaagcggaaaat
aaccttcccgaagtggccgatggggaagataaccctcccgaagtgagcgatggggaggcccacccatccgaacaatctccatc
cagccacacctcacgcagcccctctgcagccagcgaacgaagccatttcacgctgaacgcaagcgaattgggcacaacggac
gtgctggaaggggagctaaaatcggaagacgcagatgccgccgaatttgagaagcatccccccggcgataaggcgctatgga
gacctaatgactacaacgtggaagaagacgtgaggcgcgagagggttctcccctgtgcagctggtggggcagcgggtagggc
agccattagcgccacggatagtaacgaggtggtgcactacgacgggaaggtattcatcgaaagcatgtgcaaggcgaagacgt
gtgtggaggtgcacatagataaggctaagaggcgggcaggggcaactcccccccagggtgggggccaactggagggggag
aagcaggaggggaagcagcaggcggggaagcagcaggcggggaagcagcaggcggggcaaaatagtgcgctccacagg
aatgtgcaaagggggaagtataactataaaatatttttacaaaacgtgaaaaatgtgcgtaagggcgggggtagcggtggaggtg
gcgaaggcggcggtgaatgcggcagggagacacccgggatggacgtgaaaacgaacgcgctccttttcgacgtgagtgagg
agctgctgaatgattacctgtacgtccaccttgtggaggacacggccgcgcgcctcgtgctgtctttggagctgctggcccttctgc
ccttccactgcacctttgaggtgttggtaagcaggacgggagaggcgaacgaagcggatgaagcgggtgaagcgaacgaagc
agatgaagcaaacgaagtggtcggagaatcctctaagggcgaacccccccgcagacgcgccaccctcgagcggaaccgcata
aacgtagaagcattcaactgcatgaacgtgtccaggcagtacctaaacatcaccgttgatgaaaatttatgtgcagaggcaaagctt
aacattttttataatcacacgagcgacgcatacttcgacgcgtatgtggttaggcacaaccagctgaacagcagcagccacttgaa
ggacgaaatagtaaattttgatgtcaagcccaagtgtgggattttaaaacacgggagttacaacacgttcttcatcacaagggcgaa
caggaatggggtggtgtcatttaacaactcttttttgtttttaattaaaacggagcggaacctcttttcgtacatcgtcttttctcactacc
ggccggcccccagggacgcgctcgccacgaaggagcataacaagataaaggagatcctcaacgagaacaagaagaggttca
gcgtcctcttcagttcgttcaagcaggaaaagaaagaaagtagcatcttcaatcagaagaaccagattatcattgaggacatttcca
agtcgaccaacgaaattagaaatggctga
>Pknowlesi|PKNH_0510400.1: pep
(SEQ ID NO: 20)
atgtcgagggaagtgcaaaacaagttcgacctggtggatgtggagcccaaatttcttgagtttctatacgagtgcacagacaatgtg
gagactttccttgagaataaaaaaaatgtaataaaaaggaagcgattgaaaataaaaaacatttgcacaaaaactcagaacatcaa
aatatgcgaaccggattcgaaatatcttaaaattaagggaataaaaaataaaaagttggctccagggacgagtgaacaaatattcat
tgagttctccttcgctgatgtgaattttaaaactagcaaatttgtacaaacagacaacatcttggatgtagttaataacgtggagtccac
ttcgtacgttactatacgcagtgaatatacaacgctgaatatcccaatctatataaagaagagtatacctgtgtttgcctacgatgatgt
aatcaactttggagtttgcaacgccaatcgggcgtaccagtttgcgctgagggtgaagaatgtcggcacgaggaggggcacgttt
actctctccctggacgattcgccaagggaatgttcagacgaatgcgcagatgatcaacaggcggagtgtgcacagagcaggaaa
aactcaggcaaatttcacctccgtaaatatcgctctgaagacatgatgattcaacttgatcagtcttccctcgacctggacgttggag
aaacgaaaatcatcaacatctccatcagcagcatggcggaggggaaaatgcacaaagagtacagagtaatttcgaaccaacata
gctacttcgtggagaaacagagaaaaatcaccatttttgctattttcaccagcagccatacttccttcctcttcaacaatgaaaaaacg
aatttggtgaatcttcactgcttgtactatgggagtaggaaaaagtaccaagggaaaataaagaatgaaaacaactacgctatatttt
gcatcccccaggtagatgctgtcaatgtgttttactctaaggaggatcttgaagcgtatgcggtagccaagggtttggacctgagcc
ggatatgtccagatgtgtacctccaggaggaagaacacttaagcgaaggagaaaaaaaggaaaaggaaaaactgttctccatca
cgaagaagaggctaaagggagaggaacacataggcatcaccttctccagggtccaaggatacccaattgaaaagctatcatatc
aggaaatctcatttgaggtttattccaagtgtgacaccgaccttttgcacagactgagcagggacacatcctacctgctgaacttccc
cgtggtggtggagttgtcgctgcgggtggggagatcgcaccgtgaaaccggtcagggggatagcgcaacgaaacaggtgcat
agcacaacaaagcaggtgcatagcggaacaaagcatattcacagcgctaccaagcagaatcacagcgcaacggaacaggtgc
atagcgctacccttgagggcgctcacacgctcgacgaggaagttaaaatctgcttggcattctgcctcaccttcccctgcatcctac
ctagcacctaccttctcaactatgacaccataacccccaacgagggaaaaacgctcatcatcgagttaaaaaacagaaataaattta
tgaagcttaattataccctttcgaagatcccctatttaacattgagcaagagcgagggggtaatctctccccaggggaaagtagtctt
atccttaaaactccaatgtgatagtgtcaaaatgatggacgactacatgtatctatatttttgcaacaatttatatttcatcgtccttctggt
cagggggaacatcatttcaagtaacgcattacggaaaggatcatcctccattttgcttaacctgaaagcagagccatttagaaggaa
tgagacgcatggtaagattaaacagaaccatgatgaggtatatgaacaaatactcaaaaacctagaacttgaaagggtggatagg
aatttttaccaactggatggaaagctagacaaatataaatacaacgagagattcataaactttttaaaaaaaaataaaaaaaaataca
atgatgttttgaagctcatgtatgagaagagaaagaagggggaggagtcaatccagaatgcgagtccaagtgcagcctcccctga
tacaagtgaacaaatggtgaaggggaaagacagactctccaaaattttaaaggacaaatttatatacgtcaatgatccccccgtgg
agcgaatctccaacacatatatcaataagggagtctacgaacaagagaggaagaaatacatacaaatgaagacgaaaaatttgaa
aataaaaaatgaggaacgtgcgcaaggaggagaagaagaagaaccctacttgatgcagtttgacatgctagacgaggaggaac
tcaataaagtagaattcgtaaggatgatccaatatgggtatatatttcaggagcagattacacgagaaagtttgtcgtctttaaccgaa
acaaacactgcaacgtaaaagtggccctcactcatggtgataacatcacgactgatggagagaagatcttactagtgggaagaga
ttctcacaaaatgagaaatgtaaagcttaacataaaatgtgtttccagggaggaagaagttcaccaagatgataaaaactgcaatga
tatttgtctcaacagtggtaaaattggtagtcaaactggagtttctacttccttttttggggaagacataatcacaggttcatgtgccgac
ttgcataacgacttttttctcctcaaaatgtttgaagagaaaatttccttaatgattaatgattcacataagagagaggtgactctcgggg
caactctcgtcttcctaaatgtgcttgtcaatccgaccacagtctactttcgcttccacgacgaggacgtcgagatggtctgtgagcg
ccacctggttttgtacaaccccttcaacgttcccctcaacgtggggatggtttgcaaccaggcacatattgcgttggattcggagatc
ttggtacctcctaattcgcacaaaatagtccccctcaattttgtcgccacggaaagtgccaccagtgtgtctgagaccattcggctgt
acttggacgggagcaggctctacaaaagcttaaaatgcaaatttatcgccaacaagtgctcttacagagtcacccccaccgagctt
aactttgaaaacatcctcctaaacagaaattatgtgaaagagtttttcctaatcaacacgggagactccccagtggtattccgatgca
actacaaaccagattgtgtcagtttactttccaagtataactacgtaaagaaaaatgagaaagtcaaaatatctgtcctgctcaatttaa
aggaaggcaaaaaaataaaagacaaaattgtactgaccgttaggggggcgcctcagttgattctccccctgaatgttaagggcac
cccctccaatgtcctcatctgtgaagggatccacatccaacaaagaagaggagagctgaaatgctatgaagtgaaaatattgaaca
gaggatcatgtgatgaaactatccttgtagacacatcatctgtggatttcctaaatgtccaaatgggaaacaaaaaagaaacgacctt
ttctaaatgtatcagcaaagaatacaaggatgcaaataaaatggaagatgtagtaacagtgcaaaggatttacagcgaagaatacg
atgacctcctaacagatgaatgtgctaagtaccattacaacaatttcttgaagcttcacaattcgatatctgtattgtaccaaaataaag
ggcaaattctccttaaggatagaggagaagtaaaatccatgtacagagttgtggtgccatccaaatgttttctccccttgtgcattcaa
tgctactcttccaaagcgataaggaaagaaattgctttccaggatttgctccaccataacagagttacatacgacataacggaggag
ttaataaaaatagacattgaggaaggacacctggatgtagctccctcgtttctgcacttcacggatttgatcccccaccaggctgac
gaagcatacatatcatacttttctaaggagaagacgaagagaataacaattagaaatgtattcagtcaacctgtccagtgggcaatc
cgcttggatgaggagaagcgcagggaaatccacgtggttacaaacgttcacaggggtgtgccagagtttactgaacggaaggg
agggatggggaagtcgaagagaatggatagcgtcaaatcgagaggctcatcgaaatcgcaccaaacggatgaacccatctggg
agcgccaataccaagtgtaccccagcaggcaccgaggcattctacaaccgaacgaagaaacgactgtggacataacgttaagtg
gcttcaaactttgcggaaggtatgtggacgtgttagacatctcttcctccatcttcagcgaccaaaatgcgggatgcatcgacggtg
gcaacgatgccagtaacgatgccagtaacgatgccagaaacgatgccagaaacgatgtcagtaaccatgccagtgacgatgcc
agaaacgatgccagtgacatgctcagcaatttggttggagaagcggaaccagcgcagaaggaagaagcagagcgcgaggtcc
gtttctccctctacatgctcatatgttgcgacattccgaagctccagttcgacgtaacatacataaacataattcacaacaaactgaat
gattattgttctttcccttttgacatatacaaccatgggtacaactacgtcagggtagattaccatttcaagaacctgcacgcagaatgt
ttttccatctccctagacttcatcaacggaaatgaaataaacgaacaggtcaagaagctgcgcatgtcgttgaagtgcaaagcaacc
aagtccttgaagttcaagtctcacattgtctttaccgtgatggattatcaccagtacactattccccctttcgtgaacatcgacgaagga
attgatttcctattggagaaggcggccaccagttcgtccgtcatgtcgtccatcgaagagacaggcgaagtgatcgacgaagtggt
cggcgaagtgatcggcgaagtggtcggcgaagtgatcggcgaagtggtcggcgaagtgatcggcgaagtggtcggcgaagtg
atcggcgaagtggtcggcgaagtggtcggcgaaggaatccccaaacagatgcaccatatggacgattacctaccccctgaaaac
agccccaaaggagcctccaaaggtacctaccctgcagggaaacccacagaaggcataatcatggacagcagtctacaacagca
tatgaacacaacagacatccaatttgacaaacagaacgaacttttctccttcttttgtaaaaatatggaggaagtaaaaaagggtagt
actaatggaaataatatatataagggattaaaaattaataatataagaaacgtgtactacaatgaggaagaggagggcgtgtaccca
cttagagaaattattggagattaccttttcgtgtttatccaaagaattatgttacgcaaaagttacttcccccaggtgattgaaagcaca
aacgatttgtttctcttctttttggaccttctattggacctcttttcttccatctatccaaacagaaatctgcaaaacattattgtcgaactga
agaagcgtccaactgtggtggtgaaggatatgaagagagaagacttgttggatgaaatgtttacgaaaaatgtgcagacgctttgg
aaatccttaaaagaggaggaatatattccactggatcacatcatttacgaaaatttgttaccagcggatttgtaccttttccacgttttgg
acaaagtgcccgactacttccacgttgaagagaagcactatgaagataacacagaaaatctctctattaaaaacttcgaatcgttttt
gttaaatggaaataaaaaggtattctactttgagaaaatctttaaaacgcacgtaaggcttttcagttactcttgggttaccatcttcctg
gaagtactaaacaggaaaatgtttggcgcaataaatatgagctccttgacgaacctgcgtgggattaaattgtatagtatagaaaac
aagctgaacgaaaatataaaaatgtataaaattagctaccttgtaattctagatacagaagacatggaggtgaacaagcatgggtca
tttttaaatggtaaaaagggtagatccaggaggaaggaggctagcaccctaacaaaactgcacgttaatagaggtggtaccggta
cacgaaatgtggcgaaatcatctctgaatactcgtaatggtaatttgatctatagggataagagcacctcccgggggcaagacaaa
tatgcttacaattatgtgaagaaaaagacgacaaaggataaaccctcctgcaccatcaactacttcaatttttacgaaaataattttatc
gacctgaaaaaaattgtcaacaaaattagcagattctgctactccgggggggtagaggaactaccaaatggaaagcaagaaacg
gtgcaggggaagaacgaactggagagttccaaagggaatacaaacatcgagggggacgacattccaacgggaggagaaacg
aacacaccttttaagaaaaaatccacgaaggaagatgttaaggagtctaccataaaatattccaaggacaccacaaaagacgcgc
caaacggaattgtccacttcaaaagtgcagagtgcatccttttcgagtggctctattttcactacaacaatgtgtaccacgcgaaggt
ggaggataagcggtacatctttaaggaggcccgtaggaaaagcgacgatgagttgtcctatacgaagggaaattcaaatgagac
gcaagagcggggaaagccggagggatcgcgtagaaaaggaaaccccagtggaagtcctagtttcgtccccagtgactctcacc
atgttagcgcttcaggggagctggtcccaagtcaggtgcatgagaacacaaagatgagcacaagcaactttcaggtcgaaataaa
aaggaagaaaaaggaggtccaaaagaaaaaaaaggcaaaaagcatccatgaattgaaggacctagttatgatcctgtacactatc
atttctcacatcccctattacctattctttaaaaaaaaaattaaattgaactgcaaatccaaaagggattacagccagaatattgatgttc
tacttataatgctcagcgaaatgaacttgagtaacctaattagcagacaggtgatcgtgcagtttaactacatgcatatatatctgttttt
ggcgtccctgtattttatcctgccgaaatatttgccctgctactacttggtgctggacgatttgcccgcctacaaggttgacttgagctg
gataaatgatgaggtggtgaacagcaagaagggaggcaaaagtaagccaatgggtaaggcgcagcaatcatccagggggaaa
caggcaccccaaaaaaagaccaatgcacccatcgatggggagaacgaaaaaacgagttgcggagacgatcaaaccatggcag
acgaacggatcataactgtgtacaacttaaatagccacatatgtaaatatgaggtatttctcattggctgcaataagtaccaaattgat
agaaactacgtgagcattgatcccctaaagtcagaacaaataaaattgaaaatagacaagggatatgacgaaaaggtgttgaaata
tagcaaccatactagcattaaacttccatattttaagaaaaaaagacataagggagatatgtgtgacggagaagaaccactttactttga
ggacaattactctgtttcgtcctgctcctcggaagattgccaatgcccctccgacggggccgacaaagtggacgcggggattgac
gaagatagtcgggtcagccaaggggaaaattacaccatcctggtgctgcgctcggaaagcaatcgcctgcaggacgaaaagga
cagcgagaaatacatctgcattaagctggtagaactgggcgaaaaggggaagatcgaattggctcccccaggaacgaacataa
ggggagaaaaaaatgaaggaaataacgtaaacgctaatgtaaacaacggaggaactaggagcgaggttaacaaagtgcgcac
aactaattcatctcatctgaatattatgctaaatgatagcgaaacaaagtgttttaatttgaggggcaaagtgtacgaaaataagtgtat
agaattgaatttgttcaataatacggggaaggaggtggaggtaaattcaaacctgtaccacctgtacgtgaagaatttgaagaaaga
ggagaaaggaaaggaaaatttcgaaacagatgtagtgaataagtggggcgatgaccatctgggggggcacaaccaatgtgttgt
gtgttcgatagttaatgccaatttgaggaaccacttgagcgacacgtctactcactttagctgtgtccatttagttgacaaaaaccgctt
cacttgtctccaaaatgagagaaaaaaaatacaagtgcaattttgcccctttgcagaagggagttatttctgctttcttctattcaatgta
aatgacaaaaagaccaactatatcgtgtcggcatgcaaagtcaactgctcctttaacataaataacgtaaaggaggaagagattata
aagatgaactctaaaattttcaatttctcttaccatttaaaagtttgtcctgtaaatagggagtttctgaagtgtgtaaaattcatcctttgta
atttgaaccatttggacgagaggcactttttgtcttacttaagaggttacctagaggatatacataaggggaggaaatttttttctgtcct
gtgtgataatgaagacaaaatatccatcgaaaaaggcttcgcaacttttggcccactggattgtaataagtatatggagaaaattttat
attctgaaggagtagacatagatcaattgtacgaattggtgaagaaggagaccaattatacatgtactctggagatagcagagaaa
acgaatggagtgttcgaatacaactgcgctctagtaagccataggaatgtccttcaaggtaaaagtaagttatcctatgaacagatg
cgaaagttacacatcaaggagcagaaggagatatgcgaccctttctacaaagtttacaaaattatagcaagtgtgaatgatggaaat
agtagccaatcgttatccattacgtttaaaacgagcgcatttcagatggttaaaaaatccattcctgtgtataactacaccaatggagt
ggtaacgtacagacgtgtgtactcagatgtaatagacgagaataataagatagtaggttacaacattttcagttgtccagaatacata
gaaataggaaaagaagttgaatctgtgaactttgaagtgagttgttattccattatcgggttgacatgctcctgttgcatcactctgtac
aacgtaaacaacgaagaggaacagataaagataaacgtacaggcaaacgttatgaagccctacccgaaagataccatacacttg
aagttactaaatagaacgagaaaaacggcacagatagagatttacaacgacctgaatttccattgtgaatttaaggtttattcagattt
gcccatcctgtttggtgtaaagaaaatagaaatgatgccaaaacagaagaagacatacacgttcttcgtaacaagtgcacacatag
gagaatacatgggctgtattatctttaagcattacaagttgcttcgttcaggtcatggagaagaaaaaaagggagattcattctttccc
tttgctaactacttcttctggtataaattaaacatcaccgttgagttgaataaaccattgaagattttatttctcgaaacgaatgtaggaga
ggaagtcaaaaaagatattgttttaaaaaataatggaaatgaaaatgaaaattattttctcctcgcgtacatgaatgagtacattgaga
ggagacaggttcagatacccaagaatgacatttacgtgtatagcattaagtacgctccgaagatacctaactattttaatgaggatgg
caacggggaaagtgcttctggagagaacaacgatccaaccctccagagggacatccagaatttttactacccccataatggcgatt
ctgattggtgtgggccgccggggggggatgaacctgcccatgggggcaaagaaccatgggagttgtctcctaaccaaggcgct
acattaaaacacaaacataagccctttcccccaactgaaaaaaaaatattcgaagatctcattagctactgcaacaagtacattcaag
tagacattgcctacaggccacataatatagggttcttttttatctacaataaaaatgaaggaatcaattactacatcttgatgttgctatcc
aggatgaagagccacctggacgtaaggaatttctccacctgtttgtccaagtcctgcttcttacacctgcactttcagtttaatgaaga
ggacgagcgatatgtacatctgttggaagggatggagacccccccctctagtaattatcattatcaaattgtagagggggtaaaatg
tccaccgggggaaaaacacattttgatagaagaaaatttccgaggggctgtggacatgtgtcgcgcttacgaagagtatgatgata
aatggggggaggaaatgcaaaagggaaatgcaaacaactatatggatgcccccatttataaggacaacccatacaatggaggcg
caagggaccaggtgaagcacgaactcaagtgcatttacctaagcaatagaaataacttccatattgtaaaacataaggatgtaaata
gggttgttctaaagtataggccaacggtgaatacatcccaagaatgctacgccatcattaggagcaacttgcacggggacttcctct
atcggatccatggaaaatatacagtgaagagaaaaattaaggaaaaactggtcttgtacaactcatgctgtttgtacaattttaatgta
aatttgtttaaccccttcaactgcaaagtgagggtaaaatgtaggtttaaggggggaaacgcaacacagttatgtaaccacatgtgt
ggtgcgcagggaatgaaagagaaagacatagatgagaagtacatgaagtcgttccagaaggagcgagttgtactaaacaagga
aaggtattatttaaaaatattgaataaagaaaactttaaaataaaaatgaggaagcactttacaattatgatgacctacttttgtaaagc
ggtttattcgaggaggacggttctcataatgatgaagccttttcgcagggataggactcatgtggatggccctccgatgataatttac
gaatacgattttgtgtttaacaatttcttgaggagtagaaggatccaaaatgtgttgcagtgcatccccgctgtgggtgagataaagg
acgaacaaggaagggggaactgtgaccagaagcaaccatccacagagtgtgaagacctaggtgtaaatacatcatgttacttcca
ttcgagtgtggatgagccggagtctggaaaccaagcgagtggaaatttcacgatgaggcattccgagcggggtgatgtggaagc
taacctgtccgaagtgagcgatgtggaagcccaccaatccgaacaatctgaatcgatccatacctcacgcagttcctctgtagcaa
gcgaacgaagtcatttcacgctgaatgcaagtgaattgggggtaatggacgtggcggaaggggatttcaaattggaagatgccg
atacaatcgaaataggaaagaatcacacctgttttactcaccccgacgacaaggcgttattgtctcctaatgactatgatgtagaaca
tgaaataaagcacgagagggttcttccgtgtgcatcggataataaggaggttgtacactatgacgggaaaatttttatcgaaagtat
gtgcaaaacgaagacctacgtagaggtacacatagataagaccaagatggggacatgggcatcttgcccccagggtatgcccca
gggtatggatcaactggagatgcaaaataattctcttcataggaatgtggaaatgggaaattataactataaaatatttttgctaaactt
gaaaaatgtacgtaaaaacatggatactgttgatgaaactgttgttggtggtaccgggggggcaatcgggatggatgtaaaaatga
atacgcttcttttcgatgtgaatgaaggactactaaatgattacctgtatgtgcacattgtggaggacacggaggcgcgccttgttctg
actttggagctgctggcctttcttcccttccactgtagctttgacgtttttgtaagcaggaggggaggggaaggagcaacccaccat
ggggaatcaggcaaagcagaccaagcggcagcgacaaacaaagcggaagcgacaaaccaagcggcagcgacaggccaat
cagtggaagaagcctacaatgtagtcgaggaattctgcgggggaaagccccgtcacagatgcaccaccttcgagcagaatcaca
taagcgtagaaatattaaactgcatgaacgtgtctagacagtacctaaacattaccgttggtgataatttatgtggagagacaaggct
taacattttttataatcacacaagggattcatatttcgacgcctatatggttaagcataatcagctgaataacagcagtcactggaagg
atgaaattttaaattttgaagtcaagcctaaatgtggtattttaaagcatggtagttacaacaaattcttcattacaaggacgaacaaaa
atgggctcttaacttttaacaattcttttctccttttaattaaaacggagcggaatgtattttcgtacatggtcttttctcactacagggcgg
cacccagcgatgccctcgcgacggaggaacataataagataaaggagatcctcaacgagaacaagaagaagttcagcgtcctat
tcagttctttcaggaaggaaaagaaggaaagtagtatcttcaatcagaagaatcagataatcattgaagacatttcgaagtcaacaa
acgaaatcagaaatgcctaa
>Pmalariae|PmUG01_05035500.1: pep
(SEQ ID NO: 21)
atgaacgaagaagtaacgaataagtttgatcttatagatgtagaaccgaaatctctcgaatttttttatgactgtgcagaaaatgtagat
gtttttgttaaaaataagaataatataatagaaaaaaaggtcttgacaataaaaaatatatgtacaaaaacgcagaacattaaaatattt
gagtcagattcaatatatttaaaaataaaagggctaaaaaaaaaaaagttagctccaggaactagtgaaaaaatattagtacaattct
ctttttgcaattttgatacaaaaaaatgtaaatactatagtggcaatagcaatagtaatagtggtgataataatggtaatagtaaaagta
ataataataacaaaacggggttgttacgtaaactgaacaatatagagtgcacagtatatgtgaaagtacagagcgaatattcgacatt
gcatatacccatttacattaaaaagaagattcctttattcgtttttaataacataataaactttggagtatgtaaaaataatatgacttacca
tttttctcttcaagtaaggaatgaagggactaaaaaagggaccttcactatatgtaaggaaaattttatagaagaaggaaatgaacaa
agtaggaacaacgaaattgatggtggtaaacatgcattagttcgcttcaacggtaatatagaaacgaaagagaaggaagacaaaa
taatcaaaaaagaaatgagcgaaaaaaaaaataatcttatcatcgattttgacaaaacatctattgacctggatataaatcaaagtga
aataataaatatcaaaataataaatacaaaagaagaaaaaattcagcgtacgtataaagtcttaacacaagaacatagttacttctgtg
agaaaccgaaaaacataataataattgcaatttttgtaaacagtcagacttcctttatttttgacaatgtaaaaactaatcaaataaacct
taattatatgtattatggaaataacagaaaatttcaaggccgactaaaaaatgaaaataattaccccatattttgcaagtatgaaatatgt
aaagtgaatgtattctattctatacagaattttcaaaagtacgctgatgacaacaacttagatgtagggctgctcatgcacgatttaaac
gataaagcagaaaatgaattaagtgaagaagaaaaaagagaaaaagaaaagttgtttactattgcaaaaaagaggttaaaggctg
aggataacataactgttaagttagataaagaagaaggttatcctgttcaaaaattaggatatcatgaaattttatttgagctccaatcga
aatgtgatattaattttttagaaaaaataaacagaaatacttcgtatttgctcaactttcctgctgttttggagttaaatctacatattagga
aagttgatgaggataaaaaaaagggaaatgatgatactccaaatagggatggatatacagatatgcatagcagcacaaatagggg
cgatgaagaaataaaactgttcttcatcttctgcttaacatttccttgtttcgcctcaagtagttattttctaaattacgaaacagttacgtta
aatgaaggaaagacattaataatagaattaatgaataagaataagtttttaaaaataaattattgcatttctaaaattccttatctaacatt
gaataagaaagaaggttgtatcgtccctctagggaaagacattatatcgcttaaattaaaatgtgataccattaaaaatattgatgaat
atatgtacatatttttttgtaataatttatatttctttgttcttttaatatatgcacaggttaagtctgtatttgcatcaagacatgtgtcttcaac
aattttgataaacaagaaaaggacaaattacaatggggatgttgttgatagtaaattttcatcttcatctaccaaaaaaaaaaatgacg
aaatatatgaacaaatcataaaaaatttggaattagaaagagtagacagaaacttatatttagaagatgggaaactcgataagtataa
atataatgagagtttcatgaactttttaaaagataacaaatcaaaatataacgatatattaaaatatatgtataaaaaacgaaaaaagag
gggaaaaataaacaacaataataataacgataacaataacgataacaataacgataacaataacgataataataacgataacaata
acgatgacaaagtagtgcatgataaagattataagcgggaaaaaagagcagaagaaagaattaacaagatagtgaaagatctaa
atatatatcttaatgacactggaatgggtagaatggatgaaaagagcaaaatgagcaagacgaatagaacttacaaaacgggtaa
agcttgcaagattagtagcacatgcattcaacatgcggtgtatgaggagaggagaaaaaaatatatgaacatgaaaaagaaaatttt
taaagaaaaggatatagaagaaatgagtaaaaaggatgcggatttgttttttcacgagatcttagagcaacatcaacttaataatgtta
attttccaaaaataattcattttgataacgttctgttaaataataattaccaaaaacagtttgtaataaccaacagtaataaagattgcagt
gtaaaaataaatttcatacatagtgataacatagagctaaataaggacattttaattttgagcaaaaattcggacaagcatgtaaatata
aacttcaagttattatctcatactaatttaataaatagatacagaaattatcaaaatgattctatatttaagcaatctagtgaaatttttaatg
agcagttaaatgaaaaaacctcacctcaaaatgaagagataaagtgtgtgctaaatgaaaaagggaataattctacctatgatgaat
caaataaaattttcagcaattctataaacgactttttccttgaacagacatacaaagaaatgatacagctaaaaattaataatgcgtata
cagaattcatcataattaaagcgaatttagtatatttaaacgttttacttgttagcgatgttttatattttcgttttcaaggtacagatcttgg
gatgattcaggaaaataaactagtcatgtacaaccccttcaatacccctatttgcgtaagaactacttgcaacgagcagctttttaaag
tggatgccgaatttaccataccccgtaatgcttataaaactgtgcctgttaaatttattgcaccccaaaatgccagtaatgtggaagag
tttattcaaatctacttggacggcagtaggctatacaaaagtgtgaaatgcgaatgttttgtaaataaggtattttataaagtaaacaca
acagagattaatttcgagcacatacttttaaataaaaattatgaaaaggatttttacttaaccaacacaagtgactctcttctctccttcga
gtgcgtctacaagcccgattgcgttagtataatttcaaaatacaattttgttaataaaaatgaaaaatcgaaaattactgtgtgcataaat
ctgaaggagagtgaaaaaattaaagataaaattattttccgcataagaggctcggagaacttggttattcctataacggcaaagcctt
ccccatccaacgtgctcctctgcaacaacgtacaagtggcacaagaaagtaatgaaataaaaaattatgaaataaatattctaaaca
aaggtttgtgtgaggaaagcataattttagatctaagtgagctatcctttttaaatatctacacaaataacaaaggacaaaaaaaattaa
tgatgtcaaataataaggaatataaaaatgcggatgagatgatagatagccaaatgattttaagtaaaatttcaaaattagaacacga
agatatactaacagatgaatgtaccaagtattactataatcagtttataaaactttataattttataagtacgtattataagcataaaggcg
aaataaggtttagcgaaaaaggggaaaagggcgtaccccttcccaaaatgtatagaattaatattcctccaaaaagttttgcttcctt
gtattttcagtgctattataacaagtgcctaaaaaaagaaatcgcatttaatggtttattaagatataatggtaaaaatgataacagtaat
aatgataatagtaacaatggtaatagttatcataccgcgtatggaatgaaagaagaggaagtaaaaatagaaattagggccagctg
cttagaagtagaaccctattttctctactttgaagatatgcttccctctagttcagataaagcattaataacctactttacaaaggaaaaa
acgaaaattctaaaaattagaaatgcgtccagtcaggtggttcggtgggaaatagatatatgcaatgaaaaggaaaaacaaaattat
ttgatcggaataagaggtggagattgcagggagagagttgaaaagggaacggttcactccactctagaagaagaggcacaaaa
gaaggggagaaaagatatccccaagaagggaagaagcgaatggggacatatgggacagaatggagaaaagaccgtacaggc
tctaaaagtagaaaggggcacagaaagcaaagtgggaaaaagaaacgaaagaggagaaataggagagaaaggaggcgtag
gagacacaggctatatatttgataaagaaaagacagacaatacaggcaaacagaactactggcagaaatatgaaatagagctgg
gaaaaaaggaaggaacactggaaccaaaaggggaagtagagatacaagtgaaattaaaaaattttacacaaagtggacgatac
gtagatatattgtcaatatcgtacgatcctgtggaggatcatatagaaaggagtagtaatgaaaaagaaggaggctcaaaagagat
aataatatcgaactataaaatggaattgaaaaaaataaattattgtatttttatgcttctaagttgtaaaataccgagattgctcttcgatgt
aacgtatattaatatcatacataacaaattaaatgaatacattactttcccttttcatatatacaacagtggttataattatgtccgagttaat
tactaccttaataatttatacgaagaatatttctccatatcattaaattttacgaacggaaatgaaataaatgaaaaaataaaaaaagtag
atgtatgtttaaaatgtatggctatccgaaatttgaaatttaagacatatatcattataactgtgttagatcatgaccagtatgtcattccgt
tatttgtaaatattgatgaaaacattgattatctttttgataaattggatcaagaaaatgaacatctacatgtgaaccattctccttcgatgc
ctgctttgcacaatatgcatattcagtgtggaaagaaaaatttatcagagttagagaaaataaaggataacccgatcggttataggaa
taacgatcaaggtattgaggatagggatgatggatatggggatacacaacacaacatcaaccactgcagcacttacagagatgac
cgagataagcagagtgctaaagatatgtcccataccaggaggagtaacaaagagaaaacaaatggaggcgaatgcgtatacaat
tccgaatatgaatctaccagttgtacatgcagcagtgtttgttgtgatgataacatgcttagatgtaattgccaagagagaaacagtaa
atttctgtctttttacagcaaaaatgcgggccaaagggacataagaaaaagcgtatgtatatacaaagaattaaaaataaaacatata
acaaatgtatattataataaagaaaaatatttattaaacataagtgaaataactaaggattatgtttttatatttcttcaaaatattttactaa
gtaaatactatgctccaagtgttatagaaaatacaaacgatctattcctattttttattaatttattgtatgagcttttcttactatacccaaat
aaaaatatacaaaatatagttaatataataaatgacgtaaaatcttataaaaatatatcaatgcaagacttggaaaaaaataattttctcc
aggaaaaattcctaaaaaatatgaaagctcttttgaaatacttagaggaggaaaacttactagtgtatcatataatttatgaaaacctat
tacctctagatttgtatctttggtacatcttcgacaaagtacctgactacttttacctaaaaaagggcaaaggaggagaagaagaaatt
aataaaagaaaatgtatacaaattgaagatgtagaaaattttttaacaaatggaaataataaaatattttatttagatagaatttttaaaac
tcatttaaagttattcagttactcatggattactatattgttcgaacttttaaataaaaaattatttagctgtgtcaatgtaacctctttgaata
aattaagaggaataaaagtgtgtaacatagaaaataaactcaatgaaaacataaaaatttataaaataaactatctaattgttttaaata
aacaagacaaagagccaaacaaacataccatttttctaagtgagaaaagaaaaacatcaaagagaaaagaaaatagtagttcaac
aaatattactccaaaggataacaacccttacgaagataaatctgctaaagttagcagtgtaatcaatatggggaaaaataaaaataat
tacttatccaatttttatatgaaaaatggtagacccacacatcataatagtattataaagaaaaaaaaaatgaataatggtaaaaatacc
tacacgataaattattttaatttttatgaaaataattttatcgatttaaaaacagttgtaaataaaattaataatttcactcattgtgaggaatt
cgaacagggggtggaaaaggagcgagatatatctttagaaaagcatgaacaaattataaatagagatgataaggaaactacaca
gagaaatattcgaaacaaaacatattccaaaggggaggaacaggaaaaggaagagaaagacaatgtacaatttaaaagagatac
agtggtaaaggaacaaaaaccatgctttcactattttaagagtgctgaatacatactatttgaatggttgtattttcattataataatataa
attttgcaaatgtagaatataaaaggtatttatttaaagaggataatgagaaaataaaaaaacaacaacaaaaacaaaatgatttaattt
ttcctaatgtaccaatgaataaaagcaaagaggaggaagagacttcttcaaaatgtatggagaataatgatgtgccactcatattaaa
taggtccaacttttatgttgaaaaaaaaaaaaaaaaaattatggaaaaaaaagataaaataacaagtatatgcgatttaaaagatttag
taataatcatatatactataatttctcacattccgtattatttgttttttaaaaataaaataaaaacaaaatgtatatctaaaaaggattacat
atataacatagacattttgttaataattctgcatgaaatgaaattagataaattaataagtagacaagtattgattgagtttaattatattca
catatttttgttcttggcactatgtattttatattaccaagctatttgcctagttactacttagttgtggatgatttcgcctcctacacaaattg
cgttggtcttataaaggaagaaatgataagcaataacaaaaataaaaagaaaaatacttgtaatcaaaacgaaaatgaaaaggcag
gtattattaaacttaatctgactacacctgaggagcgtgttataaccatctacaatttaaataattacaagtgtaaatataatgtatttttaa
taggttgtaacaagtaccaaattgacagagatcatatagttattgatccacttaaatcagaagaaataaaattgaaaatagacaaaaat
tatgaccaaactatttgtaagtataataataacactagcattcaattttcaacctttaaaaataggagaaaaaaaaaggaaactatcgat
gaaactacaacatattttgacgaaagttattccttttcatcgtgttcatcggatgattgtaaatgcaattctgatgaagctgaaaagatta
gtgtaagtaacaatggtgatgattccttgaccccaaatgaaaattacgctatattagtcttgagggccgaaaacaactacttccctga
cgataatgatgatgaaaagtgcatttgcattaaacttatcgaaaaagacacacatgaaaagtcagatctccaggcaagtaatttaaa
gaaggaaaaaaatgagtggaatgatttaaatattaatgtaaagaattccgaaacgaggaataataatatcagtaaggtgcacctttc
gaattcctctcaattaaatgtaatgcttaataatagcgaagtaaaatgtttgaacttgagaggtaaggtatatgaaaataaatgcttgga
aataaatttgataaacaacacaggacgaaacgtagaaataaattcgaatatttatcacttatatttaaaggatttaaaaaaagaggaaa
agaaaaagaaaatttttgaaatagatataataaataatttaaacataagtaatgaccaaatatataaagtgtgtgatgtatgtacaataat
taacagcaatttaaggaatcatttgaatagaaaggaaaactattttagctgtttttatgtagataatacaatacctattattattagtagag
aaaatgaaaagaagaaaaaaaaaatacaattacactttttcccttttatggaaggttattatttttgtttcatcctatttatgttaaggatgt
aaaaacgaaatatttgatatcaatgtgcaaggtcaattgtttcttttacataaataatataaaagaggaagaaataattaaaatgaattct
caattgtatgatttttcgttcaatataaaattaaatccaatcaataaagaatttttgaaatgtgtaaaatacattttgtgcaatttgaataaaa
tggacgagaaattctttttaatctatttgaaaagttatctacaaaatgtaagcaaaaacaggagtagtttttatattacatgtgacaggaa
ggataaaatgataataaaaaaaaattttgtatcctttgaaaattttgattataataattatataagtcatattaatactccaaatttagacata
gacagattatatgatataataaaagagggaatcaattatttatgtgaactgagcataaaagaggagagcaacggtgttttcgagtata
actgcgttcttctaatgaggaaaggagaagcagagaagcagaataagaagtcgcataaacatactcagcagaaagaaggtctaa
aaacagaaaaagacgcagactgcaatatgcttagctacgaacagattggacattcccatttaagggaacaaatcggcaatcatga
aatttgttacaagttgtataaaattatagcaaatgtaaataataagaaaaataataatataaactctattataacatttaacacgaacgca
tatttagaaactaagaaaactatatcaatatacaattacacgaatagcactataacgtataagtgtacatactccgacacagtagatca
taataacaataaaataaattataaaatttttagcagtaatgaatatgtagagatacctaaagatgtagaaacgtttaattttgaagtaactt
gttattctattgtagaagtgtcttgttcatgttttattatctttactaacataaaagatgaagaagatcaaataaaaattaagcttcaagcca
atattaaaaaaccttacccaaaagaaacaattcatataaaactattaaacagaatgaaaaaaacagtacaaattgaattatataacga
actgcagtttcattgtgaattcaaaatttattctgatttgttcattttattcggtgataaaactatagaaatgctacccaaacagagaaaag
tgtataccttctgtgtgagaagtgctcacataggtgagttcgtggggtgcctcatatttaagttttaccggttgataggtgctgaaagg
ggtgaccttcacagaagtaacgttatcgttagtaacatcagcaacgttagcagtgaaagcagaagcaacgttagcggtgaaagca
gaagcaacgttagcggtgatgatacaagagagcatcttttttattattttttctggtacaaattgaacatcaccattgaactgaacaagc
caataaaaatactgtttcttgaaacatccgtgggagacaaagtaaataaagaaattgttttgaaaaataatggaaatgaaagcgagg
actacttcttactaacatacatgaatgaatatatagaaaagaagcaagttaagatacaaaaaaatggcacgtacgtccataatattaa
atacatgccaaaaataccaaattattttcataatataaaagaggcttcagacaacttcaagttaagcactaaaagaaataaatcttttca
ttattgttcggttgatcagttagataaagaagtggaagggactaaccctcttcaaagtggttataatgattttccaagtggtaaaaaca
gagattgcacatataaaggaggcaaggaaatggaccttgcaaaggaattgcacaaattttattttccgccaaatgaggagaagga
ggaagaaaagaaagaggtcgaggcgaaatgggaggaagaaaaaagggaagaagtgaatgacgacgaagatgaagcaaaat
cccagggagaagacgaggggaagggccaaaacagccacttagctgaggaagataaaaaagaaacagctaaaaaatgtgaaat
tagaaatagaagtagaagtagaagtagaaacagaagtagaaatagaaatagaagtagaagtagaagtagaaatagaagtagaaa
tagaagtagaagtagaaatagaagtagaagtagaaatagaagtagaagcagaagtagaagtagaagtagaaatagaagtagaa
gtagaaatagcagtagaagtagaagcacaagtacaagtataaatgtactcagaggtgtacgtagcagtagtggaaaagaccctca
gaggaaaagcccctcacgagatcatagtagaaggaaaaataaaagtgacatgttaaggaaaaaatgtaaaaaaaacgttgagag
agataataaaaaaatatttgaagagttaattaattattgcaaaagatatataaatacaaatattaattataagaatcagaatattggattttt
tttcatatataacaagaacgagggtataaactattacattttgatattactagcaaaaatgaggagggaattagttatagataatttctct
gcatgcttaaccaaatattgtctacttaatctccacattaatataataatgaaaataaaagatacgtaaaagatttaaatacgaataaag
gtacatgctattctgataattacagctacgaaattgtggagtgtgtaaataaaaatggtggatgggaggcagaatcagaattagcag
gaaaatgcaaagacacacaaccagacgtttcagttaataaacatgttaataggggaaaaatttatggggacacttatgatagtacca
ttagtcgtaataaccaaataaatattgatgatcataaatatgataatgctaatgtggaagaaatcataaattctcaaattaaatgcatttat
ttaagtaacaaaacgaattatagtatttttaaacatacagataaaaataatgtcattataaaatataagccaactgtaaaaacatcacaa
gaatgttatgccataataagaagcaatttatacggtgattttatttacctaattaaaggtatgtatacagttaagagaaaaataaaagag
aaaatagtaatgtataactgttgttgtttataccactttaatataaatttatttaacccactgaattgtagtatatatgtaaaatgtagattca
aaagagagaaaagtaaaaatgatgagctttatgacaacttttatttggataacaataaagtgggaaggaaagcagaaagggaaga
atgctgcttttcctccatggaacacgagaaaatattattaaataaagaaaggagttactttaaaattttaagtaatgaacattttaaaata
aaaaagagaacacatttttctatgctcatatcatacttttgtaaagaggcactatcaaaacatacgttaatagtaacgttaaagccttttg
gcacggataaggccagtgatgggacccttccaccaatcatatataaatatgatatcacttttaataatgtggtaaggcagggtatcga
agcgtattcgttgactggttccggtgcacaaagtacggatgaactcactgctaggggggatagcagcagtggaagtagcggtag
aagtagaagcggcgatggaagtagcgatcgaagcagcagtagtagccctagtcgtcgcaacagacatgcttgtgaaagtggtg
gtacatgcgaaaaaacaaagagctactctgtaaccagccaaagtagcagcaatatgctgaatgggagtgaactaagtaaaagcg
atttattccaaagagggttaaaaaaaaaatataattctaaagcaaatgagttagtagaagaacaggaaatttgtttacatgcatgtgat
gaaatattagtagaagataaagaagaaaaggacaagtatgagaggaacatgagctatgtagtagatagaaaggaagaagtagttt
actgtgatggaaaaatattcatcgaaagtataagtaaaaagaaaacatgtttgaaaataaatatagataaaagaaaaattatggaaag
agcagattataacaatatcataaacgagttagaaaactgtaaaaaggaaaagagcatattacacagaaatattgaaaaagataagt
gcaactataaaatttttttaataaatttaaaaaatgtaagagatgcaacaatggatgaacaaaataaaacaaacctccatatgaataaa
atcctctttgatatcagagagcagttactaaatgattacttatatgttcatattattgaggatacagcctccgctttggtgcttagcttaga
gctcttcgcgcatttgccattccactgtagttttttcataatgattaagagggtagaactaggaagtcgtgaggtaggaagcgacgaa
gtggaaagaggcgaaatgaacagcgaggaagaggataactacgtgctcgtggaatcccttaaaagtgagaagagaaaggaatt
gacaactatcgaaaagaattacatattcgtcgaaatttttaattatataaatttgtctaaaaaatatatcaacgttatagttgatgataactt
atgctcagaaacaaagttaaatatattttacaaccacacaaatgattcttattttgacgcatatatcgtcagttacaatcaacaaagtaat
gatccatttgcagatgaaattatgaattttgatataaagcccaagtctggaatattaaaacatagtagttacaataaatttgtaattacta
ggagtaacaaaaacaacataataacatttaatagttcattcttacttctaattaaaacggaaaagcatattttttcctacatcgtctttgct
cgctatacccctgctagcgatctccacacagcgttctctttgtatttccagcaggagcaaaataaggtaaaggaaatgctcaatgaa
aacaagaagaagttcagcgcggtattcaacacattcaaacaggaaaaaaaggagagtagcatatttaacaaaaaaggtcagatta
taattgaagatatctctaagtcaacaaatgaaatccttaactgttaa
>Povale|PocGH01_05029200.1: pep
(SEQ ID NO: 22)
atgaacgaggcgacaaataagttcgatcttatagatgtagaaccaaagtttatagaatttttttatgacaatgtagaagatgtagatac
atttgtccaaagcaaaaataacgtgatagaaaaaaagattttgaaaataaagaatgcatgtacaaaaacgcagaatgtaaacattttc
gaatcagaatcaaggtatttaaaaataaagggggtgaaaaagaaaaagttagcccctggtactagtgaaaaaattttagtggaattc
tctttctctaatttagatgtaaaaaaatatagatttggaaaaaaaaaagatatcctaaatattcttaataatgtagaatgtacggttagttta
aaaatacagagtgagtatacaacagtgcatatacccatttatataaaaaagaaagtacctatattcatttttgacaacataataaatttg
ggaatatgtaaaagtaatcgtacataccattactctctcaaagtaaagaatggtggttcgaaaagggggaaattcacaatatcgatg
gacaatattgtagaagaggaaaaaacagagcaaacacatgactacaagagggatgaaaaacagacaaaagttcactttaacggt
gacattaacaaacgaaaaaaagaagaaatagaaaggaaagatgtgcatacaagtaagaacgatttattcgtgcagtttgacaaaa
caacagtcgatttaaacataaacgaaactgcaactataaatattacaataaaaaataaaagggaagaaaagatttatcgtgaataca
aggtttatacaaaagaacatagttacttttctgaaaaaacaaaaaatgttattataatcgcaatttttactaacagccaaacatccttcatt
tttgaaaatgaaaaaaggaaccaaataaacctaaattacatgtacttgggtcataaaaaaaagttccagggacagataaaaaacga
aaacaactaccccgtattttgcacacataaagtagggaaggttaatgctttcttttctatgcaggagttcgaagcctactctgaggaga
acggcctagacattgatcagctcagtcgcggtgaggacgataacaacgaagggaaatttgtggggacgtttgttacggaatctcc
atgttcttctaccgcctactcctttagttatacacagaacacgttcacctcccttacgggaagtcgtgattttctagataccaccgtattg
tggacatccgccttgctcatttattttttgttttattttttttttctctcttcttttcccatccacatccttgcagatatgagaggcgtgacgcag
aatgagatgtgtgaagaggaaaggaaagagaaggaaaagttgttcgcgattgcaaaaaagagattgaagcaggaagaaaatata
acagttaagctgagtaaggaagatgggtataccgttgaaaaactaagttatcacgatgtttcggtggaagtgcaatcaaagtgcgat
gttcgctttttggaaaaggtgaacaggaacatgtcttaccttctaaacttccccgttgtagtggagctaatgctgtacgtctgtagtgg
agaaagtggtgttagcggtagcggggggatcaaatgttgtcacgaaagttgcgacaaccgcaacctcctggacgaagagattaa
gctgtttctcgttttttgcctaaccttcccgtgcgtggtctcgaacacgtactttctgaattacgaaacagtgagcctgaatgagggga
aaacactgataatagaagtgacgaaccgaaacaaatttttgcaagtcagttactgcttttcgaagataccttacctaactctaaacag
gaaagaggggtgcatagacccaatgtgcaaggaaataatcactctcaagttaaaatgtgacaccattaaaaaaatcgatgaatatat
atacgtgttcttttgcaacaacttatatttctttgctatgctaatgactgcagaggtgaagtctgcctacgcgctgcgtcaagcatcctct
ctgcacttggaacaggggaagggtgaaaagggtctcaccaacagaggaagcggtgaccgcgacttgggcaaccgccaggatg
acgaaatttttgaaaagataataaaaaaacttgatctagaaaaggtagataaaaatttgtacttagtggatgggaaactggacaagta
caaatataacgaaaattttatcgcttttttgaagaataataaaaaaaaatataatgacatactgaaatacatgtatgaaaagagaaagg
gaaggaggaaagacaaaactaacatggacatgaaagagaaagaggaagaatgtatggaaaagaaaaaattaaaaaaaaaaatt
tctaaagtaattaacgatatatgggtatatataaatgatacaggattaacaaaaataagtaccacttgtattcagcattctgtgtacgag
gagagaagaaaaaaatatatgcacataaaaagggaacacttggaaataaaagatacgaatagtgcaagtaatgaatgtatgaatgt
gccatgtgatgaggtattagaagaacatcagttagacaaagttatctttccaaagaacattcacttcaaccatgtgttgttaaacataa
gttataaaaaggaatttactgtacataacagcaatacagactgcaacattagattagatttcacccacagcgacaatgtgaagataga
taacgatatgttgatagtaggtagaaattcaaataaaaacattaatttggaactaaagataagtccattttttaaaaaggtggagaatat
gacaaatgagaaaggaaccactacgtgtcttttccctaccccctctctccaaatagagagagaaaataatatgttattcaacaaccca
gaaattgttatgatcccgtcagttaacgattttttccttcaacaaatatatacggaaagaatcccattaaaattgaacaatatatacgaac
aacaaataacagttaaggcaaaccttatacttctaaatgtccttatcaaggagcaaacattacacttttacttcgaaaactcggatgtcc
agatgtgttgcgaaaggcagatcatcctgtacaatccctttgacttcccagtagccgtgagtgtggcatgcaacgagtctctcttcca
aatagatgcccatattaccattcatcctaactcgtgcaaaatggtacctgtcaagttcctagcccctccaagagccggcgttttggag
gactttatttacatatatttggatggagcaaggttagtccaatgcatgtgctttgtaaacaagtgttcgtacaaaacagacataacaga
gataaactttgaaaatataattttaaacaaacattatacaaaagatttttacctaacaaatacagccgactatccgctcatattcgaatgt
tcgcacaagccagaatgcgtgtatgtccaatataagtacagttatgtccaaaaaaatgaacgcctgaaagttactgtttcggtgaact
taaaggatggtaaaaaggtcaaggacaaaattgtattctccgttaggggtgctgaaaatctcgtcatccccataagtgctaaggctg
tcccaactcacgtattgtttatgagaggattgcatataaagcaagagagctgcgagatgatggattacaagatggacatcctaaaca
agggcatatgcgaagagaaggtaatcttggatctgacagaactaaattttttgagtattagtattagtattagagagaaaaaagagaa
aaagacattaacatacaatagtaaggagtataaacatgcagaagaaaaaagagatgacttcccaattttgagagagatttttcatgta
gaatatgaagacataataacagaagaaagcacaaaatggtattataataaatttataggattatacaattttatatgtaactattgtaga
agtagagggagtatacaattcacagaaagaggagaaaagagctcacaatgtttgtatatggttaatatacccccaaagagttttgtct
ccatgtatattaattgctattacgacaagacaataaaaaaaaaaatgaaatttaacagtttgttattacaaaataaaattatatatgaaaa
aaaggaagaggagattaaagtagaaattgatagtacatgcttagacatatccccagcctttatccacttcacaaatgtactcccatca
aattcgcacagatctctcatttcttattttacaaaagagaagaaaaaaacgttaaagattaagaacgtttctgactatcctatcgagtgg
aagatttacgtatacaccaatgaacagaaaaaggttcacctgatgagaagaacagggggaaaggccagggcaaccgcaacaca
tgaggggaatcagacagctagaggtgtattcaccggggagaaagcggcaagagaaatagcgacaaagcaaataacggcaaag
cacataacggcaaagcaaataacggcaaaacaaataacggcaaaacaaatagcggaaaagcaaagcgtagccatgggggtat
cgaacagagatatagaaacaaagggagacttagtaatggggagagacgaagtggaggaggttgcgaaagcatacgagttggat
gtgagcaaacagagcggcttactgaagccagaggaagggacggagatagaggtaaaaattaaaaacgtgcttcaggggggaa
gatatgtagaaatattgggcatatcagcaacatttgtggacaatgaaatggggaaaaaaactgacagtaatgtaggagacacagaa
atcgaagtattaaagagtaccgttcgaagagaggaaaaaacttattgcgtttatatgcttctaacttgtgaaaccccgaaattattttttg
acgttacgtacataaatataatacataataaattaaatgaatggttcacctttccttttcatatttacaatagtggatatgactatgtcaga
gtgaaataccattttaacaatttgcatgaagactattttttcttatcgttagattttgaaaaaggaggtgaaattaacgaaaaagtaaaaa
aaataaatgtttctttaaaatgtaaggcatataaaaatgttaaatttaagtctaacctttctgtaagtgtgttagagcatgatcagtacaat
atacctctctttgttaacatcgatgaaaacattgattaccttttagataaagcaggcggtgtagaagaggtatctttccagagaggaaa
taaaaatggagagaaaacacaaaagggggatattcacgtgggtagaaaaatgctggataatatcgacgagagggaagactacc
gctatgataacccatggaaagaacaattggatgatactacccctttcgaaggctgcaatacagaaagcaatatatctaaacaaacaa
aagacacattgttacccttcttctgcagtaacagtgtagaaaaggatgaggaagaaattgtcaatatttataaggggttacaaattaga
aacattacaaatgtgtattataataaagaaaattttacacataacttgacagcaattttaaacgattacatatttatatttattggaaggatt
gtgttgaacaaatcttatctcccatatgtcatagaaaatacaaatgacctatttttatttttccttaatttgttaaatgatctttttttactgtacc
caaatagaagcatacaaaagttgataaacggaataaaattttatgaaaatattgccatgacgaattttgagaaaaacacattactccat
gatagcatacttcaaaatgttaaaggaatattgaagcatttaaaggaggaaaagctactcgttgatcatatcctttacgaaaatttgctt
cctgtagatgtgtacatgtactatatcttcaacaaggtaccagaacacttctacgtaaaagggaaaaaggaagaactgggaaaaaa
cgataacggtgatagtaatgaatacatttgcaatgtagatgtgaatcaatttttacttaacggaggagagggagtgttcttttttgatag
ggtattcaaaacgcatttaaaactatttagctactcgtggattacaattttcctagaatttttaaacaaaaatatgtgcagttgtgtaaatgt
aaactcgttaagtaaattgcggggaattaagttgtgcagtatagaaaacgagataaatgaaaatattaaaatgataaaaataaactac
ttaattgttttaaataagcaggatcgggaattgaacaaacacaccgcctttttcgacgagaggaaaaggaggtctcgaagaagcga
aggaaccagtagcggcaccatttgcagcggcaatggcaatgctagtagtagtggtagaggaaaaggaatgcagaggatgccag
tcggggtgattgggatggaaaaaggaaaaaagacgatgcccataaaagaggcgaagggcgaccccagaaatggcaaaaatgc
tcaccacggtaagaggatgaaagttgggcttatcaaaaatagcagtgctataaattacttcaatttttacgaaaacaacttcatcgattt
gaagaaagttgtccagaagataaacagtttgaactgcgagggaggggatgcagaggggaaaaaaagggatgaccagttaacg
ctcgaaggaagtcgtaaggagagagatacaaattcaccaaaaggtagttcacccatggaagaattaagagaggggagaaacaat
gggatactcaaaagagaagtagaaaaggaacaaacacgcaactttcaccaaattaaaggtgttgagtgtatacttttcgaatggtta
tacttccattataacaatgtgcattacagcgatgtacaaaatgaaaggtacgtatttaaggagagttggaatgaaaatgaaaaaggc
gtcgctgatttgggaaaattacccagttcgaggaaagttgtaaacgttgcagaagtgagaagggaggaagtagaagaggaggtg
acgccccaacatgaagtcaatgaggtagtgaatcaacatgaaaatagcgagggaaggcccgtaaaattaaataaaagcgattttta
tatggaaatgggaaagaaaaagaagaggaaacatttttcagagaaaaaagacaaaataacgagtgtgcatgaattaaaggatttg
gtgatgatcatatataccctaatttcacacattccatattacttattttttaaaaataaaataaaagtgcattgtaaatcggagagagacta
catcaataacgtagacattttgctagtaattttgaacgaactaaaattaaacaatttgatcagtaggaaagttttaattgagtttaattatat
tcacatgttcattttcctcacatcgttatattttatacttcccaattatttacctagctactacctacttgtagatgattcttcttcctacaaaaa
tgatatttgtgctataaacgaagaattaatacgagaaaaaaaaacgaaaaatgtacgtgtccaaagcgaaaataaaaaaataggca
ttcctaaaagtgtgagcaaggtggaagaaagggttataacgatatacaatttaaacagctacaaatgcaaatataatgtgttcctcat
cggatgcaataaataccacgttgacaaaaatttcatccacattgacccccttaaatcggaagaaataaaaataaaaatagacaaaaa
ttacgaggaggccatattcaaatacgactacagtacaagcataaaatttcccccccttaaaaatggattcccctccgacgcacctag
gtttgatgactctgaaggcaccgatgcttctactgcatcttctcatatgggtcggagtagctctacttcatgtttgtccgatgaaagcca
tgaggaagatgcaataatgggtaaaaagacaaagcatcgctcttttacgaagaatgaaaattatgctgtaatagtgttgcgctcaga
atgttatctcccggaggagagagatagcgagagatacatttgcattagacttgtggaagggagcggagaggaggagacggtaat
actggcgaataagggcaatttaaaggataagaagaatgaggggagtaacttaaacgttgatatgcgtgtttcgggcgggaggaac
gatgtgagtagggtgcgtcctacaaactcttcgcaaataaacattatgttgagcagtagtgaagtaaagtgtttcaatttaagaggca
aggtttatgaaagaaagaatttggaaataaatttgataaataatacagaaaagagggtagaagtaaattcgaatgtttatcatttatata
taagagatttaaaaaaagaagaaaaaaaaaaggacaattttgaaatggacatgttaactgtagacaaaggtacccacgaaatgtac
aatcaatgtgttgtttgttctatgattaataccaatttgaggaattacctaaatgaacatgaacatttttttagctgcttttacgtagagaat
ggaaaggatttaaccattggaaaaaatgaaaggagaaagatacaactgcacttttgcccttttgtggaaggcaactattttggttttct
cctattttgcgtaaaagacaggagaacaaaatatttgttgtccacatgcaaagtaaactgcttcttctacattaataatgtgaaagagg
acgaagtaattaaaatgaataccttatcttataacttcacttacgatatgaagttaaatcccataaataaagagtttttgaaatgtgttaaa
tttgtgctatgtactttaaataaaatggacaaaaagtattttatggcctatttgaaaagttacctaaaaagcgtcaaccaaagtaggcac
aatttttacatcatctgtgataagtatgacaaggtggctatcaaaaagagcctcttttcgttccagaatctggactatgccaggtatgta
agcggtgtagatgcaggatcatccggcgacgtggacgtggacgagctgtgcgatgcggttaaacgagacacgaactgcttttgc
aatttgaatgtgagggaggaaagcggtggggtgtatgagtacaactgcatcctgttagtgaagaaaaaccatgaagaaaaaaaaa
aagacatttcaaatttcgtaacttatgaggatgttagaaatatgagtgtgagggagcagaattgcatacatgagatgtgctatagattg
tacaaaattatagcaaatgtggacagtgaaaaggcaaataaggtattcactattgtattcaatactagcgcctatttattatcaaaaaag
agtataccaatatataattacactaatgaaaaattaacctatagatgtattcattcggaaataatagatcagcataataacaagatagac
tataaaatttttaaaaatgatgaatatgtagaaataccgaaagatatagaaacgttcaattttgaagtaatatgttattctattgttgaagtt
gtgtgttcttgtattattctcatgactaatgtagaaaatgaggaagatcaaataaaagtcaatgtaagcgctaatattaagaaaccttac
ccgaaagagactatacatataaaacttcttaatcgaatgaagaaaactgtacagatggaactatataacgagttggatttccattgtga
atttaaggtgtactcagatctaccaattctttttggggataaaaaaattgagatgttaccaaaacagagaaaggtatacacgttcttcgt
tcgaagtgcgcacattggtgaatttgttggatgtctcattttcaagtttttcaatctcgtcaacatgaagggtacccatgcttctactcgg
tatagcagtaatcaccatgatagtgatcgaccaaatagtaccgtggatagttttcccttctctgattactttttttggtacaagttaaacat
cacagtggagctaaatcagccgctaaaaattttgctccttgagacaactgtcggggatgaaataagtaaggaaatcgttttaaaaaa
taatggacgtgaaaaggaaaaatattttttgctgacatacatgcacgaatttactgaaaggacacaaattgaaataccgaaaaatgat
attcacgtttataatattaagtatgcaccaaaaataccaaactattttgataatctacgcgatacgatggaatgttacaagttggacagc
gagcaagagaagattgatcctttctgtggaacaaccatttccaattcgttaggtcgtgaaaaggaaagagtttcttctaccgaaggg
gacgctcccagttttaccggtggaggattcgacattgcacaggaactatccaagttttacttccctccgaacgaggagagcgagga
agagatgctagtggaaagtgcggtgactcgggaatcgtccagtggaagggctgtcattgggaatgtagccaatggagaggaag
ccaatggagaggaggccaatggagaggaggccaatggagaaacagtaagaggagaaaaggacgaatatgcagcttccaggg
taaaaaacctttacctgaggagtgaaacgatgaaaaacggaggagaacaacccaaaaaaacagcatccatgggtgtaaaggttc
gcagcatggagaaaaaggagcatccaattttgaggcagaatgtaaagcacaaggggaagaagaactttccacataatgagaaga
ggctcttcgaagagttaatacaatactgtaaaagatacataaatgtaagcattaattataccaatcagaatataggatttttgtttatttac
aacaaaaaggagggtgtaaattactacattttaatattgttagcaaaattaagggaccaactaacgataagtaatttctccgcgtgctt
atccaagtcttgcttcatccatctacatttcgattttaattctgaagaaaagagatatgtaagcatgttaagctcaaaggagaatgcaca
ttcgaatgagtatttttaccaggtaggggaatacgtacgacagaggggtggcagtgatgcgggaaaagataagcacaaggttaaa
tgcatttacctgagcaacagaacgaactacaccattgggggaatcgcggatgggggcgtggatgtagacgtaggtggactggtg
tgtgaacgtgaagacgaacacactgacgaaccaggcaaccacgttgtaataaggtacaaacccacgacgaacagttcaaaagg
gtgttatgccttagtgaggagcgatatatatggagactttctctatttcgtcaaaggagtgtatacagtgaagagaaaaataaaagaa
aagatgattatgtataattctggttgtctgtaccattttaacgtaaatcttttcaacccatttaattgcaaagtgtgcataaagtgtagattc
aaaaggaggagagatgaaaggggtggaggttctcatatcatttccgatagttctgagcatgaggcagagaaaaggatgaaggag
aggagtgtccattcagtatctgtgaaaaaagaaaaagttcttataaataatgaaaaaaaatattttaaaatattaagtagggaaaatttt
atagtaaacaagagaagacatttttcactactgatgacttacttttgcaataagataacatcagtacaaacattagagatcgttttaaag
cctctccaggatattacaaacagaggatcattccaatctattgcttatcaatacaagttcgttttcaagaacacccaaaaggggtctct
gatcggagaaaaacatctcgtatgtgatgtccccaatgatgaggtcacctatcacaataggtttgacgaaaattctctaacagattcg
tcaactggtggggaagaaccttatccctgtaacgttgttaaaaaggtggattacaaccatgtcgtttatacaaaattaaatgaagtattt
accactgaggagggggaagcagacagggaggaagaaaaacattcatggcgaatgatatctactagtaaatgggaagaaaatat
ggaaatgcacacaaacgaaggtattgcttctccgggttgtggttcaacaacaaccgattctgagtgcactcaagatggtgacttggt
atctaccccggtgaagaaaaccccatttggtgtatgtgaagataggaaagatgtaatggaagagcaagttgaacaggagaattgc
ggactaagtagtaaggaagtggtatactacgatgggaagttattcattgagagtatttgcaaaacaaagacacagatgctaatttaca
taaacaaaaagaaaatgatggagagagaagattatgacaaattcatagacgagttggaaaatcgaaaaagggagaaaaatatgct
acacagaaatgtcgaaaaagggaaatataattataaaatttttttaattaacctaaaaaatttaaacagcaattggggtggattaaacg
ggaaaacctgcgtaaaaatgaacaacatccttttcgatatgaacgaagaactgcttaataactacttatacatgcatattgtgaaaga
cactccaaccgacttggtgctaagtctggagctgctcgcttatcttcctttccactgtagcttctgtgttgtggtcaagcgcgtggaag
gcagaggcggggcagccatggaagaagcggaggaagtgagggaaacgggggaagcgggggaaacgggggaagcggat
gggatagattgggaagacggaatatacgctgacgcgggggagcggtgcgtgcaggtcgaatgtctcaaacgggaggaaacga
agaaatttacgatcgttgagaagaactacatatccgtcgaaattttcaactacatgaacgtgtcagaaaagtatataaatgttttcgtcg
acaggaacttgtgctccgaaacgaagttaaatattttttacagacacacaaatgattcctatttcgaagcgtacatggtcagttataac
caaatgagtaattatatcctcacagaagaaattaaaaacttcgaaataaagccccaatcaggaatattaaaacatggaagccacaat
atgtttttgattacccggataaacaaaaataaccttgtttcttttagtaattcctttcttcttttaataaaaactgaaaggaatatattttcctat
attatcttttcgcggtattctccccctcgtggcttgcgcactacgagggagcagaacatgctaaaggagatcctaaacgaaaacaag
aaaaagttcagcgttgtctttaacacattcagacaggaaaagaaggaaagtgctatatttaacaaaaaggatcaaataataattgaa
gatatttcgaagtcaacaagcgaaatgaggaactactga
>Pfalciparum|PF3D7_0418000.1: pep
(SEQ ID NO: 23)
atgaatgagatttcagagaataatttgaacattttagatgtgagtcctcgaaatgtcgaatttttttatgaatgtctggaagatgtagatg
agtttgcacaaaagaaaaaagaaaatataatagaaaagaaaagtttaaaaataaaaaacatatgtagtaaaattcaaaacatcgaaa
tatttgagactgaatataagtatttaaaaataaaaggaagtaaaaaaaaaaagttggctcctggtacatgtgaacatatatgtatagaa
ttttctttgtataataatgtagatataaaaaaatataaaaatgataaaagaaaagaagatgttttaaatataataaataatatcgaacgtac
catctttcttaaaatcaaaactgaatatacatctttaagtatacctatatatataaaaaaaaaagttgccgtatttttttatgataacattatta
attttggattatgtaaacaaaacatgacatatcattatcctatgaaggtaacaaatgtaggaaaaaaaaaaggaacctttacaatatct
agtgacatgtatcaatccatgaaagagaaggggaatcacatattttttcaggatgaaaagggggaaagaaaaaaaaaagaattttg
tatggagaacaataagggggatcatcagaacgaagataaccatgatataaaaagtaatcataatataaaaagtaatcataatataaa
aagtaatcataatataaaaaatgatgataatataaaaaatgaccatcatataaaaaataaccatcatataaaaaataatcataatataaa
aaataatcataatataaaaaataatcataatataaaaaataatcataatataaaaaataacatgaaggaaaaaaacctcttagtagatg
aaaaaaaagataacactcttattacctttgataaaacatctattacattagacattaacgaaagtaagattataaatattgaaataaaaa
atttaaaagaagaaaaagtacatcgtgaatataacatattaacattagaaaatagttactttgttgagaagcctcgaaatattattattat
agctgtttttataaatagttctacttctttttattatgataatataaaaacaaaagaaataaatatgaattatatatattatggaaataagaaa
aaaatacaaggacaaatcaaaaacgaaaataattataatatatcatatcaatataaaatgaaaaaagtgcatgctttctatacattgga
cagttttaaaaagtatatacaattaaataatttgtctgctggatctttggaagaggaattatgtgagatggaaaaactattaggacagg
aagaaaaagaagataaggaaaaaattatgtccatggcaaaaaaaatgatgaatattgaaaaaaacattcatgttaaaatgaataatg
atattgtctgcaatgtaaagaaattaagttatgaatctgtttttttcgaaatagattcaaagcatgatcttttgttcttagaaaaaataaata
aaaacaaatcataccttttaaacattccaataataatagatatcacattgtttgttaataaaagtgatgatgaaaaggataagacgaata
agataacgacaggggggataagtaaaatgcataatgacaataatatgaaaataataaataaaagaaaaaatataaataataattatg
ataataattgtggtaataattgtgatgataattatgataataattgtgatgataatcatgataataattgtgataataattgtgataataattg
tgataataattgtgataataattatgataataatcatgataataatcatgataataatcatacaaataaccaacacaataataatatttgttg
tacaaaccttttacaacttcataagagagaagaaaaagatagagactcttctattcatcatgcctcaaattgttatgaggaagaaataa
atttacatttcattttttgtttaacactcccttgcctaataacaaatatatatttttaaattataacaaagtaacgacagaagaaacgaaaa
gcatgataatagaattactaaataaaaataaatttttaaaaattaattatagcatatctaaaattccttatataacaataaataagaaagaa
gggaaaataaattcgcaagaaaaagatataattaccattaccctgaaatgtaatgtcataaaaaagattaatgaatatatgtatatttttt
tttgtaataatttatatttttttgttatttttataaatggagaagtaacacaaatatccaacattaacgatgataatatgcttacaataaaaaat
ataaaaaatatttatgataacaacatgtgtgatgaaaaaggtattacaaataatgataatacttatatattaaataataaaagtcttttaaa
aaaaaaaaaagaaaagagatatacagatgatcataataataataataatgatcatgatgataagatatttgaacgcattcttaaaaatg
tagaatgtaataaagtcaatagacatttatatgaacaagatgaacaagtagataaatataaatataatgaacattttattaattatttaaaa
aataataaaaaaaaatataacgatgttttgaaatatatgtacaaaaaaagacataattcaaaacataatattctacttaaggataatata
aaaaataaggagtctatgataattaaaaaaaattcttcttataatttaaaattttctgaagataataatataaacaaagaatataatataga
tcataaaagaacaaatgttttaaaggatatatatatatatgtaaatgaagaatatataaataaaattacaaatatatatatacctcaacatt
tctatgaagaaaaaagaaaacaatatataaatatgaaaaaaaaaaataataaacaatatttagtgggaacaatgaaaaaagaaaaat
atttaaatcaatccttatgtttaaaaaaaaataataaaataaataatattaatattattaataatgatgataattttcaaaatgtacaaattata
aaacaatatttaatatatccacaaattattcattttaattatatactattaaataaagaatttgaaacgtttattcatttacataatactaattct
atacatccaataaacatatatttttttttccatagtgataatataaaaattacttatcctgaagaaacaaaaaccacatgtcataacaaaat
taatgatagcaacatatgtactccttatcaaagtaccaaagttaccataaaacagaatacacaacaaaaaatatgcgtcacacttaatt
taaaaaattataatatttttaataatcaaaagaataaaaaatgttctttctctcaatcagaaaatatggataatgaagaagaacaaaaaat
atgtaaccatattatttcaaaaagaaatattaaaaataaggagcatcataattatttagatgatacttataaagatgaaccatataatatat
atcatcataaaacaacacctttctttgaaatatatatttattatgaatatatctccataaaaactcaagatcattatgatatagacaaaataa
aagtccaagctaacttaatattattaaatctttttataaatactaacatattatatttttctattacaaatatggatatagacatgttttatcata
actatttagttatatacaatccgtttaatatcacaattcagatgaaattaaaatataatgagcaagtcttaaaaatgaaggaccatataag
tatatatcctggtgaatacaaaatggttccagtacaatttattaccagcgaagcaacagcttgtgtagaagaatttatttatgtatacttg
gatgactttagattatacaaaagcataaaatgtaagtgctttccatccccgtgtagctataaaattaatgtgaatgaaatcaattttgata
atgtccttttaaatagaaactatttaaaaagcttttacataacaaacacaagtaactttccacttgtatttatgtgtgtttataaacccccat
atgttcatgtccttgcgaaacgtaattatgctaataaaaatgaaaaaataaaaattaccatattaataaatgtaaaagaagaaataaaa
ataaaagataaacttatattctatgtgaggcaaagggaaaatctaattctacctataagtgtcaagttaacccagtccaatatagtgatc
gtgaattgtccagatattattcaagaaagcatggaacgtaaaaggtacaacatcaatattttaaacaaagggatgtacgaagaaagc
gttattataaatttcaacgaaattccatttttgaatataaatatagaagatgaggatacaggaaacaaattaaattatataaactataaaa
acaaagaatataaacatagtgaagagaaaaaaagaaatacatttgttaatatatataatttagaatatgataataatattataatagatg
aatatataaaatattattataatgaatttattttattatataataatttatgtaataataattataaaataaaaggtatacttcatatttcatcaga
tcaaaaatgtttacaaaattgttataaaattataattccacataatacatttataactttaatgattgaatgttatataaataaaatatatgaa
acagatttatatttatatcaacatgtattattaaatttaaacaatccatttgtcaaagatgaatattataaagataaattaaaaattaatataa
aaaatgaatatttaaaatttactccttcttatctttactttgaaaatatattattatgtagttcggagaaatatttaatcacttcttttcaaaaga
ctataacaaaaaaaatacaaataataaatgtgtatcatcaaaatttaaaatgtactgtaaagatatgtccttataataaagaacagacaa
aaacatcacagcctgtcaaagtatctagtaataataataataataataataataataatagtattataccaagcgatcatgatatgatat
gtacacattccacttggtatgaaaataatcatacaaaaaaaaaacaacattatatagacataagcaatgtaccagatattttaaaagtt
aacgaagagtcttatataaatataaaagttgataatatagaaaaagaaggaagatatgtggaaatgttagaaatatgtgcagaaagt
gttaataaaaaagaaaatgaagaaaaaataaaatattctttatatatacttatcaattgtaaagatgttaaattatatttcgatgtttcatata
ttaatattatacataataaattaaatcaatataattatttctctttccatatatataatgatggttataattatgtaagagccaattatctttttaa
taatatatttaaagaacactttttcttagatctacaatttttaccaaacaaccaactaaataaaaaaaacaaaaaaatcaaagttcttttga
aatatctatcaaaaattaatatccagcttaaaacatttattacgataactgtaatggaccatgaaaaatatgacatacctttgttcataaat
atacacgaggaaattgattatctcatggaaaaggaaaataaacataagcagaattacacacattgtcataattacacacatggtcata
attacacacatggtcataattatttacacgaaacggacgagttatacgaaaaacataatgggaatgaaaatagtgatgacataaaac
atccttatccttatgatagagaaataaaaagtgaagggtctacaaataaatcccatatattatcatcacacctgtcaggtgatcacaaa
gatgaaattcctattataaataataataaaaaaaaaaacataagttattttactaaagaagaagaatttgccttatataataatatgtctat
accatatagtcttaaatataaacaaaataagaaaatggatgtagacgaaaatcaaaacatgttatccttttatcgaaaaaatatggaag
agtcaagtaatatatatactaatatatttaaaaatttacaaattgataatataagaaatccttattataatacaaatgaagaaggaataata
aatttaaaagaaattgtacatgattatatgttcatatttcttcaacatattatcatcaataaaaattattttccacaaataattgaaaacacta
atgacttatatgtatttttcttaaacattttatatgatctatcttttatatttaatagtaacaagaatatacaaaatataataaatgaactaaaa
gcatatgagcatatgcaaaatggtagtgttgacgaaaatgaatttataaaaaatatgaaaactctaacagaattgttgaaccaagaga
agttactaataaatcatataatgtatgaaaatttactacctcttcatttgtacctacaccatatttttaataacattccagaatacttttatata
aaaaaagaaagtgataaaaaggaaaaatgcaaggaaataaacaatttagacaacacaatattatatgataataaaaatgatatagat
gaagaagaatataataaggaaaaaaacaaatttctaaatatatctgatatacaatcatttttaaaaaatgacaattataaaggagataat
aatattttttatttagataatatatttaaaacatatgttaaattatttatatattcttggatcaccatattcttagatattatttttaagaaaatcttt
tctttcataaatattacatcgttatataattcgagaggtactaaattatgtactcttgaaaataatatatatgaaaatattaatatccataaaa
taaattatcttatagtattaaatatagaaaacaaaaaaattaacacgtatacatattcctgtacaaatgaaaaaacacaaaaatgcatag
ttaaaaaaaaatgtatagacgaaaaaaaaaaggaaaatgcaaatataaatgtaaaggagttatcatttaataatcataaaaacaatga
agatttaaatatacaagaaaaagcatactttcatttttatcaaaataattttatcgacataaaggaagtgcttcataaaattggacaatcc
acagacacagatcactcaaaaaaaaaccaagaaataaaagaaacaaaagaaataaaagtaacaaaagaaataaaagtaacaaa
agaaataaaagtaacaaaaaataaggattcttatcatacaaacaatctttataataatactaacaatttcaaaaacacggtgaaggaa
aaaaaacaaatcaataaaactttaaaaaaggagacacacaaaatattaaatcaaaaaaaaaaaaaaaaaaataccaatatagatga
agacaaaaagaaaactactgaaagctctacatatacagagaaatatgaacataataagcatctgaacattaaagttcaaacaaatat
aaattattatagaaagatagaagttattttatatgaatggttatattttcattataataatgtatataacaccaaagtaaaaaaacaaaaatt
tatatttacccaacaaaaaaaagacatatcaaaacataacaagttatatcttcagtatgatcaaaataaaagaaactctgaaatagaac
atacaaatcacaaagaagattattcgatgtgcgacaatgtgataacaagcaaaatgagccacatatctaatatggataatcaatatgg
taataatattacatgtgatataccatttttgttgagaaaaaaacgaacaagaagaagaagacgaagaaaaaaaaaaaaaaaaaaa
aaaaaaaaaatatttttaaaaaaaagaaagaattcatcacaagtttatatgaattaaatgattttgttattattatatatactataatttctcat
attccttattttttattttttaaaaataaaattaaagacccatgtaaatgtcaaaaagattatttatataacatagacatactattattaattttg
aacaccattaaattaaataatttaataagtagaaatgtattaatacaatttaattatgttcatatatttatattcttatcatccttatatatcatat
taccaaattatattccaaattattatttaattctagatgatttctcatcatataaaaatgacgtaagggtaataaaaggaaatttacagaaa
aacacaaggacaaataaattaaagcaacttcatatgaataataaaaaaaagaataacaataataaaaatacaagtgataaccatgtt
cataataattataatcaagaaaaacaaacaaaaaaaaatattacatgtgatgaaaaaattaatgaacgaattatttacatatataattcta
ataaatacatgtgttcatatgatatttttttgataggatcgaacaaatatactattgataaaaatcatataactatacattcaatgaagcatg
aagaaataaccatatccattgatccacattatgatgaccatattattaaatatgattgtaataacgagatttatttaaaagatattaataaa
ataaaatataaaaaaaaggataaaagttctgaacatactaatagtgagaaccgattttccgatagtagtaaaaattattcatcattatcg
tcatcattatcgtcatctttatcgtcatcgttatcgtcgtcattatcgtcatcactatcgtcatcactatcgtcatcattatcgtcatcattatc
gtcatctttatcatcttccaattcttatattcattctcggtgtgactcatcaaatattcctaaaaacatagacacaaataaaacaaattcaa
aacatataaaacacatacattctgataaaagtaacaactattcaattcttgttttgagggaatcaaactataatttattggatttaaaaacg
gcagaggaaaaattcatttgcataaaaattatagaggggaataatgaagactcggaaaaatcacttttggggaggaacaaaaataa
tataaaaaataaggataacgaatataattcggtatatgataaaacaaaagagagacaaataaaaggtgatatacataagaataataa
ttcttcagattgtggcgagatgaatattattttaaatgatagtgaagtgaaatatattaattttaaaggtatggtgtatgaaaaaaaaaata
tccaaataaatatgataaataatttcgatagaaaagtggaggttacaacaaatgtttatcatatatatttaaataatagaaaaaaggaaa
ttaagaaaaaagaaaattttgataaggatatatataatttgaaacatacaaaagttgaggaaaataacatacataataataataataata
ataataataatagtagtattccttttaatgctaattatgatatgtactatgaatgtgatgcctgtacaaatataaataatcatctaaaagcat
atataaataaagatgatataaattttcagtgtgtttctctagataaagaaaagaatatatatattggtaaagataatggtaataataaaata
aattttcatttttctcctttttttcaaggtacgtattattgttttcttttattttatgttttatgtacaaaaacaaaattgcttttatcaacttgtaaaa
tcaattgtgtcttttatataaattacataaaaccagaagaagttattaaaataaattataaatcttataactttacatatgatcttattttaaga
ccactgaataaacaattcttaaaaagtatttattacattttatgtaaattaaataatagcagtaataatgtttttttttcaatttatataaagcg
atatttagacaatatatatatgaataaagagaacttttttattttatgtgactctattaaaaaagtaaaaattaaagataagaaaatatcatt
caacaattttaactatacaaaatattttaaagatgatataataaataaaaatatagacattgatctattatgtgatatgataaaaagggat
gtcacgtattcttgtaaattagaaataaatgaagaaatgaatggtgtgtttgaatataacttttatcttttaaaggggaaacaatacaatg
tagaaaattgtttattcatgataaaagatggaggtgaaaattatgtaaacgaccaagtgtcactatatataaatgggaacaaaaatgtg
aataatatgttttcccctccaaaaaataataatgtcaacagtatgaataataataatgatgatgataatgatgacaataatgatgacaat
aatgatgataataataataataatcataataataataatcataataataataatcataataataatcataataataataataataatcataat
aataattattattattataattcctgtgatggtgatgaacttgtaccgttgtgtaaaccaaagggaccagataattaccattattgtgatgt
taagctatataaaataatatgcaatgtcaatgatgaaaataatataagaacaattaatataaatattaaagccggtatagtatacaaaaa
atatattcctttatataattatactaataataatattacatataaatgtgctttttccgaaattatcaatgataaaaatatgattattaattataat
atatttacttgtgatgaacttgttaaattaaaaaaagatatggaaatcttcaattttgaagttacatgtttttgtcctgtaggtggaataaaa
tacaattcattttttatcttaacaaatgtacttaatgaacaagataaaataaaaataaaaattcaaggatatatatcaaaacctcttcctag
agaaaatatacaaataaatgttttaaataaaataaaaaaaaatatacaaatcgaaattttcaatgaattagatattccttgtgaattcaaa
atatattctgacttgttcatattatttggagaaaaaaaaaaaattgaaatgatgcctaaacaaaaaaagctatatacattttttataaaaag
tgcacatattggtgaatttattgggtgtcttatattcaaattttataaatataaagatacaaataatgaaaataatgcttcctttttttctgatt
attttttctggtataggttaaatattgtagtacaattaaatgaacctatgaaaattttatatctagaaacttttataggacaagaaatcacaa
aagatattattataaaaaataatgcaaatcaaaaagaagaatatttcttgttattatatatcaatgaatatatcgaaaaaaaacaaataca
aatagaaaagaatgatatatatatttataaaattatatatttgccttatatacctaattatttttataatgtcgaaacatcaagttgttcacatt
ctaaaactttaacgggggaagaaaataataattattcaaatgagtttcatgaaagcaagcaattgtttatggataagggagaatatgc
atctgatcatttcatacacaatgaagatgaaaaaaatgaaaaaatgaaaaattattcattaaaaaactacataggagatataataaata
gtcaaagcaataatcatgtacaacataataatcatattataaacagtctgcacaatttttattattcaaaaggggaatataataatatgcc
acaatgtttacataaatcaaaaaataaagacacatttacacaagagtacgaagaaaataatgttaatacaagtgaccttctttttgaac
ataaacaaaaaaagaactttcaatcaaaagaaaaacaaatatttgaagaattaattaactattgtaaaaataagtatattaatataaatg
atatcaattatgataatcataatatcggtttcttttttatatataataaaaaacaaggaatcaattattatattttaatattactatccaaattta
aaaataaattgataataaataacttttatacaagtttgtcaaaggcatgttatataaatattcatttccattttcaacatgaacaagaaaga
caagtgaaacaattacaagaaaaagataaacaaaaaaaattacatagtttttcatatgaaataatcgaaaaaatggacaaaatggac
aaaatggaaaaaatagaaaaaggggaaaataaaaataaaaataaaaataacatatatagtgcatcatatgatatatgttcaaataatg
atcaacccttttcttcttctacacatgatgtttataagaatcatttaaaaaattcattattagattcatatggacaaaatcattcttcaaatatt
aaatgtatatatttgagcaataaaattaattattctattttacaaaatgaagataaatcaagtaataaaattgttattaaatacaaaccaac
aatggaaacatctcaagattgttatataatcataagaaataatttatatggagattttatttattttatgaaaggaatgtatgttgtgaaaaa
aaaaataaaagaacaaatggttttgtataactcctcttctttgtaccgtttcgctataaatttgttcaacccttttagttgtaatgtatgcgtt
aaatgtggtctaaaagaaaacaaaataacaaataaaaataataagaaaattataaaaaatataaaatatatgaaaaatataaaaaatc
acaacgtacataataataataccaacactgtaaataagaaaaacaaacacaagagctcacgttttaagggaaaccaattattattaaa
taacaaaaaaaattatttcctaattatcaataatgaaaattttataattaaagacaaaaaggattttcctctacttattatatccttctgtaaa
tatgaaccatcacatcataaaaccttaatcgtaaagttacaaccaatttataataagaatatgaataacaaacaaattccttatattatat
atgaatatagtctagtttttaataaaacaaaccaacaaaaaaatcaaataaaaagtaatacattaaatcattttgtcttatccgatttgtat
aaaaacgaatcactgacacatgaatatcaaactacagaaaatgaggaaaatgaaaatgatgtggaggaatgtcaagtgaatatac
acgagtcaaatgaaaacgaaaatgacatgtgcgagtcaggtgaaaacgaaaatgacatgtgcgagtcaggtgaaaatgaaaata
atatttgcgagtcaggtgaaaatgaaaataatatttgcgagacagctgaaaatgaatctattagcatgtacgagtcaggtgacaaata
taccacagatagccaaaagagcgatactgcaaatgagttatccattggagagttaagaagagaatatacagataaaatgagtatga
gtcaaaatggaaacgacgataagagcaaatatgattattcttatgatgatgattgttatttaataaatggtaatgatataatttattacgat
aaaaagatatttgttgaaagtatttgtaaaaagaaaacgtgtgtacgaatatacatagacaaaaagaaaatgagagaaagtcaaaat
tatgatagtttaatggatgatttagaaatatgtaaaaagaaaaagtgcatgttgcacagaaatgttgaaaaggatgaatggaattataa
aatatatttaataaaattaacaaatgtgtataataataataataataataataatagtgatgatatagtaaataagcggatgatcacaatg
aataatatattatttgaaatgaatgatgaaatgttaaatgattatatttatgtaaatgtaatagaagatacggaagaaggattattaataga
tatagagttgttggcctatttaccttttcactgtaatttttttctaatgattaaaaaaaaaatgaaagaaaagataaatggagataagaag
ggtataaatattttatcagaaacttgtaatgggaaggacattaatatattacctgaatataaagataataataaatatataataaatggtg
ttgaaaaaaattgtatatttgttgaaatatttaattaccttagaatatgtagtaaatatataaatatttttgttgataagaatttatgttcagaa
aagaagttaaatatatattacaaaaataatagtgattcttattttaatgcatatatgataaattacaatgaatatagtacagatgacgttat
ggatgagatattaaattatgatataaaaccttcttcaggtgtattaaaacataataaatataataaatttataattataaggacaaacaaa
aatggcgtggtaaatatgaataacattttccttctcttgataaaaacggaaagaaaaattttttcttacatcatattttctcattacaagcat
acaagaaccatatgtacaatggatgagtataataagatatatgatataatgaatgaaaacaaaaaaaaattcagcgaagtttttaatac
attcaaggaagaaaaaaaagaaagtgtcgtttttaatgaaaggaaccaaattattattgaagatatacaaaagtcaacaaatgaaatt
caaaatatttaa
>Pyoelii|PY17X_0720100.1: pep
(SEQ ID NO: 24)
atggggttagatgcagagaaaaacaaatttgaccttatagatgtagaacccaaaagtatagaattttattatgaccgcatagaaagct
tcgaggaatttgttgaaaaaaaaaaaaaagtgatagaaaaaaaagttataaaaataaaaaatatatgcacaaaaattcaaaacataa
aaatattcgaatctgaatcgaaatatttaaaaataaaaggaataaaaaaaaaaaaacttgctccaggtacttatgaaaaaatattagta
gaattttcattttgtgatgtaaatataaataaatataaatatgaaaaaataaaaaatattttagatgtaataaataatatagagggaactat
aaatgtgaaagtacaaagtgaatacacaacattatatattcctatatatataaaaaagaaggttccgatatttaaatttaataatgtattta
atttggggttatgtaaaacaaatttaatttatgatgtaaaattagaagtaaaaaatgttgggaataaaaaaggaactctgtctatatgtct
agacaatataaataatgaggcagcaaaaaatgaggcagtaaatgatgataataaaaatagtaataccctttcgaatagtgataataa
acatattattgaaaataatttttttatttattttgataaaaatacgatttgtttaaaccctaatgaaagtactataattaatattaaaataaaaa
ataaaaacgaagaaaagattcaaaaatcttataaaataattacacaagaacatagttatttttctcaatctcctaaaaatttaactatgat
cgcaatttttataaatagtcaagtatcatttatatatgagaatatgaaaacgaatcaaatagatttgaattatatttattttggaaattcgaa
aagaattaatggacagataaaaaacgaaaatcaatatccagttatttgtaaattaaaaaaaacacaaatcaaaatgttttatgattcca
aggagtttgcttcccatgctgttgacagaggcgtagatgtccggactcttttgagggatttggacaattttcccgaatttgatgttaatg
atgaggaaaagaaaaaaagtgaagaaaatattttagatgtaggaaataatttaaaactagaagaaaaaataaaaataaagttagag
aaacaaaatgatatatatttcatagacaaacaaagttgtactgatattttattatatatcgaaatagaatcatgcatagatattttaaataa
aattgataaaaattattcatatatgttaaactatccagtaatggcagaaataacaataaccgcaaataaatgcgatgcaaaatgtaagc
cttctactaataggaaaaaagcaattaccgatttgcgacaatttgacaaacttagaaatggtgcgaatactgtgaacggtgcgaattc
tgtgaacggtgcgaattctgtgaacggtgcgaatggtgagagtgtggaatgtagtgaaggttgcgaagaagatttaacattttatgt
atttttttgtttaacattcccatcaatcgtttcaaatatatattttttaaactatgatatggtaggacaaaatgaagaaaaaactttgattttag
aattaaataatttaaataaatttttaaaaataaattataatttttctaaaatttcgcatatacatctaaataaaaaagaaggtgttattaaccc
attgtctaaagatattattgctcttaatttaaaatgtaatgtaattaaaaatatagatgattatttatatttatttttttgtaataaattatattttttt
gtgttttttgtaaaagcacaaataaaaaaaagaatatcaataggaagatcgatagttatcaaaaatgataaaatgatcaaagctgatg
gaaaaacaaaacaaattaatagaaacacaattgagttagagaaaattacaatttccaaaaaagaagatgaaaattcaaatgaagata
taatttatgaacaaattttaaaaaagctagatttagaaaaaattgacaaaaatttatatacattagatggtaaattagacaaatataaatat
aacgaaaaatttatgtcttttttaaaggaaaataaaaaaaaatataatgatatattaaaatatatgtataataaaagaaatgagaaaaaa
gagaaacagggaataaagaacccaatacctgaaaatgatcaaaatgaaaaaattaataatataataaatgataaatttatatatataa
gtgataaacaagtaataaaaaatactaacatgtttatacataaatctatatatgaagaaaaaaggaaaaaatatattgatatgaaaaag
agaaaagagataaaaatatttaatattaaaaaaaatgatgataaaaaagaggatattcaattagaatataatgatatattagaagaaaa
ccaaattaaaaatcttatttttgataaagtaatttattttaattatgttttatttaatcaagtatatacaaaaatatttaatgtacataataaaaat
gatgaatgtaatataaaaattaattttacacatagttctaatttaaatttagacaaaaatttattatttataaaagggaattcaaaagaaatg
ataaaaactaacttacttttaaacttacctattataaaaacagatgtagaaaattgtaacaaaacaataaacattaatgaaataaaatata
gaaatatttttgataacgaaaagacaaattttcaaattttaacagatcatacatttaatcaaaaagagcaaaataatttatcattacatga
gaatgagaatctttcaaataacaataaaagttatttatttgatcataaatattatacagaaaatataacattaaagattaatgataattatg
aagatatgatagttgttaagggaaatataatacttttaaatatagttattaaaataaatacattatatttttattttaaaaattcagacatatct
atgttttgtgaaaataatttaattttatataatccctttaacattcctatccctatcacacttgaattctccaaagaaatttttgaagcaaaaa
ataaacttgttattaaccctagtgaatataagacagtggcaataaaatttataggaacacaaaatattgagcgcatggataattttatta
atttgtatttagacaataatagactttacaaaagggtaaaatgcatatttgatgtaaataaatgttcttgcaaaataaatgtaaatgaaata
aagtttgagaatataattttaaacaaaaaatatttcaagcatttttacctgaccagtacaggggatactccggttgtctttaattgtgttgc
caaaccagattgtgttaatatttattataaatataattatgttaataagaatgaaaaggttaaaattatagtctcagtaaaattgaaagaaa
ataaacaaataaaagataaaataattttgtctattcgcgggtctgataatattattatccctattattgttacaaaggtttattcgagtaattt
gttatttcttaatgatattaaaatagatcaagaaacaaacgaattaaagaggtatgaaataaaaattgtgaataaaggagggtgtgaa
gaaaatgtgattttaaatttaagtgaattaaatttaagcgaattaaattttttaaatattgaattaaataaaaaaacagaaaataatagaat
aatatataataataaagaatataaaaatgtaagaaatatgaaagatgattttatggttttaaaaaaaatatttaagacagaatatgagaat
attataaccaataaatgtgtaaaatattattttaataaattaatagatttatataattttttacacaattttagtaaaaataagtttaaaattatat
ttgtagaaaataaaaataacgatataaaaataaatgagaaaaatatgtacaaaataaaaatatcaccaaaatgttttgtttccttatatat
tcaatgttattataataatataataaaaaaagaaataaaatttaacaaaatattgtttgaaaataaatgtttacatgaattatcaaatgaagt
tataaatataaatattaaaaatagccaattaagtatatcaccaaatttgttattgtttcaaaatattataccttataattctgaacgagctttc
ataacttattataaaaaagaagggattcaaaaaataaaattaaaaaataattctagttaccctattaaattggaaatattattatatgaca
aaaaacaaaaagacagtttattggcaacatcatcagttgacaattttgtagaaatggaaagaaaacaaattattaaaaataatgaaga
gagtaatgaaaaaattgaaataaacaaaacattacctagagcagttccaaaaaaagattctaacctaaatttaaagaaaggaaatatt
caagaaaatgtaggaaacaaatcgaatgaacatgttgaaaaaatgtacgaaattgaattaaataaagagagtggaataataaaagc
aaatgaagaaattgaagttgaaataaaattaaaaaattgtaaagatataggaaagtttgtagatatattggaaataaaaacgaatgttg
taaaaaatgaacaaacagataaaatctttgatgaaaaaaaatattgtatatatattttatcagtaatcgaaattcctaaattatattttgata
ttacatatataaatataatatataataaactaaatgatacatttatttttccttttaaaatatataactatggatatagttatgtaagaataaatt
acaaatttgaaaatttatatacagattttttttttctatcattagattttttacaaggaaatgaaattgacataaaaacaaaaaaaatcaatgt
agaattaaaatgtaaatcaattaaaaatttaaaatttaaatcagaaattataattagtatattagattatgatatttataaaataccagtgttt
gtaaacattgatgaaaacattgattattttttggaaaaagtacaatatgaaaaatttgatgcattaacaagtcaggcaaataattatagtc
ttgatcaatatatgtgtaaagaaaattcaagatcaaatttgtctgaaataatagatgaagtagtctggacagataaacagttcaattctg
gcaaagctgaaaatggcaatgttgaaaatggtaattttgaaaatggcaaagctgaaaatggcaatgttgaaaatggtaattttgaaaa
tggtaataactacaacatttccaacgttagcgaacaaaataatgagttgctctctattttttgcaaaaatatatgtacagaaaattgtaat
atatttaaaaaaagatgtatatataaaaatttaaaaattaataatataagaaatgtatattataataaagaaaatgaatttattgaaataaat
gaaataccaaaagattatgtttttgtatttcttcaaaatatattattaaataaaagttgtgctcctacatttttagaaaatactaatgatttgttt
ttattttttattaatttgttaataaatttttttcatatatatccaaatagaaatatatataatttattacatgaaattaaaatataccaaaatatttc
gatgcaagactttgaaaaaaatatatttatccacgaaaatattcttgcaaatataaagcaaattttaaaaagcttaaaagaggaaaaac
ttttagtgaatcacataatttatgaaaatctacttcccatagatttgtattgttgtcatatactggataaggttcctgacaatttttacataaa
aaatgtgaaatgtaaaaaagatagttttgaaataaaagatatatataattttattaataataatgatgataaaatattatattttgatcgaat
attaaaaacacatttaaagatatttagctattcatggattacaatttttttcgaattattaagtaaagaaatatttaattgtgttaatatagaat
cgttatataaattgagaggaatcaaattatgcaatatagaaaataaaatttatgaaaatataaaaatccaaaaaataaattttttacttattt
taaataaagaagacaatgaattaaataaacatacaagcatgttagaaaatatcagaaataaaacaaaaagagaaaaaatatatggg
gataataatattaatggtggtaataaaaagataaaatattctcaaatcaatgaagaaaaaaaagaaaaaaaagaaaaaaaaattaat
aatattaaaaatggagttataaagataaggaaacaaccaactacgataaaaaaaatggggaccaaaaaaaaagacctagatttaaa
ttatttcaatttttatgaaaataatttaattgatttaaaagatttggttaataaaataaacaacttgaatttatgcagtggtgaaggagaagg
tgaaaaaggaatggaaaataaagcggtgttagaagggtgccaaaataaagcggtgttagaagggtgccaaaataaagcggcgtt
agaagggtgccaaaataaagcggcgttagaagggtgccaaaataaagcggcgttagaagggtgccaaaataaagcggcgttag
aagagtgtcaaaataaaatagctagcaccaatcaaacaaacatttctaagtattacaaaaatattgaatatatacttttcgaatggttat
attttcattacaataatgtgtattataataaggtaaaaaatgaacaatatatatttaagaataaagaaaatgaatataataaaaatgaaag
caacttaattaatgatgatactaattctaaacaatcgaattatatgaaaaaaaatgatgacaattcttctggatatataaataatgaacatt
catataaaaattttgaaaagtcaaacttttatagagaagaaaagaaaaaaaaaaatattgttagggaaaacaaattgaaaataataaa
tatatatagtttaaaaaatttagtaattatattatatacaataatttcacatataccatattttttattttttaaaaataaaataaaagtagaatgt
aaaaatagaaaggattatatgcataatattgatattttgttaataattttaaatgaaataaaattagataatttaataactagacaggttctt
atcgactttaattatattcacatatttttatttttaaattgcttatattttatattaccaaattatttacccaaatattatttagatgtagatgatata
tcatcttataaaagtgatcgagttttaataaaagaagaaattgtaaaaaaaaaaaatactataaaaaataaaaaaaatattgatttacat
aaagacaaggaaagaaaaaggaatcatataaataataatgacaaaagtgtagatcaaaatgaaagaataatattaatatataatttaa
atgataataaatgtaaatataccgtttttttaatagggtgtagtaaatatgaaattgataaggaatatatagaaattgattctcataaatca
gaagaaattaaattaaaaatagatccaaattacgacaaaaatatattcaaatatgattataatattaatataaaattcccatcttttactaa
aaaaataaataatattcacaaaaaagacaacttgttaagtcatgaaaatgttaataatactataccttaccaaaaaaatacttcaaatttt
ggagactattcttctatatcttcttctatatcttcttctatatcctcttcttctattgatagttttcagtcttctttatcttatgatggaattcaaaat
gttgaggaaaataactatgaaaataatttttatcataatgaaaattataccatattggtactacgaaaagaaagcaatctcttcccagga
gataatgacgaggacaaatttatttgcattaaattggttgaaacaaataagagcaaattaaagtcagaaatagtgaggagtaatagta
atttattggtagaaggaaaggaatggaataaattgaatagcaatttaaaatttgaagaagggaaggttaatatgaaaaatggtagtg
gaacaaattcatcaaaaataaacataatgttaaaaaatagtgaaataaaatgttttaacatgaggggtaaaatatatgaaaacaaaag
tgtagaaataaatttaataaatgatatttcgaaaaaagtgaacataaatatgaatatgtatcatatatatattaaaaatttaaaaagagaa
gaaataaaaaaagggaaatttgatatagacatattaaataataaagaaaatggtaattataaaaatgatattaatagaaatataaatga
agagaaaggatacaaggaatgtgatgtttgttctttacttaatttgaatttaaaaaaaaatttaaattgtgataaaaattattttaattgttttt
atatagaaaatatgaaaccctttgttagtagagaaaatgagaaaagaacgataaaattacatttttgtccatttatagaaggatattattt
atgttttttacttttttgtgctaaagatttaaagacaaaatgtgtaatgtcgagttgtaaattaaattgcttattttatattaataatataaagg
aagaagaagttataaaaatagattcccaattatcaaaattttcgtatcaaataaaattaattccaataaataaaagatttctaaaatgtata
caatttattttatgttatttaaacaaaatgaataataaagtttttttaagttacataaaaaattatctaaattatataaataaaataagtgacca
attttatataatttgtgataaggttggtaaaaatgggataaaggaaaaattaccatctttccaaaaatataattataatgattttataaatca
aataaatgaaaataatttatttttagacaatttttatgatcaaataaaagaaaaaaataactatttatatgaatttaacataaatgaagaaa
atatgggcgtatatgaatataacataattttgatggcaaataataaaaaaaaatataatgaagatataaataaatctatacaattatcaa
aatatgatgagttagaagtagatgagaatagtatatataatgcatgtaataaattgtataaaataattgcaaatataaataaaaaagaaa
ataataatattataaatgtaacatttaatcaaaatgcatatttattatcaaaaaaatgtgtgtctatatataattatactaataaaaatttaata
tataaatgctctaatagtgaagtattagatatgaataaaaataaaattgattataaaatatttaattatgatgaatatattgaaatagataaa
gatgtagaatcatttaattttgaaattagatgttattcggttgtagaagtagaatgttcatgttttatatttttaactaatgtagaaaatgaac
aagatgtaataaaaattaatgtaaaagcaaatattgttaaaccatatcctaaagatacaattcatttaaaaatattaaataaaatgaaaa
aagcagtaagtattgaaatatataatgaattagattttcattgtgagtataaaatttattcagatttaccaattatatttggagatcaaaaaa
tcgaaatgcttccaaagcaaaagaaagtttatactttttttgttagaagtatacatataggggaattcattgggtgtattatatttaaatgc
tttcgatttttagatgtgaataagataaaagattctagagatattattaaaaatgattctaatattacatctaaaaatttttcattggtagattc
atttttttggtataaattaaaagttacagtagatttgaacaaaccgatcaatgttttaactcttgaaagcaatgtaggagatgtattaaata
aagaaattgtgttaaaaaataatggaaataaaaaagaagaatattttttactttcctatatgaatgaatatatagaaaaaaagaaaattg
aaataccagcaaatgatgcatatgtatacacaataaaatatgctccaaaaataccaaattattttgataatattctaaaacataaaaatg
aagaagatacaaaagataccaattttgaattagatgaaaaaaaagaaaaaggttcgaaatataataaaacaaaaaaaagtgaatgt
atgaaaagaaaagatgaaataaaacaaaatgagaatataaaattttcagacatttcaaatgagttatcaaatttttattttcttcaaaatg
aagaagaagaaataaaaaatgaaggagattgtttacaaaaaaatatgacaaaaaaattaaatttggaaaaaaatgaaaaaaaaata
tttgaagagttaataaaatattgtgaaaaatatataaatgtagatataaaatatacaagtcataatattggttttttttttatatataataaaaa
tgaaggggtaagttattatatattaatattattggctagatttcgaacccaattagatattagtaatttctcttcgttattatcgaaatcgtgt
ttaatacatttgaattttgaattttccaacaaaaataaaagatatgtaaacaaattagaaacagatcaacagaaaaatacaagttattatt
accaaataggagaatatattgaggaaaataatataaataaaaataattcatacaattgtattgagaactataaattaaataatgggtttg
agttttataatagcaaaattgaaagaggcgcggacaaatcttccaacatcaaagagtttgatggtgatggaaatggaaatgaaaatg
atgatggtgatggtgatgaaaatgatgatggtgatgaaaatgatgatggtgatgaaaatgatgatggtgatgaaaatgatgatggtg
atgaaaatgatgatggtgatgatgacgatatgagcacggaacaaattgtgagacacaacataaaggggatatatctgagtaataaa
ataaattatagcatgataaattgtgataataaaaagagtggaaattatattttgattaaatataagcccactgttggaacatcacaagaa
tgttatgttataataagaagcgatttatttggagattttatttattttttaaagggtggatatatagaagaaaaaaaaataaaagagagaat
aataatacataattcattttgtttatataactttaatataaatttattcaacccattttcttcaaaaatatatgtaaaatgtattattgaaacaga
tagaagtaaaaactatgaatataataatatgaataaaataaattgtctaaaattgtacaaatcagaaaaaagtagaaatattgaaaatg
gaaaagaaaaaatattattaaatacgaaatataaatattttaaattattaagtaatgaacattttaaaataaaaaaaagaaaaaacttttct
attttttctacttatttttgtaaatatgcaaaatcgatagaaaaattgataatattattatatcctataaataataaaaaaaaagaaaagaaa
attaatcaatatataatatatgaatatgaattattttttaataatacaaaaagcgatttttttcttgaaacagatcgtataatagcagataaa
gaagaagaagaagaaaaagaagaagaagaagaaaaagaagaagaagaagaaaaagaagaagaagaagaaaaagaagaaa
aagaagaaaaagaagaaaaagaagaaaacaacttagacaatatcgaaaatgtttgtactaaacaattaaattatatttcaaatgaaat
gtcaaaatcatcatcatttagttttaatggtttagaaaaagatgggtctgctgaagatggtttagaaaaagatgggtctgctgagaatg
gtgtagaaaaagatagatctgatgaatacagtttagaggagaccgaatcggagagttatagcacaatagaagtgggttctaaaaga
aacaaccatatattatatttggataaactaagtatatcggattcatcagatgtgggaatagaagagtgttatgaagaaggaatggaaa
taaataataaatttgataaaaatatagatggaataaataataaatttgataaaaatatagatggaataaataagaaagaagttgtttattc
tgatggtaaaatatttatccaaagtatatgtaagattaaaactagtatgaaaatatttataaataaaaaaaaaattggagaaatggaaaa
atattatcagacaataaaagagttagaaaaaaaaaaacaagaaaaaaatattatgcatcgaaatattttattggaaaaatcaaattata
aaatattttttttaaatctaaacaatttaaaaaattctgataaaatagaaaaaaattgtaaagacaattatggaaatgaagttttaaataac
attctttttaatatgaatgaaaatatattaaataattttctttatataaatatagttgaggataaagaaaattatttagttattagtttggaattg
ttagctaatattctttttcattgtaatttttttataatgataaaaaaagtggaaattagggaaaataataaaaatgatgaattagatgtaaga
aatgatgaaatatatcaatatgaaaatgatgataaaatattaaaagttaaatcaataaaaagtgaaaaaataaaagaatttataacagtt
gaaaaaaattattttttttttagaaatatttaattatattaatatatcaaaaaaatgtataaatatatatatagataataatttatattctgaaaca
aatttaaatattttttataaaaataataatgattcatatttcgatgcttatatagttggttataatcaactattagacaaaaacaattcagatg
aaatacataactatactattcgaccaaaacaaggaatactgaaatataaccattttaataaatttataattactagaaccaataagataa
acattatggcctttaatagttcattcttattgttaattaaaacagagaacaatcttttttcctacatcatttcttcacactttacaccatctcat
gaattatacactgaggctgagaaaaacgtgataagagacattttaaaagagaaccgcacaaagtttagcatctttttcaacgaattta
agcaggaaaaaagagaaagcacaatatttaacaaaaaagacgaaataataatctatgacatttcagaatcaaatgaaatacaaagt
gggtaa
PY17X 070500 Protein
>PvivaxP01|PVP01_0528800.1: pep
(SEQ ID NO: 25)
MDPRQNGRLARWAGLSLCCLLLLLFNALSPCGEGRVRWRSLGEQSGIGTNACSNI
IPFYLNSHNFFNPLREINCSNNFSLRWNINYVYFEFLNEEFFINFHLHSLFVKEVQLY
EYRERSILLLKKEASHDEVKFLIRVEPDGSVHPGEMRTFYFVIQAGGGPNFSACVN
VMLELNVGMVRRYHGGAAVGQLSSGETVDVVKHTNAVKHANVVKGLYSDRPI
VEEKKSKLPAHHKNYIIVKDNYYYASSEDYYVRFEWEKEESHADLPSNRAVVVY
TVLLFTQFREHGSISFYSSLTEDITKEKNFHMNLKKLSLGKSYLNNMTVNNILRLA
HLDKECSRGCVNSVRAKNGLFLHALLENNSVYKFEVKYVRGRGSPGGLAITPDG
AHFSMEFSIVRNAAEYTMVDFIREHFTRECMHNTFFPSGIVQTGGGGEESGGSREK
PPVQGYPPKGCPNDSTQNCTAANTANPANTANPANPSDQFVIYGRMIELNDNFIF
NFDPIKLFEQEKQVSITIAEESLFNLVVTSKADSVYLNLLKRDSPHTEKVCNTYYL
NNLNDYTLFNYMAKEMDHFPHVHVNKTFEGDYTDGNYTDGELLRRKKFPLHSV
AYINCTLPKGDYHLRITVDGYNLICDSNEVSLTIYPLSLYEEKHKCDSSMNVVTDL
FYYHLRAESKKHREEYTNLISPPNGESAATVEGLSKWADHLDTDVKTYQNIYENR
WVLNNPIFQDFDFVILYERHITERVDELLVTINNSVFVDLFFVIVEPIMGKNSYYYV
YRNSRTVSKYTVQHGDLPFTIYLIASTMHYPGKQLCGFFFVDIDLVTKAKLTWEE
EEKQPEEVGLTTAAHNMISRVNYIPDLIIGYDSYSFSNFCFIPKYKKHSLIIYVKEGS
IVKINCFSENYVYIYLYDQNKRKLYDGYNQLYIESFTKGEYEIVFEFNAPNDRKDN
AFFYLQVYVFHLSLLERCAAGAAGGAAWEAAGTGKGMDTGTGTSIANDTANST
AKGTANGSQTAEAPPAGTSPNAQDVYDLSSYVEASRPDLPTDANETQVKEKNTF
WFQAPNAYYLFKRDFLLLHPNKRIVKEVILPHVPSMDPAATFLLKVELIFAKNYLP
YKLAIQDTSNSKLQYHDSYTYKNKILMTLPVVNGGPPVGNNSTGETSQRGKLFKL
YVDLYEEVNDDLRNNFCTYAYLHIIYTSEMEAFRQWGEQGLSYATVNLPLLLNN
LLIKGPPGVGEAAIGEAGVGKGGVIEGDEAGESPTHSNDRSALISLRNADLPVANQ
SANYTFELVNNNVLLFLASDDLLFDMYMYRENRDIKLSVYKFSDTERLLSGGGIT
YDEVTKKGGPPGEEIFSFNQRQIYLSVHLTRGLYIFEYERGSGPFFANLSVVWPHG
EDMPSGGYLTGEEVGLPKKEVGAILPRGGALLREEVRFLFGKEPKCGGEAKLHHG
GEKLHNLTYFWREIIKNENFKILDNSISSVEVKKGEAKNALIYKRFYICLSDEVGKV
PQEGGHSNDTHRSSQTVEKNYTLFNLSIEESAQVYVEIKPHYHFFAYPFHLNIVSK
RSFFDISSGHKTVITIDLYKGEYEIHLAFPALQREQIKDVIFDLVILIVSPQNGEGEAE
KLGNAITHVGNPPGGSPQSDVCKTYPYKPLLNKVNLVDIPENDASVRRNIVSPKY
KNKFFHLFGKFFLKGYKEEVFFTIPNGGYVLKIFCLPIDESTGEGAASEEGATNRIG
DMGSTPFGNFSDPPTNFVNSFLNVRNVFNVRVVLNHSGEANQDRDGRKDPPQNS
PLFVSPLFANEDEEFMLNLYRVDRNFKVVIKTNSYLCNYFQLVVSLYPLDFHNPV
HSYAYAQANSVEKCMKNIFDTLSVKAKLYEDIKFEQKKFPSHQYLRIETDVVNLR
SAHKEDSFSFPVVVRKGSYFKANVGYDFSLASFQMKLLKKGTTISSSSKMEVSLK
ENALNTFENISLYLEEGSYTLEIRSYALTANLDINKNFSFCFYFELELFEFSGDNKG
DAVLLDVFPHNSVPVDGSGSFGVDLIFWGRVHTKDIHLEDGQKTPVEPTSRRAIN
YANIEVHKFLLSPEEMAKVEKNFIFKFSPSCNIYVNQEKEKKLKFTLSTDDRSAST
GEGTNGVEHSAAVVTSEPNERNGQVHPESVNNSFLLEYSQKEVPPDPGRTSYLQG
TSQKESVYDIDRVIQNFRLNEKKRRDEDDSRVSDSPKGEADACIPLTILTIEIYCFEK
SYFLIFIFFLCVWFMLCAFLFVLLKLYKNWKYYRNYDVITESEEVVNLFDDDHI
>PKNOWLESI|PKNH_.: PEP
(SEQ ID NO: 26)
MDRRKNGSLARWAGLTLCCWLLLLLIIMSPWEDGRIRGRSLEEQSGIGKNVCNNII
PFYLNSHNFFNPLREINVSNNFSLRWNINYIYFEYFNDEFFINFNLHSLLVKDVKLY
EYRDRSVLLLEKDASHDEVKFFIRVEPDKSVHPGDMRTFYFVIHSGISSNFSSCVN
VMLELNVGLVSRYEWASPIYGTAINQLTSGGNTNEVRSLHSDRNIVEEKKSKLPA
HHKDYIIVKENYYYASSEDYYVRFERKKEELYANLQGNEEVIVYTVLLFTQFTEH
GSISFYSHLTEDITKEKNFHMHLKKLTFGKSYLYNMSVHNILRLAYLDNDCTKGC
VHSVRTKNGLLLHALLENNSVYKFEVKYTLGRNSPDWLAKNSDGAHFNVEFSIV
RNAAEYTMVDFIREHFTRECMHNTFFPSAIIQGEGKNVAQEYSHKGCKEGQKESC
TSSDQVDEFIIYGRMIELNDNFIFNFDPINFFVQNKQISVIISEDSLFNLVVTSKIDNV
YINLLKKDLSHGEKVCNTYYLNNLNDYTLFNYMSKEMDHFPHGHVNKAFADDY
RDDEEVRRKKFSQHNMAYINCALSKGEYFLRISVDGHNMVCDSNEVSLTIHPVGL
YEERHKCDSSMNVVTDLFYYHLRSESKKHREEFSNLISAPQGEGLTNEEGSNSIAG
EIKWPKKIEEDGKKRTYQNIYENRWILNNPIFQDFDFVILYERHITERVDELLVTIN
NSIFVDLFFVIVEPIREQNSYYYIYRNSRNVFKYKIKHGDRPFTIYLLASTVHYPGK
QLCGFFFIDIDLVTRKKLTIEEKGPDNAEPTSNAHNMISRINYIPDLIIGYDSYSFSNF
CYIPKFKKHSLIIYIKENSIVKINCFSANYVYICLYDQNRRKLYDGYNQLYIESFTKG
EYEIVLQFNASNDQKDNAFFYLQFYVFHLSLLDRCAHSAAFITGADTGTDRGIGIV
SQMRGESTSIGSEGAATPPVATTPNVQDMYDLTPYVEANRADFTMGANQTQVKD
KNTFFFQSSNVYYLFKRDLLLLHPNKRIVKELILPHVTSMDSTVTFLLKIELIFAMN
YLPYKLAIQDMSNTRLQYHDSYTYKNKILMTLPLVNSPPLVENRATGEAGQVGK
RFKLYVDLYEEVNENLRDNFCTYAYLHIMYTSEMEAFQKWDEQGISYSTVNLPLL
LNNILFKGSPGATGKAFSVGEGSNHGGDHSAMISLRNSDLPIANQSVQYTFELLNN
NVFLFLVRDNLFFDMYMYRENRDIKLNVYKFSDTEKLLSGGGITYDEVMKKGSSS
IGEEIASLNKGQIYLSMYLTRGLYIFVYERGSGPFFANLSVVWHYKEGIPSGSYLTG
EEGVGLPKEEVGVILPMGTTPIRRDDAFLREEISFLFGKEVKCNEGTKVHHGGKKL
HNLSYFWQEIIKNENFEILENSTTTVEIKKGEQSNALIYKRFYICLSDQVQKISQEGR
NSNDSTHKSSETIDKNYTLFNLNIEESAQVYVEINPHYHFFVYPFNLNIVSKRSFFDI
SSGHKTFITIELYKGEYEIHLAFPGLEREQVKDIIFDLVILIVSSRSGKNETNQRNTLT
YIGNSPGLSPQSDVCKAYPYKPLFNKVNLVDIGENDVSVKKNIISSKYKNQYFHLF
GKFFLKNYKEEVFFTIPNGYYFLKIFCVPIDESTEENISVEEGEEKNEIREVRKISVG
NFSDPSMNFVNSFLNVRNVFNIRVVQEHSGEANEDGDDKKDSSQNSSLSVSPIFAN
EDEEFMLNLYQVEKNFKVVIRTNSYFCNYFQLVVSLYPLDFYNSVQSYMYSQVN
SVEKYMKNIFDTLSVKAKLYEDIKFEQEKFPSHQYVRIETDVVNLRSVQKEDSFSL
PVVVHKGSYFKVNLGYDFSLANFQMKLAKNGVAISSSTKMEVNLKDNSLNTFEN
ISLYLEEGNYTLEIHSYHITANLNNINKNFSFFFYFELEIFEFSDDKTGDAILLDVFPH
NSMAIDRSYPFGVDIIFWGRVHAKDIHLQDDQKTHIEPTNRTAIKYANIEVQKFVL
SPEIMNKMERNFIFKISPNCNIYVNQEKEKRLKFTLSTEDKNASIGESVYGEEKGEA
ITTNERNEENGEVHPESVNNSFLLRYAEKEIPTDIRGTSYVEGTSQKDSVYDIDRVI
QNFRLNEMKRREEEDSTMGNPSKEETEACIPLTILTIEFSCFEKGYFLIFIFFLCFCF
MMWVFLFVVLKLYKNWKYYRNYDVITESDEVVNLFDDDHI
>Pmalariae|PmUG01_05036900.1: pep
(SEQ ID NO: 27)
MERRKKRKKISLLIDVLLYFILFYSMFPIEKKNERKLSENYSEAGKAFKNILPFYLN
TPQYCNLLFDMNVSGTYSIRWGVNYIYFEHLNDSFFVNFNLYSIYVKEVELYEYR
HRSFLLLKKESVENEIKFVTSIEYDKSIDAKEQRTFYFTVHSNYVQSFDTYINVTID
VNIVGHVGRGGERGSGQVPSVDEKKNDDITMVDESKNGIKQNRLPLNNNSYIIIKE
NYYYASNEDYKFNVREADNFYVDSKGNKIIVIYSVLFFVDFNKHCNISFFSLLTQG
IEQGKNFSMDLKKIKLGKNYMHNMTTESILKYSFSHKECKEGCIKSSKTKNGVFL
HTLMENNNIYKFDIVYTIYSSHSTYGNDSIYGSYSNYDISNSDRGSGHTSTSSDTIRP
NEVVHFNLEYSIVNNSKNYTINDYMKEQFYKECVHNTFFPHQIIQVGENDEHFTG
LVVTPSGKNKCSQNGGKCIDDDKKKADNSNHVAENVDFIIYGRVIEINDTFILNFD
PINLFEEEHKVGLHVSEESLFNFSLISKSESIYVNLLKKGSSQTVCNTYYLNNINDY
SLFSYITRDSATFLHKHFNDTFEVNKKDANHKFKNFSLQNILYINCVVPKGEYELK
IIVNGYNVICDPNEINLVLYPLLMYEEKHTCDSSMNALTDLFYYHLRAENRSQSGR
GSVVGGLGGTTASRVDVSGEATPERTSNSASSYARQNDDLTSEVQKTYSNIYEKK
WMMNNPLFEQDDFVILYEKSIEEQVDELVVIVNNSLFVDIFLVIVEFIGEDSYYYIY
RNSRSIVKYMLKYKNPFTIYLLASNLHYENRKLCGYFFVDIDFLKGSITVSLDGTE
DYKKMIQKINYIPSLIIGYDSYAFSNFCYVPDYKKHILTICVQENSIIKINCFTQNYIY
IYLLDQNKNKIYEGYNHLYIESFTRGEYEIIFEFNVPNDKETDASFFYLQIYIYQLSA
LEKCVFDGQTDSSSGSSSDGNGKDAQFKNYLSSNENGDNSEYDLSSFSATNNSME
LKQKGKNFFFFQNEYAYHLFKRNFLFLFPSKRTTKNLILPQVSHLNNIAFLLKLEL
VFHKNFLPYKMTIQEGTDDLLMHHSYTYKNKILLTMTILNNSVKVFKLYIDLYEDI
NEKLKNDYCPYAYLNIIYTNEGGTYKQAAFSGLNFGEISMPLLLNSILLKSFAYAR
GMGNSERNKDEDMGVVRRNDSGVRNDDWSNKHTSTISFRNSNTVTIGSQSVDYN
LKIINNSTTLFLAEHSVFFNMYMYRENNDISLKIYKYLNSDNKLEEGYGVTFDEM
NKEHVLSHSEEIMSFSNKHIYISTWLKKGLYIFKYEQSTSPFFINLSVIWNYQDDDK
NMGKNYLKRYKYGGQSLTHYNNGKTEEPPADDAISDTILKNEIYFYFDKELKCDG
RKDIFYESKKKLHRLTFFLKEVINNNIFETHSDDSMQITVKRDSRNTLIYERYCICIG
GGPKVNDMHSDGSTSAVSANEVAQGVVTQPWTGSGSRSSSSSSSSNGSSSSSSSN
GSSSSSSNRYKIYHISVEESTKVYIEIKPHFHFFIYPFRVSVTSEDFYFNIFSENRNVIN
TNLGKGEYEVILSFPQLKENKNTLQNAIFDLIILVILPEKGAIIGDKILASSYMSNRL
SGKLLEKELCNISTYKTLFNKVDLVNINENDSYVSKYIISSKFKNNYFQIFDKYYVN
NFQQEIFFTIPGESYILKIFCMPIDELGGENMYKEGTYNNTYNGNSNSTYNNNAYN
NNITYDSSVHNNSSSETMSNIYNVASNYVNAILNVRNNFSVQVVLADMDNKSNV
KNNTSNDNEKDKYDDNNSSNRNNSSNSNAFVSPLHSNENEEYMLILYKVDNNFK
LVIKTNNHECNYFKIILSLHPLSFYNSEHYFSYANYLDKYLKNLFNTLSVKTKLYE
GINYTHEKSSMYSLTRLTSPIVNLKSVNMKDLFSFPLSINKASYFKINIGYNFSLVSF
EIKLMKNSTTISCSNKVEVNLQDDTVNIFENISVYLEKGDYILQIFSFDYMGNSVNI
NKNFSFPFYFELEIFEFFQETKKSGPILLDIFPHNSVPVDAVFWGEVKGKEMFLEVH
NKKYADLRNRKIIKYGNIEIHKSFLLPEDMMNMEESFILKFTPSANIYVSEQNEKKL
NFIFSTYSTNIEQNNNSYKVVQGEDKIVMIGSGQRNGQVHEENVNKSFLFEYDQR
DITEAGESVNDSQTNIHVDKFFDLDNVIKNYRLNEKKKKREVSYTSLENSNKQND
SCIPLYLFTYKIYCFERSNYLIFIFSFFLFSVSCAFICLLIKLYNNWKYYNNYDIVVES
EEVINLFDDDDI
>Povale|PocGH01_05030600.1: pep
(SEQ ID NO: 28)
MFVCIIFCVLFLPFGRGIPRNLGDDNIKRRSQCENLVPFYFNAPQYWNMSGDINISD
YYSLRWDVNYIYFEKVNHKTFVNFNLYSIYVKNVELYEHRDRNIMILRREVMEGE
VKFVHFIDQDVSLHTLEKRIFYFIIRLKKPLHDDTCVKVGLDVNIGKAPITSFEDGM
VVHQEKKSKRPLHHKSYIIIRENYYYASNDDFYFTPEDSENDTYIDDNGAKFVIVY
SVLLFPQFDKKCNISFFSFLNGEITKKKNFSMQLRKIMLNNNYFHNMNIDSVLKHT
YNNMTCSEKCVKSIFTKNGLLIHNLLENNYMYKFEIVYKLTENLNKDQGNYENN
TVNGWTYNINSKTYFNMEFSVVMSLKDQSFIDYVKEYFNSECIHNSFFVKKIVQIG
IYKGRNDNVISSFSMKKCQEKDKHRCNFTELNNEYVIYGRIIQLNDSYVLNFDPIN
LFREEEKVHIHISEDSLFNFTLTTTLENIYVNLLKGNSTQNVCNTYYLHHINEYNIFS
FQSKDKAPFMHYHFNTTFGDKKKYSKDHRLKKFSLQNVVYINCVLPKGEYDLRI
VMNDYISICQPNDVQLTIFPLHIYEERHTCGREMHVLTDLIYYHLRGEGRRGGSSG
EGNRPIKNELTEPSSEQSRTISHNVYETMWELNNPLFENFDFVVLHENIIDEEVDEI
VVTVNTSAFVDIFLVITESVGEESYYYVYKNSRGVAKYGLKFGGPFHMYLLAPSL
HYEDRKICGFFFVDVDLVLLGKKAKGKTPRREDHTVGEETLSSVDEPQGRNVVQ
RTIHLPELIIGYDTYAFSNFCYVPSYKKHVLTIYVQENSIVKINCFTQNPIYIYLLNSE
QKKIHDGYNHLYVESFTKGEYHVVFEFNLQSEKEASSFFYLQIYIYHFNLLERCAF
DNPSEALSSSEELPLSSALRIDPLRSDPSQSNDVYDFSIYNEMSTAKDGIKVVDPTSF
LFNDEHSYFFFKNMFIVIFSNKIISKKVILPRVATHLEASPFMLKIELIFHNNFLPLKL
RVQDITNSLLYHSSYTYKNKIILTLTLSNGRVRVLNLLIDMYEDVSDSLKSNYCPY
AYLHFVYTNEVEAYRKIGASGLSHGAIDLPLLLNSLLLRAVWGVDDKGEEIRINTS
SDNVDSASTPPIEGGVTFRSPVFPVMVNQSVNYTIEMVNNDTMLFIADNDVFINIY
LSKENGIVVLKMYHCLSTGILEKGSGIQYDEVHKNFLSISKEIASLSGKHVYLSTYL
ERGLYVLKFERDSSPIHVNVSVIWNSNREDPTIIGYVKRQEHANKLVNYASREVLP
VMSMHRMNVSAQGTASATDISPAGDPILRKETHLFFGKELHCSGVKQVFRESKKV
ASVPFFFDEIVKTKLFAVYSGRTTHILVKKNADNMLIYERFYVCLGRETNGYYEQ
YSGEASRDESDQHNEGKYPLFGITVEDVAQIYVEIKPHYHFFIYPFELNVTSSSSYF
SHSSGGKNFVSTNLGKGEYEIDLRFPEWTNNDIKSVMFDFVILLTFPIWEAYDTEV
LSKKGTKMLLRDDTCNVSMYRSLFNKVDLINTTENDIIVSKNIISPKFKNKYFHVF
DKYFIRDYQQEIFFTIPSGGYILKIFAVVDSEIGEGNDQGEGFPGESHPQGTGRVVD
FPANYVNSFLNVRNVFNIRIVESDLNGLGGKNNNVEDKKEEISYVAPVYSNESED
YILVVYKVYTNFKMIIKTNNYDCNYFQLILNLHSVNSYSDVHHFSHAYNFNSYLK
NIFDTLTVKAKLHDGINFEQKKLSTYNHVSITTSIVYLKNINPLELFSYPLSIKKGSY
FKISVGYNFTQAHFEAKLGKNGEPICSGSKEKVNLKDDTINVFESISLFLDEGEYSL
QILSHDLIEDSVNISKKFSFPFYLELDIFEFMHEKIENPILLDIFPHNSVPLDRNYPFFV
DLIFWGELRGRAVYAEDGKQKRVQLRSGRMHTHGNVEIHKLILPPEEMDHMENN
FVILFDAKESNQMNREKEKRLYFSFSTISTNVLSKNSSYEPIREKQNVMSESKEGN
RQIHPESVNTSFLLEYGQRDAKRDDSIQENSNKDNFFDLDTVIRNYRLSQKKDKEY
MPVSGEAPPKGESSTCIPLSIFALEIYCFEKNYFLLFLFVVFILCISCAFLFLLIKLWK
NWTYYSSYDVITENEEEVTLFDDDI
>Pfalciparum|PF3D7_0419400.1: pep
(SEQ ID NO: 29)
MRKKKKTCLVLLILSFLCFFYSSLSYDEDYDEEYNYEDKNSDYECNNLIPLFLNNL
EYSNISQEISLSKTFSLKYPVHYMYFKYSSDQSIFVNYNIYNSLSFGYVKKIELYEY
EIIKNESVLLLQAEKDSDKNIKILYTITKIDNRKNYEGEKDQRLFYFIIYTTIENDNDI
IKSCINVNMSIHIRIIKDDTQTNNHNNNNIIINNNNNYYYNYVHNNYGMRGIMPNH
LYNHLLPLHNKSYIIIKDNYYYCTNEDYYYNMNNIEDYYYDEKSGDKVVVIYSVL
LFIEFKKEANIKFTSFLKHDINKYNNFVIHIKKIDIHNNYLNNMDMEHILKVGNNEE
YINGNKKKKSMECEEDCVESYITSQGIYIDTILNNNHLYRLDILYKMGKNEINNKT
TEIIKKENNNNNNNNNNNNNNNNNNNNKNNNNNNNNNNNKNNNNNNYYDYD
NDKMGDIKNMKDNGKDPFLSSLLFFNFELSIYNITDNYIPMNYINKRLYTECSNNS
HAPNKIVQTKEENMLNIFYTNECSKNIEKCSLYDVNEYIIYNNKVIDIHDNFLLNFD
IINSFKNEQIIDIYINEDSIFNFSMLIKSDHIFVNLLKNNSSEKICNTYYMNNINDYNI
YDYKKNNKSDFFHKHYNDSFLYNQNMNYNNTFEYQNYYQHDHHLKNFPLQNII
YINCILKKGYYDLKIILNGYNNICESNDFNLLIYPMSLYKNQHQCDDKMNQVTNL
FNNILKRKYQHNISHVPNQIQTFDQIYQNKWILNNHIFEYFDFVIIYEKELLPQQYK
EIMVTINNSPGLDLFFILVENVQGQKYYHIYKDTRKKNKYILKDKHPCTLYVLVST
LLYTTQDVCGFFFVDIEYVDNMKLLNQNDHPNDIMNGTYQHNMIQKINYIPTIIIG
YDSYSYSHFCYVPYYKKHKLTILLKPKTIVKINCFTHNYIYIYLLDKSNNNNNNKN
NNDNNKKKLYEGYNHLYIESFVEGEYEIIFEFNTQNDKNMNSFFYLQIYIYNLNTL
DKCLFFNNNMNYIKESSINKFINTNNYDTFDLSKYEYNDIIINKNVPRVIKKENNMF
FIYQTINFYYLYKSFFLYILPTQHKDGNITNEQDKNRIKKKIILPKINYLSNQNFILKI
ELSFHKNIFPYKMYIDENYDDIIMISTNTYRNKNIMLLKILNNNTNYINIYIDTYEDI
NDEIRKNYCSYAYFNITYTNQTEEETVDRQQINLYNKITLPLLLNNILSKDYVPMIN
KNIVENKNDQTYKADHLYYEDDNIGVINSVSNNYFSGENKNMGDIKNKMNNEY
GSVHTEQMVHFQNNDNLNNNNNIIFGNVYPYLENHLRGIISLKNSNSLILNQSVNY
NFEVVNNNYTLLIIPNDAFLNMYIRNNDDDDDDDNDKNNNNNNNNSNNNNSNSS
KKNITLNVYRILDVPKLTNEGISFNEVKKQILSLSHPFLTFTDKYINVYRYLERGLYI
FKFDDDSNKENSSYTFRNSSNIYKNNINNYNYHSSDSIKPLSFFMHFNIMPIYTNDI
EYKDNIKNNNYNDKTFNYQGHDKNMYPSVIHNYIFKNELLYYFNKEYSCDKENM
LFFEHKKIDDVSNFLEHTWNNIPFEIYINDKLDITLNRDNYNTLILKRFFICFEKEKN
VNNNDNINNSNNSYVNNTDNINESQDDSYINIFNDTIKKEYLLFNIVLNVKGNIYIE
IQPHYHYFIYPFILTIKSKNNPTQLNKSHDKLFLTTDLGVGEYEIYITFLSNNHYKN
VKMKKVMFDLFIFIDILKNKNIQLGKNNLYLPYGNHLDDTIDFKIYDNTCKTDNY
KRLFHEVKLLENNNSSNVHMNESYIKNKNNIISTNMNTNYFHIYDTYYMKVREHE
ISFEIPKEAYILKIFCIHIDEQNYDEDITSGDFYNNTNAYNNNNNYGNNNNYYKIEQ
TDNLSHFSFNYLNTYFDIQNLFHINVITNEDSKFFSDEHTNDQDLYVKPFFSNEENE
HILNWYKIDKNFKLVIKKNNYDCTYFKLIISLYPMDYFNSIDYLKYNNYLNKYFTN
IFDTLSLKVNQYQDISFEQIKNKNYLYDYLKTNVIFLKSLNSRDLFSYPLTIKMPSY
VKINFGYNFTLTSFEIKLLKNNMIIATSNKVQANINNNNINIFENVSIYLEKGNYVV
QIYSYDLIEKSSNIINKLSFPFYAEIEIVQFVNDQIDEPILLDVFPHNSVIVNRNYPFFV
DLIFWGRANENISVLDTKENNIKLNNTKLIKYGNVEIHKYVLSSEQMRSLENNFTL
KLQSNVHISAWMEKRMNFSFTNEGWNYDGQTEHGKLMKNGIKTNNIVDESSKIS
KGEKLNETVLLEYGKREIKNESEGESKSENKIEGNSNNESDGESEKRSFLQNGVIE
KSNLFDLNEVIKNFRYNKKKKKEEEDFLLNGSYMNSNDKCFSLSIFSFEIYCFEKF
YFFIFIFILTVLWISCAFIFLIIKIYKNWKGYRNYDIVGEMDEVIGLFHGDDL
>Pyoelii|PY17X_0721500.1: pep
(SEQ ID NO: 30)
MNKRKGKVWLFLFIICLIVINFKISYEQNNERNLSEITKNEIEYERCQNLVPFYLNN
PEYYTMLNEINVSNEYSIKFDITYIYFEYINCNFYINLKLYSKDVNKVELYEYVDN
KGILIFSENSKDNLIKYLFHVQDDKNSNKNENKNENKNENKRVFYFIIYSNKIETSN
ECVNIRLDIGIGPVVLEKGDTKIESTKKGDIQKEGRQNEGRQKEDRQIEHHNSYIIIR
DNYRYATDGYFNFSLKNNKDFYIYENGKKYGIIYNVIIILEFNEENNINFFSLLSQG
KNKWNNFSMQLKKIKLDKNYLHNMNIEYIIKHANADMTCNDMCIKSDESKNGIIF
LNALLSNNYIYNFQIRYAENENENNYINNDINYDNENKSNKYLGRNSVESKKEMF
NQDNIIYFNFEYNIVNNVDKYTMIDYLKGHFNHENIQNTFFFDKIIQIKDNNINKNN
ILPIIGFNKECVGENKKECNKYEINNNYIIYGNTIEIDDNFILNFDPNNLFKEETKVNI
TIEEESFFMFTLESKSENIFVNLLKKNSTENVCNTYYLKHIKDYNLFSYLTKNEKNI
LKYDKTNFSRHHIKNLSKKIIIYIKCILLEGEYDLKINFEEYNKKFESNNINLIIQPLHI
YEKVHSCDRKMNIITDMFYYNLRTENVNKKNDNNVISYKNIYENMWELNNSLFD
DFDFIILYENEIFENVDKIIVTINNSIFSDIFIIIFETVKDETHYYIYKNSKSTIEYEIKNK
LNNKISIYVLTPSLHYSGRKICGFFFVDIDFIENKKESIGEITLFNNMYKSNRSYIGSI
NHIPYLIIGYDTYSFSNFCYIPDNKEHVLIIFIEKESIIKINCFMENYVYIELYERKHIG
GEIKKIKLFEEYQQVYIESFTKGEYEIVFKFNVQNDREMSNFFYLQIYIYQINLLDK
CVFHNEDDFGEIAEGSSSVYDLNEVDRKIKSKNGKKNKEIGYFNDNNGFIFENESS
YYLFKRYLLFLFPKKIDEKIEKKIILPETNNSGFVLKAELVFHKNFLPYKLSLEEDG
ENILAHESNIYKNKIIITFNILNKNVKYVKLYIYLYDNVHEKLIKDYCPYAYLNIVY
THELEKYREHENDEYQYDTMHLPLFLNNILLKRYEYSDDVEEEDVEIGKVETESG
NDKNVRVVPFDKKNGMCIGNQKIEYKFITSNNNMMFFLAERDAFLNIYIYKEGKE
DIMLNIYKYKNISKFEKNEILPYDEINKDIQSCCEEIFSHSNVNDINISTYFEKGLYIF
KFSEKLPYINMIFSVIWVEIPNVNDIIMGSNINNKYEINRYIEENESKFYFSKELKCG
DKKGIFYDENKLKDLEKFVIENMFEKNLYTIYFGNKKKEITISNDSKNIVMYERIHI
CFGNEIFNHQVINNTNYIKENEESHIILTLNVEKECQVYVEVKPHFYYFIYPFKINIL
SKNSYFKVSNEYKKYVYANIEQGEYNIEISFKMENYINNKIKKIDGVIFDFIIFIYNT
SNENKLKNIDFSDEEKKQMVSLKNETSNMNKLYTRKIYKNIFKKVNLKNLNENGI
VVNEKITTHKSKYSYFFCYDKYIIKNHQEIFFTLVDNVDYILKVYLAPIYELSGNNN
SKNINNDFEHFENFENFENFENSDNNSESSSLYNFSTNYLNYTLNVRNVFNIEVVN
ESMNNDYIPEKVIKEKRLKNKIIEKKTPDSNILYTNEDDEYILNVYEVNKNFKLVIK
NYNNIDSNIELVLSLVPLKYYKMNMNTKYANEYINSYFKSIFNTISVKTNLYSGIIF
ENREHSTHIYTRVETSVVYLKNIIMEETFSFPLHVKKGSYIKLNIGYDFSRVNFDVK
IMRNNKKVSTSNKIKGNMDNGKINIFENISLFLEEGEYTFQIWFYNLTDNYIHINKN
MGFPFYFSLEIFEFAENKNNTSVLLDVYPHRSVYVDKAYPFNIDLIFWSEEKREKH
GFMEDEMKKIVYLRVGEIIKSGKIEIQKSYLLPEDMSNLKNNMTLKFDLKDNVDM
ISENNNENNNGNNNENNNKNNRYLFTFLNNDVKENEVAIETNNTNLNNQEKLKQ
IHNESVNKSILLEYKKEDSEKIEEKDSYISHNVNQDMFYDIDKAIKNYRSRENNES
KEITSSIFNKPFSDPDDKSCIYIPIFLIEIYCFEKNNLFYFIFILFIFFMTSAFIFLLIKFYK
NWKYYRNYESFKEYDETISLFDDDDI
PY17X 070500 DNA
>PvivaxP01|PVP01_0528800.1: pep
(SEQ ID NO: 31)
atggatccccggcaaaatggtcgccttgcccgctgggcggggttatcgctctgctgcctgttgctcctcctgtttaacgctctgtctc
cctgcggggagggccgcgtcagatggagaagtctcggagagcaaagcggcatcgggacaaacgcatgcagcaatatcatccc
gttttacctgaacagtcataatttttttaaccccctgcgggaaatcaactgctcaaataatttctccctaaggtggaacatcaactacgt
gtatttcgaatttctcaacgaagagtttttcataaactttcacctccacagtttgttcgtcaaggaggtgcagctgtacgagtaccgaga
gaggagcattttacttttgaagaaggaggcctcgcatgatgaagtgaaatttcttattcgtgttgaaccggatggaagtgtccacccg
ggggaaatgagaactttttattttgtcattcaggcgggggggggcccaaatttcagcgcgtgtgtgaatgtaatgctggagttgaat
gtcggcatggtgaggaggtaccacgggggggccgcggttggtcagctgagtagtggcgaaacggtcgatgtggtgaaacaca
ccaatgcggtgaaacacgccaatgtggtgaaaggcctctactctgatagacccatcgtggaggagaaaaagagcaaactgccg
gcgcaccacaaaaattacatcatcgtgaaggacaactactactacgcttcgagtgaggactactacgttcggttcgaatgggagaa
ggaggagtcccatgccgatctgccatcgaaccgagcagtggtcgtctacacagtcctgttgttcacccaatttagggaacacggc
agtataagcttctactccagtttaacagaagatataacgaaagagaaaaacttccacatgaatttgaagaagttatcgttggggaaga
gctatctcaacaacatgactgttaataacattttgaggttggctcatctggataaggagtgctccagggggtgtgtcaacagtgtgag
ggcaaaaaatggattgttcctgcacgccctgctggagaataactccgtgtacaaatttgaggtcaagtacgttagggggaggggg
tccccaggggggctagccattacccccgatggggctcatttcagcatggagttttccatcgtgaggaatgcagcggagtacaccat
ggtggacttcatccgggagcacttcaccagggagtgcatgcacaatacgttcttcccgagtgggatcgtgcaaacggggggagg
cggcgaagaaagcggtggaagcagggagaagccccccgtccagggatacccaccaaaagggtgcccaaacgattcgacgca
gaattgcaccgccgctaacacggctaaccccgctaacacggctaaccccgctaacccgagtgaccaattcgtcatatacggccg
gatgatcgagctgaacgataacttcattttcaattttgacccgattaagttgttcgagcaggaaaagcaagtgagcataaccatcgct
gaggagagtctcttcaatttggttgttacatccaaggcggatagcgtgtacctcaatttgcttaaaagggattcgccccacacggag
aaggtctgcaacacgtattacttgaataacctaaacgattacactctctttaactacatggcgaaggagatggatcacttcccccatgt
gcatgtcaacaagacgtttgagggggactacacagatggtaattatacagatggggagctgctgcggaggaagaagttcccgct
ccacagtgtggcctacataaactgcaccctgcccaagggggactaccatttgaggataaccgtcgatgggtataacctgatctgcg
attcgaacgaagtcagtttgaccatttaccccttgagcctgtacgaggaaaaacacaaatgcgatagcagcatgaatgtggtgacg
gatttgttttactaccacttgagggcggagagtaagaagcaccgggaggagtacaccaatttgattagcccacctaatggggaga
gtgccgccactgtggaaggactctccaaatgggcagaccacctcgacacagatgtgaagacgtaccagaatatctacgaaaaca
ggtgggtgctgaacaacccgatatttcaagacttcgattttgtgattctgtacgagaggcacatcacggagagggtggacgagctg
ctcgtcacgataaacaactccgtcttcgtggacctcttctttgtcattgtggaacctatcatggggaaaaactcctactattacgtctac
agaaattctaggaccgtttctaagtatacggttcaacatggggacctccccttcaccatttatctgatcgcctccaccatgcactaccc
ggggaagcagctgtgcggttttttcttcgtcgacattgacctggtgaccaaggcgaagttaacatgggaggaggaggagaaaca
accagaggaggtaggactcacaaccgctgctcacaacatgattagccgggtcaactacatcccagatttgatcatcggctacgact
cctactcattcagcaacttttgcttcatcccaaagtataagaagcatagcctcatcatctacgtaaaggaaggctccattgtcaaaata
aattgcttcagcgagaattatgtgtatatttacctgtacgatcagaacaagaggaagctctacgatgggtacaaccagctgtacatcg
agtccttcacgaagggggagtacgaaattgtcttcgagtttaacgcgcccaacgaccgcaaggacaatgcgtttttctacctccag
gtgtacgtcttccacttgagtctgctggagaggtgcgccgcaggggcggcggggggagcggcgtgggaagcggcgggaaca
ggaaaaggtatggacacaggcaccggcacaagcatcgccaatgacaccgccaatagcaccgccaaaggaaccgccaatggtt
cccaaactgcggaggcgccccccgcgggaacctcgcccaacgcacaggacgtgtacgacctgagctcctacgtcgaagcgag
tcggccggatctccccacggatgctaacgaaacccaagtgaaggaaaagaacactttctggtttcaagccccaaacgcgtactac
ctattcaagagggacacctactgctccatccgaacaaaaggattgtaaaggaggtgatcctcccccacgtgcccagcatggaccc
cgcagctaccttcctcctgaaggtagaactcatttttgcaaagaattatttgccctataagttggccatccaggacacgtccaacagta
aactgcagtaccatgatagttacacctacaagaataaaattctgatgactcttcccgtggtgaatggcggccccccggtgggcaac
aactccacgggggagacctcccagcgggggaagctcttcaagctgtatgtggacctgtacgaagaggtgaacgacgatttaagg
aacaacttttgcacgtacgcctacctgcatattatctacacgagcgagatggaggcctttcggcagtggggcgagcagggcctgtc
ctacgccacggtgaacttgcccctcctgctgaataacctgcttataaagggtccccccggcgtgggggaagctgccataggcgaa
gccggtgtaggcaaaggtggcgtaatcgaaggtgacgaagctggcgaatcgcctacccacagcaacgatcgctccgcgctcat
ctcgctgcgcaacgccgacctgcccgtcgcgaaccagagcgcaaactacaccttcgaactggtcaacaacaatgtgctcctcttc
ctagccagcgatgacctcctttttgatatgtacatgtacagggaaaacagagacataaaactgagtgtgtacaaattttcagacacg
gagaggctactctccgggggaggaatcacttacgatgaagtaacgaagaaagggggtcccccaggagaagaaattttttccttca
accagaggcagatatacctatcggtgcatctaaccaggggtctgtacatctttgagtatgaacgggggtcgggcccctttttcgcca
acctgtctgtggtgtggccgcacggggaggacatgccaagtggggggtacctcaccggggaggaggtgggcttgccgaagaa
ggaagttggagcgatccttcccaggggaggtgccctattgagggaggaagtccgtttcctctttgggaaggaacccaagtgcgg
cggggaagcaaaactgcaccatggaggagagaagctgcacaatttgacctacttctggcgagaaattataaaaaatgaaaatttta
aaatcctagacaatagcatctccagcgtggaagtgaaaaaaggcgaagcaaaaaatgccctaatttataagcgcttttatatatgcc
tatcggacgaagtggggaaggtcccccaagaggggggacactcgaatgatacgcacaggagtagccaaacggttgaaaagaa
ttacacgctttttaatttaagcatagaagagagcgcccaagtgtacgtagagataaaaccccactatcacttctttgcctacccattcc
acctcaacatcgtttcgaagcgatccttctttgacatctccagtgggcataaaaccgtcattacgattgacctgtacaagggggagta
cgaaatacatctggcctttccagccctgcagagggagcaaataaaagacgtcatatttgacttggtcatactgattgtgtcgcccca
aaacggggaaggagaagcggagaagttggggaatgcaataacccatgtaggtaaccctcctggagggtccccccaaagtgac
gtctgcaaaacgtacccatataaaccccttctgaacaaagtcaatctggtggacatccccgaaaatgatgcgtcagtgaggaggaa
tatagtctccccgaagtataaaaataaattttttcatctttttggaaaatttttcctcaaagggtataaggaagaagtcttctttaccatccc
aaatggtggctacgttttgaagattttttgcctccccattgatgagtcaacgggtgaaggtgccgcctcggaggagggggcaacaa
atcgaattggcgacatggggagcaccccctttgggaatttctcagaccccccaacgaactttgtaaactcctttttgaacgtgcgaa
atgtgtttaatgttagggtcgttctgaaccactctggggaagccaaccaggatagagatggcaggaaagatcccccacagaatag
ccccctttttgtttcacccctctttgccaacgaagatgaggagttcatgttgaacctgtaccgagttgatagaaacttcaaagtggtgat
caaaacgaatagctatttgtgtaattacttccagctagtggtgagtctgtaccccctggacaccacaaccctgtgcacagctatgcgt
atgcccaggcgaactctgtggagaagtgtatgaagaatatttttgacaccctatctgttaaggcgaaactgtatgaggatataaaattt
gagcagaagaaattcccctctcatcagtaccttcggatagaaacggatgttgtcaatttgaggagcgcccacaaggaggactcctt
ttcgttccccgtggttgtgcggaaggggagctacttcaaagcgaacgtaggctacgacttttcgctagccagcttccaaatgaagct
cctgaaaaagggcacaaccatctcaagcagcagcaagatggaagtcagtctgaaggaaaacgccctgaacacgtttgaaaatat
tagcctttacctggaggagggaagctacactctggagattcgctcctacgcgctcactgcaaatttggacataaataaaaacttcag
cttttgcttctatttcgagctcgagcttttcgagttttccggcgacaacaagggggacgcggttctgctggatgtctttccgcacaact
cggtgcccgtcgatgggagcggctcctttggggtggacctcattttctggggaagagtacacacgaaggacatacacctggagg
acggccagaagacgcccgtggagccaaccagcagaagagcaatcaattacgcgaacattgaggtccataaattccttttatcccc
tgaagaaatggccaaagtggaaaagaattttatttttaaattttccccaagttgtaatatctatgttaatcaggaaaaggaaaaaaagct
aaagtttaccctctcgactgatgacaggagtgcttccaccggagaaggcactaatggagtggagcacagtgcagcagttgtaacg
agcgaaccgaatgagcgaaatggccaggtgcacccagagagtgtaaataattccttcctcttggagtattcgcagaaggaagtcc
cgcccgaccctggcagaacaagctaccttcagggaacttcccaaaaggagagcgtgtacgacatcgaccgtgtcattcagaactt
ccgactgaatgaaaagaaaagaagagacgaagacgactcccgtgtgagcgattccccgaagggagaagcagacgcgtgtatc
ccgctgaccatactgaccatcgaaatttactgcttcgagaaaagctactttctcatttttattttttttttgtgcgtttggtttatgctgtgcg
cgttcctgttcgtccttttaaaattgtacaaaaactggaagtactacaggaattacgacgtcattacggagagtgaggaggtggtcaa
cctgttcgacgacgatcacatatag
>Pknowlesi|PKNH_0512100.1: pep
(SEQ ID NO: 32)
atggatcgcaggaaaaatgggagccttgcccgttgggcggggttaactctctgctgttggttgctcctactgcttatcattatgtccc
cctgggaggatggccgcatccgagggaggagtcttgaagagcaaagcggcatcgggaaaaacgtatgtaacaatatcatcccg
ttttacctgaacagccataattttttcaaccctctgagggaaataaatgtctcaaataatttctccctaaggtggaacatcaactatatat
acttcgaatacttcaacgatgagttctttataaattttaacctacacagtttattggtaaaggatgtgaaactatatgagtacagagaca
ggagcgttctccttttggagaaagatgcctcccacgatgaagtgaagttttttattcgtgtggaaccggacaaaagtgtacaccctg
gggatatgagaaccttttattttgtcatacattcggggattagttcaaatttcagctcatgtgttaatgtaatgttggagttgaatgtcggc
ttggtgagtaggtacgagtgggcttcgccaatttatgggacagccattaatcaacttactagcggtggaaacaccaatgaggtgag
aagcctccattctgataggaatatcgtggaggagaagaaaagtaaactccctgcacatcacaaggattacatcattgtgaaggaga
attactactacgcttcgagtgaagattactacgttcgattcgaacgaaagaaggaggagctttatgctaatttgcaagggaacgaag
aagtgattgtctacactgtccttttatttactcagttcaccgaacatgggagtataagtttctattcccacttaacagaagatataacaaa
agaaaagaatttccacatgcatttgaaaaagttaaccttcggaaagagttacctgtacaacatgagcgtgcataacattttaaggttg
gcttatctggataacgattgcaccaagggctgtgtccacagtgtgcgaacaaaaaatgggttgttattgcacgccctattggagaat
aactccgtgtataaatttgaggttaagtacactttgggaaggaattctccagattggttagccaagaattctgatggtgctcatttcaac
gtagaattttccatagtgaggaatgcagcggagtacaccatggtggacttcattcgtgaacacttcaccagggagtgtatgcacaac
accttcttcccgagcgcaatcatccaaggggagggaaaaaatgtcgctcaggaatattcacacaaagggtgcaaagaggggca
gaaagagagttgtacttcctctgatcaagtcgatgaattcatcatttacgggaggatgatcgaactgaacgataattttattttcaatttt
gatcctattaatttctttgtgcaaaataaacaaattagcgtaatcatcagtgaggacagtctgttcaatttggttgttacatctaagataga
taacgtttacataaatttgcttaaaaaggatttatcccatggagagaaggtttgtaacacgtactacttgaataacctaaacgattacac
gctttttaattacatgtcgaaagagatggaccattttccacatggacatgtgaataaagcgtttgctgatgattatagggatgatgaag
aggtacggaggaagaagttctctcagcacaacatggcctacataaactgcgccttatccaagggggaatacttcttaagaataagc
gttgatgggcataatatggtttgcgactctaacgaagtcagtctaactattcacccggtgggtttatatgaggaaagacacaaatgcg
atagctccatgaatgttgtgactgacctgttctactaccatttgagatctgaaagtaagaagcatagagaggagttttccaacctcatc
agcgcacctcagggggagggtctaacgaacgaagagggttccaactcgatagcgggtgagataaaatggccaaagaaaatcg
aagaagacggaaaaaaaagaacataccagaatatctacgaaaacagatggattttgaacaacccgatatttcaagatttcgactttg
tgattctgtatgagaggcacatcactgagagggtggatgaattactcgtgacgataaacaactccatcttcgtggacctcttctttgta
attgtagaacccatcagagagcagaactcctactattacatctatagaaattctaggaacgttttcaagtataagataaaacatgggg
atcgtccgttcaccatttatctacttgcctccactgtgcactatccaggaaagcagttatgcggttttttcttcattgacattgacttggtg
actaggaagaaattaacaatagaagagaaaggaccagataatgcggagcccacaagcaatgcacacaacatgatcagcaggat
caactatatccccgacttgatcatcggttacgactcctactcgtttagcaacttctgctacatcccaaagtttaaaaagcatagcctgat
catctacataaaggaaaactctattgtaaaaataaattgctttagcgccaactatgtgtacatttgcctgtacgaccagaataggagaa
aactttacgatggctataatcagctgtacattgaatctttcacaaagggagagtacgaaatcgttctgcagtttaatgcgtcgaacgac
cagaaggacaacgcgtttttctacctccagttttatgtcttccacttgagtttgctcgataggtgcgctcactctgcagcgttcatcaca
ggtgcagacacaggtacagacagaggcattggcatagtcagtcaaatgagaggggaatccacttcgattggatcagagggtgca
gcgacgccccccgtagcgaccacaccgaatgtacaggacatgtacgacctgaccccctatgtcgaagccaatcgggcggatttc
accatgggtgctaatcaaactcaagtgaaagacaaaaacaccttcttttttcagagctcaaatgtgtattacctattcaagagggactt
actgcttctccatccgaacaaaaggattgtgaaggagctgattctcccccatgtgacgagcatggattccacagttacattcctcctg
aagatagaactaatttttgcaatgaattatttgccatataagctagccatccaggatatgtcaaacaccagactacagtaccatgatag
ttacacgtataagaataaaatcctgatgactcttcccttagtgaatagcccccccttggtggaaaatagggccacgggtgaggccg
gccaggtagggaaacgcttcaagctgtacgtagacctgtacgaagaagttaacgaaaatttaagggacaacttttgcacgtatgcc
tacctgcacattatgtacacaagtgaaatggaggcttttcagaaatgggatgagcagggcatttcctactccacggtgaacttacctc
tccttctgaataatatacttttcaagggttctcctggtgcaacagggaaagcatttagtgtaggtgaagggtctaaccatggtggagat
cactccgcgatgatttcgcttcgcaactccgatctgcccatagcgaaccagagcgtgcagtacacctttgaattgctgaacaacaat
gtattcctattcctagtcagggataaccttttttttgatatgtacatgtacagggaaaacagagatataaagttgaatgtgtataaattttc
cgacacggagaaattactttctgggggaggaatcacctacgatgaagtgatgaagaaagggtcgtcatcgataggagaagagat
tgcttcactcaacaaggggcagatatacctatcgatgtatctaacgaggggtttatacatttttgtgtatgaacgtggatcaggcccttt
tttcgccaatctgtctgtggtgtggcattacaaggaaggcataccaagtgggtcttacctcacaggagaagaaggagtgggtttgc
cgaaggaggaagttggagtgatccttcccatgggcacaacaccaattcggagggatgatgcctttctgagggaagaaatcagttt
cttatttggtaaggaagttaagtgcaacgaagggacaaaggtacaccatggcggtaagaagctgcacaatttgtcttacttttggca
agaaattataaaaaatgaaaatttcgaaattctcgaaaatagtacgaccaccgtggaaataaaaaaaggtgaacaaagcaatgctct
tatttacaagcgtttttatatatgcttatcggatcaagtgcagaaaatttcacaagagggaaggaactcaaatgattctacacacaaga
gtagcgaaaccattgataaaaattacacgctttttaatttaaatatagaagagagtgcccaagtgtatgttgagataaacccccactat
catttctttgtttacccctttaatctaaatatcgtttcgaaaagatccttcttcgacatttccagtggacataaaaccttcattacgattgag
ctatacaaaggagagtacgaaatacatttggcctttcccgggctggagagagaacaagtaaaagatattatattcgacttggttatac
ttattgtgtcttctagaagcgggaaaaacgaaaccaatcagaggaatacactaacatatataggtaactctcctggactatcccccca
aagtgatgtatgcaaggcgtacccatacaaaccccttttcaacaaagtcaatctggtggacattggggagaatgacgtatcagtga
agaaaaatattatttcctcaaaatataaaaatcaatattttcacatttcggaaaatttttcctgaaaaactacaaggaagaagttttcttta
ccatcccaaatggttactactttttgaaaattttttgtgtccctattgatgagtcaacagaagaaaatatttctgtagaggaaggggaag
aaaaaaatgaaattagagaggtgagaaagatctccgttggaaatttctcagacccttcaatgaactttgtaaactcattttgaatgtac
gaaatgtgtttaatattagggttgttcaggaacattcgggagaagccaacgaggatggagatgacaagaaagattcatcacagaac
agctccctttccgtgtcacccatatttgctaacgaagatgaagagttcatgttgaatctgtaccaagtggagaaaaattttaaagtggt
aatcagaacgaatagctatttctgtaattattttcagctagtggtaagtctctaccccctggatttttacaactctgtgcagagctatatgt
attcccaggtgaactctgtggaaaaatatatgaagaatatttttgacaccttgtctgtgaaggcaaaattatatgaagatataaaatttg
agcaggagaaatttccttcccatcagtacgtcagaatagaaacggatgttgtgaatttgaggagcgtccaaaaggaggattcctttt
ctcttcccgtggttgtacataaggggagttacttcaaagtcaatctggggtatgatttctcgctagctaacttccaaatgaagctagcg
aaaaatggtgtggctatttcaagtagcaccaagatggaagtcaacctgaaggataacagtcttaatacatttgaaaatattagtctata
cctggaagagggaaattacacactggagattcattcttatcacatcactgcaaatttgaacaacataaataagaacttcagcttcttctt
ttatttcgagctcgagatttttgaattttctgacgacaagaccggggacgcaattctgctggatgtgtttccgcacaactcgatggctat
cgacaggagctacccctttggcgttgatataatattctggggaagagtacacgcaaaagacatacatctgcaggatgatcaaaaga
cacacatagagccgaccaacagaacagcaataaagtatgcgaacattgaggttcagaaatttgttctatccccagaaataatgaac
aaaatggaaagaaattttatttttaaaatttcaccaaattgtaatatctatgttaatcaggaaaaagagaaaaggttaaagtttaccctttc
gactgaagataaaaatgcttccatcggagaaagcgtttatggagaggagaagggtgaagctattacaacgaatgaacggaatga
agaaaatggcgaggtccacccagagagtgtaaataattccttcctcttgaggtatgcggagaaggaaatcccgaccgacattcgc
ggaacaagctacgtggaaggaacttcccaaaaggacagtgtatacgacatcgaccgtgtcatacagaacttccgactgaacgaa
atgaaaagaagggaagaagaggattccaccatggggaatccctcgaaggaagaaacggaagcgtgtattccgctgaccatact
gacaatagaattttcctgcttcgagaaaggttactttctcatttttattttttttttgtgtttttgctttatgatgtgggtgttcctatttgtggtttt
aaaattatacaaaaactggaagtactacaggaattacgatgtcattacggagagtgatgaagtggtcaacctgttcgacgacgacc
acatatag
>Pmalariae|PmUG01_05036900.1: pep
(SEQ ID NO: 33)
atggaaagacgaaagaaaaggaaaaaaatttctctcctcattgatgtattactttactttattttgttctattccatgtttcctattgaaaaa
aaaaatgaaagaaaacttagtgaaaattacagtgaagcaggaaaagcttttaagaatattctccccttttatttgaatactcctcagtac
tgtaatctattatttgacatgaacgtatcaggtacttattcaataaggtggggtgttaattatatatacttcgaacacttgaatgatagttttt
ttgtaaattttaacttgtatagtatatatgtaaaagaggtagaattgtacgagtacagacataggagttttctattattaaagaaagaatc
agtagaaaatgaaataaaatttgtaacgtccattgagtatgataagagtatagatgcaaaggaacagaggactttttattttactgtgc
attcaaattatgtgcagagctttgacacgtacataaatgtaacaattgatgtaaacatagttggacatgttggtaggggaggagaaag
aggtagtggtcaggtgccatctgttgatgaaaagaaaaatgacgatataacaatggttgatgaatcaaaaaatgggataaaacaaa
atagacttcctctaaataacaatagttacattataataaaggaaaattattattacgcatctaatgaagactataagttcaacgtaagag
aagctgacaatttttatgtggactcgaaggggaacaaaataattgtaatatatagtgttttattttttgtcgattttaacaaacattgtaata
taagtttcttctctcttctaacacaagggatagaacagggaaaaaacttttctatggacttaaaaaaaataaaattaggtaaaaattatat
gcacaatatgacaacagagagtatattaaaatattcattttcccataaagagtgcaaagaaggttgcattaagagtagcaaaacaaa
aaatggcgtgtttttacacacattaatggagaataataacatatataagttcgacatcgtgtacaccatatacagcagtcacagcacct
acggcaatgacagcatttacggaagttacagcaattatgacatttcgaattctgatcgaggaagtggccatacgagcacctctagc
gatacgatacgccctaacgaggttgtccattttaaccttgaatactctattgtgaacaactcaaaaaattacacaattaatgactacatg
aaagaacaattttataaagaatgcgttcataatactttttcccccatcagattattcaagtaggggagaatgacgaacattttaccggtt
tagtggttactccgtcgggtaaaaataagtgctcacagaatggcggaaagtgcatcgacgatgataaaaagaaagcagataatag
taaccacgtagcagagaatgttgatttcataatttacggaagagtgatagaaataaacgatacattcattttaaattttgatccaattaat
ttattcgaagaagaacataaggttggtttacatgtgagtgaggagtctttgtttaatttcagccttatttctaagtcggaaagtatatatgt
aaatttgctaaaaaaagggtcatctcaaactgtttgtaatacttattatttgaacaatataaatgattacagtctgtttagttatataacaag
agacagtgctacttttttacataaacatttcaatgatacatttgaggtgaacaagaaggatgcgaatcataagtttaaaaatttttctttac
aaaatattttatacatcaactgtgtcgtaccaaaaggggaatacgaattgaagattattgttaatggttataatgtaatttgtgatccaaa
cgaaattaatttagtcctttatcccttgctcatgtatgaggaaaaacatacgtgtgacagtagcatgaatgcacttaccgatttgttttact
accacttgagagcggaaaataggagccaaagtggaagaggaagcgtagttggcggtttaggaggcactactgcatcgagagtc
gacgtgtctggtgaagccacccccgagagaacctcaaacagtgcatccagctacgcaaggcagaatgacgatctgacgtcaga
agtgcaaaaaacgtatagtaatatttacgagaaaaagtggatgatgaacaatcccctctttgagcaagacgattttgtaatattatacg
aaaagagcattgaagaacaagtagacgagttggttgttattgtaaacaactcattgtttgttgattttttttttagtaatagtagaatttata
ggggaggacagttattattatatatatcgaaactcaagaagcatagtgaaatatatgcttaagtataaaaacccttttactatatatttgtt
ggcatcaaatttacattacgagaatagaaaattatgtggttatttttttgtagatattgattttttaaaagggtctataacagtatcattggat
ggtacagaggattataaaaagatgattcaaaaaattaattatataccaagtcttataataggatatgactcctacgcctttagtaactttt
gctacgttccagattacaaaaaacatattcttacgatatgtgttcaagaaaattcaatcataaaaataaattgctttacacaaaattatatt
tatatttatttattagatcaaaataaaaataaaatatatgaagggtataaccatttatatattgaatcatttactaggggagaatatgaaat
cattttcgagtttaacgtgccaaatgataaagaaacagatgcttcttttttttatttacaaatatatatctaccaactgagtgctttagaaaa
atgtgtatttgatggccaaactgatagtagtagtggcagcagtagcgatggaaatggtaaagatgcccaattcaagaactacttatc
atcgaacgaaaatggagacaattctgaatatgaccttagctcctttagtgcaactaataacagcaaaatagaattaaagcaaaaggg
gaaaaacttttttttttttcaaaatgaatacgcatatcatctttttaaaagaaattttctttttctttttccaagtaaaagaacgacaaaaaattt
aattttacctcaagtgtcacatttaaataatattgcttttttgctaaaactggaacttgtttttcataaaaatttcttaccttataaaatgaccat
tcaagaaggcacagatgatttgttaatgcatcatagttatacgtataaaaataaaattttgctaactatgacaattttgaataatagtgta
aaagtttttaagttgtatatagacctatatgaagatataaatgaaaaattaaaaaatgattattgcccctatgcctatttgaatattatctat
accaatgagggaggaacatacaagcaagctgctttttcaggtttaaactttggtgaaatcagtatgcccttacttctaaatagcatatt
gctaaaaagcttcgcgtatgctaggggcatgggcaatagcgaacgtaacaaagacgaagatatgggggtggtcagacgcaacg
atagcggtgtcagaaatgatgactggagcaacaaacacacttcgacaatatcatttagaaactcaaacacagttactattggtagcc
aaagcgtggattataatctaaaaattattaataatagtacaacgcttttcttggctgaacatagtgttttttttaatatgtacatgtacaggg
aaaataacgatatatcattgaaaatttataaatatttaaatagtgataacaaattagaggaaggatacggagtaacatttgatgaaatg
aataaggagcatgttttatcccattcggaggaaattatgtctttctcaaataaacacatatatatatcaacctggttaaaaaaaggcttgt
atatatttaaatatgaacaaagtacatctccctttttcattaatttgagtgtaatatggaattatcaagatgatgataaaaacatggggaa
gaattatcttaaaagatataaatatggaggacagtcattaacacattataataatgggaagactgaagaaccccctgccgatgatgct
attagtgatactattttgaaaaatgaaatttatttttattttgataaagaactaaaatgtgatggaagaaaagatatattttatgaaagcaaa
aaaaaactacatcggttaactttttttttaaaagaggtcataaataacaatatttttgaaactcattctgatgatagtatgcagataactgt
aaagagggactcaaggaatacgttaatttatgagagatattgtatatgcatagggggtggtccgaaagtgaatgacatgcattccga
tggttcaaccagtgcggtaagtgctaatgaagtcgcccagggtgtagtaacgcaaccgtggaccggtagtggtagtagaagcag
cagtagtagcagtagcagtaacggcagcagtagtagcagtagcagtaacggcagcagtagcagcagcagtaataggtacaaaa
tttatcacattagtgtagaggagagtaccaaggtgtacatcgaaataaagccgcattttcatttttttttatttatccattccgcgtaagtgta
acatcagaggatttttattttaacatttttagtgaaaataggaatgttattaataccaatttgggaaaaggagagtacgaagtaattctgt
cttttccacaattaaaagaaaataaaaatacattacaaaatgctatatttgatcttattatattagtaattttgcctgaaaagggtgcaata
ataggggataaaattttagctagttcatatatgagtaataggttaagtgggaaattgttagaaaaggagttgtgtaatataagcacatat
aaaactttatttaataaagtagatttagtaaatataaatgaaaatgattcatatgtaagtaaatatattatttcatcaaaatttaaaaataact
atttccaaatttttgacaagtattatgttaataattttcaacaagaaatattttttacaattccaggtgagagttacattttgaaaattttctgc
atgcctatagatgagctgggaggtgaaaatatgtacaaagagggtacttacaataatacatataatggtaatagtaatagtacgtata
acaataatgcatataataataacataacttacgatagtagtgtccacaataatagcagtagtgaaacgatgagcaacatttacaatgt
ggcttctaactacgtaaatgctattttgaatgtacgtaacaattttagcgttcaagttgtactggctgatatggataataagtctaatgtta
agaataacacctcgaatgataatgagaaggataaatatgatgataataacagtagtaatcgtaataacagtagtaatagtaatgcctt
cgtttcaccgctacattcaaatgagaatgaagagtatatgctaattttatacaaagtggacaacaattttaaactggtaataaaaacaa
ataatcatgaatgtaattacttcaagataattttaagtcttcatcccctcagtttttataacagtgaacattatttctcatatgctaattaccta
gataaatatttgaaaaatttattcaataccttgtctgttaagaccaagttgtatgaaggtataaactatacacatgaaaagtcgtccatgt
acagcctcacacgtttaacttcacctattgttaatttgaaaagtgtcaatatgaaggaccttttttcctttcccttgtccattaataaggcta
gctatttcaagataaatattggctacaacttttcgctagtcagttttgaaataaagttaatgaagaacagtacgactatttcatgtagtaat
aaggtggaagtaaacttacaagatgacactgtgaacatatttgaaaatattagtgtatatttagaaaaaggagattatatattacagatc
ttttcctttgattatatgggaaattctgttaatataaataaaaattttagtttccattttattttgagcttgaaatttttgagttttttcaagaaac
taagaagagcggccctatcctcttggatattttcccgcacaattcagtccctgttgacgcagttttttggggagaagtaaaaggaaaa
gaaatgttcttggaggtgcataacaagaaatatgcagatttaagaaataggaaaataattaaatatggaaatattgaaatacataaatc
ttttttattacctgaagatatgatgaacatggaggaaagttttattttaaaatttaccccaagtgcgaatatatatgtgagtgaacaaaat
gaaaaaaaattaaactttatcttttcaacttatagtacaaatattgaacagaacaacaacagttacaaagtggtgcaaggggaagaca
agattgtgatgataggatcagggcaaagaaatggacaagtacatgaagaaaatgtgaacaagtctttcttgtttgaatatgatcaaa
gggacattactgaagcaggagaaagtgttaatgattctcagacgaatattcacgtagataaattttttgaccttgacaatgttattaaaa
attaccgacttaacgaaaaaaaaaaaaaaagagaagtatcttacacgtcgctcgagaatagtaataagcagaatgactcgtgtatc
cctctatacttatttacatataaaatttactgtttcgaaaggagtaattatcttattttcattttttcttttttccttttttccgtgtcttgtgcttttat
atgtttactaataaaattgtacaataattggaagtattacaacaattatgatattgttgttgaaagtgaagaagtaataaatctttttgatga
cgatgacatataa
>Povale|PocGH01_05030600.1: pep
(SEQ ID NO: 34)
atgtttgtttgtatcatcttctgcgtcttgtttctcccatttgggagaggaatacccagaaatcttggggatgacaacataaaaagaaga
agccagtgcgagaacttggtaccattttattttaacgccccacaatattggaacatgtctggggacattaacatttcagattattattcat
tgagatgggatgttaactacatatattttgaaaaggttaatcataaaacatttgtaaatttcaacctctatagtatttatgtgaagaatgtc
gaactttatgaacatagagatagaaacattatgatactaagacgtgaagttatggaaggggaagtaaaatttgtccattttatagatca
ggatgtaagtttacatacattggaaaagagaattttttattttattatacgtttgaagaaaccccttcatgatgacacatgtgtaaaagta
gggttagatgtaaatataggaaaggctcctattacaagtttcgaagatggtatggttgtccaccaggagaaaaaaagcaaaagacc
attgcaccataaaagttatattataattagggagaactattattatgcctctaatgacgatttttactttacaccagaggatagtgaaaat
gatacatacattgatgataatggtgccaaatttgtaattgtatacagtgtactattatttcctcaatttgataaaaaatgtaatataagttttt
tctcattttaaatggagagataacaaaaaagaaaaatttctcaatgcaattgagaaaaataatgttaaacaataattattttcataatatg
aatatagatagtgtattaaaacatacatataataatatgacatgttcagaaaaatgtgtaaaaagtatatttacgaaaaatggattattaa
tacataatttattggaaaataattacatgtacaaatttgaaatagtttacaagttgacagaaaatttaaataaagatcaagggaattatga
aaataatacagtaaatggttggacatataatataaatagtaagacttatttcaatatggaattttccgttgtaatgagtttaaaagatcaat
cgtttatcgattatgtaaaagaatacttcaacagtgaatgtatacataattctttttttgtgaaaaaaattgtacagattggaatatataaag
gtagaaatgacaatgttatttcttccttttcaatgaaaaaatgtcaagaaaaagacaaacatagatgcaacttcacagagttaaacaat
gaatatgtaatatatggaagaattatacaactgaatgatagttatgtcttaaacttcgacccaattaacttattcagagaagaagaaaaa
gtgcatatacacattagcgaagactctcttttcaattttaccctaactaccacgttggaaaacatttatgtaaatttgttaaaggggaact
ctacccaaaatgtatgcaacacttactatctacaccatataaatgagtacaatatttttagctttcaatctaaagacaaagccccttttat
gcactatcattttaacacaacatttggagataaaaaaaagtatagtaaggatcacagattaaagaaattttccttacagaatgttgtata
cataaactgtgttttgcccaagggggagtatgatctacggattgtcatgaatgattatatctccatttgccaaccgaacgatgtacagt
tgactattttccctctccatatctacgaggagcggcacacctgtggtagagaaatgcacgtgctcacggacctcatctactaccacct
caggggggaagggcgaagaggtgggagtagcggtgaggggaataggccgatcaaaaatgaacttaccgaaccatcgtccga
acaatcaaggacaatctcccacaacgtgtacgaaactatgtgggagctgaataacccgcttttcgaaaacttcgatttcgtcgtcctt
cacgaaaatatcatcgatgaggaggtggatgagatcgtcgtgacagttaatacctccgcatttgtagacattttcctcgtgataacag
aatcagtgggggaagagagctactattatgtgtataagaactcgagaggcgttgcaaagtatgggctgaagtttggggggccgttt
cacatgtacctactggctcccagtttgcactacgaggataggaagatttgcggctttttctttgtcgatgttgaccttgttttgttgggta
agaaggcaaaggggaagacaccacgcagagaagatcacactgtgggggaggagaccctttcctcggtggacgaaccccaag
gaaggaacgtagttcagaggactattcacctgccagagttgatcataggatatgacacttatgccttttcaaacttctgttatgtcccat
cttacaaaaaacacgttctcacaatttacgtacaggaaaattctattgtaaaaataaattgctttacgcaaaaccctatttacatctatttg
ttaaacagtgagcagaagaaaatacatgatgggtataaccatctatatgtagagtctttcaccaaaggggaatatcacgttgtttttga
gtttaatttgcagagtgagaaagaagcaagttcctttttctacttacaaatatatatttaccactttaatttgttggaaaggtgcgcttttga
taatccctctgaggctttgtcaagctcggaggagcttccgcttagcagtgcgctgcgtattgatccgttgcgaagtgacccttcacaa
agtaatgatgtatacgattttagcatttacaacgagatgagcacggcaaaggatggaatcaaagtggtagatcccacttcttttttattc
aacgatgaacattcctatttcttttttaaaaatatgtttattgtcatattttccaacaaaataatatcaaaaaaggttatattaccccgagtag
caactcacttggaagcctctccatttatgttaaaaattgaattgatttttcacaataactttttacccctcaaattaagagttcaagatatta
caaacagtcttctatatcatagtagttacacatacaaaaataaaataatattaacacttaccctttcgaatggtagagtcagagttcttaa
tctccttattgacatgtatgaagatgtaagcgatagtctcaagagtaactactgcccctacgcttatctacactttgtgtacacaaacga
ggtggaggcataccggaagattggagcaagtgggttatcccatggcgcgatagatttgccactgctcttgaacagtctcctcctga
gggctgtgtggggagtcgacgacaaaggggaagaaataagaataaatacctcctccgataatgtcgatagtgcttcaacccctcc
gattgagggaggtgtaaccttcagaagtccagtctttcccgtcatggtaaaccagagtgtgaactataccatcgaaatggttaataac
gacacgatgcttttcatcgcggataatgacgtttttattaacatttacttgagtaaagaaaatggaattgttgtattgaaaatgtatcattg
cttgagcacaggtatactagagaagggtagtggaatacagtatgacgaggtgcataagaactttctttccatttcgaaagaaattgc
atccttatctggtaaacatgtttacctttccacctacttggaaagaggtttgtacgtgttaaagttcgaacgagattcttctcccattcatg
ttaacgtgagtgttatatggaactctaatagagaggatccaaccataataggttacgtcaaacgacaagaacatgcaaacaaactg
gtaaattatgccagtcgagaagtcttacctgtgatgagtatgcacagaatgaacgtgtcagcacaaggcacggcaagcgctacag
atatatccccagcgggagatcccattttgcgaaaggaaacacatttatttttcggaaaggaactacattgctctggcgtgaagcaagt
atttcgtgaaagtaaaaaagtggcaagtgtacctttttttttcgatgaaattgtgaagaccaaattgtttgcagtttactctggacgtaca
acacacattttggtgaagaagaatgcagataatatgttaatttacgagaggttttatgtctgtctggggagggaaaccaatggctact
atgaacagtacagcggggaggcctctcgcgatgagagtgatcaacacaacgagggaaaatacccccttttcggcatcactgtgg
aggatgtcgcccagatatacgtggagataaagccacactaccatttttttatatacccttttgaactaaatgttacatcaagcagttcct
atttcagccattcgagtggagggaaaaactttgtgagtacgaaccttggaaagggagagtacgagattgatttacgttttcccgagt
ggacaaataatgatataaaaagtgtcatgttcgattttgtcatattattgacatttccaatttgggaagcatatgatacagaagtgctttc
caaaaagggtacaaaaatgcttcttagggatgatacatgtaatgtaagtatgtacagaagtttgtttaacaaagtcgatttaattaacac
aacagaaaatgatatcattgtaagtaaaaatataatttcccccaaatttaagaataagtacttccatgtatttgataaatatttcattaggg
attaccaacaggagatattttttacaataccaagtggtggttatatattaaaaatcttcgctgtggtggattcagagattggagaagga
aatgatcagggagaaggttttccgggagagagtcaccctcagggtacgggacgtgtggtagattttccagcaaattatgtgaactc
ctttttaaatgtgcgcaacgtttttaacattcgaattgtcgaaagtgatttgaatgggttaggaggaaagaataacaatgttgaagataa
gaaagaggaaatttcttatgtggcaccagtatactcaaatgaaagcgaagattacatactagttgtgtataaggtttataccaattttaa
aatgataattaaaactaataactatgattgtaattatttccaacttattttaaatctccattcagtaaactcttacagtgatgtacatcactttt
ctcatgcatataattttaacagttacttaaaaaacatttttgataccttaactgttaaggcaaaactgcatgatggcataaattttgagcag
aagaaattgtcaacatataatcatgtttccataaccacttctattgtttacctgaagaatatcaatccgctggaattgttctcctacccact
gtctatcaaaaagggaagctacttcaagataagcgtagggtataacttcacccaagcgcatttcgaagcaaaactggggaagaat
ggtgaacctatttgcagcggtagtaaggaaaaagttaacttgaaagatgataccattaacgtttttgagagtattagcctatttttagac
gaaggggaatattcattgcaaattttgtcacatgatttaatagaagactctgttaatataagtaaaaaatttagttttcctttctaccttgag
cttgacatattcgagtttatgcatgagaagattgaaaatccaatccttctggacatttttcctcacaactctgttcccctggacaggaatt
atcctttctttgttgaccttatattttggggagaattgagaggaagagcagtttatgcggaggatggaaaacagaaaagggtgcaact
aagaagtggaagaatgcatacccatggaaatgtagagatacataagttaattttacccccagaagagatggatcacatggagaata
atttcgttatattgtttgatgcaaaagaaagtaatcaaatgaatagagaaaaggagaaacggttgtatttctccttttccactatttccac
aaatgtgttgagtaagaatagtagttatgaaccgataagggaaaaacaaaatgtaatgtctgaatcgaaggaagggaatcgacaaa
tacatccggaaagtgttaacacctcgtttttgctggaatatgggcagagggatgctaagcgtgacgactccatacaggagaactcc
aacaaagacaattttttcgacctggacacggttattaggaactaccggctgagccagaagaaggataaggaatatatgcctgtttca
ggcgaggctccccctaaaggggaaagcagcacttgcatcccactgtctatatttgcccttgagatttactgttttgaaaaaaattatttt
ctccttttccttttcgtggtgttcattttatgcatatcgtgtgccttcctattcctactgataaagttgtggaaaaattggacatactacagc
agttatgacgtcattacggaaaacgaagaggaggtaacactatcgacgatgacatatag
>Pfalciparum|PF3D7_0419400.1: pep
(SEQ ID NO: 35)
atgagaaaaaagaagaaaacgtgtttggttcttttaattttaagtttcttatgttttttttattcatctttatcttatgatgaagattacgatgag
gaatacaattatgaagataagaacagtgattatgaatgtaataatttaattcctttgtttttaaataatttagaatatagtaatatatctcag
gaaatatctttgtctaagactttttctttaaaatatcctgtgcattatatgtattttaaatattcatctgatcaaagcatttttgtaaattataata
tatacaattctttatcttttggatatgtaaaaaaaatagaattatatgaatatgaaattattaagaatgaaagtgtgttattattacaagcgg
aaaaagattctgataaaaatattaaaattttgtataccataacaaaaatagataataggaaaaactatgaaggggagaaggatcaaa
ggttattttattttattatatatacgacaatagaaaatgataatgatataataaagagttgtataaatgtaaatatgagcatacatataaga
attataaaggatgacacacaaacaaataatcataacaataataatattattattaataataataataattattattataattatgtgcataat
aattatgggatgaggggtatcatgccaaatcatttatataatcatcttctacctttacataataaaagttatattataataaaagataattat
tattattgtactaacgaagattattattataacatgaataatatagaagattattattatgatgaaaagagtggcgacaaagttgtggttat
atattctgttttattatttatagaatttaaaaaagaggcgaatataaaatttacatcattcttaaaacatgatataaataagtacaacaatttt
gttatacatataaaaaaaatagatatccataataattatttaaacaatatggatatggaacatatcttgaaggttggaaataatgaggaa
tatataaatggtaataagaagaagaaaagtatggaatgtgaagaagattgtgtagaaagttacataacttcacaaggaatatatatag
atacaatattaaataataatcatttgtatagattagatatattatataagatgggaaaaaatgaaataaataataagaccactgaaattatt
aaaaaggaaaacaacaataataacaataacaataataacaataacaataataataacaataataataacaagaacaataacaataat
aataacaataataataacaagaacaataataataataattattatgattatgataatgacaaaatgggagatataaaaaatatgaaaga
caatggaaaagatcctttcttatcatcattattattttttaattttgaattatccatatataacattacagataattatattcctatgaattatatt
aataaacgattatacaccgaatgttcaaataattctcatgctccaaataagattgttcaaactaaggaagagaatatgttaaatatatttt
atacaaatgaatgctcaaagaatatagagaaatgtagtttatatgatgttaatgaatatataatatataataataaagtcattgatatacat
gataattttcttttaaactttgatataataaattcttttaaaaatgaacaaattatagatatatatataaatgaagattctatttttaatttttctat
gctcattaaatctgatcatatttttgttaacttattaaaaaataactcttcagaaaaaatatgtaatacatattatatgaataatattaatgatt
ataatatatatgattataaaaaaaataacaaatctgatttttttcataaacattataatgatagttttttatataatcaaaatatgaattataata
acacctttgaatatcaaaattattatcaacatgatcatcatttaaaaaatttccctcttcaaaatattatttatattaattgtatattaaagaag
ggatattatgatttaaaaattattcttaatggttataataatatatgtgaaagtaatgatttcaatttattaatatatcccatgtctctatataaa
aatcaacaccaatgtgatgataaaatgaaccaagtaactaatttatttaataatattttaaaacgtaaataccaacataatattagtcatg
taccaaatcaaatccaaacctttgatcaaatttatcaaaataaatggattttaaataatcatatatttgaatattttgattttgttataatatat
gaaaaagaattactaccacaacaatataaagaaattatggttactataaataattctccaggtcttgatctattctttattcttgtagaaaa
tgtacaaggtcaaaaatactatcatatatataaagatacacgaaaaaaaaataaatacatattaaaggataaacacccatgtactttgt
atgtacttgtatcaacattattatatactacccaagatgtttgtggattcttttttgtcgatatagaatatgtagataatatgaagttattaaat
caaaacgatcatccaaatgatataatgaatggaacatatcaacataatatgatacaaaaaattaattatatacctactataattatagga
tatgattcgtatagttatagtcatttttgttatgtaccatattacaaaaaacataaacttactatattattaaaaccaaaaacgatagtcaaa
ataaattgcttcacacataattatatatatatttatttattagataaatcaaataataacaacaacaacaaaaataataatgataataataa
gaagaagttatatgaaggttataatcatctatatatcgaatcttttgttgaaggggaatatgaaattatatttgaatttaatacgcaaaatg
ataaaaatatgaattcatttttttatttacaaatatatatatataatttaaacacattagataaatgccttttctttaataataatatgaattatat
aaaagaaagctccataaataaatttataaatacaaataactatgatacatttgatcttagtaaatatgaatataatgatattattataaata
aaaatgtacccagagtaattaaaaaagaaaataatatgttcttcatttatcaaacaattaatttttattatctctataaatcattctttctttata
ttctaccaacacaacataaagatggaaatataacaaacgaacaagataagaatagaattaaaaaaaaaattatattaccaaaaatta
attatttatcaaatcagaatttcatattaaaaattgaattgtcttttcataaaaatatattcccatacaaaatgtatatcgatgaaaattatgat
gatattatcatgattagtacaaacacgtatagaaataaaaatattatgttattaaaaatattaaataataatacaaattatataaatatatat
atagatacgtatgaagatattaatgatgaaataagaaaaaattattgttcatatgcatatttcaatattacatatacaaatcaaacagagg
aagaaactgtggatagacaacagataaatttatataataaaatcactttacctttattattaaataatattttatctaaggactatgttccaa
tgattaataaaaatatagtagaaaataagaatgaccaaacatataaagctgatcatttatattatgaggatgataatattggtgtaataa
atagtgtatccaataattatttctctggtgagaacaaaaatatgggggatatcaaaaataagatgaataatgagtatggtagtgttcata
cagaacagatggtacattttcaaaataatgataaccttaacaataataataatatcatatttggtaatgtttatccttatcttgaaaatcatc
ttagaggtatcatatctctcaaaaatagcaactctttaatattaaatcaaagtgttaattataattttgaagtagttaacaataattatacact
tttgataataccaaatgatgcttttctaaacatgtacataagaaataatgatgatgatgatgatgatgataatgataaaaataataataat
aataataataatagtaataataataatagtaatagtagtaaaaagaacattactttgaatgtgtaccgaattttagatgtacctaaactca
caaatgaaggtatttcctttaatgaagtcaagaaacaaattctgtccttatctcacccgttcttaacgtttaccgataagtatataaatgta
tatagatatttagaaagaggattatatatatttaaatttgatgacgattctaataaggaaaattcaagttatacctttagaaatagtagcaa
tatatataagaacaatataaataattataattatcattctagtgatagtattaagccattatccttttttatgcattttaatatcatgcctattta
cacgaatgatatagaatacaaggataatatcaaaaataataattataatgataaaacgttcaattatcaagggcatgataaaaatatgt
acccttctgtcattcataactatatatttaagaatgaattattatattatttcaataaagaatacagctgtgataaagagaatatgttatttttt
gaacataaaaagatagatgatgtatctaattttttagaacatacttggaataatataccatttgaaatttatataaatgacaaactagaca
ttacattaaatcgagataactacaacacattaatattaaaaagattctttatatgttttgaaaaagaaaaaaatgtaaacaataatgacaa
tattaataatagtaataattcctatgtaaataatactgataatattaatgaatcacaagatgactcttatataaacatttttaatgatacaatt
aaaaaagaatatctcctttttaatatcgtattaaatgtaaaaggtaatatatatatagaaatacaaccacattatcattattttatatatccat
ttattttaaccataaaatcgaaaaataatcccacacaacttaataaatctcatgataaattatttttaactaccgatcttggcgtaggtgaa
tatgaaatctatataacgtttctttcgaataatcattataaaaatgtaaaaatgaaaaaggtcatgtttgatttatttatttttattgatatttta
aaaaataaaaatattcaacttggaaaaaataatttatacttaccatatggaaaccatcttgatgacactatagattttaaaatatatgata
atacatgtaaaacggataattataaaagattatttcatgaagtaaaattattagaaaataataatagttccaatgttcatatgaatgaatc
atatataaaaaacaaaaataatattatcagtacaaatatgaatacgaattattttcatatttatgatacatattatatgaaagtacgtgagc
acgaaatttcttttgagataccaaaagaggcgtatattcttaaaattttttgtatacacatagatgaacaaaattatgatgaagacataac
aagtggagatttttacaataatacaaacgcttataataataataataattatggaaataataataattattataaaattgaacaaactgata
atttatctcatttttcttttaattatttaaacacctattttgatatccaaaatttattccatattaatgtaataacaaatgaagacagtaaatttttt
tctgatgaacatacaaatgatcaagatttatatgttaaaccgtttttttccaatgaagaaaatgaacatatattaaactggtataaaatag
ataagaattttaaattagttataaaaaaaaataattatgattgtacatatttcaagctaattataagtttatatcctatggattattttaatagt
attgattatttaaaatataataattatcttaataaatattttacaaatatatttgacaccttatcacttaaggtcaaccaatatcaagacatat
cctttgagcaaattaaaaataagaattatttatatgactatttaaaaacaaatgtgatttttttaaaaagtttgaattctagggatcttttttctt
atccgcttacgattaaaatgccgagctatgtaaaaatcaattttgggtacaacttcactttaaccagctttgaaatcaaactcttgaaaa
acaatatgataatcgctacaagcaacaaagtacaagccaatataaataataacaatatcaacatatttgaaaatgtgagcatttatttg
gaaaagggaaattatgttgtccaaatttattcttatgatttaattgaaaagtcttctaatataataaacaaactcagttttcctttttatgcgg
aaattgaaattgtgcagtttgtaaacgaccaaattgacgaacctatattgttggatgtgttccctcataattcggtcattgtgaatagaaa
ctaccccttctttgttgatttaattttttggggaagagcaaatgaaaacatatccgtgttggatacaaaagaaaataatatcaaattaaat
aataccaagttaataaaatatggaaatgttgagatacacaaatatgttttgtcatctgaacaaatgaggtctctggaaaataattttactt
taaagcttcagtccaatgttcatataagtgcatggatggagaaaagaatgaattttagctttacaaatgagggttggaattacgatgg
acaaacagaacatgggaaattaatgaaaaatggaataaaaacaaataatattgttgatgaatcttctaaaatatccaagggagaaaa
attaaatgagactgtgttattggaatatggcaagagggaaataaaaaatgagagtgagggtgaaagtaagagtgagaataagattg
aaggtaatagtaataatgagagtgatggtgagagtgagaaaaggagtttcttacagaacggtgtaatagaaaaaagcaacctttttg
atctgaatgaagttattaaaaattttcgatataacaagaaaaagaaaaaagaagaagaagatttccttttgaacggatcatatatgaatt
caaatgataaatgtttctctttgtctatattcagttttgagatatattgttttgaaaaattttatttttttatttttatctttatcttaaccgtattatg
gatatcttgtgcctttattttttttaattataaaaatatataaaaactggaaaggttatagaaattatgacatcgttggtgaaatggatgaag
tgataggtctttttcatggagatgatttataa
>Pyoelii|PY17X_0721500.1: pep
(SEQ ID NO: 36)
atgaataagagaaaagggaaagtgtggcttttcctttttataatctgtttgatagttattaattttaagatttcttatgaacaaaataatgaa
aggaatcttagtgaaataacaaaaaatgaaatcgaatatgaaagatgtcaaaatttagtacctttttatttaaataacccagaatattata
ctatgttaaatgaaattaatgtatcaaatgaatattccataaaatttgatataacatatatatattttgaatatataaattgtaatttttatataa
atctaaaattatatagtaaagatgttaataaagttgaattatatgaatatgtagataataaaggtattttgatattttctgaaaatagtaaag
acaatttaataaaatatttatttcatgtgcaagatgataagaatagcaacaaaaatgaaaataaaaatgaaaataaaaatgaaaataag
cgcgttttttattttattatatattcaaataaaatcgaaacatctaatgaatgtgttaatataagattagatataggaatagggccggttgtt
ttggaaaagggggatacaaaaatagagagtacaaaaaagggggatatacaaaaagaggggagacaaaatgagggtagacaaa
aagaggatagacagatagaacatcataatagctacataataattagagataattatagatatgcaacagatggttattttaatttttcttt
aaaaaataataaagatttttatatttatgaaaatggtaaaaaatatggaatcatatataatgttataattattctcgaatttaatgaagaaaa
taatataaattttttttcacttttatcacaaggaaaaaataaatggaataatttttctatgcaattaaaaaaaataaaattagataaaaattat
ttacataatatgaatatagaatatataataaaacatgcgaatgctgatatgacttgtaatgatatgtgtataaaaagtgatgaatcgaaa
aatgggataatatttttaaatgctttattaagtaataattatatttataattttcaaataagatatgctgaaaatgaaaatgaaaataattaca
taaataatgatattaactatgataatgaaaataaaagtaataaatatttgggtagaaatagtgtagagagtaaaaaagaaatgtttaatc
aagataatataatttattttaattttgaatataatatagttaacaatgttgataaatatacaatgatagactatttaaaaggccattttaatcat
gaaaatattcagaatacatttttttttgataaaataattcaaataaaagataataatataaataaaaataatatattgccaataattggattt
aataaagaatgtgtaggagaaaataaaaaagaatgtaataaatatgagataaataataattatataatttatggaaatactattgaaata
gatgataattttattttaaattttgatcctaataatttatttaaagaagaaacaaaagtgaacataacaatcgaagaagaatctttttttatgt
ttacactagaatcaaaatcagaaaatatttttgtaaatttattaaaaaaaaattcaacagaaaatgtttgtaatacatattatttaaagcata
taaaagattataatttatttagctatttaactaaaaatgaaaaaaatattttgaaatatgataaaactaattttagtagacatcatataaaaa
atctatcaaagaaaattataatatatataaaatgtattcttttggaaggagaatatgaccttaagataaattttgaagaatataataaaaa
atttgaatcaaataatattaatttaataattcagccattacatatatatgaaaaggtacattcatgtgatcgtaaaatgaatataataacag
atatgttttattataatcttcgaactgaaaatgtgaataaaaaaaatgataataatgttatatcttataaaaatatatatgaaaatatgtggg
aattaaataattcattatttgatgattttgattttataatattatatgaaaatgagatatttgaaaatgttgataaaataatagttactataaata
attcaatattctctgatatatttattataatttttgaaacagtaaaagatgaaacacattattatatatacaaaaattctaaatcaacaattga
atatgaaataaaaaataaattaaataataaaataagtatatatgtattaacaccaagtttacattatagtggtagaaaaatatgtggtttttt
ttttgttgatatagattttatagaaaataaaaaggaatctattggagaaataacattatttaataatatgtataaatcaaatagaagttatatt
ggaagtattaatcatattccatatttaattataggatatgatacatattcatttagtaatttctgttacataccagataataaggaacatgttc
ttataatttttattgaaaaggaatctataataaaaataaattgttttatggaaaattatgtatatatagaactatatgaaagaaaacatattg
gtggtgaaataaaaaaaattaaattatttgaagagtatcaacaagtatatattgaatcatttactaagggagagtatgagattgtgtttaa
atttaatgtacaaaatgatagagaaatgagcaattttttttatttacaaatatatatatatcaaataaatttattggataaatgtgtttttcata
atgaagatgattttggcgaaatagcggagggttcgagttctgtatatgatctaaatgaagttgatagaaaaataaaaagtaaaaatgg
taaaaaaaataaagaaattggatattttaatgataataatggctttatatttgaaaatgaatcttcttactatttgtttaaacgatatttactat
ttctttttccaaaaaaaattgatgaaaaaattgaaaaaaaaataatattaccagaaactaataattctgggtttgtattaaaagctgaatta
gtgatcataaaaattttttaccttataaattatctttagaagaagatggggaaaatatattagcccatgaaagtaatatatataaaaataa
aattataataacttttaatattttaaataaaaatgtaaaatatgtaaaattatatatatatctgtatgataatgtacatgaaaaattaattaaag
attattgcccatatgcttatttaaatattgtttatacacatgagttggaaaaatatagagaacatgaaaacgatgaatatcaatatgacac
tatgcatttgcccctttttttgaataacatattgttgaaaaggtacgaatattctgatgatgtggaagaggaagatgtagaaattggtaa
agtagaaacggaaagtgggaatgataaaaatgtaagagtagttccttttgacaaaaaaaatggaatgtgtattgggaaccaaaaga
tagaatataaatttatcacaagtaataataatatgatgttttttttagcagaaagagatgcttttttaaatatatacatttataaagaaggaa
aagaagatattatgttaaatatttataaatataaaaatatatcaaaatttgaaaaaaatgaaatattaccatatgatgaaataaataaaga
tatacaatcttgttgtgaagaaatattttcacattctaatgttaatgatataaatatatcaacttattttgaaaaagggttatatatatttaaatt
ttctgaaaaattgccatatataaatatgatatttagtgtaatatgggtggaaataccaaatgttaatgatataattatgggttcaaatataa
ataataaatatgaaattaatagatatattgaagaaaatgaatctaaattttattttagtaaagaattaaagtgtggtgataaaaagggaat
attttatgatgaaaacaaattgaaagatttagaaaaatttgttatagaaaatatgtttgaaaaaaatttatatacaatttattttggaaataa
aaaaaaagaaataacaatttcaaatgattcaaaaaatatagtaatgtatgaaagaatacatatatgttttggaaatgaaatatttaatcat
caagttattaataatacaaattatattaaagaaaatgaagagtctcatattattttgactttaaatgttgaaaaagaatgtcaagtatatgt
agaagtaaaacctcatttttattttttttatatatccatttaaaataaatatattatcaaaaaattcatattttaaagtatcaaatgaatataaaa
aatatgtgtatgcaaatattgaacaaggggaatataatatagagatttcttttaaaatggaaaattatataaataataaaataaagaaaa
ttgatggtgttatatttgattttataatatttatatataatacatcaaatgaaaataaattaaaaaatattgatttttcagatgaagaaaaaaa
acagatggtatcattaaaaaatgagacatctaatatgaataaattatatactcgaaaaatttataaaaatattttcaaaaaagttaatctta
aaaatttaaatgaaaatggaattgtagtaaatgaaaaaataactacacataaatcgaaatatagctattttttttgttatgataaatatatta
ttaaaaatcatcaggaaatattttttacattagtagataatgtggattatattttgaaagtttatttagcaccaatctatgaactaagtggaa
ataataattcgaaaaatataaataatgattttgaacattttgaaaattttgaaaattttgaaaatttcgaaaatagtgacaataatagcgaa
tcttcatctctttataatttttctacaaattatttaaactatacattaaacgttcgaaatgtttttaatattgaggtggtaaatgagagtatgaa
taatgattatattcctgaaaaggtaattaaagagaaaagattaaaaaataaaataattgaaaaaaaaacaccggattcaaatatattat
atacaaacgaggatgatgaatatatattaaatgtatatgaggttaataaaaatttcaagttggttattaaaaactacaataatatagatag
taatattgaattagttttaagtttagttcctttaaaatattataaaatgaatatgaacacaaaatatgccaatgaatatattaacagctatttt
aaaagtatttttaatacgatatctgttaaaacaaatttatatagtggtataatttttgagaatagagaacattctactcacatatatacacgt
gtagaaacttcggtggtgtatttaaaaaatattatcatggaagaaacattttctttccctttgcatgttaagaagggtagctacatcaagc
taaacattggatatgacttttcaagggttaactttgatgtcaaaattatgagaaataataagaaggtctcaactagtaataaaattaaag
gaaatatggataatggaaaaataaatatttttgaaaacattagtttgtttttagaagaaggagagtatacatttcaaatatggttttacaac
ttaactgacaattatattcatataaataagaatatgggttttcctttttatttttcacttgaaatttttgagtttgctgaaaataaaaataatact
tcagtgttattagatgtgtatcctcaccgttcagtatatgtagacaaagcttacccctttaatattgatcttattttttggtccgaggaaaa
gagagagaaacatggctttatggaagatgaaatgaaaaaaatagtatatttaagagttggtgaaataataaaatcaggaaaaataga
aatccaaaaaagttatttattaccagaagatatgagcaatcttaaaaataacatgactttaaaatttgatttaaaagataatgttgatatg
ataagtgaaaataacaatgaaaataacaatggaaataacaatgaaaataacaataaaaataatagatatcttttcacttttttaaataac
gatgtaaaagaaaatgaagtagctatagaaacgaataatacaaatcttaataatcaagaaaaacttaaacaaattcataatgaaagtg
taaataaatcgattttacttgaatataagaaagaagattctgaaaagatagaagaaaaggatagttacatttctcataatgtgaatcaa
gacatgttttatgatatagataaagctatcaaaaattatcgttctagagaaaacaacgaatcgaaagaaataacatcttctatttttaata
aaccattctcagacccagatgataaatcctgtatatacatacctatatttttaatagaaatatactgtttcgaaaaaaataatttgttttattt
tatattcattttgtttattttttttatgactagtgcatttatatttttattaataaaattttacaaaaattggaaatattacagaaattatgaaagttt
caaagaatatgatgaaacaattagtctttttgatgatgatgatatataa
PY17X 070600 Protein
>PvivaxP01|PVP01_0528900.1: pep
(SEQ ID NO: 37)
MNTSNSDLKKENLWDGHDVSLHKGDTSRNTYSNNSGSNGKKTKKKKNEKSNFL
ANEYKDTFHVEGLPTRNNNVNIYYGNEEDVNHSAINDDDDIFGSGGNTFLNKNLT
TVNLSDMKRGGGPPHGTSNSNGIANFDGSFGVAAYSGYNLSNNTGSPLNLSIAQG
VANQMGSAANESGSRGYHSNKSGMYHQRGPLEQEFNAGDVHETPLGNVPSGKM
NIINLSDNENDDDWGVETASRSSLEGGGNNSFDLFPFFKSLNRIRTKLLCYYDVDS
DVIIYRCMCALLPYLKVDKSYDSMDSLDDIEKNAGEASAGRSRRDQRNGRSGVK
GGSKSGGNNYGSNGGSNDGVASADENEADDENANIRKISNVNDAFDYYDNKLSI
EGNPDLYGFVWVNIFISFTIFFLFNWKNIFFGDSPDGATSSSEETNNQAYVTQNKL
NILYTTLIFLYLFNTQTPLLIYVTNFFVTKRVFPIRLSFLISLMSYNNVILFPLILLYKF
TLINTSLSFVLFLCSALRFFIFAYYMMSSLFYIHKYTIRTFRNNFSDNVIYMYYGIFC
LSYLLLYLQLRNYIFSYL
>Pknowlesi|PKNH_0512200.1: pep
(SEQ ID NO: 38)
MNKTNRDLEEENLWDDHDVNLHKGNTSRNTYSNNSGSNGKKTKKNGRSNFMA
NEYKDTFHLKDLPRSNNKVNIYYGNEEDIIDCAINEDDDIFGSGGNTSLNKNLTTV
NLSEMKRGGGPPQGISNSNGITNSDGSVSVAAYYGYNLNNNTGNPPNFSTGQGVT
NQIIIDANDSGNRRYHSNNKDMYHQRAPLEQKFNAGDVHDMNFRNAPSGKMNIF
NLSDNENNDDWGVETASRSSPEGGENNSFDLFPFFKGLNQIRTKLLCYYDVDSDV
IIYRCMCALLPYLNVDKSYDSMDSLDDLEKHACQPRSRRSNKDKRNVKSGDKNY
GSSHDVASADENEVDDENAHIRKISNVNDAFDYYDNKLSIERNPDLYGFVWVNIF
ISFTIFFLFNWKNIFFGDSSGIDFTPEDDITWSSEEINNQAYITQNKLNILYTTLIFLYL
FNTFTPLSIYMTNFIVTKRTFPIRLSFLISLMSYNNIILFPLILFYKFTLIKTSLSFILFFC
SSFRFFIFAHYMLSSLFYIHKYTIRTFRNNFSDNIIYAYYGIFSLSYLLLYLLLRNYIF
SYL
>Pmalariae|PmUG01_05037000.1: pep
(SEQ ID NO: 39)
MANEEYNRKISWDEDGFIGINEDNLSSIRHNNSVNEKSSLLITNECKDSFLIDDFSR
RPKNMMSVYYENDDEDNIFGGGENSSLNKNLKTLNLSDIKKKGVKSDSNNLNYG
SNNSNGRSSSNNNTRSITNGGSSSSPFQGLMSNVNDALNFVPNSSTITSNKLEDPAN
TDCSSSNNYNSNNNSNVYHHNSFFEKEYSNTNNVGENKNNYENYEHSLSGKMNII
NLSENINVDNKDSNNNNSFDLFPFFKSLNSIRTKLLSYYDLDNDVVIYRCMCALLP
YFNVYKTYDFMNNFIDIEKNEHEMDDKNRDNANINETENDDYDDENENITKINN
MNNNFNNYNNKLSIEKNPDIYAFVWLNIFISFVIFFLFNIKNMFFSNSIYVDSTSTGT
TSNGGAIYDDTQDSDVIINMKNYMKENKLNILYNSLLFIYSFNIFIPVLVHVTNYFV
TKKVYPIKLSFLISLMSYNNIILLPVIFIYKYLIIETTSTFFFWFFTLLRFILFVFYMTTS
IFYIYKYVNKMLRIHFESNIVYLNYAIFLFSYMSFYLILKSYILNYL
>Povale|PocGH01_05030700.1: pep
(SEQ ID NO: 40)
MGGGNDGVYGHDYPGRGGSNSRNGHANVHMNTQSTSRNSSSNRELERDNERAN
LLASQYKDSFHIDDLSRRNNYAVGDTGGGGNVEGIFGKGSDGGFSNENLKTLNLN
DIKRGVHGNNAFLSSSNNMGYSLDFAVEGTVVGDKINTVSAFPSATTVTSGVTAS
QQVNGIFEKAYSMSDMDKQHGNNISGKMNIIDLSANGNEEKRGDIHSGNNSFELF
PFFKSLSAIRNKVLCYYDVDNDVIIYRCMCALVPYLQVDKTFECANNFNDIENNV
NQTGNRNMNTLNSLEKETYDENEHIRNISNIHDAFDSYDNKLSVLNNPDVYGFV
WLNMLITCIVFFLFNLKNIFYSKVSFIDVDTYTKDTQLMQDYIETNKLTILYSTILFV
YLFTVLVPTVVHVTNYLLTKKIMPIKLSFLISLMSYNNITLIPVIFMHKLTSIETNTPF
LLFVCSTLRFLVFLFYIITSVFYIYKYTIRVLRNNFADNITHFNYAIFAISYMSFYFLL
RSYIFNYL
>Pfalciparum|PF3D7_0419500.1: pep
(SEQ ID NO: 41)
MVNQDDKFKKPKNEIWENDEIYKKKNKCVNSTNDNSFMNMNGKNNFPLEDEYK
DIFQINNFSKNTDHNKNNVHLINNHNMKHNNNFITNEESEKNSLLSNKDLIIFNLQ
DIKNDGNMKRFDHTNNTFQTKSNTTTNNNNHRNSLDVILSNSNMNPIETNQLNNV
LKNDNTLNMYENNSYYEKNIQGKMNIINLSDNDINQDDDKRNSFDIFPFFKKFKGI
RTKLLSYYDIDTDVVIYRCMCALFPYLNVDKNYDVINNIYDIEKNCVDTNENGFD
NNTSTKEYTSQEKNMNTNNSKTNVRNNDKMNKEKTNLFDDETYDEENVRKSSN
VNDALDYYDNKLGLEKNPDIYSFVWLNLFISFLVFFLFNIKNVFFNDINNNISTNHI
SNNKNNHILDNQSKLNILYNTIFFIYSFNIFIPIIIYLTIYFKTKKIPPFKLIYLISLLSYN
NIMLLPIIFIYKIIIINTSINLVLYLYAILRFLIFIFYINTSIFYIYKYTNNIFFNHFTTDLI
YVLYAIFFLSYVSFYILLKYYIFNNL
>Pyoelii|PY17X_0721600.1: pep
(SEQ ID NO: 42)
MGNERNNQNYNDNDEKSRLVQNEFNNDFLINSNSRVDNNNSNIYYTNKNDKYD
QNDKYDQTDKYDNEIIFGSNIRNIQNGNLKTFNLNDINKESSNNSRNKNSNFTLNN
SNKNLPSHFLNFSEDIITDNINKNERKNGIQNESQNESQNNATTILDAQNNPPFLLN
EIHKNSNFYQTEGFEKEHNFFSEKQNNTQTNIMGKMNIINLSDKTSEDNFDENNEN
TPFGFFPLFKKLNNIRTKLLRFYDIDNDIIIYRCMCALFPYCDVDKKSYFINNFDDIE
QGHINSKIQNDCKTTEYSTDINNYTSNISDNENYDENENIRNVTCINDDAFDYYDN
KLNIIQNPDLYGFIWLNIFISFIYFFIFNLNNTIFNTTININNDYINSYIYQNKLNVLYN
TLFFIYLFNILLPIFILLANYFITKKKFAIKLLSLISLASYNNIILLPLILIYKFTIIDTTILI
IQYISSFIRLLLFIFYVATSLMYIYKFTIKIYRNNFSNEITYFNYFIFTISYISLYFILKSY
VFNYL
PY17X 070600 DNA
>PvivaxP01|PVP01_0528900.1: pep
(SEQ ID NO: 43)
atgaacacatcaaatagcgatttgaaaaaggaaaatttatgggacggccacgatgtgagcttacacaaaggcgatacgagccgca
acacgtatagcaacaatagtggcagtaatgggaagaagacgaaaaagaagaagaatgaaaagtcgaatttcttggccaatgaata
caaggacaccttccacgtggagggtctacctacgcgtaacaataacgtgaatatatattatggcaatgaggaagacgtcaatcaca
gtgccatcaacgacgacgatgacatttttgggagtggaggaaacactttccttaataagaatttaacaacggtcaatttgagtgacat
gaaaaggggaggagggcccccccatggtacaagcaactcaaatggaatcgccaattttgatggcagtttcggtgtagcggcata
cagcggatataacctaagcaacaacactggtagccccctcaacctttcgattgcccagggtgtagctaatcaaatgggaagcgcc
gcgaacgagagcggcagtcgaggatatcacagcaataagagcggcatgtatcaccagaggggccccctcgaacaagaattca
atgccggagatgtgcacgaaacgcccttgggaaacgtcccgagcggaaaaatgaacataattaatctgagcgacaacgaaaatg
acgacgactggggggtcgaaaccgcctcgagaagcagccttgagggcggaggaaacaactcgtttgatctgttccccttttttaaa
agtctgaaccgaatcagaacaaagctcttgtgctactacgacgtggacagcgacgtgatcatatacaggtgcatgtgcgcgctgct
gccttacctgaaggtggacaagtcgtacgattccatggacagcttggacgacattgagaagaacgcgggagaggcgagcgccg
ggcgcagccgcagggaccagcgcaatgggagaagtggcgtcaaaggtggcagcaaaagtggtggcaacaattacggtagca
atggcggcagcaatgacggcgtcgccagcgcggacgaaaacgaagccgacgacgagaacgcgaacataagaaaaatcagc
aacgtgaatgacgccttcgactactatgacaacaagctgagcatagaggggaacccagacctgtatggatttgtgtgggtgaacat
cttcatttccttcaccattttttttcttttcaactggaaaaatatctttttcggggactcccccgatggtgccacctcgagtagcgaagaaa
caaacaaccaagcgtacgtcacgcagaacaaattaaacattctgtacaccactttaatttttttatatctcttcaatacgcaaacgccttt
attaatatacgtcacaaatttctttgtcacgaaaagggtgtttcccatcaggttatcctttctcatttcgctaatgagttataacaacgtaat
tttgttccccctcattttgttatacaaatttaccttaataaatacgagcctttcgtttgttctcttcctttgcagtgccctgcgtttttttatttttg
cctactacatgatgtcctccctattttatatacacaaatacaccatccgcacttttcgcaacaatttttctgataacgtcatttatatgtact
atggaattttttgcctgtcctaccttttgttataccttcagttgaggaactacatattcagttatttgtga
>Pknowlesi|PKNH_0512200.1: pep
(SEQ ID NO: 44)
atgaacaaaacaaatagggatttggaagaggaaaatttatgggatgatcacgatgtgaacttacacaaaggcaatacgagccgca
acacgtatagcaacaatagtggcagtaatgggaagaaaacgaagaagaatgggaggtccaatttcatggccaatgaatacaaag
acaccttccacctgaaggaccttcccaggagtaacaataaagtgaatatatattatggcaatgaggaagatatcattgactgcgcca
tcaacgaagacgatgacatttttgggagtggaggaaacacttctctgaataagaatttaacaacggttaatttgagtgaaatgaaaag
gggaggaggccccccccaaggtataagcaattcaaatggaatcaccaattctgatggcagtgtcagtgtagcggcatattacgga
tataacctgaacaacaacacgggtaacccccctaacttttcgactggccagggtgtaactaatcaaataataatcgacgcgaatgat
agcggaaatcgaagataccacagcaataataaggacatgtaccaccagagggcccccctcgaacaaaaattcaatgccggaga
tgtgcacgatatgaacttcagaaacgccccgagcgggaaaatgaacatatttaatctaagcgacaacgaaaataacgacgactgg
ggggttgaaacagcatcgagaagcagccctgagggcggagaaaacaattcgttcgacctgtttcctttttttaaaggtctaaaccaa
atcagaacaaagctgttgtgttactacgacgtggacagcgatgtgataatatacagatgcatgtgtgctcttctgccttacctgaacgt
ggacaagtcgtacgattccatggacagcttggatgaccttgaaaagcacgcgtgtcaaccgagaagccgacgcagcaacaagg
acaagcgcaatgtaaaaagtggcgacaaaaattatggcagtagtcacgatgttgccagtgcagatgaaaacgaagtagacgacg
agaacgcgcacataagaaaaataagcaacgtgaatgacgccttcgattactatgacaacaaactgagcatagaaagaaacccag
acctgtatggatttgtatgggtgaatatcttcatttccttcaccattttttttctttttaattggaaaaacatattttttggtgattcctccggtat
cgactttacacccgaagatgacatcacctggagtagcgaagaaataaacaaccaagcatacatcacccagaataaattaaacattc
tctacaccactttaatttttttataccttttcaatacgttcactcctttatcaatatacatgacaaatttcattgtcacaaaaagaacgttccc
aatcaggttatcctttctcatttcattaatgagttataataatataattttattccctctcatattgttttacaaatttaccttgataaaaacaag
tctgtcattcattctcttcttttgtagttccttccgtttttttatttttgcccactatatgctgtcttccttattttacatacacaaatataccatcc
gcacttttcgcaataatttttctgataacatcatttatgcctactatggaattttttccctgtcctaccttctgttataccttctgttgaggaac
tacatattcagttatttgtga
>Pmalariae|PmUG01_05037000.1: pep
(SEQ ID NO: 45)
atggcgaatgaagaatacaacagaaagattagctgggatgaagatggtttcattggaataaacgaagataaccttagtagcattag
gcacaataatagtgtcaatgaaaagtccagcttgttaattacaaatgaatgtaaagatagttttcttattgatgacttttcaagaagacca
aaaaatatgatgagtgtatattatgaaaatgatgacgaggataatatttttggtgggggggaaaacagctcgttaaataaaaatttaaa
aacgcttaatttgagtgatataaaaaaaaagggggtaaaaagtgatagcaataatttaaattatggtagtaataacagtaacggtaga
agtagcagtaataacaatactagaagtatcactaatggtggtagtagtagcagtccatttcaagggctcatgagtaacgtgaatgac
gctcttaactttgtcccaaatagtagtactataacaagtaataaactggaagaccctgcaaatacagattgttcgtctagtaataattac
aacagtaataacaacagtaatgtatatcatcacaattccttttttgaaaaggaatatagtaacacaaataatgtaggagaaaataaaaa
caattatgaaaattatgaacatagtctaagcggaaaaatgaacataatcaatttaagcgaaaatataaatgtagataataaggatagc
aataataacaactcttttgatctatttcccttttttaaaagcttaaatagtattagaacaaaattattatcatattatgatttagataatgatgta
gtaatatataggtgtatgtgtgcactactaccttattttaatgtatataaaacatatgactttatgaacaattttattgatatagaaaagaat
gaacatgaaatggatgataaaaatagggataatgcaaatattaacgaaacggaaaatgatgattatgatgatgaaaatgaaaatata
acaaaaataaataacatgaataataactttaataattataacaacaaattaagtattgaaaaaaacccagatatatatgcctttgtttggt
tgaatatattcatctcctttgtaattttttttcttttcaacataaaaaatatgttttttagtaacagcatatatgttgatagtacttccacaggta
ctactagtaatggtggtgcaatatacgatgatacgcaggatagtgatgtgataattaatatgaaaaattacatgaaggaaaacaaatt
gaacattctctacaattcattactttttatctattcatttaacatttttatacctgtacttgtgcatgtaactaactattttgttacaaaaaaagt
atatccaattaaattgtcatttcttatttctttaatgagttataataacataattttattaccagtgatttttatttacaagtatttaattatagaaa
ctacttcaacattttttttttggtttttcacacttttacgatttattctttttgttttttatatgactacatccatattttatatatataaatatgtcaac
aaaatgttgcgcattcattttgaaagtaatattgtatatctcaattacgctatttttttattttcgtacatgtccttttatctcatattaaagagtt
acatactcaattatttataa
>Povale|PocGH01_05030700.1: pep
(SEQ ID NO: 46)
atgggtggtggtaacgacggtgtttatggtcatgattatcctggtaggggtggaagtaactcgcgcaacgggcatgcaaatgtgca
catgaacacacaaagtacatcccgcaattcgtccagtaacagagaattagaaagagataatgaaagagctaacctactggcgagt
cagtataaagatagttttcacattgatgacttgtcacggaggaataattacgccgttggtgacactggtggaggtggcaatgtggaa
ggaatttttggaaagggctccgatggtggtttctcaaatgagaacttgaaaacactaaatttaaatgatataaagaggggagtgcat
ggaaataacgcgtttttaagcagttctaacaacatggggtattccctcgactttgcggttgagggaactgtagttggggacaaaata
aataccgtttcagctttccctagtgctactactgtaacaagcggagttactgcttcccagcaggtgaatggcatttttgaaaaggcata
tagcatgagtgatatggacaaacaacatggaaataacataagtggaaaaatgaacataattgatttaagtgcgaatggaaatgagg
aaaaaagaggagatatacatagtggaaataattctttcgagttatttcccttttttaaaagtctcagtgctattagaaacaaagtattatgt
tattatgacgtggacaatgatgttattatttacagatgcatgtgtgcattagttccgtacttacaggtagacaaaacgttcgaatgtgcta
acaattttaatgatatagaaaataatgtaaaccaaactggtaatagaaatatgaacacactgaacagtttagaaaaagagacttatga
cgaaaacgaacatatacgaaatataagtaatatacacgatgcgtttgattcttatgataacaaattgagtgttctaaataacccagatgt
atacggttttgtctggttaaatatgcttataacgtgtatagttttttttctattcaatttgaaaaacattttttatagtaaggtatctttcattgat
gtagatacttacacaaaagacacccaacttatgcaagattacattgaaacaaacaaactgacaatactatacagtacaattcttttcgt
atatctctttaccgttttggtcccaacagtggtacatgttactaattaccttctaacaaaaaaaataatgccaataaaattatcgtttcttat
atccctaatgagttacaacaatataaccttaatacccgttatctttatgcacaaacttacaagcatagaaacaaatacaccttttcttcttt
tcgtttgttctactctacgttttcttgtctttcttttttacataattacatctgttttttatatatataaatatactataagagtattacgtaacaattt
tgccgacaatattacccatttcaattacgctatttttgcaatttcttacatgtctttttattttctcctaagaagttacatattcaactatctatg
a
>Pfalciparum|PF3D7_0419500.1: pep
(SEQ ID NO: 47)
atggttaatcaagatgataaatttaagaaacctaaaaatgaaatttgggaaaatgatgagatatataaaaaaaaaaacaaatgtgtta
attcaaccaatgataattcttttatgaatatgaatggaaagaataattttcctttagaagatgaatataaggatatatttcagattaataattt
ttctaagaatactgatcataataaaaataatgtgcatcttataaataatcataatatgaaacataataataattttataacaaatgaggaat
cagaaaaaaactctctcttgtcaaataaagatttaattatttttaatttacaagatattaaaaatgatggtaatatgaaaaggtttgatcata
ctaataatacatttcaaacaaaatctaatactactacaaataataataatcacagaaatagtttagatgttattttgtctaattctaatatga
atccaatcgaaacgaaccaattaaataatgtattaaaaaatgataatacattaaatatgtatgaaaataattcatattatgaaaaaaatat
acaaggaaaaatgaatattataaatttaagtgataatgatataaatcaggatgatgataaaagaaattcttttgatatcttccccttcttta
aaaaatttaaaggtataagaacaaaattattaagttattatgatatagatacagacgttgtcatatatagatgtatgtgtgccttatttccat
atttaaatgttgacaaaaattatgatgttataaataatatatatgatattgaaaaaaattgtgtagatacaaatgaaaatggtttcgataat
aatacaagtacaaaagaatatacatcacaagaaaagaacatgaatacaaacaacagcaaaaccaatgttagaaataatgataaaat
gaataaagagaaaactaacctttttgatgacgaaacatatgatgaagaaaatgtaagaaaatcaagcaatgtaaatgatgctttagat
tattatgataataaactagggttagaaaaaaatccagatatttattccttcgtatggttaaatttatttatatcctttctagtgttttttcttttta
atataaaaaatgtatttttcaatgatattaataataatatatcaacaaatcacatatcaaataataagaacaatcatattttagataatcaaa
gcaaattaaatattttatataatactatatttttcatatactcatttaatatatttattcccattataatatatctaacaatttatttcaaaaccaaa
aaaataccacctttcaaattaatatatcttatatctttattaagttataataatattatgttattaccaatcatctttatttataaaattattattata
aatacctccataaatttagtactctatctttatgcaattctacgtttccttatcttcattttttatattaatacatctattttttatatttataaatata
caaacaatatattttttaatcattttactacagatttgatatatgtactttatgcaatctttttcctttcatatgtatctttttatattcttctaaaata
ttacatatttaataatttataa
>Pyoelii|PY17X_0721600.1: pep
(SEQ ID NO: 48)
atgggtaatgaacgtaataatcaaaattataatgataatgatgaaaaatcaagattagtacaaaatgaatttaataacgattttcttataa
attcaaattcaagggtagacaacaataatagtaatatttattatacaaataaaaatgataaatatgatcaaaatgataaatatgatcaaa
ctgataaatatgataatgaaattatttttggatcaaatattagaaatatccaaaatggtaatttaaaaacatttaatttaaatgatataaata
aagaatcaagtaataacagtcgaaataaaaattcaaactttactttaaataattcaaataaaaatctcccttctcatttcctcaatttttctg
aggatattataactgacaatatcaataaaaacgaaagaaaaaatggaatccaaaatgaaagccaaaacgaaagccagaataacg
ctacaaccattttagacgcacaaaataaccctccttttttattaaatgaaattcataagaattcaaatttttatcaaacggaaggtttcgaa
aaagaacacaattttttttctgaaaaacaaaataatacccaaacaaatataatgggaaaaatgaatataataaatctaagtgacaaaa
caagtgaagataattttgatgaaaataatgaaaacacaccttttggtttttttcctttatttaaaaagttaaataatataagaactaaattgtt
acgtttttatgatatagataacgatataattatatatcgatgtatgtgtgcattatttccatattgtgatgtagacaaaaaatcttattttatta
ataattttgatgatattgaacaaggccatatcaactcaaaaatacaaaatgattgtaaaactacagaatattccacagatataaataatt
atacatcaaatatttcagacaatgaaaattatgatgaaaatgaaaatatcagaaatgtaacatgtattaatgatgatgcttttgattattat
gataataaattaaacattatacaaaatccagacttatatggatttatatggttaaatatatttatttcttttatatatttttttatttttaatttaaat
aatactatatttaataccacaataaatattaacaatgattatataaatagctatatttatcaaaataaattaaatgttttatataatacattattt
tttatttatttgtttaatatattacttcctatttttattttactagctaattattttattacaaaaaaaaaattcgctattaaattattatctttaatttct
ttagctagctataataatattattttattgcctttaattcttatttataaatttacaattatagacacaactattcttattatacaatatatatcatc
atttatacgtttattactttttattttttatgttgctacatcattaatgtatatatataaatttactataaaaatatatcgtaacaatttttcaaatga
aattatttattttaattatttcatttttactatttcttatatctctttatattttatcctcaaatcttatgtatttaattatttataa

### III. CONCLUSION

Many modifications and other embodiments of the inventions set forth herein will come to mind to one skilled in the art to which the inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

### BIBLIOGRAPHY

Wellems T E, Panton L J, Gluzman I Y, do Rosario V E, Gwadz, R W, Walker-Jonah A, Krogstad D J. Chloroquine resistance not linked to mdr-like genes in a Plasmodium falciparum cross. Nature. 1990 May 17; 345(6272):253-255.
Vaidya A B, Muratova O, Guinet F, Keister D, Wellems T E, Kaslow D C. A genetic locus on Plasmodium falciparum chromosome 12 linked to a defect in mosquito-infectivity and male gametogenesis. Mol Biochem Parasitol. 1995 January; 69(1):65-71.
Su X, Kirkman L A, Fujioka H, Wellems T E. Complex polymorphisms in an approximately 330 kDa protein are linked to chloroquine-resistant P. falciparum in Southeast Asia and Africa.Cell. 1997 Nov. 28; 91(5):593-603.
Carlton J, Mackinnon M, Walliker D. A chloroquine resistance locus in the rodent malaria parasite Plasmodium chabaudi. Mol Biochem Parasitol. 1998 May 15; 93(1):57-72.
Nair S, Williams J T, Brockman A, Paiphun L, Mayxay M, Newton P N, Guthmann J P, Smithuis F M, Hien T T, White N J, Nosten F, Anderson T J. A selective sweep driven by pyrimethamine treatment in southeast asian malaria parasites. Mol Biol Evol. 2003 September; 20(9):1526-1536.
Miotto O, Amato R, Ashley E A, MacInnis B, Almagro-Garcia J, Amaratunga C, Lim P, Mead D, Oyola S O, Dhorda M, Imwong M, Woodrow C, Manske M, Stalker J, Drury E, Campino S, Amenga-Etego L, Thanh T N, Tran H T, Ringwald P, Bethell D, Nosten F, Phyo A P, Pukrittayakamee S, Chotivanich K, Chuor C M, Nguon C, Suon S, Sreng S, Newton P N, Mayxay M, Khanthavong M, Hongvanthong B, Htut Y, Han K T, Kyaw M P, Faiz M A, Fanello C I, Onyamboko M, Mokuolu O A, Jacob C G, Takala-Harrison S, Plowe C V, Day N P, Dondorp A M, Spencer C C, McVean G, Fairhurst R M, White N J, Kwiatkowski D P. Genetic architecture of artemisinin-resistant Plasmodium falciparum. Nat Genet. 2015 March; 47(3):226-234.
Culleton R, Martinelli A, Hunt P, Carter R. Linkage group selection: rapid gene discovery in malaria parasites. Genome Res. 2005 January; 15(1):92-97.
Pattaradilokrat S, Culleton R L, Cheesman S J, Carter R. Gene encoding erythrocyte binding ligand linked to blood stage multiplication rate phenotype in Plasmodium yoelii yoelii. Proc Natl Acad Sci USA. 2009 Apr. 28; 106(17):7161-7166.
Michelmore R W, Paran I, Kesseli R V. Identification of markers linked to disease-resistance genes by bulked segregant analysis: A rapid method to detect markers in specific genomic regions by using segregating populations. PNAS. 1991 November; 88:9828-9832.
Martinelli A, Cheesman S, Hunt P, Culleton R, Raza A, Mackinnon M, Carter R. A genetic approach to the de novo identification of targets of strain-specific immunity in malaria parasites. Proc Natl Acad Sci USA. 2005 Jan. 18; 102(3):814-819.
Cheesman S, O'Mahony E, Pattaradilokrat S, Degnan K, Knott S, Carter R. A single parasite gene determines strain-specific protective immunity against malaria: the role of the merozoite surface protein I. Int J Parasitol. 2010 July; 40(8):951-961.
Hunt P, Martinelli A, Modrzynska K, Borges S, Creasey A, Rodrigues L, Beraldi D, Loewe L, Fawcett R, Kumar S, Thomson M, Trivedi U, Otto T D, Pain A, Blaxter M, Cravo P. Experimental evolution, genetic analysis and genome re-sequencing reveal the mutation conferring artemisinin resistance in an isogenic lineage of malaria parasites. BMC Genomics. 2010 Sep. 16; 11:499.
Blake D P, Billington K J, Copestake S L, Oakes R D, Quail M A, Wan K L, Shirley M W, Smith A L. Genetic mapping identifies novel highly protective antigens for an apicomplexan parasite. PLoS Pathog. 2011 Feb. 10; 7(2):e1001279.
Ehrenreich I M, Torabi N, Jia Y, Kent J, Martis S, Shapiro J A, Gresham D, Caudy A A, Kruglyak L. Dissection of genetically complex traits with extremely large pools of yeast segregants. Nature. 2010 Apr. 15; 464(7291):1039-42.
Wolyn D J, Borevitz J O, Loudet O, Schwartz C, Maloof J, Ecker J R, Berry C C, Chory J. Light-response quantitative trait loci identified with composite interval and eXtreme array mapping in Arabidopsis thaliana. Genetics. 2004 June; 167(2):907-17.
Wenger J W, Schwartz K, Sherlock G. Bulk segregant analysis by high-throughput sequencing reveals a novel xylose utilization gene from Saccharomyces cerevisiae. PLoS Genet. 2010 May 13; 6(5):e1000942.
Ehrenreich I M, Bloom J, Torabi N, Wang X, Jia Y, Kruglyak L. Genetic architecture of highly complex chemical resistance traits across four yeast strains. PLoS Genet. 2012; 8(3):e1002570.
Modrzynska K, Creasey A, Loewe L, Cezard T, Trindade Borges S, Martinelli A, Rodrigues L, Cravo P, Blaxter M, Carter R, Hunt P. Quantitative genome re-sequencing defines multiple mutations conferring chloroquine resistance in rodent malaria. BMC Genomics. 2012 Mar. 21; 13:106.
Illingworth C J, Parts L, Schiffels S, Liti G, Mustonen V. Quantifying selection acting on a complex trait using allele frequency time series data. Mol Biol Evol. 2012 April; 29(4):1187-1197.
Illingworth C J, Mustonen V. Quantifying selection in evolving populations using time-resolved genetic data. Journal of Statistical Mechanics: Theory and Experiment. 2013: P01004.
Edwards M D, Gifford D K. High-resolution genetic mapping with pooled sequencing. BMC Bioinformatics. 2012 Apr. 19; 13 Suppl 6:S8.
Abkallo H M, Tangena J A, Tang J, Kobayashi N, Inoue M, Zoungrana A, Colegrave N, Culleton R. Within-host competition does not select for virulence in malaria parasites; studies with Plasmodium yoelii. PLoS Pathog. 2015 Feb. 6; 11(2):e1004628.
Vázquez-García I, Salinas F, Li J, Fischer A, Barré B, Hallin J, Bergström A, Alonso-Perez E, Warringer J, Mustonen V, Liti G. Background-dependent effects of selection on subclonal heterogeneity. Preprint at bioRxiv, https://doi.org/10.1101/039859.
Holder, A A. The carboxy-terminus of merozoite surface protein 1: structure, specific antibodies and immunity to malaria. Parasitology. 2009 October; 136(12):1445-1456.
Proellocks N I, Kovacevic S, Ferguson D J, Kats L M, Morahan B J, Black C G, Waller K L, Coppel R L. Plasmodium falciparum Pf34, a novel GPI-anchored rhoptry protein found in detergent-resistant microdomains. Int J Parasitol. 2007 September; 37(11):1233-1241.
Otsuki H, Kaneko O, Thongkukiatkul A, Tachibana M, Iriko H, Takeo S, Tsuboi T, Torii M. Single amino acid substitution in Plasmodium yoelii erythrocyte ligand determines its localization and controls parasite virulence. Proc Natl Acad Sci USA. 2009 Apr. 28; 106(17):7167-7172.
Sim B K, Chitnis C E, Wasniowska K, Hadley T J, Miller L H. Receptor and ligand domains for invasion of erythrocytes by Plasmodium falciparum. Science. 1994 Jun. 24; 264(5167):1941-1944.
Mayer D C G, Kaneko O, Hudson-Taylor D E, Reid M E, Miller L H. Characterization of a Plasmodium falciparum erythrocyte binding protein paralogous to EBA-175. Proc Natl Acad Sci USA. 2001 Apr. 24; 98(9):5222-5227.
Van Buskirk K M, Sevova E, Adams J H. Conserved residues in the Plasmodium vivax Duffy-binding protein ligand domain are critical for erythrocyte receptor recognition. Proc Natl Acad Sci USA. 2004 Nov. 2; 101(44):15754-15759.
Culleton R, Kaneko O. Erythrocyte binding ligands in malaria parasites: intracellular trafficking and parasite virulence. Acta Trop. 2010 June; 114(3):131-137.
Molina-Cruz A, Garver L S, Alabaster A, Bangiolo L, Haile A, Winikor J, Ortega C, van Schaijk B C, Sauerwein R W, Taylor-Salmon E, Barillas-Mury C. The human malaria parasite Pfs47 gene mediates evasion of the mosquito immune system. Science. 2013 May 24; 340(6135):984-7.
Vaughan A M, Pinapati R S, Cheeseman I H, Camargo N, Fishbaugher M, Checkley L A, Nair S, Hutyra C A, Nosten F H, Anderson T J, Ferdig M T, Kappe S H. Plasmodium falciparum genetic crosses in a humanized mouse model. Nat Methods. 2015 July; 12(7):631-3.
Inoue M, Tang J, Miyakoda M, Kaneko O, Yui K, Culleton R. The species specificity of immunity generated by live whole organism immunisation with erythrocytic and pre-erythrocytic stages of rodent malaria parasites and implications for vaccine development. Int J Parasitol. 2012 August; 42(9):859-70.
Abkallo H M, Liu W, Hokama S, Ferreira P E, Nakazawa S, Maeno Y, Quang N T, Kobayashi N, Kaneko O, Huffman M A, Kawai S, Marchand R P, Carter R, Hahn B H, Culleton R. DNA from pre-erythrocytic stage malaria parasites is detectable by PCR in the faeces and blood of hosts. Int J Parasitol. 2014 June; 44(7):467-473.
Team RDC. R: A language and environment for statistical computing. 2014: http://www.R-project.org/.
Bolger A M, Lohse M, Usadel B. Trimmomatic: a flexible trimmer for Illumina sequence data. Bioinformatics. 2014 Aug. 1; 30(15):2114-2120.
Li H, Durbin R. Fast and accurate short read alignment with Burrows-Wheeler transform. Bioinformatics. 2009 Jul. 15; 25(14):1754-1760.
Li H, Handsaker B, Wysoker A, Fennell T, Ruan J, Homer N, Marth G, Abecasis G, Durbin R; 1000 Genome Project Data Processing Subgroup. The Sequence Alignment/Map format and SAMtools. Bioinformatics. 2009 Aug. 15; 25(16):2078-2079.
Fischer A, Vázquez-García I, Illingworth C J, Mustonen V. High-Definition Reconstruction of Clonal Composition in Cancer. Cell Rep. 2014 Jun. 12; 7(5):1740-1752.
Cingolani P, Platts A, Coon M, Nguyen T, Wang L, Land S J, Lu X, Ruden D M. A program for annotating and predicting the effects of single nucleotide polymorphisms, SnpEff: SNPs in the genome of Drosophila melanogaster strain w1118; iso-2; iso-3. Fly. 2012 6(2):90-92.
Schwarz G Estimating the Dimension of a Model. The Annals of Statistics. 1978; 6: 461-464.
Fernandez-Becerra C, de Azevedo M F, Yamamoto M M, del Portillo H A. Plasmodium falciparum: new vector with bi-directional promoter activity to stably express transgenes. Exp Parasitol. 2003 January-February; 103(1-2): 88-91.
Sakura T1, Yahata K, Kaneko O. The upstream sequence segment of the C-terminal cysteine-rich domain is required for microneme trafficking of Plasmodium falciparum erythrocyte binding antigen 175. Parasitol Int. 2013 April; 62(2):157-164.
Kim D, Pertea G, Trapnell C, Pimentel H, Kelley R, Salzberg S L. TopHat2: accurate alignment of transcriptomes in the presence of insertions, deletions and gene fusions. Genome Biol. 2013 Apr. 25; 14(4):R36.
Rutherford K, Parkhill J, Crook J, Horsnell T, Rice P, Rajandream M A, Barrell B. Artemis: sequence visualization and annotation. Bioinformatics. 2000 October; 16(10):944-945.
Mutungi J K, Yahata K, Sakaguchi M, Kaneko O. Expression and localization of rhoptry neck protein 5 in merozoites and sporozoites of Plasmodium yoelii. Parasitol Int. 2014 December; 63(6):794-801.
Batchelor J D1, Zahm J A, Tolia N H. Dimerization of Plasmodium vivax DBP is induced upon receptor binding and drives recognition of DARC. Nat Struct Mol Biol. 2011 Jul. 10; 18(8):908-914.
Biasini M, Bienert S, Waterhouse A, Arnold K, Studer G, Schmidt T, Kiefer F, Gallo Cassarino T, Bertoni M, Bordoli L, Schwede T. SWISS-MODEL: modelling protein tertiary and quaternary structure using evolutionary information. Nucleic Acids Res. 2014 July; 42(Web Server issue):W252-258.
Kiefer F, Arnold K, Kunzli M, Bordoli L, Schwede T. The SWISS-MODEL Repository and associated resources. Nucleic Acids Res. 2009 January; 37(Database issue):D3873-92.
Arnold K, Bordoli L, Kopp J, Schwede T. The SWISS-MODEL Workspace: A web-based environment for protein structure homology modelling. Bioinformatics. 2006 Jan. 15; 22(2):195-201.
Guex N, Peitsch M C, Schwede T. Automated comparative protein structure modeling with SWISS-MODEL and Swiss-PdbViewer: A historical perspective. Electrophoresis. 2009 June; 30 Suppl 1:5162-5173.
Lin D H, Malpede B M, Batchelor J D, Tolia N H. Crystal and Solution Structures of Plasmodium falciparum Erythrocyte Binding Antigen 140 Reveal Determinants of Receptor Specificity during Erythrocyte Invasion. J Biol Chem. 2012 Oct. 26; 287(44):36830-36836.
Ceroni A, Passerini A, Vullo A, Frasconi P. DISULFIND: a Disulfide Bonding State and Cysteine Connectivity Prediction Server. Nucleic Acids Res. 2006 Jul. 1; 34(Web Server issue):W177-181.
Vullo A, Frasconi P. Disulfide connectivity prediction using recursive neural networks and evolutionary information. Bioinformatics. 2004 Mar. 22; 20(5):653-659.
Frasconi P, Passerini A, Vullo A. A two-stage SVM architecture for predicting the disulfide bonding state of cysteines. Proc. IEEE Workshop on Neural Networks for Signal Processing. 2002: 25-34.
Ceroni A, Passerini A, Vullo A. Predicting the disulfide bonding state of cysteines with combinations of kernel machines. Journal of VLSI Signal Processing. 2003; 35: 287-295.
Craig D B, Dombkowski A A. Disulfide by Design 2.0: a web-based tool for disulfide engineering in proteins. BMC Bioinformatics. 2013 Dec. 1; 14:346.

1. An immunogenic composition against Plasmodium comprising:
(A) all or part of the nucleotide sequence:
(i) PY17X_0721800 found in genomic location PyI7X-07-v2: 799,281-800,081 (+) or an ortholog thereof, or
(ii) PY17X_0720100 found in genomic location PyI7X-07-v2: 727,812-742,672 (+), or
(iii) PY17X_0721500 found in genomic location Py17X-07-v2: 784,994-791,991 (+),
on chromosome 7 of Plasmodium yoelii or an ortholog thereof in Plasmodium falciparum or (B) a polypeptide encoded by all or part of the nucleotide sequence:
(i) PY17X_0721800 or an ortholog thereof,
(ii) PY17X_0720100 or an ortholog thereof; or
(iii) PY17X_0721500 or an ortholog thereof,
in Plasmodium falciparum.

2. An immunogenic composition against Plasmodium comprising an immunogenic polypeptide, wherein the immunogenic polypeptide is encoded by a nucleic acid sequence with at least 75% sequence identity to a sequence selected from the group consisting of: SEQ ID NOs: 7, 8,9, 10, 11, 12, 19, 20, 21, 22, 23, 24, 31, 32, 33, 34, 35, 36 or a fragment thereof.

3. The immunogenic composition of claim 2, wherein the immunogenic polypeptide is encoded by a nucleic acid sequence with at least 80% sequence identity to a sequence selected from the group consisting of: SEQ ID NOs: 7, 8, 9, 10, 11, 12, or a fragment thereof.

4. The immunogenic composition of claim 3, wherein the immunogenic polypeptide is encoded by a nucleic acid sequence with at least 90% sequence identity to a sequence selected from the group consisting of: SEQ ID NOs: 7, 8, 9, 10, 11, 12, or a fragment thereof.

5. The immunogenic composition of claim 1 comprising all or part of the nucleotide sequence PY17X_0720100 found in genomic location PyI7X-07-v2: 727,812-742,672 (+) on chromosome 7 of Plasmodium yoelii or an ortholog thereof in Plasmodium falciparum or a polypeptide encoded by all or part of the nucleotide sequence PY17X_0720100 or an ortholog thereof in Plasmodium falciparum.

6. The immunogenic composition of claim 2 comprising an immunogenic polypeptide, wherein the immunogenic polypeptide is encoded by a nucleic acid sequence with at least 75% sequence identity to a sequence selected from the group consisting of: SEQ ID NOs: 19, 20, 21, 22, 23, 24, or a fragment thereof.

7. The immunogenic composition of claim 6, wherein the immunogenic polypeptide is encoded by a nucleic acid sequence with at least 80% sequence identity to a sequence selected from the group consisting of: SEQ ID NOs: 19, 20, 21, 22, 23, 24, or a fragment thereof.

8. The immunogenic composition of claim 7, wherein the immunogenic polypeptide is encoded by a nucleic acid sequence with at least 90% sequence identity to a sequence selected from the group consisting of: SEQ ID NOs: 19, 20, 21, 22, 23, 24, or a fragment thereof.

9. The immunogenic composition of claim 1 comprising all or part of the nucleotide sequence PY17X_0721500 found in genomic location PyI7X-07-v2: 784,994-791,991 (+) on chromosome 7 of Plasmodium yoelii or an ortholog thereof in Plasmodium falciparum or a polypeptide encoded by all or part of the nucleotide sequence PY17X_0721500 or an ortholog thereof in Plasmodium falciparum.

10. The immunogenic composition of claim 2 comprising an immunogenic polypeptide, wherein the immunogenic polypeptide is encoded by a nucleic acid sequence with at least 75% sequence identity to a sequence selected from the group consisting of: SEQ ID Nos: 31, 32, 33, 34, 35, 36, or a fragment thereof.

11. The immunogenic composition of claim 10, wherein the immunogenic polypeptide is encoded by a nucleic acid sequence with at least 80% sequence identity to a sequence selected from the group consisting of: SEQ ID Nos: 31, 32, 33, 34, 35, 36, or a fragment thereof.

12. The immunogenic composition of claim 11, wherein the immunogenic polypeptide is encoded by a nucleic acid sequence with at least 90% sequence identity to a sequence selected from the group consisting of: SEQ ID Nos: 31, 32, 33, 34, 35, 36, or a fragment thereof.

13. The immunogenic composition of claim 1, wherein the immunogenic composition comprises an adjuvant.

14. The immunogenic composition of claim 13, wherein the adjuvant comprises a granulocyte/macrophage colony-stimulating factor (GM-CSF) protein, a nucleotide molecule encoding a GM-CSF protein, saponin QS21, monophosphoryl lipid A, or an unmethylated CpG-containing oligonucleotide.

15. The immunogenic composition of claim 1, wherein the immunogenic composition is against Plasmodium falciparum.

16. A method of immunizing a subject against Plasmodium, comprising administering an immunogenic amount of the immunogenic composition of claim 1.

17. A method of eliciting an immune response in a subject against Plasmodium, comprising administering an immunogenic amount of the immunogenic composition claim 1.

18. The method of claim 16, wherein the Plasmodium is Plasmodium falciparum.

19. (canceled)

20. A kit, comprising a container, wherein the container comprises at least one dose of an immunogenic composition of claim 2.


SEQUENCE LISTING (88 sequences)

SEQ ID NO: 1 - protein, 363 aa, Artificial Sequence
  Synthetic
MMNVFFCLSCFLVIKTLFQSCPAKCNELNVSGKGNMLYDYLFTPEVKAPKDDGGKEQDTLAGNKQMSYKTSEVQIKHHEEVHTGGMENGDHRKELKEHMDQLKMLHKSLKSKDRNTYGQVKPSGGESVHSNDGALGDTHTGGDQTSEESLAKYVQNEIKNNEKLNKEKKSYDEINLLEDNSKKLQNDIHTWLQSVKNITEKTSKLKDIKTQLLNNIASLNETLTEEIENINEIKKLQKEQNEIFSENWLYFLPSTSDSLASEGRDGSFQVLNYLEKFSRRDAPQVSSEPASGELQNGGLPNGGLPNGGLPNGGFPNGGLPNGGPPNDGNVRKAHSHTDDGAKSSDSRSFCSVLVLLAIAFLLS

SEQ ID NO: 2 - protein, 339 aa, Artificial Sequence
  Synthetic
MMNVFVCLSFILVISIFLQSNPAKCNELNISEKGKMLYDYFFTPKVRGPKDGGTDQDVHTGDKHLSYTTSEAQIRHHEQVHIGGMKNNDHKKELKEHMDQLKMLHKSLKDKDKNAHDPELKVSGGESVHNTDGTLGDTHANGEETSDDSLAKYVQEEIRKNEKLNNEKKSYDEINILEDNSKKLQNDIHTWLQSVKNISEKTNKLKDIKTQLLNNIASLNETLTEEIENIKEIKKLQMEQNQIFSENWLYFLPSTFDNLASHGQDGNFQVLNYLEKFNRRDGLHVSGEKANLEQPSGTLANEGNVRKQNAHTDGGVKSSDFHSFCNVIVLLAVVTFLLS

SEQ ID NO: 3 - protein, 381 aa, Artificial Sequence
  Synthetic
MYVRTAVCCIRLHVTDNTFSVPMNFQKCKVFFLFSHLFLFLDFPLFNIIMNCGFSFFCILIINIILSINLIVECNKLMLNDKDKMTVDAILSKEVKENKGFNIQQVEKGNAISHKKLEMNINHHNVNDNDLKDHMDKLRKLHKDLKNSSSSSSNSNNSSTNSSNSSSKAYSKGDILKVEDQNSSNKTDKNNHSTNGKGEKDNNDDSLRKYILQEINNNRKLDTEKKSYEEINLLEENSKKLQNDIHTWLKSVNDIEEKSKKLKDIKSQLLNNIASLNQTLTEEIENINDIKKLQKEQNDIFSENWLYFFPSTPDYLIEEKNKSNYNILNYLESFSKKNKQQSYESNIRKQQKPHHYDNTNSSDIFHFLSYFVLLMFFFFFS

SEQ ID NO: 4 - protein, 311 aa, Artificial Sequence
  Synthetic
MILRFSFLCVLVINVLSTNLFVLCNKLKINGNGKSVNESFFPDGTKNESHMNKNIMKKGREREIPRKETQANIGHHNFHENELKKNIEMLKQMQGDLKNGKTPLHKKAINGADKGGHIGEWDREDELRKYVMEEIRNNSKLDREKKSSEEIKLLEDKSIQLQNDIHTWLQSIHNIEEKSTKLKDIKKELLHNITSLNETLVEEIENINDIKKLQKEQNEIFAENWLYFFPSISDNVSEDGSDSNHNILNYLEMYNKKDKEKQKESEMERGVWKEHLRENATMKQYGESSKSAGMGCVVNHILFLLGVIFLF

SEQ ID NO: 5 - protein, 325 aa, Artificial Sequence
  Synthetic
MYGTFLKGSIFYLCIFFPFFSCECNNIKLNDKENINFESYFNKRTNEENVLNKNVSKEMGDTFVAHKAIELNINHHHVNNDKEFNNNNNNKHQPYYHNEHDKKFSESLKAHMDHLKILNNDLKQHIDKKERNEIYENNDLKKYIIKEIQNNKYLNKEKKSSEDIQILEEHSKKLQKEIHEWLESVNNIEEKSNILKNIKSQLLNNIASLNHTLSEEIKNINDIKELQKQQNDLFSENWLYFLPSSSDYLLNEKKKNLYDNQDNSMKDDINNNDKYNIFNYLQNVQDKDNQYEVMKQDNNNIHSGSSTHNHLLLTCIIFLLILLIL

SEQ ID NO: 6 - protein, 266 aa, Artificial Sequence
  Synthetic
MNYNFLFFSILIINIFSTHLISKCNKLKLSSKRKSNQDALIPNEGNYMNNNIIDGEKNNKENINEEIIKQDGKIDNGENYIEIKLDYNKNDRINNEKINTNINHIDNVQNEYDNIKEMEKMTKSYEDMSILEENSKKLQNDIHSWIKSVHSIEEKTNTLKNIKNDLLNNITSLNKTLLEEIENINEIKKLQNEQNEIFSENLLYFFPSMPEKLQENVEKDYNILNYLEIYNKKDNLKKVDTNISSTCMFSFFSYLILFSATVFFFL

SEQ ID NO: 7 - DNA, 1089 bp, Artificial Sequence
  Synthetic
atgatgaatgttttcttctgcctctcgtgctttttggtaataaaaaccctcttccagagctgcccagccaaatgcaacgaattaaatgtaagtggcaaggggaatatgctgtacgattatttgttcacccctgaggtgaaggccccaaaggatgatggggggaaggagcaggacaccctggcagggaacaagcaaatgtcctacaaaacatcagaggtacaaattaagcaccacgaagaagtgcacacaggagggatggaaaatggtgatcataggaaggagctcaaggagcatatggaccagctaaaaatgctacataaaagtttaaaaagcaaagatcgaaatacatacggtcaggtaaaaccaagtggaggcgaaagtgtacacagtaatgatggcgcactgggggatacccatactgggggggaccaaacgagtgaagagagtctggccaaatatgtgcaaaacgaaataaaaaacaacgagaaattaaataaagaaaaaaaaagctacgacgaaattaacctactggaggataattcaaaaaaattacaaaatgatattcacacctggctccagtctgtaaaaaatataacggaaaagacgagcaaattgaaggacattaaaacgcagctgctgaacaacatcgcctcgttaaatgaaaccctgacggaagaaattgaaaatataaacgaaattaaaaagttgcaaaaggaacagaatgaaatattttccgaaaattggctctacttcctcccctccacgtcggacagcttggccagcgagggacgcgacggcagctttcaagtcctcaactacttggagaagtttagcaggagggacgctccgcaagttagtagtgagcccgcgagtggggaactccaaaatggggggcttcctaatgggggccttccaaatgggggtcttccaaatgggggtttcccaaacgggggtctcccaaacgggggtcccccaaacgatgggaacgttcgtaaggcgcactctcacaccgacgacggggcgaagtcgtccgactcgcgcagcttctgcagcgtgctcgtcctgctcgcgatcgcctttctgctcagc

SEQ ID NO: 8 - DNA, 1020 bp, Artificial Sequence
  Synthetic
atgatgaatgttttcgtttgtctctcgttcattctggttataagcatattcctccagagcaacccggccaaatgcaacgaattaaatataagtgaaaaggggaagatgttgtacgattatttctttacccctaaggtaaggggcccaaaggatgggggtacggaccaggatgtccacacaggggataagcacctgtcgtacacaacatcggaagcacaaattaggcaccacgaacaagtgcacataggaggaatgaaaaataatgaccacaaaaaggaactgaaagagcatatggatcagctaaaaatgctacataagagtttaaaagacaaggacaaaaatgcacacgatcctgagttgaaagtaagtggaggcgaaagtgtgcacaatactgatggcacactgggggatacccatgctaatggagaggaaacgagtgatgacagcctggcgaaatatgtgcaagaagaaataaggaaaaacgagaaattaaataatgaaaaaaagagctatgacgaaattaacatactcgaggataattcgaagaaattacaaaatgatattcatacctggcttcagtcagtaaaaaacatatcggaaaagacaaacaaattgaaggacattaagacgcagcttctgaacaacatcgcctcattaaatgagacacttacggaagaaattgaaaacataaaagaaataaaaaagttacaaatggagcaaaatcaaatattttcggaaaattggctctactttttgccttccacatttgacaaccttgctagccacgggcaagacggcaattttcaagtactgaactatttggagaagtttaacaggagggatggtcttcacgtgagtggtgagaaggcgaatctggaacaaccaagcggaacacttgcaaatgagggaaacgttcgtaagcagaatgcgcacaccgacggaggggtgaagtcgtccgactttcacagcttctgcaacgtgatcgttctactggcagtggttacctttctgctcagctag

SEQ ID NO: 9 - DNA, 1146 bp, Artificial Sequence
  Synthetic
atgtatgtacgcactgctgtatgttgtattcgtcttcacgtaacggacaatacgtttagtgtccctatgaattttcagaaatgtaaagttttctttttattttcccatctttttctttttttagattttcccctttttaatattataatgaattgcggtttttcttttttttgtattttaataataaatataattctttcaataaatttaatcgttgaatgtaataaattaatgttaaatgacaaagacaaaatgacagtggatgctattctttcaaaagaggtgaaagagaacaaaggattcaacatacaacaagtagaaaaaggaaacgccatttcgcacaaaaaacttgaaatgaacattaaccatcacaatgtgaatgacaatgatttgaaggatcatatggacaagctaaggaaattacacaaagatttaaaaaatagtagtagtagtagtagtaatagtaataatagtagtactaatagtagtaatagtagtagtaaagcatattcaaaaggggatatactaaaagttgaagatcagaatagtagtaataagacagataagaataatcatagtacaaatggaaaaggggagaaggataataacgatgatagcttaagaaaatacattttacaagaaataaataacaacagaaaattagatacagagaaaaaaagctatgaagaaataaatttgttagaagaaaattcaaaaaaactacaaaatgatattcatacttggttaaagtcagtaaatgacatagaagaaaagtccaaaaaactaaaagatattaaaagtcaactattaaataatattgcatctttaaatcaaactttaacagaagaaatagaaaatataaatgatattaaaaagttacaaaaagaacaaaatgacattttttctgaaaattggttatactttttcccctcaacacctgactatttaatagaagaaaaaaataaatcgaattataatattttaaattatttagaatcctttagtaaaaaaaataaacaacagagttacgaaagtaatataaggaaacaacaaaaacctcatcattatgataatactaattctagtgatattttccactttttaagctatttcgtcctcttaatgtttttttttttttttagttaa

SEQ ID NO: 10 - DNA, 936 bp, Artificial Sequence
  Synthetic
atgattttgagattttcctttttgtgcgttttggtaattaacgtactttcaaccaatttgttcgtcctatgcaacaaattgaaaataaatggaaacgggaaatcagtaaatgaatcgttcttcccagatgggacaaaaaacgaaagtcatatgaataaaaatattatgaagaaaggacgagaaagagaaatcccgagaaaggaaacacaggcaaacattggtcatcacaattttcatgaaaacgagctgaagaaaaatatcgagatgttaaagcaaatgcaaggggatttaaaaaacgggaaaactccattacacaaaaaagcaatcaatggagcagacaagggggggcacattggtgaatgggatagagaagacgaactgagaaaatatgttatggaagaaatacgtaataatagcaaattggacagagaaaaaaaaagttctgaggaaattaagttattagaggataaatcaatacaactacaaaacgatatacatacatggttacaatcaattcataatatagaggaaaagtctactaaattgaaagacataaaaaaggaattgttacataacataacctcattaaatgagacattagtagaagagatagaaaatattaatgatattaaaaagctacaaaaggagcaaaatgaaatatttgcagaaaattggctctattttttcccctctatatcggataacgtatcagaagatgggtcggacagtaaccataacattcttaactacctagaaatgtacaataaaaaggacaaagaaaaacagaaagagtcagaaatggaaagaggcgtgtggaaagaacatcttcgtgaaaacgccactatgaagcaatatggcgaaagttcaaaatcagcaggtatgggttgcgttgtaaaccatatccttttccttttaggagttatattcctcttttga

SEQ ID NO: 11 - DNA, 978 bp, Artificial Sequence
  Synthetic
atgtatggtacatttttgaaggggtccattttttacctgtgtatatttttcccatttttttcgtgtgagtgtaataatataaaattaaacgataaagagaatataaattttgagagttattttaataaaaggacaaatgaagaaaatgtattaaataaaaacgtatcgaaagaaatgggggatacgtttgttgcacataaggctatagaattaaacattaatcatcaccacgttaataatgataaagaatttaataataataataataataaacatcagccttattatcataatgagcatgataagaaattttctgaaagtttaaaagcacatatggatcaccttaagatattaaataatgatttaaaacaacatatagataaaaaagagagaaatgaaatatatgaaaataatgatttaaaaaaatatataataaaagagatacaaaataataaatatttaaataaagaaaagaaaagcagtgaagatattcaaatattagaagagcattcaaaaaaattacaaaaagaaattcatgaatggttagaatctgttaataatattgaagagaaatcaaatattttaaaaaatatcaaaagtcaattattaaataatatagcttctttaaatcatacgctctcagaagaaataaaaaatattaacgatataaaagaattacaaaaacaacaaaatgatttattttctgaaaattggttatattttcttccatcctcatcagattatctcttaaacgaaaaaaaaaaaaatttatatgataatcaagataatagtatgaaggatgatataaataataatgacaaatataatatttttaattatttacaaaacgttcaagataaggataaccaatatgaagttatgaaacaagacaataataatatacatagtggttcctctactcataatcatctattattaacttgtataatttttttgttaatacttttaattttataa

SEQ ID NO: 12 - DNA, 801 bp, Artificial Sequence
  Synthetic
atgaattataattttttatttttttcgattttaataataaatatattttcaacacatttaataagcaaatgtaacaaactgaaattaagttcaaaaagaaaaagtaaccaggatgctcttattccaaatgagggaaattatatgaacaataatataattgatggtgaaaaaaataataaggaaaatataaatgaagaaataattaaacaggatggaaaaatagacaatggagaaaattatatagaaattaaattagattataataaaaacgatagaataaataatgagaaaataaatacaaatattaatcatattgataatgtacaaaatgaatatgataatattaaagaaatggaaaaaatgacaaaaagctatgaagatatgagtattttagaagaaaattcaaaaaaattacaaaatgatatacattcatggataaaatctgtacatagtattgaagaaaagacaaatacattaaaaaatataaaaaatgatttattaaataatattacatcattaaataaaacattattagaagaaattgaaaatattaatgaaattaaaaaacttcaaaatgaacaaaatgaaatattttctgaaaatttgttgtattttttcccatcaatgcctgaaaaattacaagaaaatgtagaaaaagattataatattttaaattatttagaaatttacaataaaaaagataatttaaaaaaagttgatacaaatatatcatctacttgtatgtttagtttttttagttatttaattttgttttcagcaacagttttcttttttttataa

SEQ ID NO: 13 - protein, 4453 aa, Artificial Sequence
  Synthetic
MPGETQNTFDLVDVEPKFLEFHYEGADSVEAFLENKKKVIKRKGLKIKNICTKTQNIKICECDSKCLKIKGLKKKKLAPGTSEQILVEFSFADVNFKESKRGKQEDILEVVNNVEATSYVTIQSEYTTLSIPIYIKKSIPVFAYDDVINFGVCNANRTYHFALRVKNVGTRRGAFTLSLGDSPGGNADECEDGKRAESAQSGENSVKQHRRNCHSEDMLIRLDQSSLELDVGETKTINITVSSKVEGKMHREYIVVSNQHSYFVEKQRNINVLAIFTNSHTSFLFENGKTNLLNLHCLYYGSRKKYQGKIKNENNYGIFCTPRVDAVHVFYSKEELQTFAAARGLDLGRICRDVFLEEEERLSEGEKKEREKLFAITKKRLKGEEHIGVTFCRGEGYPVERLSYQEVTFEIQSKSDAGLLHRLSRDTAYLLNFPVVVELSLRVGRSRREASQVGITTKQAHRSTSQVCSSAKQEHRSTSQVCSSAKLPHSSSKLPPLSTKLPHSSTEVGDSHPCDEEVKLWAAFCLTFPCVLPSTYLLSYGSIAPNEGKTVILELTNRNKFLKVSYTLSKIPYLTLSKKEGVIPPQGKALLSLKLQCDSLKMVEEYMYIYFCNNLFFFVLLVRGNITSANALRKGTSSILLDPKGDSLKRNTPPQGDSKQDRDQVYEQILHNLELEKVSRNYYQLDGKLDKYKYNERFINFLKKNKKKYNDVLKLMYEERKKSESVRSGTQSQGSPDKSGKIIKGGTDKLSKIMTDKFIYVNDPPVDRISNTCIEKGVYERERKKYIQMKTTNWGVKNGEPSQGGEEQPYLLQFDMLDEEELKRVQFVRVIQYGNIFLGKEYTRKFAVFNRNKHCSVRVALTHSENVTTEGDNPLVIHRDSHKMGIIKLNVKRFSTAEEPHHDAVSDGDFRSQTGVSTPLFEAVNLPTGGGTSTPEGNTPTALCADPCNDFFLLKTFQEKISLTVNDSHEREVTLGATLVFLNILVHPHTLHFRFHDEDVGMFCERHLVLYNPFNVPLRVGMACNRACVQLDAEISVPPNSHKIVPVKFVATESATSVAEAIRLYLDGSRLYRSLTCKWIANKCSYRVTPAELNFENVLLNKTYVKEFFLINTGDSPVVFRCSYKPDCVNLLSKHNYVKKNEKVKISVLLNLKECKKMKDKVVLAVRGAPQLLLPLSAKGTPSNVLISGGLRIRQRRGELKCYEMKIANRGPCEEAILLDTSPVGFLNIQMGHNNERTFSESTGKESKDASKRENVLTVERTFNQEYDDLLTDECAKYQYNNFLRLHNLVSSCYQNKGQILFNEKRERKNIYRVVVPSKCFLPLYIQCYSSRALTKEITLHSLLHCNRAAYECKEESITVDIEEGHLDVSPPFLLFTDLLPQKADEAYITYFSRERRKRLTIRNVLSQPVQWAVRLDEEKRRELYTVRGAHRGGEQIAERRGEAGKSRRVGSDIKGSAIGGSAKRGEAPETCVTQGSAIGGSAKRGGATKSPESDEPAEERQYQVHPSRERGILQPNEETTVEIKLSGFKQCGRYVDVLDISSSPVGPPHGIDSEEAQEQREAAEGETHFALYMLIRCDVPKLQFDVTYINITHNKLNECISFSFGIYSHGYHYVRVDSHLKNPHAECFSISLHFIDGNEINERVKKLRVQLKCKASRWLTFKSSIVLSVMNHHQYALPLFVSIDEGVDFLLGRASGEGSGETSGETSGEISGETSGVTSGETSGEISGETTKGATLERSLHQHLNTTDLPFDKQNELLSFFCKNVEESKGGSTNGDIYKGLKIGKIKNVYCHEEEGVYPLSEIVGDYLFVFFQKMLLGKSSFPQVIESTNDLFLFFVDLLFDLFSATYPNRNLQNAIAEVKRHRAVVVKDMERQDLLDERLAKHVKALWKCLQEVGTGRLRLAVHHPATLYATSSPRYVSCHFITPLLFTPLQECLPVDHLLYENLLPADLYLPHILANVPDHFHVEEKDEEDYGDSAGHLSMRDVESFLLNGNKKVFYFERIFKTHVRLFSYSWVTIFLEILNRKFFGRINISSLVTLRGIKLYSIENKLNENIKMCKISYLVILDTQDREVNKHVSFLSEKKGRSRRKDVTTLTNLQVVSGGTATLRTGGQSINTRNGNPTDGDKGTPQRREKYSPNYVKKRTAKDKPCCAINYFNFYENNFIDLKKIANKISGFCSPGGEEAAQGEAGAPIAQRTPSRPFKKEPTKETAKQTSPNAPNGIVHFQSAECILFEWLYFHYNNVHYAKVEDKRYVFREARGGGDEELSHTNGAMKGGEPEGARRRGSPSGSTNCAPSGNPRFAPSGNPSFTPSGNPSFTTSGNPSFTPSGNPRFAPSGNPSFTTSGNPSFTPSPVGKNPKMSRINFQVEIKRKKKEAEKKKKVKGIDELKDLVMVLYTIISHIPYYLFFKNKIKVKCKSKRDYNTNVEILLIMLSEMKLSSLISRGVLAQFNHLHIYLFLASLYFILPSYLPGYYLILDDFPAYEVDLGLITDEVVNGRKKWKQQPGLRQPPTKGKQQTGLKQPPGKSPLPPQSKANPHTDGGGEKTGCGDGHSLANERIITVYNLNSHKCKYEVFLIGCHKYQLDRNHLCIDPLKAEQIKLKIDRGYDEQALSYSYHTSIKLSSFKKKRDKGDMCDGEEPHLEDSCSVSSCSSEECQCPSDGADKVDAGTSGESRPTPGEDYSILVLRAEGNHLQDEQHSEKYICIKLVERDEKGKIELTSPGASIRGKQNEGKNVKANLHNGVPRSEVNKMRTANSSQLNIMLNDSEVKCFNLRGKVYERKCLEFNLCNNTGKEVEVRSYLYHVYGKDLKEEKRRGNLEADVVSSWGNSQCAVCSLVSANLRRHLSGTPTHFSCFHLSGAPPHFCCLQNERKKIQVQFCPFAEGSYSCFLLLNVNDKKSNYVVSACKVNCFFNVKNVKEEEVIKMRSQTFSFSYQLEVCPLNREFLHCVKFILCNLNHLEESHFLAYVRGYLKGIQHGGDNFYILCDNEDKVTIERGVATFGVLDAEKYVEKAAHSEGANVEKLHQMVKRDVNYMCLLQIAEQTSGVFEYNCALVRQRGAPRGRNHLSYEELRNLHIKEQQEWFDPFYKVYKIVANVNDAGNTQSLSITFKTSAFLMAKKTIPLYNYTNGVVTYRRVLSDVIDENNNVVDYQIFSCPEYVEIGKDVEFAQYEVSCYSIIGLTCSCCITLYNVNDEEDRIKINVQANVMKPYPKDTIHLKLLNRMKKTAQVEIYNELNSHCEFKVFSDLPILFGGKKIKMLPKERKAYTFFVTSAHIGEYMGCIIFKFHKLLRSGGAEEKTRDASFPFANYFFWYKLNITVELNKPLKILFLETNVGEEVTKEIVLKNNGTQDEDYFLLAYMNEYIERTQVQVPRNDIYVYSIRYAPKMPNCFEGASSGGAPLGGSAALGENHPTVRGDLQNVYFPQNGDSEWCGPREEALRGKPSTDYPNEEAPSRTPSAEVPPNHGAAPKRKPFPPNEKKIFEELISYCKKYIQVDIPYRPQNIGFFFIYNKNEGINYYILMLLSRLRSELEVGRFSTCLSKSCLLHLHFHFNDEDKRYVHLLQEKGDPRAGNYHYEIGEGVKHPQGGGNALKGENFQQGTPHTGRAYQTYDDEWGEEIHRGDAKNGKDAPICKDNPCSGSGSDQEKHELKCIYLSNRKNFFTVNHEDVGRIVLKYRPTVGTSQECYAIVRSNLHGDFLYRISGTYTVKRRIKEKLVFYNSCCLYSFNVNLFNPFDCKVRVKCRFKGGGPSQACSGRGGVGGAEEGAEEGTDERDTERNCLTSMQTERVLLNNERNYLKMMNKENFKIKMRRHFTIMMTYFCKEVYSRRAVLVVMKPLRRGDATREDVPPMITYEYDFVFNNLLRSGRLPGALRCVPIAGEGKRAGEKGNRGEERSQKGEDPGVSTSCNFHSGVDALRIGSRVGGDFTRRSEGVEAENNLPEVADGEDNPPEVSDGEAHPSEQSPSSHTSRSPSAASERSHFTLNASELGTTDVLEGELKSEDADAAEFEKHPPGDKALWRPNDYNVEEDVRRERVLPCAAGGAAGRAAISATDSNEVVHYDGKVFIESMCKAKTCVEVHIDKAKRRAGATPPQGGGQLEGEKQEGKQQAGKQQAGKQQAGQNSALHRNVQRGKYNYKIFLQNVKNVRKGGGSGGGGEGGGECGRETPGMDVKTNALLFDVSEELLNDYLYVHLVEDTAARLVLSLELLALLPFHCTFEVLVSRTGEANEADEAGEANEADEANEVVGESSKGEPPRRRATLERNRINVEAFNCMNVSRQYLNITVDENLCAEAKLNIFYNHTSDAYFDAYVVRHNQLNSSSHLKDEIVNFDVKPKCGILKHGSYNTFFITRANRNGVVSFNNSFLFLIKTERNLFSYIVFSHYRPAPRDALATKEHNKIKEILNENKKRFSVLFSSFKQEKKESSIFNQKNQIIIEDISKSTNEIRNG

SEQ ID NO: 14 - protein, 4409 aa, Artificial Sequence
  Synthetic
MSREVQNKFDLVDVEPKFLEFLYECTDNVETFLENKKNVIKRKRLKIKNICTKTQNIKICEPDSKYLKIKGIKNKKLAPGTSEQIFIEFSFADVNFKTSKFVQTDNILDVVNNVESTSYVTIRSEYTTLNIPIYIKKSIPVFAYDDVINFGVCNANRAYQFALRVKNVGTRRGTFTLSLDDSPRECSDECADDQQAECAQSRKNSGKFHLRKYRSEDMMIQLDQSSLDLDVGETKIINISISSMAEGKMHKEYRVISNQHSYFVEKQRKITIFAIFTSSHTSFLFNNEKTNLVNLHCLYYGSRKKYQGKIKNENNYAIFCIPQVDAVNVFYSKEDLEAYAVAKGLDLSRICPDVYLQEEEHLSEGEKKEKEKLFSITKKRLKGEEHIGITFSRVQGYPIEKLSYQEISFEVYSKCDTDLLHRLSRDTSYLLNFPVVVELSLRVGRSHRETGQGDSATKQVHSTTKQVHSGTKHIHSATKQNHSATEQVHSATLEGAHTLDEEVKICLAFCLTFPCILPSTYLLNYDTITPNEGKTLIIELKNRNKFMKLNYTLSKIPYLTLSKSEGVISPQGKVVLSLKLQCDSVKMMDDYMYLYFCNNLYFIVLLVRGNIISSNALRKGSSSILLNLKAEPFRRNETHGKIKQNHDEVYEQILKNLELERVDRNFYQLDGKLDKYKYNERFINFLKKNKKKYNDVLKLMYEKRKKGEESIQNASPSAASPDTSEQMVKGKDRLSKILKDKFIYVNDPPVERISNTYINKGVYEQERKKYIQMKTKNLKIKNEERAQGGEEEEPYLMQFDMLDEEELNKVEFVRMIQYGYIFLGADYTRKFVVFNRNKHCNVKVALTHGDNITTDGEKILLVGRDSHKMRNVKLNIKCVSREEEVHQDDKNCNDICLNSGKIGSQTGVSTSFFGEDIITGSCADLHNDFFLLKMFEEKISLMINDSHKREVTLGATLVFLNVLVNPTTVYFRFHDEDVEMVCERHLVLYNPFNVPLNVGMVCNQAHIALDSEILVPPNSHKIVPLNFVATESATSVSETIRLYLDGSRLYKSLKCKFIANKCSYRVTPTELNFENILLNRNYVKEFFLINTGDSPVVFRCNYKPDCVSLLSKYNYVKKNEKVKISVLLNLKEGKKIKDKIVLTVRGAPQLILPLNVKGTPSNVLICEGIHIQQRRGELKCYEVKILNRGSCDETILVDTSSVDFLNVQMGNKKETTFSKCISKEYKDANKMEDVVTVQRIYSEEYDDLLTDECAKYHYNNFLKLHNSISVLYQNKGQILLKDRGEVKSMYRVVVPSKCFLPLCIQCYSSKAIRKEIAFQDLLHHNRVTYDITEELIKIDIEEGHLDVAPSFLHFTDLIPHQADEAYISYFSKEKTKRITIRNVFSQPVQWAIRLDEEKRREIHVVTNVHRGVPEFTERKGGMGKSKRMDSVKSRGSSKSHQTDEPIWERQYQVYPSRHRGILQPNEETTVDITLSGFKLCGRYVDVLDISSSIFSDQNAGCIDGGNDASNDASNDARNDARNDVSNHASDDARNDASDMLSNLVGEAEPAQKEEAEREVRFSLYMLICCDIPKLQFDVTYINIIHNKLNDYCSFPFDIYNHGYNYVRVDYHFKNLHAECFSISLDFINGNEINEQVKKLRMSLKCKATKSLKFKSHIVFTVMDYHQYTIPLFVNIDEGIDFLLEKAATSSSVMSSIEETGEVIDEVVGEVIGEVVGEVIGEVVGEVIGEVVGEVIGEVVGEVVGEGIPKQMHHMDDYLPPENSPKGASKGTYPAGKPTEGIIMDSSLQQHMNTTDIQFDKQNELFSFFCKNMEEVKKGSTNGNNIYKGLKINNIRNVYYNEEEEGVYPLREIIGDYLFVFIQRIMLRKSYFPQVIESTNDLFLFFLDLLLDLFSSIYPNRNLQNIIVELKKRPTVVVKDMKREDLLDEMFTKNVQTLWKSLKEEEYIPLDHIIYENLLPADLYLFHVLDKVPDYFHVEEKHYEDNTENLSIKNFESFLLNGNKKVFYFEKIFKTHVRLFSYSWVTIFLEVLNRKMFGAINMSSLTNLRGIKLYSIENKLNENIKMYKISYLVILDTEDMEVNKHGSFLNGKKGRSRRKEASTLTKLHVNRGGTGTRNVAKSSLNTRNGNLIYRDKSTSRGQDKYAYNYVKKKTTKDKPSCTINYFNFYENNFIDLKKIVNKISRFCYSGGVEELPNGKQETVQGKNELESSKGNTNIEGDDIPTGGETNTPFKKKSTKEDVKESTIKYSKDTTKDAPNGIVHFKSAECILFEWLYFHYNNVYHAKVEDKRYIFKEARRKSDDELSYTKGNSNETQERGKPEGSRRKGNPSGSPSFVPSDSHHVSASGELVPSQVHENTKMSTSNFQVEIKRKKKEVQKKKKAKSIHELKDLVMILYTIISHIPYYLFFKKKIKLNCKSKRDYSQNIDVLLIMLSEMNLSNLISRQVIVQFNYMHIYLFLASLYFILPKYLPCYYLVLDDLPAYKVDLSWINDEVVNSKKGGKSKPMGKAQQSSRGKQAPQKKTNAPIDGENEKTSCGDDQTMADERIITVYNLNSHICKYEVFLIGCNKYQIDRNYVSIDPLKSEQIKLKIDKGYDEKVLKYSNHTSIKLPYFKKKRHKGDMCDGEEPHFEDNYSVSSCSSEDCQCPSDGADKVDAGIDEDSRVSQGENYTILVLRSESNRLQDEKDSEKYICIKLVELGEKGKIELAPPGTNIRGEKNEGNNVNANVNNGGTRSEVNKVRTTNSSHLNIMLNDSETKCFNLRGKVYENKCIELNLFNNTGKEVEVNSNLYHLYVKNLKKEEKGKENFETDVVNKWGDDHLGGHNQCVVCSIVNANLRNHLSDTSTHFSCVHLVDKNRFTCLQNERKKIQVQFCPFAEGSYFCFLLFNVNDKKTNYIVSACKVNCSFNINNVKEEEIIKMNSKIFNFSYHLKVCPVNREFLKCVKFILCNLNHLDERHFLSYLRGYLEDIHKGRKFFSVLCDNEDKISIEKGFATFGPLDCNKYMEKILYSEGVDIDQLYELVKKETNYTCTLEIAEKTNGVFEYNCALVSHRNVLQGKSKLSYEQMRKLHIKEQKEICDPFYKVYKIIASVNDGNSSQSLSITFKTSAFQMVKKSIPVYNYTNGVVTYRRVYSDVIDENNKIVGYNIFSCPEYIEIGKEVESVNFEVSCYSIIGLTCSCCITLYNVNNEEEQIKINVQANVMKPYPKDTIHLKLLNRTRKTAQIEIYNDLNFHCEFKVYSDLPILFGVKKIEMMPKQKKTYTFFVTSAHIGEYMGCIIFKHYKLLRSGHGEEKKGDSFFPFANYFFWYKLNITVELNKPLKILFLETNVGEEVKKDIVLKNNGNENENYFLLAYMNEYIERRQVQIPKNDIYVYSIKYAPKIPNYFNEDGNGESASGENNDPTLQRDIQNFYYPHNGDSDWCGPPGGDEPAHGGKEPWELSPNQGATLKHKHKPFPPTEKKIFEDLISYCNKYIQVDIAYRPHNIGFFFIYNKNEGINYYILMLLSRMKSHLDVRNFSTCLSKSCFLHLHFQFNEEDERYVHLLEGMETPPSSNYHYQIVEGVKCPPGEKHILIEENFRGAVDMCRAYEEYDDKWGEEMQKGNANNYMDAPIYKDNPYNGGARDQVKHELKCIYLSNRNNFHIVKHKDVNRVVLKYRPTVNTSQECYAIIRSNLHGDFLYRIHGKYTVKRKIKEKLVLYNSCCLYNFNVNLFNPFNCKVRVKCRFKGGNATQLCNHMCGAQGMKEKDIDEKYMKSFQKERVVLNKERYYLKILNKENFKIKMRKHFTIMMTYFCKAVYSRRTVLIMMKPFRRDRTHVDGPPMIIYEYDFVFNNFLRSRRIQNVLQCIPAVGEIKDEQGRGNCDQKQPSTECEDLGVNTSCYFHSSVDEPESGNQASGNFTMRHSERGDVEANLSEVSDVEAHQSEQSESIHTSRSSSVASERSHFTLNASELGVMDVAEGDFKLEDADTIEIGKNHTCFTHPDDKALLSPNDYDVEHEIKHERVLPCASDNKEVVHYDGKIFIESMCKTKTYVEVHIDKTKMGTWASCPQGMPQGMDQLEMQNNSLHRNVEMGNYNYKIFLLNLKNVRKNMDTVDETVVGGTGGAIGMDVKMNTLLFDVNEGLLNDYLYVHIVEDTEARLVLTLELLAFLPFHCSFDVFVSRRGGEGATHHGESGKADQAAATNKAEATNQAAATGQSVEEAYNVVEEFCGGKPRHRCTTFEQNHISVEILNCMNVSRQYLNITVGDNLCGETRLNIFYNHTRDSYFDAYMVKHNQLNNSSHWKDEILNFEVKPKCGILKHGSYNKFFITRTNKNGLLTFNNSFLLLIKTERNVFSYMVFSHYRAAPSDALATEEHNKIKEILNENKKKFSVLFSSFRKEKKESSIFNQKNQIIIEDISKSTNEIRNA

SEQ ID NO: 15 - protein, 4671 aa, Artificial Sequence
  Synthetic
MNEEVTNKFDLIDVEPKSLEFFYDCAENVDVFVKNKNNIIEKKVLTIKNICTKTQNIKIFESDSIYLKIKGLKKKKLAPGTSEKILVQFSFCNFDTKKCKYYSGNSNSNSGDNNGNSKSNNNNKTGLLRKLNNIECTVYVKVQSEYSTLHIPIYIKKKIPLFVFNNIINFGVCKNNMTYHFSLQVRNEGTKKGTFTICKENFIEEGNEQSRNNEIDGGKHALVRFNGNIETKEKEDKIIKKEMSEKKNNLIIDFDKTSIDLDINQSEIINIKIINTKEEKIQRTYKVLTQEHSYFCEKPKNIIIIAIFVNSQTSFIFDNVKTNQINLNYMYYGNNRKFQGRLKNENNYPIFCKYEICKVNVFYSIQNFQKYADDNNLDVGLLMHDLNDKAENELSEEEKREKEKLFTIAKKRLKAEDNITVKLDKEEGYPVQKLGYHEILFELQSKCDINFLEKINRNTSYLLNFPAVLELNLHIRKVDEDKKKGNDDTPNRDGYTDMHSSTNRGDEEIKLFFIFCLTFPCFASSSYFLNYETVTLNEGKTLIIELMNKNKFLKINYCISKIPYLTLNKKEGCIVPLGKDIISLKLKCDTIKNIDEYMYIFFCNNLYFFVLLIYAQVKSVFASRHVSSTILINKKRTNYNGDVVDSKFSSSSTKKKNDEIYEQIIKNLELERVDRNLYLEDGKLDKYKYNESFMNFLKDNKSKYNDILKYMYKKRKKRGKINNNNNNDNNNDNNNDNNNDNNNDNNNDDKVVHDKDYKREKRAEERINKIVKDLNIYLNDTGMGRMDEKSKMSKTNRTYKTGKACKISSTCIQHAVYEERRKKYMNMKKKIFKEKDIEEMSKKDADLFFHEILEQHQLNNVNFPKIIHFDNVLLNNNYQKQFVITNSNKDCSVKINFIHSDNIELNKDILILSKNSDKHVNINFKLLSHTNLINRYRNYQNDSIFKQSSEIFNEQLNEKTSPQNEEIKCVLNEKGNNSTYDESNKIFSNSINDFFLEQTYKEMIQLKINNAYTEFIIIKANLVYLNVLLVSDVLYFRFQGTDLGMIQENKLVMYNPFNTPICVRTTCNEQLFKVDAEFTIPRNAYKTVPVKFIAPQNASNVEEFIQIYLDGSRLYKSVKCECFVNKVFYKVNTTEINFEHILLNKNYEKDFYLTNTSDSLLSFECVYKPDCVSIISKYNFVNKNEKSKITVCINLKESEKIKDKIIFRIRGSENLVIPITAKPSPSNVLLCNNVQVAQESNEIKNYEINILNKGLCEESIILDLSELSFLNIYTNNKGQKKLMMSNNKEYKNADEMIDSQMILSKISKLEHEDILTDECTKYYYNQFIKLYNFISTYYKHKGEIRFSEKGEKGVPLPKMYRINIPPKSFASLYFQCYYNKCLKKEIAFNGLLRYNGKNDNSNNDNSNNGNSYHTAYGMKEEEVKIEIRASCLEVEPYFLYFEDMLPSSSDKALITYFTKEKTKILKIRNASSQVVRWEIDICNEKEKQNYLIGIRGGDCRERVEKGTVHSTLEEEAQKKGRKDIPKKGRSEWGHMGQNGEKTVQALKVERGTESKVGKRNERGEIGEKGGVGDTGYIFDKEKTDNTGKQNYWQKYEIELGKKEGTLEPKGEVEIQVKLKNFTQSGRYVDILSISYDPVEDHIERSSNEKEGGSKEIIISNYKMELKKINYCIFMLLSCKIPRLLFDVTYINIIHNKLNEYITFPFHIYNSGYNYVRVNYYLNNLYEEYFSISLNFTNGNEINEKIKKVDVCLKCMAIRNLKFKTYIIITVLDHDQYVIPLFVNIDENIDYLFDKLDQENEHLHVNHSPSMPALHNMHIQCGKKNLSELEKIKDNPIGYRNNDQGIEDRDDGYGDTQHNINHCSTYRDDRDKQSAKDMSHTRRSNKEKTNGGECVYNSEYESTSCTCSSVCCDDNMLRCNCQERNSKFLSFYSKNAGQRDIRKSVCIYKELKIKHITNVYYNKEKYLLNISEITKDYVFIFLQNILLSKYYAPSVIENTNDLFLFFINLLYELFLLYPNKNIQNIVNIINDVKSYKNISMQDLEKNNFLQEKFLKNMKALLKYLEEENLLVYHIIYENLLPLDLYLWYIFDKVPDYFYLKKGKGGEEEINKRKCIQIEDVENFLTNGNNKIFYLDRIFKTHLKLFSYSWITILFELLNKKLFSCVNVTSLNKLRGIKVCNIENKLNENIKIYKINYLIVLNKQDKEPNKHTIFLSEKRKTSKRKENSSSTNITPKDNNPYEDKSAKVSSVINMGKNKNNYLSNFYMKNGRPTHHNSIIKKKKMNNGKNTYTINYFNFYENNFIDLKTVVNKINNFTHCEEFEQGVEKERDISLEKHEQIINRDDKETTQRNIRNKTYSKGEEQEKEEKDNVQFKRDTVVKEQKPCFHYFKSAEYILFEWLYFHYNNINFANVEYKRYLFKEDNEKIKKQQQKQNDLIFPNVPMNKSKEEEETSSKCMENNDVPLILNRSNFYVEKKKKKIMEKKDKITSICDLKDLVIIIYTIISHIPYYLFFKNKIKTKCISKKDYIYNIDILLIILHEMKLDKLISRQVLIEFNYIHIFLFLASMYFILPSYLPSYYLVVDDFASYTNCVGLIKEEMISNNKNKKKNTCNQNENEKAGIIKLNLTTPEERVITIYNLNNYKCKYNVFLIGCNKYQIDRDHIVIDPLKSEEIKLKIDKNYDQTICKYNNNTSIQFSTFKNRRKKKETIDETTTYFDESYSFSSCSSDDCKCNSDEAEKISVSNNGDDSLTPNENYAILVLRAENNYFPDDNDDEKCICIKLIEKDTHEKSDLQASNLKKEKNEWNDLNINVKNSETRNNNISKVHLSNSSQLNVMLNNSEVKCLNLRGKVYENKCLEINLINNTGRNVEINSNIYHLYLKDLKKEEKKKKIFEIDIINNLNISNDQIYKVCDVCTIINSNLRNHLNRKENYFSCFYVDNTIPIIISRENEKKKKKIQLHFFPFMEGYYFCFILFYVKDVKTKYLISMCKVNCFFYINNIKEEEIIKMNSQLYDFSFNIKLNPINKEFLKCVKYILCNLNKMDEKFFLIYLKSYLQNVSKNRSSFYITCDRKDKMIIKKNFVSFENFDYNNYISHINTPNLDIDRLYDIIKEGINYLCELSIKEESNGVFEYNCVLLMRKGEAEKQNKKSHKHTQQKEGLKTEKDADCNMLSYEQIGHSHLREQIGNHEICYKLYKIIANVNNKKNNNINSIITFNTNAYLETKKTISIYNYTNSTITYKCTYSDTVDHNNNKINYKIFSSNEYVEIPKDVETFNFEVTCYSIVEVSCSCFIIFTNIKDEEDQIKIKLQANIKKPYPKETIHIKLLNRMKKTVQIELYNELQFHCEFKIYSDLFILFGDKTIEMLPKQRKVYTFCVRSAHIGEFVGCLIFKFYRLIGAERGDLHRSNVIVSNISNVSSESRSNVSGESRSNVSGDDTREHLFYYFFWYKLNITIELNKPIKILFLETSVGDKVNKEIVLKNNGNESEDYFLLTYMNEYIEKKQVKIQKNGTYVHNIKYMPKIPNYFHNIKEASDNFKLSTKRNKSFHYCSVDQLDKEVEGTNPLQSGYNDFPSGKNRDCTYKGGKEMDLAKELHKFYFPPNEEKEEEKKEVEAKWEEEKREEVNDDEDEAKSQGEDEGKGQNSHLAEEDKKETAKKCEIRNRSRSRSRNRSRNRNRSRSRSRNRSRNRSRSRNRSRSRNRSRSRSRSRSRNRSRSRNSSRSRSTSTSINVLRGVRSSSGKDPQRKSPSRDHSRRKNKSDMLRKKCKKNVERDNKKIFEELINYCKRYINTNINYKNQNIGFFFIYNKNEGINYYILILLAKMRRELVIDNFSACLTKYCLLNLHINFNNENKRYVKDLNTNKGTCYSDNYSYEIVECVNKNGGWEAESELAGKCKDTQPDVSVNKHVNRGKIYGDTYDSTISRNNQINIDDHKYDNANVEEIINSQIKCIYLSNKTNYSIFKHTDKNNVIIKYKPTVKTSQECYAIIRSNLYGDFIYLIKGMYTVKRKIKEKIVMYNCCCLYHFNINLFNPLNCSIYVKCRFKREKSKNDELYDNFYLDNNKVGRKAEREECCFSSMEHEKILLNKERSYFKILSNEHFKIKKRTHFSMLISYFCKEALSKHTLIVTLKPFGTDKASDGTLPPIIYKYDITFNNVVRQGIEAYSLTGSGAQSTDELTARGDSSSGSSGRSRSGDGSSDRSSSSSPSRRNRHACESGGTCEKTKSYSVTSQSSSNMLNGSELSKSDLFQRGLKKKYNSKANELVEEQEICLHACDEILVEDKEEKDKYERNMSYVVDRKEEVVYCDGKIFIESISKKKTCLKINIDKRKIMERADYNNIINELENCKKEKSILHRNIEKDKCNYKIFLINLKNVRDATMDEQNKTNLHMNKILFDIREQLLNDYLYVHIIEDTASALVLSLELFAHLPFHCSFFIMIKRVELGSREVGSDEVERGEMNSEEEDNYVLVESLKSEKRKELTTIEKNYIFVEIFNYINLSKKYINVIVDDNLCSETKLNIFYNHTNDSYFDAYIVSYNQQSNDPFADEIMNFDIKPKSGILKHSSYNKFVITRSNKNNIITFNSSFLLLIKTEKHIFSYIVFARYTPASDLHTAFSLYFQQEQNKVKEMLNENKKKFSAVFNTFKQEKKESSIFNKKGQIIIEDISKSTNEILNC

SEQ ID NO: 16 - protein, 4464 aa, Artificial Sequence
  Synthetic
MNEATNKFDLIDVEPKFIEFFYDNVEDVDTFVQSKNNVIEKKILKIKNACTKTQNVNIFESESRYLKIKGVKKKKLAPGTSEKILVEFSFSNLDVKKYRFGKKKDILNILNNVECTVSLKIQSEYTTVHIPIYIKKKVPIFIFDNIINLGICKSNRTYHYSLKVKNGGSKRGKFTISMDNIVEEEKTEQTHDYKRDEKQTKVHFNGDINKRKKEEIERKDVHTSKNDLFVQFDKTTVDLNINETATINITIKNKREEKIYREYKVYTKEHSYFSEKTKNVIIIAIFTNSQTSFIFENEKRNQINLNYMYLGHKKKFQGQIKNENNYPVFCTHKVGKVNAFFSMQEFEAYSEENGLDIDQLSRGEDDNNEGKFVGTFVTESPCSSTAYSFSYTQNTFTSLTGSRDFLDTTVLWTSALLIYFLFYFFFLSSFPIHILADMRGVTQNEMCEEERKEKEKLFAIAKKRLKQEENITVKLSKEDGYTVEKLSYHDVSVEVQSKCDVRFLEKVNRNMSYLLNFPVVVELMLYVCSGESGVSGSGGIKCCHESCDNRNLLDEEIKLFLVFCLTFPCVVSNTYFLNYETVSLNEGKTLIIEVTNRNKFLQVSYCFSKIPYLTLNRKEGCIDPMCKEIITLKLKCDTIKKIDEYIYVFFCNNLYFFAMLMTAEVKSAYALRQASSLHLEQGKGEKGLTNRGSGDRDLGNRQDDEIFEKIIKKLDLEKVDKNLYLVDGKLDKYKYNENFIAFLKNNKKKYNDILKYMYEKRKGRRKDKTNMDMKEKEEECMEKKKLKKKISKVINDIWVYINDTGLTKISTTCIQHSVYEERRKKYMHIKREHLEIKDTNSASNECMNVPCDEVLEEHQLDKVIFPKNIHFNHVLLNISYKKEFTVHNSNTDCNIRLDFTHSDNVKIDNDMLIVGRNSNKNINLELKISPFFKKVENMTNEKGTTTCLFPTPSLQIERENNMLFNNPEIVMIPSVNDFFLQQIYTERIPLKLNNIYEQQITVKANLILLNVLIKEQTLHFYFENSDVQMCCERQIILYNPFDFPVAVSVACNESLFQIDAHITIHPNSCKMVPVKFLAPPRAGVLEDFIYIYLDGARLVQCMCFVNKCSYKTDITEINFENIILNKHYTKDFYLTNTADYPLIFECSHKPECVYVQYKYSYVQKNERLKVTVSVNLKDGKKVKDKIVFSVRGAENLVIPISAKAVPTHVLFMRGLHIKQESCEMMDYKMDILNKGICEEKVILDLTELNFLSISISIREKKEKKTLTYNSKEYKHAEEKRDDFPILREIFHVEYEDIITEESTKWYYNKFIGLYNFICNYCRSRGSIQFTERGEKSSQCLYMVNIPPKSFVSMYINCYYDKTIKKKMKFNSLLLQNKIIYEKKEEEIKVEIDSTCLDISPAFIHFTNVLPSNSHRSLISYFTKEKKKTLKIKNVSDYPIEWKIYVYTNEQKKVHLMRRTGGKARATATHEGNQTARGVFTGEKAAREIATKQITAKHITAKQITAKQITAKQIAEKQSVAMGVSNRDIETKGDLVMGRDEVEEVAKAYELDVSKQSGLLKPEEGTEIEVKIKNVLQGGRYVEILGISATFVDNEMGKKTDSNVGDTEIEVLKSTVRREEKTYCVYMLLTCETPKLFFDVTYINIIHNKLNEWFTFPFHIYNSGYDYVRVKYHFNNLHEDYFFLSLDFEKGGEINEKVKKINVSLKCKAYKNVKFKSNLSVSVLEHDQYNIPLFVNIDENIDYLLDKAGGVEEVSFQRGNKNGEKTQKGDIHVGRKMLDNIDEREDYRYDNPWKEQLDDTTPFEGCNTESNISKQTKDTLLPFFCSNSVEKDEEEIVNIYKGLQIRNITNVYYNKENFTHNLTAILNDYIFIFIGRIVLNKSYLPYVIENTNDLFLFFLNLLNDLFLLYPNRSIQKLINGIKFYENIAMTNFEKNTLLHDSILQNVKGILKHLKEEKLLVDHILYENLLPVDVYMYYIFNKVPEHFYVKGKKEELGKNDNGDSNEYICNVDVNQFLLNGGEGVFFFDRVFKTHLKLFSYSWITIFLEFLNKNMCSCVNVNSLSKLRGIKLCSIENEINENIKMIKINYLIVLNKQDRELNKHTAFFDERKRRSRRSEGTSSGTICSGNGNASSSGRGKGMQRMPVGVIGMEKGKKTMPIKEAKGDPRNGKNAHHGKRMKVGLIKNSSAINYFNFYENNFIDLKKVVQKINSLNCEGGDAEGKKRDDQLTLEGSRKERDTNSPKGSSPMEELREGRNNGILKREVEKEQTRNFHQIKGVECILFEWLYFHYNNVHYSDVQNERYVFKESWNENEKGVADLGKLPSSRKVVNVAEVRREEVEEEVTPQHEVNEVVNQHENSEGRPVKLNKSDFYMEMGKKKKRKHFSEKKDKITSVHELKDLVMIIYTLISHIPYYLFFKNKIKVHCKSERDYINNVDILLVILNELKLNNLISRKVLIEFNYIHMFIFLTSLYFILPNYLPSYYLLVDDSSSYKNDICAINEELIREKKTKNVRVQSENKKIGIPKSVSKVEERVITIYNLNSYKCKYNVFLIGCNKYHVDKNFIHIDPLKSEEIKIKIDKNYEEAIFKYDYSTSIKFPPLKNGFPSDAPRFDDSEGTDASTASSHMGRSSSTSCLSDESHEEDAIMGKKTKHRSFTKNENYAVIVLRSECYLPEERDSERYICIRLVEGSGEEETVILANKGNLKDKKNEGSNLNVDMRVSGGRNDVSRVRPTNSSQINIMLSSSEVKCFNLRGKVYERKNLEINLINNTEKRVEVNSNVYHLYIRDLKKEEKKKDNFEMDMLTVDKGTHEMYNQCVVCSMINTNLRNYLNEHEHFFSCFYVENGKDLTIGKNERRKIQLHFCPFVEGNYFGFLLFCVKDRRTKYLLSTCKVNCFFYINNVKEDEVIKMNTLSYNFTYDMKLNPINKEFLKCVKFVLCTLNKMDKKYFMAYLKSYLKSVNQSRHNFYIICDKYDKVAIKKSLFSFQNLDYARYVSGVDAGSSGDVDVDELCDAVKRDTNCFCNLNVREESGGVYEYNCILLVKKNHEEKKKDISNFVTYEDVRNMSVREQNCIHEMCYRLYKIIANVDSEKANKVFTIVFNTSAYLLSKKSIPIYNYTNEKLTYRCIHSEIIDQHNNKIDYKIFKNDEYVEIPKDIETFNFEVICYSIVEVVCSCIILMTNVENEEDQIKVNVSANIKKPYPKETIHIKLLNRMKKTVQMELYNELDFHCEFKVYSDLPILFGDKKIEMLPKQRKVYTFFVRSAHIGEFVGCLIFKFFNLVNMKGTHASTRYSSNHHDSDRPNSTVDSFPFSDYFFWYKLNITVELNQPLKILLLETTVGDEISKEIVLKNNGREKEKYFLLTYMHEFTERTQIEIPKNDIHVYNIKYAPKIPNYFDNLRDTMECYKLDSEQEKIDPFCGTTISNSLGREKERVSSTEGDAPSFTGGGFDIAQELSKFYFPPNEESEEEMLVESAVTRESSSGRAVIGNVANGEEANGEEANGEEANGETVRGEKDEYAASRVKNLYLRSETMKNGGEQPKKTASMGVKVRSMEKKEHPILRQNVKHKGKKNFPHNEKRLFEELIQYCKRYINVSINYTNQNIGFLFIYNKKEGVNYYILILLAKLRDQLTISNFSACLSKSCFIHLHFDFNSEEKRYVSMLSSKENAHSNEYFYQVGEYVRQRGGSDAGKDKHKVKCIYLSNRTNYTIGGIADGGVDVDVGGLVCEREDEHTDEPGNHVVIRYKPTTNSSKGCYALVRSDIYGDFLYFVKGVYTVKRKIKEKMIMYNSGCLYHFNVNLFNPFNCKVCIKCRFKRRRDERGGGSHIISDSSEHEAEKRMKERSVHSVSVKKEKVLINNEKKYFKILSRENFIVNKRRHFSLLMTYFCNKITSVQTLEIVLKPLQDITNRGSFQSIAYQYKFVFKNTQKGSLIGEKHLVCDVPNDEVTYHNRFDENSLTDSSTGGEEPYPCNVVKKVDYNHVVYTKLNEVFTTEEGEADREEEKHSWRMISTSKWEENMEMHTNEGIASPGCGSTTTDSECTQDGDLVSTPVKKTPFGVCEDRKDVMEEQVEQENCGLSSKEVVYYDGKLFIESICKTKTQMLIYINKKKMMEREDYDKFIDELENRKREKNMLHRNVEKGKYNYKIFLINLKNLNSNWGGLNGKTCVKMNNILFDMNEELLNNYLYMHIVKDTPTDLVLSLELLAYLPFHCSFCVVVKRVEGRGGAAMEEAEEVRETGEAGETGEADGIDWEDGIYADAGERCVQVECLKREETKKFTIVEKNYISVEIFNYMNVSEKYINVFVDRNLCSETKLNIFYRHTNDSYFEAYMVSYNQMSNYILTEEIKNFEIKPQSGILKHGSHNMFLITRINKNNLVSFSNSFLLLIKTERNIFSYIIFSRYSPPRGLRTTREQNMLKEILNENKKKFSVVFNTFRQEKKESAIFNKKDQIIIEDISKSTSEMRNY

SEQ ID NO: 17 - protein, 4891 aa, Artificial Sequence
  Synthetic
MNEISENNLNILDVSPRNVEFFYECLEDVDEFAQKKKENIIEKKSLKIKNICSKIQNIEIFETEYKYLKIKGSKKKKLAPGTCEHICIEFSLYNNVDIKKYKNDKRKEDVLNIINNIERTIFLKIKTEYTSLSIPIYIKKKVAVFFYDNIINFGLCKQNMTYHYPMKVTNVGKKKGTFTISSDMYQSMKEKGNHIFFQDEKGERKKKEFCMENNKGDHQNEDNHDIKSNHNIKSNHNIKSNHNIKNDDNIKNDHHIKNNHHIKNNHNIKNNHNIKNNHNIKNNHNIKNNMKEKNLLVDEKKDNTLITFDKTSITLDINESKIINIEIKNLKEEKVHREYNILTLENSYFVEKPRNIIIIAVFINSSTSFYYDNIKTKEINMNYIYYGNKKKIQGQIKNENNYNISYQYKMKKVHAFYTLDSFKKYIQLNNLSAGSLEEELCEMEKLLGQEEKEDKEKIMSMAKKMMNIEKNIHVKMNNDIVCNVKKLSYESVFFEIDSKHDLLFLEKINKNKSYLLNIPIIIDITLFVNKSDDEKDKTNKITTGGISKMHNDNNMKIINKRKNINNNYDNNCGNNCDDNYDNNCDDNHDNNCDNNCDNNCDNNCDNNYDNNHDNNHDNNHTNNQHNNNICCTNLLQLHKREEKDRDSSIHHASNCYEEEINLHFIFCLTLPCLITNIYFLNYNKVTTEETKSMIIELLNKNKFLKINYSISKIPYITINKKEGKINSQEKDIITITLKCNVIKKINEYMYIFFCNNLYFFVIFINGEVTQISNINDDNMLTIKNIKNIYDNNMCDEKGITNNDNTYILNNKSLLKKKKEKRYTDDHNNNNNDHDDKIFERILKNVECNKVNRHLYEQDEQVDKYKYNEHFINYLKNNKKKYNDVLKYMYKKRHNSKHNILLKDNIKNKESMIIKKNSSYNLKFSEDNNINKEYNIDHKRTNVLKDIYIYVNEEYINKITNIYIPQHFYEEKRKQYINMKKKNNKQYLVGTMKKEKYLNQSLCLKKNNKINNINIINNDDNFQNVQIIKQYLIYPQIIHFNYILLNKEFETFIHLHNTNSIHPINIYFFFHSDNIKITYPEETKTTCHNKINDSNICTPYQSTKVTIKQNTQQKICVTLNLKNYNIFNNQKNKKCSFSQSENMDNEEEQKICNHIISKRNIKNKEHHNYLDDTYKDEPYNIYHHKTTPFFEIYIYYEYISIKTQDHYDIDKIKVQANLILLNLFINTNILYFSITNMDIDMFYHNYLVIYNPFNITIQMKLKYNEQVLKMKDHISIYPGEYKMVPVQFITSEATACVEEFIYVYLDDFRLYKSIKCKCFPSPCSYKINVNEINFDNVLLNRNYLKSFYITNTSNFPLVFMCVYKPPYVHVLAKRNYANKNEKIKITILINVKEEIKIKDKLIFYVRQRENLILPISVKLTQSNIVIVNCPDIIQESMERKRYNINILNKGMYEESVIINFNEIPFLNINIEDEDTGNKLNYINYKNKEYKHSEEKKRNTFVNIYNLEYDNNIIIDEYIKYYYNEFILLYNNLCNNNYKIKGILHISSDQKCLQNCYKIIIPHNTFITLMIECYINKIYETDLYLYQHVLLNLNNPFVKDEYYKDKLKINIKNEYLKFTPSYLYFENILLCSSEKYLITSFQKTITKKIQIINVYHQNLKCTVKICPYNKEQTKTSQPVKVSSNNNNNNNNNNSIIPSDHDMICTHSTWYENNHTKKKQHYIDISNVPDILKVNEESYINIKVDNIEKEGRYVEMLEICAESVNKKENEEKIKYSLYILINCKDVKLYFDVSYINIIHNKLNQYNYFSFHIYNDGYNYVRANYLFNNIFKEHFFLDLQFLPNNQLNKKNKKIKVLLKYLSKINIQLKTFITITVMDHEKYDIPLFINIHEEIDYLMEKENKHKQNYTHCHNYTHGHNYTHGHNYLHETDELYEKHNGNENSDDIKHPYPYDREIKSEGSTNKSHILSSHLSGDHKDEIPIINNNKKKNISYFTKEEEFALYNNMSIPYSLKYKQNKKMDVDENQNMLSFYRKNMEESSNIYTNIFKNLQIDNIRNPYYNTNEEGIINLKEIVHDYMFIFLQHIIINKNYFPQIIENTNDLYVFFLNILYDLSFIFNSNKNIQNIINELKAYEHMQNGSVDENEFIKNMKTLTELLNQEKLLINHIMYENLLPLHLYLHHIFNNIPEYFYIKKESDKKEKCKEINNLDNTILYDNKNDIDEEEYNKEKNKFLNISDIQSFLKNDNYKGDNNIFYLDNIFKTYVKLFIYSWITIFLDIIFKKIFSFINITSLYNSRGTKLCTLENNIYENINIHKINYLIVLNIENKKINTYTYSCTNEKTQKCIVKKKCIDEKKKENANINVKELSFNNHKNNEDLNIQEKAYFHFYQNNFIDIKEVLHKIGQSTDTDHSKKNQEIKETKEIKVTKEIKVTKEIKVTKNKDSYHTNNLYNNTNNFKNTVKEKKQINKTLKKETHKILNQKKKKKNTNIDEDKKKTTESSTYTEKYEHNKHLNIKVQTNINYYRKIEVILYEWLYFHYNNVYNTKVKKQKFIFTQQKKDISKHNKLYLQYDQNKRNSEIEHTNHKEDYSMCDNVITSKMSHISNMDNQYGNNITCDIPFFVEKKTNKKKKTKKKKKKKKKKNIFKKKKEFITSLYELNDFVIIIYTIISHIPYFLFFKNKIKDPCKCQKDYLYNIDILLLILNTIKLNNLISRNVLIQFNYVHIFIFLSSLYIILPNYIPNYYLILDDFSSYKNDVRVIKGNLQKNTRTNKLKQLHMNNKKKNNNNKNTSDNHVHNNYNQEKQTKKNITCDEKINERIIYIYNSNKYMCSYDIFLIGSNKYTIDKNHITIHSMKHEEITISIDPHYDDHIIKYDCNNEIYLKDINKIKYKKKDKSSEHTNSENRFSDSSKNYSSLSSSLSSSLSSSLSSSLSSSLSSSLSSSLSSSLSSSLSSSNSYIHSRCDSSNIPKNIDTNKTNSKHIKHIHSDKSNNYSILVLRESNYNLLDLKTAEEKFICIKIIEGNNEDSEKSLLGRNKNNIKNKDNEYNSVYDKTKERQIKGDIHKNNNSSDCGEMNIILNDSEVKYINFKGMVYEKKNIQINMINNFDRKVEVTTNVYHIYLNNRKKEIKKKENFDKDIYNLKHTKVEENNIHNNNNNNNNNSSIPFNANYDMYYECDACTNINNHLKAYINKDDINFQCVSLDKEKNIYIGKDNGNNKINFHFSPFFQGTYYCFLLFYVLCTKTKLLLSTCKINCVFYINYIKPEEVIKINYKSYNFTYDLILRPLNKQFLKSIYYILCKLNNSSNNVFFSIYIKRYLDNIYMNKENFFILCDSIKKVKIKDKKISFNNFNYTKYFKDDIINKNIDIDLLCDMIKRDVTYSCKLEINEEMNGVFEYNFYLLKGKQYNVENCLFMIKDGGENYVNDQVSLYINGNKNVNNMFSPPKNNNVNSMNNNNDDDNDDNNDDNNDDNNNNNHNNNNHNNNNHNNNHNNNNNNHNNNYYYYNSCDGDELVPLCKPKGPDNYHYCDVKLYKIICNVNDENNIRTININIKAGIVYKKYIPLYNYTNNNITYKCAFSEIINDKNMIINYNIFTCDELVKLKKDMEIFNFEVTCFCPVGGIKYNSFFILTNVLNEQDKIKIKIQGYISKPLPRENIQINVLNKIKKNIQIEIFNELDIPCEFKIYSDLFILFGEKKKIEMMPKQKKLYTFFIKSAHIGEFIGCLIFKFYKYKDTNNENNASFFSDYFFWYRLNIVVQLNEPMKILYLETFIGQEITKDIIIKNNANQKEEYFLLLYINEYIEKKQIQIEKNDIYIYKIIYLPYIPNYFYNVETSSCSHSKTLTGEENNNYSNEFHESKQLFMDKGEYASDHFIHNEDEKNEKMKNYSLKNYIGDIINSQSNNHVQHNNHIINSLHNFYYSKGEYNNMPQCLHKSKNKDTFTQEYEENNVNTSDLLFEHKQKKNFQSKEKQIFEELINYCKNKYININDINYDNHNIGFFFIYNKKQGINYYILILLSKFKNKLIINNFYTSLSKACYINIHFHFQHEQERQVKQLQEKDKQKKLHSFSYEIIEKMDKMDKMEKIEKGENKNKNKNNIYSASYDICSNNDQPFSSSTHDVYKNHLKNSLLDSYGQNHSSNIKCIYLSNKINYSILQNEDKSSNKIVIKYKPTMETSQDCYIIIRNNLYGDFIYFMKGMYVVKKKIKEQMVLYNSSSLYRFAINLFNPFSCNVCVKCGLKENKITNKNNKKIIKNIKYMKNIKNHNVHNNNTNTVNKKNKHKSSRFKGNQLLLNNKKNYFLIINNENFIIKDKKDFPLLIISFCKYEPSHHKTLIVKLQPIYNKNMNNKQIPYIIYEYSLVFNKTNQQKNQIKSNTLNHFVLSDLYKNESLTHEYQTTENEENENDVEECQVNIHESNENENDMCESGENENDMCESGENENNICESGENENNICETAENESISMYESGDKYTTDSQKSDTANELSIGELRREYTDKMSMSQNGNDDKSKYDYSYDDDCYLINGNDIIYYDKKIFVESICKKKTCVRIYIDKKKMRESQNYDSLMDDLEICKKKKCMLHRNVEKDEWNYKIYLIKLTNVYNNNNNNNNSDDIVNKRMITMNNILFEMNDEMLNDYIYVNVIEDTEEGLLIDIELLAYLPFHCNFFLMIKKKMKEKINGDKKGINILSETCNGKDINILPEYKDNNKYIINGVEKNCIFVEIFNYLRICSKYINIFVDKNLCSEKKLNIYYKNNSDSYFNAYMINYNEYSTDDVMDEILNYDIKPSSGVLKHNKYNKFIIIRTNKNGVVNMNNIFLLLIKTERKIFSYIIFSHYKHTRTICTMDEYNKIYDIMNENKKKFSEVFNTFKEEKKESVVFNERNQIIIEDIQKSTNEIQNI

SEQ ID NO: 18 - protein, 4433 aa, Artificial Sequence
  Synthetic
MGLDAEKNKFDLIDVEPKSIEFYYDRIESFEEFVEKKKKVIEKKVIKIKNICTKIQNIKIFESESKYLKIKGIKKKKLAPGTYEKILVEFSFCDVNINKYKYEKIKNILDVINNIEGTINVKVQSEYTTLYIPIYIKKKVPIFKFNNVFNLGLCKTNLIYDVKLEVKNVGNKKGTLSICLDNINNEAAKNEAVNDDNKNSNTLSNSDNKHIIENNFFIYFDKNTICLNPNESTIINIKIKNKNEEKIQKSYKIITQEHSYFSQSPKNLTMIAIFINSQVSFIYENMKTNQIDLNYIYFGNSKRINGQIKNENQYPVICKLKKTQIKMFYDSKEFASHAVDRGVDVRTLLRDLDNFPEFDVNDEEKKKSEENILDVGNNLKLEEKIKIKLEKQNDIYFIDKQSCTDILLYIEIESCIDILNKIDKNYSYMLNYPVMAEITITANKCDAKCKPSTNRKKAITDLRQFDKLRNGANTVNGANSVNGANSVNGANGESVECSEGCEEDLTFYVFFCLTFPSIVSNIYFLNYDMVGQNEEKTLILELNNLNKFLKINYNFSKISHIHLNKKEGVINPLSKDIIALNLKCNVIKNIDDYLYLFFCNKLYFFVFFVKAQIKKRISIGRSIVIKNDKMIKADGKTKQINRNTIELEKITISKKEDENSNEDIIYEQILKKLDLEKIDKNLYTLDGKLDKYKYNEKFMSFLKENKKKYNDILKYMYNKRNEKKEKQGIKNPIPENDQNEKINNIINDKFIYISDKQVIKNTNMFIHKSIYEEKRKKYIDMKKRKEIKIFNIKKNDDKKEDIQLEYNDILEENQIKNLIFDKVIYFNYVLFNQVYTKIFNVHNKNDECNIKINFTHSSNLNLDKNLLFIKGNSKEMIKTNLLLNLPIIKTDVENCNKTININEIKYRNIFDNEKTNFQILTDHTFNQKEQNNLSLHENENLSNNNKSYLFDHKYYTENITLKINDNYEDMIVVKGNIILLNIVIKINTLYFYFKNSDISMFCENNLILYNPFNIPIPITLEFSKEIFEAKNKLVINPSEYKTVAIKFIGTQNIERMDNFINLYLDNNRLYKRVKCIFDVNKCSCKINVNEIKFENIILNKKYFKHFYLTSTGDTPVVFNCVAKPDCVNIYYKYNYVNKNEKVKIIVSVKLKENKQIKDKIILSIRGSDNIIIPIIVTKVYSSNLLFLNDIKIDQETNELKRYEIKIVNKGGCEENVILNLSELNLSELNFLNIELNKKTENNRIIYNNKEYKNVRNMKDDFMVLKKIFKTEYENIITNKCVKYYFNKLIDLYNFLHNFSKNKFKIIFVENKNNDIKINEKNMYKIKISPKCFVSLYIQCYYNNIIKKEIKFNKILFENKCLHELSNEVININIKNSQLSISPNLLLFQNIIPYNSERAFITYYKKEGIQKIKLKNNSSYPIKLEILLYDKKQKDSLLATSSVDNFVEMERKQIIKNNEESNEKIEINKTLPRAVPKKDSNLNLKKGNIQENVGNKSNEHVEKMYEIELNKESGIIKANEEIEVEIKLKNCKDIGKFVDILEIKTNVVKNEQTDKIFDEKKYCIYILSVIEIPKLYFDITYINIIYNKLNDTFIFPFKIYNYGYSYVRINYKFENLYTDFFFLSLDFLQGNEIDIKTKKINVELKCKSIKNLKFKSEIIISILDYDIYKIPVFVNIDENIDYFLEKVQYEKFDALTSQANNYSLDQYMCKENSRSNLSEIIDEVVWTDKQFNSGKAENGNVENGNFENGKAENGNVENGNFENGNNYNISNVSEQNNELLSFYCKNICTENCNIFKKRCIYKNLKINNIRNVYYNKENEFIEINEIPKDYVFVFLQNILLNKSCAPTFLENTNDLFLFFINLLINFFHIYPNRNIYNLLHEIKIYQNISMQDFEKNIFIHENILANIKQILKSLKEEKLLVNHIIYENLLPIDLYCCHILDKVPDNFYIKNVKCKKDSFEIKDIYNFINNNDDKILYFDRILKTHLKIFSYSWITIFFELLSKEIFNCVNIESLYKLRGIKLCNIENKIYENIKIQKINFLLILNKEDNELNKHTSMLENIRNKTKREKIYGDNNINGGNKKIKYSQINEEKKEKKEKKINNIKNGVIKIRKQPTTIKKMGTKKKDLDLNYFNFYENNLIDLKDLVNKINNLNLCSGEGEGEKGMENKAVLEGCQNKAVLEGCQNKAALEGCQNKAALEGCQNKAALEGCQNKAALEECQNKIASTNQTNISKYYKNIEYILFEWLYFHYNNVYYNKVKNEQYIFKNKENEYNKNESNLINDDTNSKQSNYMKKNDDNSSGYINNEHSYKNFEKSNFYREEKKKKNIVRENKLKIINIYSLKNLVIILYTIISHIPYFLFFKNKIKVECKNRKDYMHNIDILLIILNEIKLDNLITRQVLIDFNYIHIFLFLNCLYFILPNYLPKYYLDVDDISSYKSDRVLIKEEIVKKKNTIKNKKNIDLHKDKERKRNHINNNDKSVDQNERIILIYNLNDNKCKYTVFLIGCSKYEIDKEYIEIDSHKSEEIKLKIDPNYDKNIFKYDYNINIKFPSFTKKINNIHKKDNLLSHENVNNTIPYQKNTSNFGDYSSISSSISSSISSSSIDSFQSSLSYDGIQNVEENNYENNFYHNENYTILVLRKESNLFPGDNDEDKFICIKLVETNKSKLKSEIVRSNSNLLVEGKEWNKLNSNLKFEEGKVNMKNGSGTNSSKINIMLKNSEIKCFNMRGKIYENKSVEINLINDISKKVNINMNMYHIYIKNLKREEIKKGKFDIDILNNKENGNYKNDINRNINEEKGYKECDVCSLLNLNLKKNLNCDKNYFNCFYIENMKPFVSRENEKRTIKLHFCPFIEGYYLCFLLFCAKDLKTKCVMSSCKLNCLFYINNIKEEEVIKIDSQLSKFSYQIKLIPINKRFLKCIQFILCYLNKMNNKVFLSYIKNYLNYINKISDQFYIICDKVGKNGIKEKLPSFQKYNYNDFINQINENNLFLDNFYDQIKEKNNYLYEFNINEENMGVYEYNIILMANNKKKYNEDINKSIQLSKYDELEVDENSIYNACNKLYKIIANINKKENNNIINVTFNQNAYLLSKKCVSIYNYTNKNLIYKCSNSEVLDMNKNKIDYKIFNYDEYIEIDKDVESFNFEIRCYSVVEVECSCFIFLTNVENEQDVIKINVKANIVKPYPKDTIHLKILNKMKKAVSIEIYNELDFHCEYKIYSDLPIIFGDQKIEMLPKQKKVYTFFVRSIHIGEFIGCIIFKCFRFLDVNKIKDSRDIIKNDSNITSKNFSLVDSFFWYKLKVTVDLNKPINVLTLESNVGDVLNKEIVLKNNGNKKEEYFLLSYMNEYIEKKKIEIPANDAYVYTIKYAPKIPNYFDNILKHKNEEDTKDTNFELDEKKEKGSKYNKTKKSECMKRKDEIKQNENIKFSDISNELSNFYFLQNEEEEIKNEGDCLQKNMTKKLNLEKNEKKIFEELIKYCEKYINVDIKYTSHNIGFFFIYNKNEGVSYYILILLARFRTQLDISNFSSLLSKSCLIHLNFEFSNKNKRYVNKLETDQQKNTSYYYQIGEYIEENNINKNNSYNCIENYKLNNGFEFYNSKIERGADKSSNIKEFDGDGNGNENDDGDGDENDDGDENDDGDENDDGDENDDGDENDDGDDDDMSTEQIVRHNIKGIYLSNKINYSMINCDNKKSGNYILIKYKPTVGTSQECYVIIRSDLFGDFIYFLKGGYIEEKKIKERIIIHNSFCLYNFNINLFNPFSSKIYVKCIIETDRSKNYEYNNMNKINCLKLYKSEKSRNIENGKEKILLNTKYKYFKLLSNEHFKIKKRKNFSIFSTYFCKYAKSIEKLIILLYPINNKKKEKKINQYIIYEYELFFNNTKSDFFLETDRIIADKEEEEEKEEEEEKEEEEEKEEEEEKEEKEEKEEKEENNLDNIENVCTKQLNYISNEMSKSSSFSFNGLEKDGSAEDGLEKDGSAENGVEKDRSDEYSLEETESESYSTIEVGSKRNNHILYLDKLSISDSSDVGIEECYEEGMEINNKFDKNIDGINNKFDKNIDGINKKEVVYSDGKIFIQSICKIKTSMKIFINKKKIGEMEKYYQTIKELEKKKQEKNIMHRNILLEKSNYKIFFLNLNNLKNSDKIEKNCKDNYGNEVLNNILFNMNENILNNFLYINIVEDKENYLVISLELLANIPFHCNFFIMIKKVEIRENNKNDELDVRNDEIYQYENDDKILKVKSIKSEKIKEFITVEKNYIFLEIFNYINISKKCINIYIDNNLYSETNLNIFYKNNNDSYFDAYIVGYNQLLDKNNSDEIHNYTIRPKQGILKYNHFNKFIITRTNKINIMAFNSSFLLLIKTENNLFSYIISSHFTPSHELYTEAEKNVIRDILKENRTKFSIFFNEFKQEKRESTIFNKKDEIIIYDISESNEIQSG

SEQ ID NO: 19 - DNA, 13362 bp, Artificial Sequence
  Synthetic
atgccgggggaaacgcaaaacacgttcgacctggtggacgtggagcccaaatttctggagttccactacgagggggcagacagcgtggaagccttcctcgaaaacaaaaaaaaagtaataaaaaggaaggggctgaaaataaaaaacatttgcacgaagacgcaaaacattaagatatgcgaatgcgattccaagtgtctcaaaattaaggggttgaaaaagaagaagttggctcctgggacgagcgagcaaattttggtcgagttttcctttgcggacgtaaattttaaagagagcaaacgtgggaagcaagaagacatcctggaggtagttaacaacgtagaggcaacatcctacgtgactatacagagcgaatataccacgctgagcattccaatctacataaagaagagcatccccgtgtttgcctacgacgatgtgataaactttggagtttgcaacgccaatcggacgtaccattttgccttgagggtgaagaatgtgggtacgaggaggggcgcctttacgctgtccctgggtgactcgccaggtggaaatgcagacgaatgcgaagatggtaaacgggcggagagtgcacagagcggggaaaactcagtcaaacagcaccgccgaaactgccactctgaagacatgctgattcggctggaccagtcttccctcgagctggacgtgggagaaacgaagaccatcaacatcaccgtcagcagcaaagtggaggggaaaatgcacagggagtacatagtcgtttcgaaccaacatagctacttcgtggagaagcagaggaacatcaacgtcttggccattttcaccaacagccatacgtccttcctcttcgaaaatggaaaaacgaatctcctaaatctccactgcttgtactatgggagtaggaaaaagtaccaggggaaaataaaaaatgaaaacaactacggcatattttgcaccccccgagtggatgccgtccatgtgttttactccaaggaggagctccaaacgtttgcggcagccagagggttggacctgggccggatctgccgagatgtattccttgaggaggaagaacgcctaagcgaaggagaaaaaaaggaaagggagaaactattcgccatcacgaagaagaggctaaagggggaggagcacataggagtcactttctgcaggggggagggctacccagttgaaaggctgtcctatcaagaggtcacatttgagattcagtcgaagagtgacgccggcctcttgcacagactcagcagggacacggcctacctgctcaacttccccgtcgtggtggagttgtctctgcgggtggggcgctcgcgccgcgaggccagccaggttggcatcactacaaagcaggcgcacagatcaaccagccaggtttgcagctctgcaaagcaggagcacagatcaaccagtcaggtttgcagctctgcaaaactgccgcatagctcatccaaactgccgcccctctcaacaaaactgccacacagctcaaccgaagtgggggactcccacccctgcgacgaggaggttaaactgtgggcggcgttctgcctgaccttcccctgcgtcctccccagcacctacctcctaagctacggcagcatcgcccccaacgaggggaaaacggtcatcctcgagctaacaaacaggaataaatttctaaaggttagctacaccctttcgaagatcccctacttgacactaagcaaaaaggagggggtaatccccccccaggggaaggcactcttatcgctaaagctccagtgtgacagcctcaaaatggtggaagaatacatgtacatatatttttgcaacaatctgtttttctttgtccttctggtgagggggaacatcacgtctgctaatgcactacggaaggggacatcctccattctgctcgacccaaaaggagactcactgaagaggaacactcccccacagggggactccaaacaggaccgagaccaggtatatgagcagatcctccacaatttagaactcgaaaaagtgagcaggaattactaccaactagatgggaagctagacaaatataagtacaacgaaaggtttatcaattttttaaaaaaaaataaaaaaaagtacaacgatgttttgaagctcatgtatgaggagaggaagaaaagcgagtcagtcagaagtgggacccaaagtcaaggctctcctgacaaaagtgggaaaataataaagggggggacagataaactctccaaaattatgacggacaaatttatatacgtaaatgaccctccagtggacagaatctccaacacgtgcatcgagaagggggtctacgaacgagagaggaagaaatacatccaaatgaagaccacaaattggggagtaaaaaatggggaaccttcccaaggaggggaggagcaaccctacttgttacaattcgacatgctagatgaggaagaactgaagagagtgcaattcgtaagggtcattcagtatggtaatatctttctgggaaaggagtacacgagaaagtttgccgtctttaacaggaacaagcactgcagtgtaagggtggccctcactcatagtgaaaacgtcacgactgagggggacaaccctttggtaattcatagggactctcacaaaatgggaatcatcaaactgaacgttaagcggttttcaacagcggaagagcctcatcatgatgcggttagcgatggggattttcgaagccaaacgggagtgtctacccccctttttgaggccgtcaatttgccaaccggtggaggcacttccacaccggagggaaacacacctacagctctgtgcgccgacccatgcaacgacttcttcctcctcaaaacgttccaagagaaaatttccctaacagttaacgactcacatgagagggaggtgactctcggggcgactctcgtctttcttaacatcctcgtccacccacacactctccactttcgcttccacgatgaggacgtcgggatgttttgcgagcgccacctcgttttgtataaccccttcaacgtccctctgcgcgtggggatggcctgcaaccgggcgtgcgtccagttggacgcggagatctcggtaccccccaattcgcacaaaatagtccccgtcaagtttgtcgccacggaaagtgccaccagcgtggcggaggccattcggctgtacctggacgggagcaggctctacagaagcttaacctgcaagtggatcgccaacaagtgctcctacagagtgacccccgccgagctcaactttgaaaacgtccttctaaacaaaacttatgtgaaagagtttttcctaatcaacacgggagactcccctgtggtgttccgatgcagctacaaacccgattgcgtgaatttactctcgaagcataactacgtgaagaagaatgagaaggtcaaaatatcggtgctgctcaatttgaaggaatgtaaaaaaatgaaggacaaggttgtgctggccgttaggggcgcgcctcagttgctcctccccctgagtgccaagggcaccccctccaacgtcctcatcagcggcgggctgcgcatccgccaaaggagaggagagctcaagtgctacgaaatgaaaatagcgaacagaggaccatgtgaggaagccatcctcctagacacatcccctgtgggctttctaaacatccaaatgggccacaacaacgaaaggaccttctccgagagtaccggcaaagagtccaaggatgccagtaaaagagaaaatgtcctaacggtggagaggaccttcaaccaagagtatgatgacctcctgacggatgaatgtgccaaatatcaatacaacaattttttaaggcttcacaatttggtgtcctcctgctatcaaaataaagggcaaatcctttttaatgaaaagagggagcgaaaaaacatctacagagttgtggtgccatccaagtgcttcctccccttgtacattcaatgctactcctcgagggcgctcacgaaagaaattacactacatagtttgctccactgtaatagagctgcatacgaatgtaaggaagaatcaatcaccgtagatattgaggaagggcacttggacgtgtctcccccttttctccttttcaccgatttgctcccgcagaaagctgacgaagcttacataacatatttttcgagagagaggaggaagagactgacaattagaaatgtgctgagtcaacctgtgcagtgggcagtccgcttggatgaggagaagcgcagggaactctacacggttaggggcgctcacaggggtggggagcaaattgctgagcggaggggagaggcggggaagtcgaggagagtggggagtgacataaaagggagcgccatagggggtagcgccaagcggggggaagcacccgaaacgtgtgtcacacaggggagcgccatagggggtagtgccaaacggggaggcgcaaccaaatcgcccgagtcggatgaacccgcggaggagcgccagtaccaagtgcaccccagcagggagagaggcattctacagccaaacgaagaaaccacggtggaaataaagctaagcggcttcaagcagtgcgggaggtacgtggacgtgctggacatctcttcctccccagtgggtcccccccacggtatcgacagcgaagaggcgcaagagcaaagggaagcagcagaaggggagacacatttcgccctctacatgctcatacggtgcgacgtcccgaagctccagttcgacgtgacctacataaacataactcacaacaagttgaatgagtgcatttcattctccttcggaatatacagccatgggtaccactacgtgagggtcgattcgcatctgaagaacccccacgcagagtgcttttccatctccctccacttcatcgacggaaatgaaataaacgaacgggtgaagaagctgcgcgtgcagctcaagtgcaaagcgtccaggtggctgacattcaaatccagcatcgtccttagcgtgatgaaccaccaccagtacgcccttccgctcttcgtgagcatcgacgagggggtggacttcctcctgggcagggccagcggtgagggcagcggtgagacaagcggtgagacaagcggtgagatcagcggggagacaagcggtgtgaccagcggtgagacaagcggtgagatcagcggtgagaccaccaaaggcgcaaccctagagagaagcctgcaccaacatttgaacacaacagacctcccatttgacaagcaaaacgaactcctctccttcttttgcaaaaatgtggaggaatcaaaagggggaagcaccaacggagatatatacaaaggactcaaaatagggaaaatcaaaaatgtgtattgccatgaggaagagggggtgtacccactgagcgaaatcgttggagattaccttttcgtgtttttccaaaaaatgctattaggtaaaagttccttcccccaggtgattgaaagcaccaacgatttgtttctcttcttcgtggacctcctgttcgacctcttttccgccacctacccaaatagaaatctgcaaaatgctattgccgaggttaagaggcaccgagctgtcgtggtgaaggacatggagcgacaggacttgctggatgagaggttggcgaaacatgtgaaggccctctggaagtgcttgcaggaggtaggcacagggaggctccgtttggccgtacatcacccggctactttgtatgccacttcatcaccccgctacgtttcatgccacttcatcaccccgctgcttttcacacctcttcaggaatgcctcccagtggaccacctcctttacgaaaacctgctgccagcggatctgtaccttccccacattctggccaacgtgccggaccacttccacgttgaagagaaggacgaggaggactatggtgatagcgcgggtcacctctccatgcgagacgtcgagtcatttttgctaaacggaaataaaaaagtattctactttgagcgaatttttaaaacgcacgtaaggctattcagctactcctgggttaccatcttcctggagatactaaataggaagttctttggccggatcaacatcagctcgttggtcactctacgtgggattaaactctacagcatagaaaacaagctaaacgaaaatataaaaatgtgtaaaattagctaccttgtaattttagatactcaagacagggaggtgaacaagcacgtctcatttttaagtgaaaagaagggcaggtccaggagaaaggacgtcaccacgttgaccaacctgcaggttgttagcggcggtactgctacgctacgcacaggaggacaatctataaacactcgtaatggcaatcccactgatggggataaaggtaccccccagagaagagaaaaatattcgcccaattatgtgaagaaaaggacggccaaggacaaaccctgctgcgccatcaactacttcaatttttacgaaaataatttcatcgatttgaagaaaattgccaacaaaattagcggcttctgctcccccgggggggaagaagcggcgcagggggaagccggcgcgccgatcgcgcagcgcacgccgagcaggccttttaagaaagagcccactaaggagaccgctaagcaaacctccccaaacgcgccaaacggaattgtgcacttccaaagcgcggagtgcatcctgttcgagtggctctatttccactacaacaatgtgcactacgcgaaggtggaggataagcggtacgtgttcagggaggcccgggggggaggcgacgaggagttgtcccacacgaatggggcgatgaaggggggagagccggagggggcgcgcaggagaggaagccccagtggaagcaccaattgcgccccaagtggcaatccgcgcttcgccccaagtggcaatccgagcttcaccccaagtggcaatccgagcttcaccacaagtggcaatccgagcttcaccccaagtggcaatccgcgcttcgccccaagtggcaatccgagcttcaccacaagtggcaatccgagcttcaccccaagcccagttgggaaaaacccgaagatgagcagaatcaacttccaggtcgaaataaaaaggaagaaaaaggaggccgaaaaaaagaaaaaagtgaaaggcatcgacgaattgaaggacctagtgatggtcctctacaccatcatttcgcacatcccctattacctattctttaagaacaaaattaaagtgaaatgcaagtcaaaaagggattacaacacaaacgtggaaattctcctcataatgctcagcgagatgaagttgagcagcctaatcagtagaggggtgctcgcgcagtttaaccacctccacatctacctgttcttggcctctctgtatttcatcctgccgagctatttgcccggctactacttgatcctggacgacttccccgcgtacgaggtggacttgggcttgataactgacgaggttgtgaacggccgcaagaagtggaagcagcaaccgggattgaggcagccgccaaccaaggggaagcagcaaacgggattgaagcagccgccaggtaagagcccactaccgccccaaagcaaagccaacccacacaccgatggggggggcgaaaaaaccggttgcggcgacggacactccctggcaaacgagcgaatcatcacggtgtacaatttaaatagccacaaatgcaaatatgaggtgtttctcattggctgccacaagtaccaactggatagaaaccacctctgcattgaccccctgaaggcggaacaaataaaattaaaaatagacagggggtatgacgaacaggcgttgagctatagctaccacactagcattaagctttcatcatttaagaaaaaaagagacaaaggggacatgtgcgacggggaggaaccccacctggaggacagctgctccgtttcgtcctgctcctcggaagagtgccagtgcccctccgacggggccgacaaggtggacgcggggaccagcggcgagagtaggcccaccccaggggaagattactccattttggtgctgcgggcggaaggcaaccacctgcaggacgaacagcacagcgagaagtacatctgcatcaagctggtagaacgggacgaaaaggggaaaatcgaattgacctccccaggagcgagcataagggggaagcaaaatgagggaaagaacgtaaaagctaatctacacaatggagtacccaggagcgaggttaacaaaatgagaacggccaattcgtcgcagctaaatattatgctaaacgatagcgaagtaaagtgctttaatttgaggggcaaagtgtacgaaaggaaatgcctggaatttaatttgtgcaacaacacggggaaggaggtggaggtgcgttcgtatttgtaccacgtgtatgggaaggatttgaaggaggagaagcgaaggggaaatttggaagcggacgtagtgagttcatgggggaacagccaatgcgcggtgtgctccttagttagtgccaacttgaggcgccacttgagcggcacgccgacccacttcagctgcttccacttgagcggcgcgccgccccacttctgctgcctccaaaatgaaaggaaaaaaatacaagtgcagttctgcccctttgcagaagggagttactcctgcttcctcctgctcaacgtgaatgacaaaaagagcaactacgtggtgtctgcatgcaaagtcaactgcttctttaacgtaaagaatgtgaaggaggaggaggtcataaagatgaggtcccaaactttcagcttctcataccagttggaagtttgccccctaaatagggagttcctccactgtgtgaagttcatcctgtgtaatttgaaccacctggaggagagccattttctggcctacgtaaggggctacctgaagggtatacaacatgggggggacaatttttatatcctgtgtgataatgaagataaggtgaccatcgaaaggggggttgcaacgtttggagtgcttgacgccgagaagtacgtggagaaggccgcacattctgaaggcgcaaatgtagagaagctacaccagatggtgaagagggatgtcaattatatgtgcttacttcagatagctgagcagactagtggagtgttcgaatacaactgcgctctggtgaggcagaggggtgccccccgaggtaggaaccacctatcctatgaggagctgcgcaatctgcacatcaaggagcagcaggaatggttcgaccctttctacaaagtgtacaaaattgtagccaacgtgaatgacgcagggaatacccaatcgttgtccatcacgttcaagacgagtgcctttctgatggccaaaaagaccattcctctgtacaactacacaaacggggtggtaacctacaggcgagtgctctcagatgtgatcgacgaaaataacaacgtggtagattaccaaatttttagctgcccagaatatgtagaaataggaaaagacgtggaatttgcccaatacgaagttagctgttactctatcatcgggttgacatgctcctgctgcatcactctgtacaatgtgaatgatgaggaggatcggataaagataaacgtgcaggcaaatgttatgaagccctacccgaaagataccatccacttgaagctactaaatagaatgaaaaaaactgcgcaggtggaaatctacaatgagctaaactcccactgtgaatttaaggtcttctcagatttgccaatcttatttggtgggaagaaaataaaaatgctcccaaaagagaggaaggcctacaccttcttcgtcaccagtgcacacataggtgaatacatggggtgcatcatcttcaagtttcacaagctgctccgttcaggtggtgcagaagaaaaaacgagggacgcctccttcccctttgcgaactacttcttctggtacaaactcaacatcaccgtggaattgaacaaaccgctaaaaatcttatttttagaaacaaatgtaggagaggaagtcaccaaagaaattgtcttgaaaaataatggcactcaggatgaggactattttttgctcgcctacatgaatgagtacatcgagaggacgcaggtccaggtgcccaggaatgacatctacgtgtacagcattaggtacgctccgaaaatgcccaactgctttgagggggcgtccagcgggggggcccctctaggggggagtgccgctctaggggagaatcacccaaccgtccggggggacctccaaaatgtttacttcccgcagaatggggactccgagtggtgtggcccaagggaagaggccctccgtgggaagccctcaacggattaccccaatgaagaggccccttcccggacgccctcagcggaagtgccccccaaccatggtgctgccccgaagcgcaagcccttcccccccaacgagaagaaaatattcgaagagctgatcagctactgcaagaagtacatccaagtggacatcccctacaggccccagaacatagggttctttttcatctacaacaaaaatgagggaataaattactacattttgatgttgctctccaggctgaggagcgaattggaggtggggaggttctccacctgcctgtcaaagtcctgcctcctccatttgcacttccattttaatgacgaagataagcgatacgttcatctgctgcaagagaagggggacccccgtgctgggaattatcattacgaaattggggagggggtcaaacatccccaggggggaggaaatgctttaaagggagagaatttccaacaaggtaccccccacacaggtcgcgcttaccaaacgtatgatgatgaatggggggaggagatccacaggggggatgcaaaaaacgggaaagatgcccccatttgtaaggacaacccatgcagtgggagcggatcggaccaggagaaacacgaactgaagtgcatctacctaagcaacagaaagaacttcttcacagtaaaccatgaagatgtaggcagaattgttctaaagtacagaccgacggtgggtacatcccaagaatgctacgccatcgttaggagcaacctccacggggacttcctctaccgcatcagcggaacgtacacggtgaagaggagaataaaggaaaagctggttttctacaactcctgctgtttgtacagttttaatgtaaatctgttcaacccgttcgactgcaaggtgagggtaaagtgtaggttcaagggggggggcccgagccaggcgtgcagcggaagaggtggcgtggggggagcggaagagggagcggaagagggaacagacgagagagacacagagaggaactgcctcacctcgatgcagacggagcgagttctgctcaacaacgaacggaattacctaaagatgatgaataaggaaaactttaaaataaaaatgaggaggcacttcaccattatgatgacgtacttttgcaaagaagtttattctaggagggcggtgctcgtggtgatgaagccgcttcgcaggggggatgccactcgtgaggacgtccccccgatgattacttacgagtacgattttgtctttaacaatttgttgaggagtggcaggcttccgggtgcgctgcggtgcgtccccattgcaggtgagggtaagagagcgggagaaaaggggaatcgcggcgaggagcgctcccaaaaaggagaagacccaggtgtaagcacatcatgcaacttccattctggtgtggacgcactgcggattggcagccgcgtgggtggagatttcacgaggcgttccgaaggggtcgaagcggaaaataaccttcccgaagtggccgatggggaagataaccctcccgaagtgagcgatggggaggcccacccatccgaacaatctccatccagccacacctcacgcagcccctctgcagccagcgaacgaagccatttcacgctgaacgcaagcgaattgggcacaacggacgtgctggaaggggagctaaaatcggaagacgcagatgccgccgaatttgagaagcatccccccggcgataaggcgctatggagacctaatgactacaacgtggaagaagacgtgaggcgcgagagggttctcccctgtgcagctggtggggcagcgggtagggcagccattagcgccacggatagtaacgaggtggtgcactacgacgggaaggtattcatcgaaagcatgtgcaaggcgaagacgtgtgtggaggtgcacatagataaggctaagaggcgggcaggggcaactcccccccagggtgggggccaactggagggggagaagcaggaggggaagcagcaggcggggaagcagcaggcggggaagcagcaggcggggcaaaatagtgcgctccacaggaatgtgcaaagggggaagtataactataaaatatttttacaaaacgtgaaaaatgtgcgtaagggcgggggtagcggtggaggtggcgaaggcggcggtgaatgcggcagggagacacccgggatggacgtgaaaacgaacgcgctccttttcgacgtgagtgaggagctgctgaatgattacctgtacgtccaccttgtggaggacacggccgcgcgcctcgtgctgtctttggagctgctggcccttctgcccttccactgcacctttgaggtgttggtaagcaggacgggagaggcgaacgaagcggatgaagcgggtgaagcgaacgaagcagatgaagcaaacgaagtggtcggagaatcctctaagggcgaacccccccgcagacgcgccaccctcgagcggaaccgcataaacgtagaagcattcaactgcatgaacgtgtccaggcagtacctaaacatcaccgttgatgaaaatttatgtgcagaggcaaagcttaacattttttataatcacacgagcgacgcatacttcgacgcgtatgtggttaggcacaaccagctgaacagcagcagccacttgaaggacgaaatagtaaattttgatgtcaagcccaagtgtgggattttaaaacacgggagttacaacacgttcttcatcacaagggcgaacaggaatggggtggtgtcatttaacaactcttttttgtttttaattaaaacggagcggaacctcttttcgtacatcgtcttttctcactaccggccggcccccagggacgcgctcgccacgaaggagcataacaagataaaggagatcctcaacgagaacaagaagaggttcagcgtcctcttcagttcgttcaagcaggaaaagaaagaaagtagcatcttcaatcagaagaaccagattatcattgaggacatttccaagtcgaccaacgaaattagaaatggctga

SEQ ID NO: 20 - DNA, 13230 bp, Artificial Sequence
  Synthetic
atgtcgagggaagtgcaaaacaagttcgacctggtggatgtggagcccaaatttcttgagtttctatacgagtgcacagacaatgtggagactttccttgagaataaaaaaaatgtaataaaaaggaagcgattgaaaataaaaaacatttgcacaaaaactcagaacatcaaaatatgcgaaccggattcgaaatatcttaaaattaagggaataaaaaataaaaagttggctccagggacgagtgaacaaatattcattgagttctccttcgctgatgtgaattttaaaactagcaaatttgtacaaacagacaacatcttggatgtagttaataacgtggagtccacttcgtacgttactatacgcagtgaatatacaacgctgaatatcccaatctatataaagaagagtatacctgtgtttgcctacgatgatgtaatcaactttggagtttgcaacgccaatcgggcgtaccagtttgcgctgagggtgaagaatgtcggcacgaggaggggcacgtttactctctccctggacgattcgccaagggaatgttcagacgaatgcgcagatgatcaacaggcggagtgtgcacagagcaggaaaaactcaggcaaatttcacctccgtaaatatcgctctgaagacatgatgattcaacttgatcagtcttccctcgacctggacgttggagaaacgaaaatcatcaacatctccatcagcagcatggcggaggggaaaatgcacaaagagtacagagtaatttcgaaccaacatagctacttcgtggagaaacagagaaaaatcaccatttttgctattttcaccagcagccatacttccttcctcttcaacaatgaaaaaacgaatttggtgaatcttcactgcttgtactatgggagtaggaaaaagtaccaagggaaaataaagaatgaaaacaactacgctatattttgcatcccccaggtagatgctgtcaatgtgttttactctaaggaggatcttgaagcgtatgcggtagccaagggtttggacctgagccggatatgtccagatgtgtacctccaggaggaagaacacttaagcgaaggagaaaaaaaggaaaaggaaaaactgttctccatcacgaagaagaggctaaagggagaggaacacataggcatcaccttctccagggtccaaggatacccaattgaaaagctatcatatcaggaaatctcatttgaggtttattccaagtgtgacaccgaccttttgcacagactgagcagggacacatcctacctgctgaacttccccgtggtggtggagttgtcgctgcgggtggggagatcgcaccgtgaaaccggtcagggggatagcgcaacgaaacaggtgcatagcacaacaaagcaggtgcatagcggaacaaagcatattcacagcgctaccaagcagaatcacagcgcaacggaacaggtgcatagcgctacccttgagggcgctcacacgctcgacgaggaagttaaaatctgcttggcattctgcctcaccttcccctgcatcctacctagcacctaccttctcaactatgacaccataacccccaacgagggaaaaacgctcatcatcgagttaaaaaacagaaataaatttatgaagcttaattataccctttcgaagatcccctatttaacattgagcaagagcgagggggtaatctctccccaggggaaagtagtcttatccttaaaactccaatgtgatagtgtcaaaatgatggacgactacatgtatctatatttttgcaacaatttatatttcatcgtccttctggtcagggggaacatcatttcaagtaacgcattacggaaaggatcatcctccattttgcttaacctgaaagcagagccatttagaaggaatgagacgcatggtaagattaaacagaaccatgatgaggtatatgaacaaatactcaaaaacctagaacttgaaagggtggataggaatttttaccaactggatggaaagctagacaaatataaatacaacgagagattcataaactttttaaaaaaaaataaaaaaaaatacaatgatgttttgaagctcatgtatgagaagagaaagaagggggaggagtcaatccagaatgcgagtccaagtgcagcctcccctgatacaagtgaacaaatggtgaaggggaaagacagactctccaaaattttaaaggacaaatttatatacgtcaatgatccccccgtggagcgaatctccaacacatatatcaataagggagtctacgaacaagagaggaagaaatacatacaaatgaagacgaaaaatttgaaaataaaaaatgaggaacgtgcgcaaggaggagaagaagaagaaccctacttgatgcagtttgacatgctagacgaggaggaactcaataaagtagaattcgtaaggatgatccaatatgggtatatatttcttggagcagattacacgagaaagtttgtcgtctttaaccgaaacaaacactgcaacgtaaaagtggccctcactcatggtgataacatcacgactgatggagagaagatcttactagtgggaagagattctcacaaaatgagaaatgtaaagcttaacataaaatgtgtttccagggaggaagaagttcaccaagatgataaaaactgcaatgatatttgtctcaacagtggtaaaattggtagtcaaactggagtttctacttccttttttggggaagacataatcacaggttcatgtgccgacttgcataacgacttttttctcctcaaaatgtttgaagagaaaatttccttaatgattaatgattcacataagagagaggtgactctcggggcaactctcgtcttcctaaatgtgcttgtcaatccgaccacagtctactttcgcttccacgacgaggacgtcgagatggtctgtgagcgccacctggttttgtacaaccccttcaacgttcccctcaacgtggggatggtttgcaaccaggcacatattgcgttggattcggagatcttggtacctcctaattcgcacaaaatagtccccctcaattttgtcgccacggaaagtgccaccagtgtgtctgagaccattcggctgtacttggacgggagcaggctctacaaaagcttaaaatgcaaatttatcgccaacaagtgctcttacagagtcacccccaccgagcttaactttgaaaacatcctcctaaacagaaattatgtgaaagagtttttcctaatcaacacgggagactccccagtggtattccgatgcaactacaaaccagattgtgtcagtttactttccaagtataactacgtaaagaaaaatgagaaagtcaaaatatctgtcctgctcaatttaaaggaaggcaaaaaaataaaagacaaaattgtactgaccgttaggggggcgcctcagttgattctccccctgaatgttaagggcaccccctccaatgtcctcatctgtgaagggatccacatccaacaaagaagaggagagctgaaatgctatgaagtgaaaatattgaacagaggatcatgtgatgaaactatccttgtagacacatcatctgtggatttcctaaatgtccaaatgggaaacaaaaaagaaacgaccttttctaaatgtatcagcaaagaatacaaggatgcaaataaaatggaagatgtagtaacagtgcaaaggatttacagcgaagaatacgatgacctcctaacagatgaatgtgctaagtaccattacaacaatttcttgaagcttcacaattcgatatctgtattgtaccaaaataaagggcaaattctccttaaggatagaggagaagtaaaatccatgtacagagttgtggtgccatccaaatgttttctccccttgtgcattcaatgctactcttccaaagcgataaggaaagaaattgctttccaggatttgctccaccataacagagttacatacgacataacggaggagttaataaaaatagacattgaggaaggacacctggatgtagctccctcgtttctgcacttcacggatttgatcccccaccaggctgacgaagcatacatatcatacttttctaaggagaagacgaagagaataacaattagaaatgtattcagtcaacctgtccagtgggcaatccgcttggatgaggagaagcgcagggaaatccacgtggttacaaacgttcacaggggtgtgccagagtttactgaacggaagggagggatggggaagtcgaagagaatggatagcgtcaaatcgagaggctcatcgaaatcgcaccaaacggatgaacccatctgggagcgccaataccaagtgtaccccagcaggcaccgaggcattctacaaccgaacgaagaaacgactgtggacataacgttaagtggcttcaaactttgcggaaggtatgtggacgtgttagacatctcttcctccatcttcagcgaccaaaatgcgggatgcatcgacggtggcaacgatgccagtaacgatgccagtaacgatgccagaaacgatgccagaaacgatgtcagtaaccatgccagtgacgatgccagaaacgatgccagtgacatgctcagcaatttggttggagaagcggaaccagcgcagaaggaagaagcagagcgcgaggtccgtttctccctctacatgctcatatgttgcgacattccgaagctccagttcgacgtaacatacataaacataattcacaacaaactgaatgattattgttctttcccttttgacatatacaaccatgggtacaactacgtcagggtagattaccatttcaagaacctgcacgcagaatgtttttccatctccctagacttcatcaacggaaatgaaataaacgaacaggtcaagaagctgcgcatgtcgttgaagtgcaaagcaaccaagtccttgaagttcaagtctcacattgtctttaccgtgatggattatcaccagtacactattccccttttcgtgaacatcgacgaaggaattgatttcctattggagaaggcggccaccagttcgtccgtcatgtcgtccatcgaagagacaggcgaagtgatcgacgaagtggtcggcgaagtgatcggcgaagtggtcggcgaagtgatcggcgaagtggtcggcgaagtgatcggcgaagtggtcggcgaagtgatcggcgaagtggtcggcgaagtggtcggcgaaggaatccccaaacagatgcaccatatggacgattacctaccccctgaaaacagccccaaaggagcctccaaaggtacctaccctgcagggaaacccacagaaggcataatcatggacagcagtctacaacagcatatgaacacaacagacatccaatttgacaaacagaacgaacttttctccttcttttgtaaaaatatggaggaagtaaaaaagggtagtactaatggaaataatatatataagggattaaaaattaataatataagaaacgtgtactacaatgaggaagaggagggcgtgtacccacttagagaaattattggagattaccttttcgtgtttatccaaagaattatgttacgcaaaagttacttcccccaggtgattgaaagcacaaacgatttgtttctcttctttttggaccttctattggacctcttttcttccatctatccaaacagaaatctgcaaaacattattgtcgaactgaagaagcgtccaactgtggtggtgaaggatatgaagagagaagacttgttggatgaaatgtttacgaaaaatgtgcagacgctttggaaatccttaaaagaggaggaatatattccactggatcacatcatttacgaaaatttgttaccagcggatttgtaccttttccacgttttggacaaagtgcccgactacttccacgttgaagagaagcactatgaagataacacagaaaatctctctattaaaaacttcgaatcgtttttgttaaatggaaataaaaaggtattctactttgagaaaatctttaaaacgcacgtaaggcttttcagttactcttgggttaccatcttcctggaagtactaaacaggaaaatgtttggcgcaataaatatgagctccttgacgaacctgcgtgggattaaattgtatagtatagaaaacaagctgaacgaaaatataaaaatgtataaaattagctaccttgtaattctagatacagaagacatggaggtgaacaagcatgggtcatttttaaatggtaaaaagggtagatccaggaggaaggaggctagcaccctaacaaaactgcacgttaatagaggtggtaccggtacacgaaatgtggcgaaatcatctctgaatactcgtaatggtaatttgatctatagggataagagcacctcccgggggcaagacaaatatgcttacaattatgtgaagaaaaagacgacaaaggataaaccctcctgcaccatcaactacttcaatttttacgaaaataattttatcgacctgaaaaaaattgtcaacaaaattagcagattctgctactccgggggggtagaggaactaccaaatggaaagcaagaaacggtgcaggggaagaacgaactggagagttccaaagggaatacaaacatcgagggggacgacattccaacgggaggagaaacgaacacaccttttaagaaaaaatccacgaaggaagatgttaaggagtctaccataaaatattccaaggacaccacaaaagacgcgccaaacggaattgtccacttcaaaagtgcagagtgcatccttttcgagtggctctattttcactacaacaatgtgtaccacgcgaaggtggaggataagcggtacatctttaaggaggcccgtaggaaaagcgacgatgagttgtcctatacgaagggaaattcaaatgagacgcaagagcggggaaagccggagggatcgcgtagaaaaggaaaccccagtggaagtcctagtttcgtccccagtgactctcaccatgttagcgcttcaggggagctggtcccaagtcaggtgcatgagaacacaaagatgagcacaagcaactttcaggtcgaaataaaaaggaagaaaaaggaggtccaaaagaaaaaaaaggcaaaaagcatccatgaattgaaggacctagttatgatcctgtacactatcatttctcacatcccctattacctattctttaaaaaaaaaattaaattgaactgcaaatccaaaagggattacagccagaatattgatgttctacttataatgctcagcgaaatgaacttgagtaacctaattagcagacaggtgatcgtgcagtttaactacatgcatatatatctgtttttggcgtccctgtattttatcctgccgaaatatttgccctgctactacttggtgctggacgatttgcccgcctacaaggttgacttgagctggataaatgatgaggtggtgaacagcaagaagggaggcaaaagtaagccaatgggtaaggcgcagcaatcatccagggggaaacaggcaccccaaaaaaagaccaatgcacccatcgatggggagaacgaaaaaacgagttgcggagacgatcaaaccatggcagacgaacggatcataactgtgtacaacttaaatagccacatatgtaaatatgaggtatttctcattggctgcaataagtaccaaattgatagaaactacgtgagcattgatcccctaaagtcagaacaaataaaattgaaaatagacaagggatatgacgaaaaggtgttgaaatatagcaaccatactagcattaaacttccatattttaagaaaaaaagacataagggagatatgtgtgacggagaagaaccacactttgaggacaattactctgtttcgtcctgctcctcggaagattgccaatgcccctccgacggggccgacaaagtggacgcggggattgacgaagatagtcgggtcagccaaggggaaaattacaccatcctggtgctgcgctcggaaagcaatcgcctgcaggacgaaaaggacagcgagaaatacatctgcattaagctggtagaactgggcgaaaaggggaagatcgaattggctcccccaggaacgaacataaggggagaaaaaaatgaaggaaataacgtaaacgctaatgtaaacaacggaggaactaggagcgaggttaacaaagtgcgcacaactaattcatctcatctgaatattatgctaaatgatagcgaaacaaagtgttttaatttgaggggcaaagtgtacgaaaataagtgtatagaattgaatttgttcaataatacggggaaggaggtggaggtaaattcaaacctgtaccacctgtacgtgaagaatttgaagaaagaggagaaaggaaaggaaaatttcgaaacagatgtagtgaataagtggggcgatgaccatctgggggggcacaaccaatgtgttgtgtgttcgatagttaatgccaatttgaggaaccacttgagcgacacgtctactcactttagctgtgtccatttagttgacaaaaaccgcttcacttgtctccaaaatgagagaaaaaaaatacaagtgcaattttgcccctttgcagaagggagttatttctgctttcttctattcaatgtaaatgacaaaaagaccaactatatcgtgtcggcatgcaaagtcaactgctcctttaacataaataacgtaaaggaggaagagattataaagatgaactctaaaattttcaatttctcttaccatttaaaagtttgtcctgtaaatagggagtttctgaagtgtgtaaaattcatcctttgtaatttgaaccatttggacgagaggcactttttgtcttacttaagaggttacctagaggatatacataaggggaggaaatttttttctgtcctgtgtgataatgaagacaaaatatccatcgaaaaaggcttcgcaacttttggcccactggattgtaataagtatatggagaaaattttatattctgaaggagtagacatagatcaattgtacgaattggtgaagaaggagaccaattatacatgtactctggagatagcagagaaaacgaatggagtgttcgaatacaactgcgctctagtaagccataggaatgtccttcaaggtaaaagtaagttatcctatgaacagatgcgaaagttacacatcaaggagcagaaggagatatgcgaccctttctacaaagtttacaaaattatagcaagtgtgaatgatggaaatagtagccaatcgttatccattacgtttaaaacgagcgcatttcagatggttaaaaaatccattcctgtgtataactacaccaatggagtggtaacgtacagacgtgtgtactcagatgtaatagacgagaataataagatagtaggttacaacattttcagttgtccagaatacatagaaataggaaaagaagttgaatctgtgaactttgaagtgagttgttattccattatcgggttgacatgctcctgttgcatcactctgtacaacgtaaacaacgaagaggaacagataaagataaacgtacaggcaaacgttatgaagccctacccgaaagataccatacacttgaagttactaaatagaacgagaaaaacggcacagatagagatttacaacgacctgaatttccattgtgaatttaaggtttattcagatttgcccatcctgtttggtgtaaagaaaatagaaatgatgccaaaacagaagaagacatacacgttcttcgtaacaagtgcacacataggagaatacatgggctgtattatctttaagcattacaagttgcttcgttcaggtcatggagaagaaaaaaagggagattcattctttccctttgctaactacttcttctggtataaattaaacatcaccgttgagttgaataaaccattgaagattttatttctcgaaacgaatgtaggagaggaagtcaaaaaagatattgttttaaaaaataatggaaatgaaaatgaaaattattttctcctcgcgtacatgaatgagtacattgagaggagacaggttcagatacccaagaatgacatttacgtgtatagcattaagtacgctccgaagatacctaactattttaatgaggatggcaacggggaaagtgcttctggagagaacaacgatccaaccctccagagggacatccagaatttttactacccccataatggcgattctgattggtgtgggccgccggggggggatgaacctgcccatgggggcaaagaaccatgggagttgtctcctaaccaaggcgctacattaaaacacaaacataagccctttcccccaactgaaaaaaaaatattcgaagatctcattagctactgcaacaagtacattcaagtagacattgcctacaggccacataatatagggttcttttttatctacaataaaaatgaaggaatcaattactacatcttgatgttgctatccaggatgaagagccacctggacgtaaggaatttctccacctgtttgtccaagtcctgcttcttacacctgcactttcagtttaatgaagaggacgagcgatatgtacatctgttggaagggatggagacccccccctctagtaattatcattatcaaattgtagagggggtaaaatgtccaccgggggaaaaacacattttgatagaagaaaatttccgaggggctgtggacatgtgtcgcgcttacgaagagtatgatgataaatggggggaggaaatgcaaaagggaaatgcaaacaactatatggatgcccccatttataaggacaacccatacaatggaggcgcaagggaccaggtgaagcacgaactcaagtgcatttacctaagcaatagaaataacttccatattgtaaaacataaggatgtaaatagggttgttctaaagtataggccaacggtgaatacatcccaagaatgctacgccatcattaggagcaacttgcacggggacttcctctatcggatccatggaaaatatacagtgaagagaaaaattaaggaaaaactggtcttgtacaactcatgctgtttgtacaattttaatgtaaatttgtttaaccccttcaactgcaaagtgagggtaaaatgtaggtttaaggggggaaacgcaacacagttatgtaaccacatgtgtggtgcgcagggaatgaaagagaaagacatagatgagaagtacatgaagtcgttccagaaggagcgagttgtactaaacaaggaaaggtattatttaaaaatattgaataaagaaaactttaaaataaaaatgaggaagcactttacaattatgatgacctacttttgtaaagcggtttattcgaggaggacggttctcataatgatgaagccttttcgcagggataggactcatgtggatggccctccgatgataatttacgaatacgattttgtgtttaacaatttcttgaggagtagaaggatccaaaatgtgttgcagtgcatccccgctgtgggtgagataaaggacgaacaaggaagggggaactgtgaccagaagcaaccatccacagagtgtgaagacctaggtgtaaatacatcatgttacttccattcgagtgtggatgagccggagtctggaaaccaagcgagtggaaatttcacgatgaggcattccgagcggggtgatgtggaagctaacctgtccgaagtgagcgatgtggaagcccaccaatccgaacaatctgaatcgatccatacctcacgcagttcctctgtagcaagcgaacgaagtcatttcacgctgaatgcaagtgaattgggggtaatggacgtggcggaaggggatttcaaattggaagatgccgatacaatcgaaataggaaagaatcacacctgttttactcaccccgacgacaaggcgttattgtctcctaatgactatgatgtagaacatgaaataaagcacgagagggttcttccgtgtgcatcggataataaggaggttgtacactatgacgggaaaatttttatcgaaagtatgtgcaaaacgaagacctacgtagaggtacacatagataagaccaagatggggacatgggcatcttgcccccagggtatgccccagggtatggatcaactggagatgcaaaataattctcttcataggaatgtggaaatgggaaattataactataaaatatttttgctaaacttgaaaaatgtacgtaaaaacatggatactgttgatgaaactgttgttggtggtaccgggggggcaatcgggatggatgtaaaaatgaatacgcttcttttcgatgtgaatgaaggactactaaatgattacctgtatgtgcacattgtggaggacacggaggcgcgccttgttctgactttggagctgctggcctttcttcccttccactgtagctttgacgtttttgtaagcaggaggggaggggaaggagcaacccaccatggggaatcaggcaaagcagaccaagcggcagcgacaaacaaagcggaagcgacaaaccaagcggcagcgacaggccaatcagtggaagaagcctacaatgtagtcgaggaattctgcgggggaaagccccgtcacagatgcaccaccttcgagcagaatcacataagcgtagaaatattaaactgcatgaacgtgtctagacagtacctaaacattaccgttggtgataatttatgtggagagacaaggcttaacattttttataatcacacaagggattcatatttcgacgcctatatggttaagcataatcagctgaataacagcagtcactggaaggatgaaattttaaattttgaagtcaagcctaaatgtggtattttaaagcatggtagttacaacaaattcttcattacaaggacgaacaaaaatgggctcttaacttttaacaattcttttctccttttaattaaaacggagcggaatgtattttcgtacatggtcttttctcactacagggcggcacccagcgatgccctcgcgacggaggaacataataagataaaggagatcctcaacgagaacaagaagaagttcagcgtcctattcagttctttcaggaaggaaaagaaggaaagtagtatcttcaatcagaagaatcagataatcattgaagacatttcgaagtcaacaaacgaaatcagaaatgcctaa

SEQ ID NO: 21 - DNA, 14016 bp, Artificial Sequence
  Synthetic
atgaacgaagaagtaacgaataagtttgatcttatagatgtagaaccgaaatctctcgaatttttttatgactgtgcagaaaatgtagatgtttttgttaaaaataagaataatataatagaaaaaaaggtcttgacaataaaaaatatatgtacaaaaacgcagaacattaaaatatttgagtcagattcaatatatttaaaaataaaagggctaaaaaaaaaaaagttagctccaggaactagtgaaaaaatattagtacaattctctttttgcaattttgatacaaaaaaatgtaaatactatagtggcaatagcaatagtaatagtggtgataataatggtaatagtaaaagtaataataataacaaaacggggttgttacgtaaactgaacaatatagagtgcacagtatatgtgaaagtacagagcgaatattcgacattgcatatacccatttacattaaaaagaagattcctttattcgtttttaataacataataaactttggagtatgtaaaaataatatgacttaccatttttctcttcaagtaaggaatgaagggactaaaaaagggaccttcactatatgtaaggaaaattttatagaagaaggaaatgaacaaagtaggaacaacgaaattgatggtggtaaacatgcattagttcgcttcaacggtaatatagaaacgaaagagaaggaagacaaaataatcaaaaaagaaatgagcgaaaaaaaaaataatcttatcatcgattttgacaaaacatctattgacctggatataaatcaaagtgaaataataaatatcaaaataataaatacaaaagaagaaaaaattcagcgtacgtataaagtcttaacacaagaacatagttacttctgtgagaaaccgaaaaacataataataattgcaatttttgtaaacagtcagacttcctttatttttgacaatgtaaaaactaatcaaataaaccttaattatatgtattatggaaataacagaaaatttcaaggccgactaaaaaatgaaaataattaccccatattttgcaagtatgaaatatgtaaagtgaatgtattctattctatacagaattttcaaaagtacgctgatgacaacaacttagatgtagggctgctcatgcacgatttaaacgataaagcagaaaatgaattaagtgaagaagaaaaaagagaaaaagaaaagttgtttactattgcaaaaaagaggttaaaggctgaggataacataactgttaagttagataaagaagaaggttatcctgttcaaaaattaggatatcatgaaattttatttgagctccaatcgaaatgtgatattaattttttagaaaaaataaacagaaatacttcgtatttgctcaactttcctgctgttttggagttaaatctacatattaggaaagttgatgaggataaaaaaaagggaaatgatgatactccaaatagggatggatatacagatatgcatagcagcacaaataggggcgatgaagaaataaaactgttcttcatcttctgcttaacatttccttgtttcgcctcaagtagttattttctaaattacgaaacagttacgttaaatgaaggaaagacattaataatagaattaatgaataagaataagtttttaaaaataaattattgcatttctaaaattccttatctaacattgaataagaaagaaggttgtatcgtccctctagggaaagacattatatcgcttaaattaaaatgtgataccattaaaaatattgatgaatatatgtacatatttttttgtaataatttatatttctttgttcttttaatatatgcacaggttaagtctgtatttgcatcaagacatgtgtcttcaacaattttgataaacaagaaaaggacaaattacaatggggatgttgttgatagtaaattttcatcttcatctaccaaaaaaaaaaatgacgaaatatatgaacaaatcataaaaaatttggaattagaaagagtagacagaaacttatatttagaagatgggaaactcgataagtataaatataatgagagtttcatgaactttttaaaagataacaaatcaaaatataacgatatattaaaatatatgtataaaaaacgaaaaaagaggggaaaaataaacaacaataataataacgataacaataacgataacaataacgataacaataacgataataataacgataacaataacgatgacaaagtagtgcatgataaagattataagcgggaaaaaagagcagaagaaagaattaacaagatagtgaaagatctaaatatatatcttaatgacactggaatgggtagaatggatgaaaagagcaaaatgagcaagacgaatagaacttacaaaacgggtaaagcttgcaagattagtagcacatgcattcaacatgcggtgtatgaggagaggagaaaaaaatatatgaacatgaaaaagaaaatttttaaagaaaaggatatagaagaaatgagtaaaaaggatgcggatttgttttttcacgagatcttagagcaacatcaacttaataatgttaattttccaaaaataattcattttgataacgttctgttaaataataattaccaaaaacagtttgtaataaccaacagtaataaagattgcagtgtaaaaataaatttcatacatagtgataacatagagctaaataaggacattttaattttgagcaaaaattcggacaagcatgtaaatataaacttcaagttattatctcatactaatttaataaatagatacagaaattatcaaaatgattctatatttaagcaatctagtgaaatttttaatgagcagttaaatgaaaaaacctcacctcaaaatgaagagataaagtgtgtgctaaatgaaaaagggaataattctacctatgatgaatcaaataaaattttcagcaattctataaacgactttttccttgaacagacatacaaagaaatgatacagctaaaaattaataatgcgtatacagaattcatcataattaaagcgaatttagtatatttaaacgttttacttgttagcgatgttttatattttcgttttcaaggtacagatcttgggatgattcaggaaaataaactagtcatgtacaaccccttcaatacccctatttgcgtaagaactacttgcaacgagcagctttttaaagtggatgccgaatttaccataccccgtaatgcttataaaactgtgcctgttaaatttattgcaccccaaaatgccagtaatgtggaagagtttattcaaatctacttggacggcagtaggctatacaaaagtgtgaaatgcgaatgttttgtaaataaggtattttataaagtaaacacaacagagattaatttcgagcacatacttttaaataaaaattatgaaaaggatttttacttaaccaacacaagtgactctcttctctccttcgagtgcgtctacaagcccgattgcgttagtataatttcaaaatacaattttgttaataaaaatgaaaaatcgaaaattactgtgtgcataaatctgaaggagagtgaaaaaattaaagataaaattattttccgcataagaggctcggagaacttggttattcctataacggcaaagccttccccatccaacgtgctcctctgcaacaacgtacaagtggcacaagaaagtaatgaaataaaaaattatgaaataaatattctaaacaaaggtttgtgtgaggaaagcataattttagatctaagtgagctatcctttttaaatatctacacaaataacaaaggacaaaaaaaattaatgatgtcaaataataaggaatataaaaatgcggatgagatgatagatagccaaatgattttaagtaaaatttcaaaattagaacacgaagatatactaacagatgaatgtaccaagtattactataatcagtttataaaactttataattttataagtacgtattataagcataaaggcgaaataaggtttagcgaaaaaggggaaaagggcgtaccccttcccaaaatgtatagaattaatattcctccaaaaagttttgcttccttgtattttcagtgctattataacaagtgcctaaaaaaagaaatcgcatttaatggtttattaagatataatggtaaaaatgataacagtaataatgataatagtaacaatggtaatagttatcataccgcgtatggaatgaaagaagaggaagtaaaaatagaaattagggccagctgcttagaagtagaaccctattttctctactttgaagatatgcttccctctagttcagataaagcattaataacctactttacaaaggaaaaaacgaaaattctaaaaattagaaatgcgtccagtcaggtggttcggtgggaaatagatatatgcaatgaaaaggaaaaacaaaattatttgatcggaataagaggtggagattgcagggagagagttgaaaagggaacggttcactccactctagaagaagaggcacaaaagaaggggagaaaagatatccccaagaagggaagaagcgaatggggacatatgggacagaatggagaaaagaccgtacaggctctaaaagtagaaaggggcacagaaagcaaagtgggaaaaagaaacgaaagaggagaaataggagagaaaggaggcgtaggagacacaggctatatatttgataaagaaaagacagacaatacaggcaaacagaactactggcagaaatatgaaatagagctgggaaaaaaggaaggaacactggaaccaaaaggggaagtagagatacaagtgaaattaaaaaattttacacaaagtggacgatacgtagatatattgtcaatatcgtacgatcctgtggaggatcatatagaaaggagtagtaatgaaaaagaaggaggctcaaaagagataataatatcgaactataaaatggaattgaaaaaaataaattattgtatttttatgcttctaagttgtaaaataccgagattgctcttcgatgtaacgtatattaatatcatacataacaaattaaatgaatacattactttcccttttcatatatacaacagtggttataattatgtccgagttaattactaccttaataatttatacgaagaatatttctccatatcattaaattttacgaacggaaatgaaataaatgaaaaaataaaaaaagtagatgtatgtttaaaatgtatggctatccgaaatttgaaatttaagacatatatcattataactgtgttagatcatgaccagtatgtcattccgttatttgtaaatattgatgaaaacattgattatctttttgataaattggatcaagaaaatgaacatctacatgtgaaccattctccttcgatgcctgctttgcacaatatgcatattcagtgtggaaagaaaaatttatcagagttagagaaaataaaggataacccgatcggttataggaataacgatcaaggtattgaggatagggatgatggatatggggatacacaacacaacatcaaccactgcagcacttacagagatgaccgagataagcagagtgctaaagatatgtcccataccaggaggagtaacaaagagaaaacaaatggaggcgaatgcgtatacaattccgaatatgaatctaccagttgtacatgcagcagtgtttgttgtgatgataacatgcttagatgtaattgccaagagagaaacagtaaatttctgtctttttacagcaaaaatgcgggccaaagggacataagaaaaagcgtatgtatatacaaagaattaaaaataaaacatataacaaatgtatattataataaagaaaaatatttattaaacataagtgaaataactaaggattatgtttttatatttcttcaaaatattttactaagtaaatactatgctccaagtgttatagaaaatacaaacgatctattcctattttttattaatttattgtatgagcttttcttactatacccaaataaaaatatacaaaatatagttaatataataaatgacgtaaaatcttataaaaatatatcaatgcaagacttggaaaaaaataattttctccaggaaaaattcctaaaaaatatgaaagctcttttgaaatacttagaggaggaaaacttactagtgtatcatataatttatgaaaacctattacctctagatttgtatctttggtacatcttcgacaaagtacctgactacttttacctaaaaaagggcaaaggaggagaagaagaaattaataaaagaaaatgtatacaaattgaagatgtagaaaattttttaacaaatggaaataataaaatattttatttagatagaatttttaaaactcatttaaagttattcagttactcatggattactatattgttcgaacttttaaataaaaaattatttagctgtgtcaatgtaacctctttgaataaattaagaggaataaaagtgtgtaacatagaaaataaactcaatgaaaacataaaaatttataaaataaactatctaattgttttaaataaacaagacaaagagccaaacaaacataccatttttctaagtgagaaaagaaaaacatcaaagagaaaagaaaatagtagttcaacaaatattactccaaaggataacaacccttacgaagataaatctgctaaagttagcagtgtaatcaatatggggaaaaataaaaataattacttatccaatttttatatgaaaaatggtagacccacacatcataatagtattataaagaaaaaaaaaatgaataatggtaaaaatacctacacgataaattattttaatttttatgaaaataattttatcgatttaaaaacagttgtaaataaaattaataatttcactcattgtgaggaattcgaacagggggtggaaaaggagcgagatatatctttagaaaagcatgaacaaattataaatagagatgataaggaaactacacagagaaatattcgaaacaaaacatattccaaaggggaggaacaggaaaaggaagagaaagacaatgtacaatttaaaagagatacagtggtaaaggaacaaaaaccatgctttcactattttaagagtgctgaatacatactatttgaatggttgtattttcattataataatataaattttgcaaatgtagaatataaaaggtatttatttaaagaggataatgagaaaataaaaaaacaacaacaaaaacaaaatgatttaatttttcctaatgtaccaatgaataaaagcaaagaggaggaagagacttcttcaaaatgtatggagaataatgatgtgccactcatattaaataggtccaacttttatgttgaaaaaaaaaaaaaaaaaattatggaaaaaaaagataaaataacaagtatatgcgatttaaaagatttagtaataatcatatatactataatttctcacattccgtattatttgttttttaaaaataaaataaaaacaaaatgtatatctaaaaaggattacatatataacatagacattttgttaataattctgcatgaaatgaaattagataaattaataagtagacaagtattgattgagtttaattatattcacatatttttgttcttggcttctatgtattttatattaccaagctatttgcctagttactacttagttgtggatgatttcgcctcctacacaaattgcgttggtcttataaaggaagaaatgataagcaataacaaaaataaaaagaaaaatacttgtaatcaaaacgaaaatgaaaaggcaggtattattaaacttaatctgactacacctgaggagcgtgttataaccatctacaatttaaataattacaagtgtaaatataatgtatttttaataggttgtaacaagtaccaaattgacagagatcatatagttattgatccacttaaatcagaagaaataaaattgaaaatagacaaaaattatgaccaaactatttgtaagtataataataacactagcattcaattttcaacctttaaaaataggagaaaaaaaaaggaaactatcgatgaaactacaacatattttgacgaaagttattccttttcatcgtgttcatcggatgattgtaaatgcaattctgatgaagctgaaaagattagtgtaagtaacaatggtgatgattccttgaccccaaatgaaaattacgctatattagtcttgagggccgaaaacaactacttccctgacgataatgatgatgaaaagtgcatttgcattaaacttatcgaaaaagacacacatgaaaagtcagatctccaggcaagtaatttaaagaaggaaaaaaatgagtggaatgatttaaatattaatgtaaagaattccgaaacgaggaataataatatcagtaaggtgcacctttcgaattcctctcaattaaatgtaatgcttaataatagcgaagtaaaatgtttgaacttgagaggtaaggtatatgaaaataaatgcttggaaataaatttgataaacaacacaggacgaaacgtagaaataaattcgaatatttatcacttatatttaaaggatttaaaaaaagaggaaaagaaaaagaaaatttttgaaatagatataataaataatttaaacataagtaatgaccaaatatataaagtgtgtgatgtatgtacaataattaacagcaatttaaggaatcatttgaatagaaaggaaaactattttagctgtttttatgtagataatacaatacctattattattagtagagaaaatgaaaagaagaaaaaaaaaatacaattacactttttcccttttatggaaggttattatttttgtttcatcctattttatgttaaggatgtaaaaacgaaatatttgatatcaatgtgcaaggtcaattgtttcttttacataaataatataaaagaggaagaaataattaaaatgaattctcaattgtatgatttttcgttcaatataaaattaaatccaatcaataaagaatttttgaaatgtgtaaaatacattttgtgcaatttgaataaaatggacgagaaattctttttaatctatttgaaaagttatctacaaaatgtaagcaaaaacaggagtagtttttatattacatgtgacaggaaggataaaatgataataaaaaaaaattttgtatcctttgaaaattttgattataataattatataagtcatattaatactccaaatttagacatagacagattatatgatataataaaagagggaatcaattatttatgtgaactgagcataaaagaggagagcaacggtgttttcgagtataactgcgttcttctaatgaggaaaggagaagcagagaagcagaataagaagtcgcataaacatactcagcagaaagaaggtctaaaaacagaaaaagacgcagactgcaatatgcttagctacgaacagattggacattcccatttaagggaacaaatcggcaatcatgaaatttgttacaagttgtataaaattatagcaaatgtaaataataagaaaaataataatataaactctattataacatttaacacgaacgcatatttagaaactaagaaaactatatcaatatacaattacacgaatagcactataacgtataagtgtacatactccgacacagtagatcataataacaataaaataaattataaaatttttagcagtaatgaatatgtagagatacctaaagatgtagaaacgtttaattttgaagtaacttgttattctattgtagaagtgtcttgttcatgttttattatctttactaacataaaagatgaagaagatcaaataaaaattaagcttcaagccaatattaaaaaaccttacccaaaagaaacaattcatataaaactattaaacagaatgaaaaaaacagtacaaattgaattatataacgaactgcagtttcattgtgaattcaaaatttattctgatttgttcattttattcggtgataaaactatagaaatgctacccaaacagagaaaagtgtataccttctgtgtgagaagtgctcacataggtgagttcgtggggtgcctcatatttaagttttaccggttgataggtgctgaaaggggtgaccttcacagaagtaacgttatcgttagtaacatcagcaacgttagcagtgaaagcagaagcaacgttagcggtgaaagcagaagcaacgttagcggtgatgatacaagagagcatcttttttattattttttctggtacaaattgaacatcaccattgaactgaacaagccaataaaaatactgtttcttgaaacatccgtgggagacaaagtaaataaagaaattgttttgaaaaataatggaaatgaaagcgaggactacttcttactaacatacatgaatgaatatatagaaaagaagcaagttaagatacaaaaaaatggcacgtacgtccataatattaaatacatgccaaaaataccaaattattttcataatataaaagaggcttcagacaacttcaagttaagcactaaaagaaataaatcttttcattattgttcggttgatcagttagataaagaagtggaagggactaaccctcttcaaagtggttataatgattttccaagtggtaaaaacagagattgcacatataaaggaggcaaggaaatggaccttgcaaaggaattgcacaaattttattttccgccaaatgaggagaaggaggaagaaaagaaagaggtcgaggcgaaatgggaggaagaaaaaagggaagaagtgaatgacgacgaagatgaagcaaaatcccagggagaagacgaggggaagggccaaaacagccacttagctgaggaagataaaaaagaaacagctaaaaaatgtgaaattagaaatagaagtagaagtagaagtagaaacagaagtagaaatagaaatagaagtagaagtagaagtagaaatagaagtagaaatagaagtagaagtagaaatagaagtagaagtagaaatagaagtagaagcagaagtagaagtagaagtagaaatagaagtagaagtagaaatagcagtagaagtagaagcacaagtacaagtataaatgtactcagaggtgtacgtagcagtagtggaaaagaccctcagaggaaaagcccctcacgagatcatagtagaaggaaaaataaaagtgacatgttaaggaaaaaatgtaaaaaaaacgttgagagagataataaaaaaatatttgaagagttaattaattattgcaaaagatatataaatacaaatattaattataagaatcagaatattggattttttttcatatataacaagaacgagggtataaactattacattttgatattactagcaaaaatgaggagggaattagttatagataatttctctgcatgcttaaccaaatattgtctacttaatctccacattaattttaataatgaaaataaaagatacgtaaaagatttaaatacgaataaaggtacatgctattctgataattacagctacgaaattgtggagtgtgtaaataaaaatggtggatgggaggcagaatcagaattagcaggaaaatgcaaagacacacaaccagacgtttcagttaataaacatgttaataggggaaaaatttatggggacacttatgatagtaccattagtcgtaataaccaaataaatattgatgatcataaatatgataatgctaatgtggaagaaatcataaattctcaaattaaatgcatttatttaagtaacaaaacgaattatagtatttttaaacatacagataaaaataatgtcattataaaatataagccaactgtaaaaacatcacaagaatgttatgccataataagaagcaatttatacggtgattttatttacctaattaaaggtatgtatacagttaagagaaaaataaaagagaaaatagtaatgtataactgttgttgtttataccactttaatataaatttatttaacccactgaattgtagtatatatgtaaaatgtagattcaaaagagagaaaagtaaaaatgatgagctttatgacaacttttatttggataacaataaagtgggaaggaaagcagaaagggaagaatgctgcttttcctccatggaacacgagaaaatattattaaataaagaaaggagttactttaaaattttaagtaatgaacattttaaaataaaaaagagaacacatttttctatgctcatatcatacttttgtaaagaggcactatcaaaacatacgttaatagtaacgttaaagccttttggcacggataaggccagtgatgggacccttccaccaatcatatataaatatgatatcacttttaataatgtggtaaggcagggtatcgaagcgtattcgttgactggttccggtgcacaaagtacggatgaactcactgctaggggggatagcagcagtggaagtagcggtagaagtagaagcggcgatggaagtagcgatcgaagcagcagtagtagccctagtcgtcgcaacagacatgcttgtgaaagtggtggtacatgcgaaaaaacaaagagctactctgtaaccagccaaagtagcagcaatatgctgaatgggagtgaactaagtaaaagcgatttattccaaagagggttaaaaaaaaaatataattctaaagcaaatgagttagtagaagaacaggaaatttgtttacatgcatgtgatgaaatattagtagaagataaagaagaaaaggacaagtatgagaggaacatgagctatgtagtagatagaaaggaagaagtagtttactgtgatggaaaaatattcatcgaaagtataagtaaaaagaaaacatgtttgaaaataaatatagataaaagaaaaattatggaaagagcagattataacaatatcataaacgagttagaaaactgtaaaaaggaaaagagcatattacacagaaatattgaaaaagataagtgcaactataaaatttttttaataaatttaaaaaatgtaagagatgcaacaatggatgaacaaaataaaacaaacctccatatgaataaaatcctctttgatatcagagagcagttactaaatgattacttatatgttcatattattgaggatacagcctccgctttggtgcttagcttagagctcttcgcgcatttgccattccactgtagttttttcataatgattaagagggtagaactaggaagtcgtgaggtaggaagcgacgaagtggaaagaggcgaaatgaacagcgaggaagaggataactacgtgctcgtggaatcccttaaaagtgagaagagaaaggaattgacaactatcgaaaagaattacatattcgtcgaaatttttaattatataaatttgtctaaaaaatatatcaacgttatagttgatgataacttatgctcagaaacaaagttaaatatattttacaaccacacaaatgattcttattttgacgcatatatcgtcagttacaatcaacaaagtaatgatccatttgcagatgaaattatgaattttgatataaagcccaagtctggaatattaaaacatagtagttacaataaatttgtaattactaggagtaacaaaaacaacataataacatttaatagttcattcttacttctaattaaaacggaaaagcatattttttcctacatcgtctttgctcgctatacccctgctagcgatctccacacagcgttctctttgtatttccagcaggagcaaaataaggtaaaggaaatgctcaatgaaaacaagaagaagttcagcgcggtattcaacacattcaaacaggaaaaaaaggagagtagcatatttaacaaaaaaggtcagattataattgaagatatctctaagtcaacaaatgaaatccttaactgttaa

SEQ ID NO: 22 - DNA, 13395 bp, Artificial Sequence
  Synthetic
atgaacgaggcgacaaataagttcgatcttatagatgtagaaccaaagtttatagaatttttttatgacaatgtagaagatgtagatacatttgtccaaagcaaaaataacgtgatagaaaaaaagattttgaaaataaagaatgcatgtacaaaaacgcagaatgtaaacattttcgaatcagaatcaaggtatttaaaaataaagggggtgaaaaagaaaaagttagcccctggtactagtgaaaaaattttagtggaattctctttctctaatttagatgtaaaaaaatatagatttggaaaaaaaaaagatatcctaaatattcttaataatgtagaatgtacggttagtttaaaaatacagagtgagtatacaacagtgcatatacccatttatataaaaaagaaagtacctatattcatttttgacaacataataaatttgggaatatgtaaaagtaatcgtacataccattactctctcaaagtaaagaatggtggttcgaaaagggggaaattcacaatatcgatggacaatattgtagaagaggaaaaaacagagcaaacacatgactacaagagggatgaaaaacagacaaaagttcactttaacggtgacattaacaaacgaaaaaaagaagaaatagaaaggaaagatgtgcatacaagtaagaacgatttattcgtgcagtttgacaaaacaacagtcgatttaaacataaacgaaactgcaactataaatattacaataaaaaataaaagggaagaaaagatttatcgtgaatacaaggtttatacaaaagaacatagttacttttctgaaaaaacaaaaaatgttattataatcgcaatttttactaacagccaaacatccttcatttttgaaaatgaaaaaaggaaccaaataaacctaaattacatgtacttgggtcataaaaaaaagttccagggacagataaaaaacgaaaacaactaccccgtattttgcacacataaagtagggaaggttaatgctttcttttctatgcaggagttcgaagcctactctgaggagaacggcctagacattgatcagctcagtcgcggtgaggacgataacaacgaagggaaatttgtggggacgtttgttacggaatctccatgttcttctaccgcctactcctttagttatacacagaacacgttcacctcccttacgggaagtcgtgattttctagataccaccgtattgtggacatccgccttgctcatttattttttgttttattttttttttctctcttcttttcccatccacatccttgcagatatgagaggcgtgacgcagaatgagatgtgtgaagaggaaaggaaagagaaggaaaagttgttcgcgattgcaaaaaagagattgaagcaggaagaaaatataacagttaagctgagtaaggaagatgggtataccgttgaaaaactaagttatcacgatgtttcggtggaagtgcaatcaaagtgcgatgttcgctttttggaaaaggtgaacaggaacatgtcttaccttctaaacttccccgttgtagtggagctaatgctgtacgtctgtagtggagaaagtggtgttagcggtagcggggggatcaaatgttgtcacgaaagttgcgacaaccgcaacctcctggacgaagagattaagctgtttctcgttttttgcctaaccttcccgtgcgtggtctcgaacacgtactttctgaattacgaaacagtgagcctgaatgaggggaaaacactgataatagaagtgacgaaccgaaacaaatttttgcaagtcagttactgcttttcgaagataccttacctaactctaaacaggaaagaggggtgcatagacccaatgtgcaaggaaataatcactctcaagttaaaatgtgacaccattaaaaaaatcgatgaatatatatacgtgttcttttgcaacaacttatatttctttgctatgctaatgactgcagaggtgaagtctgcctacgcgctgcgtcaagcatcctctctgcacttggaacaggggaagggtgaaaagggtctcaccaacagaggaagcggtgaccgcgacttgggcaaccgccaggatgacgaaatttttgaaaagataataaaaaaacttgatctagaaaaggtagataaaaatttgtacttagtggatgggaaactggacaagtacaaatataacgaaaattttatcgcttttttgaagaataataaaaaaaaatataatgacatactgaaatacatgtatgaaaagagaaagggaaggaggaaagacaaaactaacatggacatgaaagagaaagaggaagaatgtatggaaaagaaaaaattaaaaaaaaaaatttctaaagtaattaacgatatatgggtatatataaatgatacaggattaacaaaaataagtaccacttgtattcagcattctgtgtacgaggagagaagaaaaaaatatatgcacataaaaagggaacacttggaaataaaagatacgaatagtgcaagtaatgaatgtatgaatgtgccatgtgatgaggtattagaagaacatcagttagacaaagttatctttccaaagaacattcacttcaaccatgtgttgttaaacataagttataaaaaggaatttactgtacataacagcaatacagactgcaacattagattagatttcacccacagcgacaatgtgaagatagataacgatatgttgatagtaggtagaaattcaaataaaaacattaatttggaactaaagataagtccattttttaaaaaggtggagaatatgacaaatgagaaaggaaccactacgtgtcttttccctaccccctctctccaaatagagagagaaaataatatgttattcaacaacccagaaattgttatgatcccgtcagttaacgattttttccttcaacaaatatatacggaaagaatcccattaaaattgaacaatatatacgaacaacaaataacagttaaggcaaaccttatacttctaaatgtccttatcaaggagcaaacattacacttttacttcgaaaactcggatgtccagatgtgttgcgaaaggcagatcatcctgtacaatccctttgacttcccagtagccgtgagtgtggcatgcaacgagtctctcttccaaatagatgcccatattaccattcatcctaactcgtgcaaaatggtacctgtcaagttcctagcccctccaagagccggcgttttggaggactttatttacatatatttggatggagcaaggttagtccaatgcatgtgctttgtaaacaagtgttcgtacaaaacagacataacagagataaactttgaaaatataattttaaacaaacattatacaaaagatttttacctaacaaatacagccgactatccgctcatattcgaatgttcgcacaagccagaatgcgtgtatgtccaatataagtacagttatgtccaaaaaaatgaacgcctgaaagttactgtttcggtgaacttaaaggatggtaaaaaggtcaaggacaaaattgtattctccgttaggggtgctgaaaatctcgtcatccccataagtgctaaggctgtcccaactcacgtattgtttatgagaggattgcatataaagcaagagagctgcgagatgatggattacaagatggacatcctaaacaagggcatatgcgaagagaaggtaatcttggatctgacagaactaaattttttgagtattagtattagtattagagagaaaaaagagaaaaagacattaacatacaatagtaaggagtataaacatgcagaagaaaaaagagatgacttcccaattttgagagagatttttcatgtagaatatgaagacataataacagaagaaagcacaaaatggtattataataaatttataggattatacaattttatatgtaactattgtagaagtagagggagtatacaattcacagaaagaggagaaaagagctcacaatgtttgtatatggttaatatacccccaaagagttttgtctccatgtatattaattgctattacgacaagacaataaaaaaaaaaatgaaatttaacagtttgttattacaaaataaaattatatatgaaaaaaaggaagaggagattaaagtagaaattgatagtacatgcttagacatatccccagcctttatccacttcacaaatgtactcccatcaaattcgcacagatctctcatttcttattttacaaaagagaagaaaaaaacgttaaagattaagaacgtttctgactatcctatcgagtggaagatttacgtatacaccaatgaacagaaaaaggttcacctgatgagaagaacagggggaaaggccagggcaaccgcaacacatgaggggaatcagacagctagaggtgtattcaccggggagaaagcggcaagagaaatagcgacaaagcaaataacggcaaagcacataacggcaaagcaaataacggcaaaacaaataacggcaaaacaaatagcggaaaagcaaagcgtagccatgggggtatcgaacagagatatagaaacaaagggagacttagtaatggggagagacgaagtggaggaggttgcgaaagcatacgagttggatgtgagcaaacagagcggcttactgaagccagaggaagggacggagatagaggtaaaaattaaaaacgtgcttcaggggggaagatatgtagaaatattgggcatatcagcaacatttgtggacaatgaaatggggaaaaaaactgacagtaatgtaggagacacagaaatcgaagtattaaagagtaccgttcgaagagaggaaaaaacttattgcgtttatatgcttctaacttgtgaaaccccgaaattattttttgacgttacgtacataaatataatacataataaattaaatgaatggttcacctttccttttcatatttacaatagtggatatgactatgtcagagtgaaataccattttaacaatttgcatgaagactattttttcttatcgttagattttgaaaaaggaggtgaaattaacgaaaaagtaaaaaaaataaatgtttctttaaaatgtaaggcatataaaaatgttaaatttaagtctaacctttctgtaagtgtgttagagcatgatcagtacaatatacctctctttgttaacatcgatgaaaacattgattaccttttagataaagcaggcggtgtagaagaggtatctttccagagaggaaataaaaatggagagaaaacacaaaagggggatattcacgtgggtagaaaaatgctggataatatcgacgagagggaagactaccgctatgataacccatggaaagaacaattggatgatactacccctttcgaaggctgcaatacagaaagcaatatatctaaacaaacaaaagacacattgttacccttcttctgcagtaacagtgtagaaaaggatgaggaagaaattgtcaatatttataaggggttacaaattagaaacattacaaatgtgtattataataaagaaaattttacacataacttgacagcaattttaaacgattacatatttatatttattggaaggattgtgttgaacaaatcttatctcccatatgtcatagaaaatacaaatgacctatttttatttttccttaatttgttaaatgatctttttttactgtacccaaatagaagcatacaaaagttgataaacggaataaaattttatgaaaatattgccatgacgaattttgagaaaaacacattactccatgatagcatacttcaaaatgttaaaggaatattgaagcatttaaaggaggaaaagctactcgttgatcatatcctttacgaaaatttgcttcctgtagatgtgtacatgtactatatcttcaacaaggtaccagaacacttctacgtaaaagggaaaaaggaagaactgggaaaaaacgataacggtgatagtaatgaatacatttgcaatgtagatgtgaatcaatttttacttaacggaggagagggagtgttcttttttgatagggtattcaaaacgcatttaaaactatttagctactcgtggattacaattttcctagaatttttaaacaaaaatatgtgcagttgtgtaaatgtaaactcgttaagtaaattgcggggaattaagttgtgcagtatagaaaacgagataaatgaaaatattaaaatgataaaaataaactacttaattgttttaaataagcaggatcgggaattgaacaaacacaccgcctttttcgacgagaggaaaaggaggtctcgaagaagcgaaggaaccagtagcggcaccatttgcagcggcaatggcaatgctagtagtagtggtagaggaaaaggaatgcagaggatgccagtcggggtgattgggatggaaaaaggaaaaaagacgatgcccataaaagaggcgaagggcgaccccagaaatggcaaaaatgctcaccacggtaagaggatgaaagttgggcttatcaaaaatagcagtgctataaattacttcaatttttacgaaaacaacttcatcgatttgaagaaagttgtccagaagataaacagtttgaactgcgagggaggggatgcagaggggaaaaaaagggatgaccagttaacgctcgaaggaagtcgtaaggagagagatacaaattcaccaaaaggtagttcacccatggaagaattaagagaggggagaaacaatgggatactcaaaagagaagtagaaaaggaacaaacacgcaactttcaccaaattaaaggtgttgagtgtatacttttcgaatggttatacttccattataacaatgtgcattacagcgatgtacaaaatgaaaggtacgtatttaaggagagttggaatgaaaatgaaaaaggcgtcgctgatttgggaaaattacccagttcgaggaaagttgtaaacgttgcagaagtgagaagggaggaagtagaagaggaggtgacgccccaacatgaagtcaatgaggtagtgaatcaacatgaaaatagcgagggaaggcccgtaaaattaaataaaagcgatttttatatggaaatgggaaagaaaaagaagaggaaacatttttcagagaaaaaagacaaaataacgagtgtgcatgaattaaaggatttggtgatgatcatatataccctaatttcacacattccatattacttattttttaaaaataaaataaaagtgcattgtaaatcggagagagactacatcaataacgtagacattttgctagtaattttgaacgaactaaaattaaacaatttgatcagtaggaaagttttaattgagtttaattatattcacatgttcattttcctcacatcgttatattttatacttcccaattatttacctagctactacctacttgtagatgattcttcttcctacaaaaatgatatttgtgctataaacgaagaattaatacgagaaaaaaaaacgaaaaatgtacgtgtccaaagcgaaaataaaaaaataggcattcctaaaagtgtgagcaaggtggaagaaagggttataacgatatacaatttaaacagctacaaatgcaaatataatgtgttcctcatcggatgcaataaataccacgttgacaaaaatttcatccacattgacccccttaaatcggaagaaataaaaataaaaatagacaaaaattacgaggaggccatattcaaatacgactacagtacaagcataaaatttcccccccttaaaaatggattcccctccgacgcacctaggtttgatgactctgaaggcaccgatgcttctactgcatcttctcatatgggtcggagtagctctacttcatgtttgtccgatgaaagccatgaggaagatgcaataatgggtaaaaagacaaagcatcgctcttttacgaagaatgaaaattatgctgtaatagtgttgcgctcagaatgttatctcccggaggagagagatagcgagagatacatttgcattagacttgtggaagggagcggagaggaggagacggtaatactggcgaataagggcaatttaaaggataagaagaatgaggggagtaacttaaacgttgatatgcgtgtttcgggcgggaggaacgatgtgagtagggtgcgtcctacaaactcttcgcaaataaacattatgttgagcagtagtgaagtaaagtgtttcaatttaagaggcaaggtttatgaaagaaagaatttggaaataaatttgataaataatacagaaaagagggtagaagtaaattcgaatgtttatcatttatatataagagatttaaaaaaagaagaaaaaaaaaaggacaattttgaaatggacatgttaactgtagacaaaggtacccacgaaatgtacaatcaatgtgttgtttgttctatgattaataccaatttgaggaattacctaaatgaacatgaacatttttttagctgcttttacgtagagaatggaaaggatttaaccattggaaaaaatgaaaggagaaagatacaactgcacttttgcccttttgtggaaggcaactattttggttttctcctattttgcgtaaaagacaggagaacaaaatatttgttgtccacatgcaaagtaaactgcttcttctacattaataatgtgaaagaggacgaagtaattaaaatgaataccttatcttataacttcacttacgatatgaagttaaatcccataaataaagagtttttgaaatgtgttaaatttgtgctatgtactttaaataaaatggacaaaaagtattttatggcctatttgaaaagttacctaaaaagcgtcaaccaaagtaggcacaatttttacatcatctgtgataagtatgacaaggtggctatcaaaaagagcctcttttcgttccagaatctggactatgccaggtatgtaagcggtgtagatgcaggatcatccggcgacgtggacgtggacgagctgtgcgatgcggttaaacgagacacgaactgcttttgcaatttgaatgtgagggaggaaagcggtggggtgtatgagtacaactgcatcctgttagtgaagaaaaaccatgaagaaaaaaaaaaagacatttcaaatttcgtaacttatgaggatgttagaaatatgagtgtgagggagcagaattgcatacatgagatgtgctatagattgtacaaaattatagcaaatgtggacagtgaaaaggcaaataaggtattcactattgtattcaatactagcgcctatttattatcaaaaaagagtataccaatatataattacactaatgaaaaattaacctatagatgtattcattcggaaataatagatcagcataataacaagatagactataaaatttttaaaaatgatgaatatgtagaaataccgaaagatatagaaacgttcaattttgaagtaatatgttattctattgttgaagttgtgtgttcttgtattattctcatgactaatgtagaaaatgaggaagatcaaataaaagtcaatgtaagcgctaatattaagaaaccttacccgaaagagactatacatataaaacttcttaatcgaatgaagaaaactgtacagatggaactatataacgagttggatttccattgtgaatttaaggtgtactcagatctaccaattctttttggggataaaaaaattgagatgttaccaaaacagagaaaggtatacacgttcttcgttcgaagtgcgcacattggtgaatttgttggatgtctcattttcaagtttttcaatctcgtcaacatgaagggtacccatgcttctactcggtatagcagtaatcaccatgatagtgatcgaccaaatagtaccgtggatagttttcccttctctgattactttttttggtacaagttaaacatcacagtggagctaaatcagccgctaaaaattttgctccttgagacaactgtcggggatgaaataagtaaggaaatcgttttaaaaaataatggacgtgaaaaggaaaaatattttttgctgacatacatgcacgaatttactgaaaggacacaaattgaaataccgaaaaatgatattcacgtttataatattaagtatgcaccaaaaataccaaactattttgataatctacgcgatacgatggaatgttacaagttggacagcgagcaagagaagattgatcctttctgtggaacaaccatttccaattcgttaggtcgtgaaaaggaaagagtttcttctaccgaaggggacgctcccagttttaccggtggaggattcgacattgcacaggaactatccaagttttacttccctccgaacgaggagagcgaggaagagatgctagtggaaagtgcggtgactcgggaatcgtccagtggaagggctgtcattgggaatgtagccaatggagaggaagccaatggagaggaggccaatggagaggaggccaatggagaaacagtaagaggagaaaaggacgaatatgcagcttccagggtaaaaaacctttacctgaggagtgaaacgatgaaaaacggaggagaacaacccaaaaaaacagcatccatgggtgtaaaggttcgcagcatggagaaaaaggagcatccaattttgaggcagaatgtaaagcacaaggggaagaagaactttccacataatgagaagaggctcttcgaagagttaatacaatactgtaaaagatacataaatgtaagcattaattataccaatcagaatataggatttttgtttatttacaacaaaaaggagggtgtaaattactacattttaatattgttagcaaaattaagggaccaactaacgataagtaatttctccgcgtgcttatccaagtcttgcttcatccatctacatttcgattttaattctgaagaaaagagatatgtaagcatgttaagctcaaaggagaatgcacattcgaatgagtatttttaccaggtaggggaatacgtacgacagaggggtggcagtgatgcgggaaaagataagcacaaggttaaatgcatttacctgagcaacagaacgaactacaccattgggggaatcgcggatgggggcgtggatgtagacgtaggtggactggtgtgtgaacgtgaagacgaacacactgacgaaccaggcaaccacgttgtaataaggtacaaacccacgacgaacagttcaaaagggtgttatgccttagtgaggagcgatatatatggagactttctctatttcgtcaaaggagtgtatacagtgaagagaaaaataaaagaaaagatgattatgtataattctggttgtctgtaccattttaacgtaaatcttttcaacccatttaattgcaaagtgtgcataaagtgtagattcaaaaggaggagagatgaaaggggtggaggttctcatatcatttccgatagttctgagcatgaggcagagaaaaggatgaaggagaggagtgtccattcagtatctgtgaaaaaagaaaaagttcttataaataatgaaaaaaaatattttaaaatattaagtagggaaaattttatagtaaacaagagaagacatttttcactactgatgacttacttttgcaataagataacatcagtacaaacattagagatcgttttaaagcctctccaggatattacaaacagaggatcattccaatctattgcttatcaatacaagttcgttttcaagaacacccaaaaggggtctctgatcggagaaaaacatctcgtatgtgatgtccccaatgatgaggtcacctatcacaataggtttgacgaaaattctctaacagattcgtcaactggtggggaagaaccttatccctgtaacgttgttaaaaaggtggattacaaccatgtcgtttatacaaaattaaatgaagtatttaccactgaggagggggaagcagacagggaggaagaaaaacattcatggcgaatgatatctactagtaaatgggaagaaaatatggaaatgcacacaaacgaaggtattgcttctccgggttgtggttcaacaacaaccgattctgagtgcactcaagatggtgacttggtatctaccccggtgaagaaaaccccatttggtgtatgtgaagataggaaagatgtaatggaagagcaagttgaacaggagaattgcggactaagtagtaaggaagtggtatactacgatgggaagttattcattgagagtatttgcaaaacaaagacacagatgctaatttacataaacaaaaagaaaatgatggagagagaagattatgacaaattcatagacgagttggaaaatcgaaaaagggagaaaaatatgctacacagaaatgtcgaaaaagggaaatataattataaaatttttttaattaacctaaaaaatttaaacagcaattggggtggattaaacgggaaaacctgcgtaaaaatgaacaacatccttttcgatatgaacgaagaactgcttaataactacttatacatgcatattgtgaaagacactccaaccgacttggtgctaagtctggagctgctcgcttatcttcctttccactgtagcttctgtgttgtggtcaagcgcgtggaaggcagaggcggggcagccatggaagaagcggaggaagtgagggaaacgggggaagcgggggaaacgggggaagcggatgggatagattgggaagacggaatatacgctgacgcgggggagcggtgcgtgcaggtcgaatgtctcaaacgggaggaaacgaagaaatttacgatcgttgagaagaactacatatccgtcgaaattttcaactacatgaacgtgtcagaaaagtatataaatgttttcgtcgacaggaacttgtgctccgaaacgaagttaaatattttttacagacacacaaatgattcctatttcgaagcgtacatggtcagttataaccaaatgagtaattatatcctcacagaagaaattaaaaacttcgaaataaagccccaatcaggaatattaaaacatggaagccacaatatgtttttgattacccggataaacaaaaataaccttgtttcttttagtaattcctttcttcttttaataaaaactgaaaggaatatattttcctatattatcttttcgcggtattctccccctcgtggcttgcgcactacgagggagcagaacatgctaaaggagatcctaaacgaaaacaagaaaaagttcagcgttgtctttaacacattcagacaggaaaagaaggaaagtgctatatttaacaaaaaggatcaaataataattgaagatatttcgaagtcaacaagcgaaatgaggaactactga

SEQ ID NO: 23 - DNA, 14676 bp, Artificial Sequence
  Synthetic
atgaatgagatttcagagaataatttgaacattttagatgtgagtcctcgaaatgtcgaatttttttatgaatgtctggaagatgtagatgagtttgcacaaaagaaaaaagaaaatataatagaaaagaaaagtttaaaaataaaaaacatatgtagtaaaattcaaaacatcgaaatatttgagactgaatataagtatttaaaaataaaaggaagtaaaaaaaaaaagttggctcctggtacatgtgaacatatatgtatagaattttctttgtataataatgtagatataaaaaaatataaaaatgataaaagaaaagaagatgttttaaatataataaataatatcgaacgtaccatctttcttaaaatcaaaactgaatatacatctttaagtatacctatatatataaaaaaaaaagttgccgtatttttttatgataacattattaattttggattatgtaaacaaaacatgacatatcattatcctatgaaggtaacaaatgtaggaaaaaaaaaaggaacctttacaatatctagtgacatgtatcaatccatgaaagagaaggggaatcacatattttttcaggatgaaaagggggaaagaaaaaaaaaagaattttgtatggagaacaataagggggatcatcagaacgaagataaccatgatataaaaagtaatcataatataaaaagtaatcataatataaaaagtaatcataatataaaaaatgatgataatataaaaaatgaccatcatataaaaaataaccatcatataaaaaataatcataatataaaaaataatcataatataaaaaataatcataatataaaaaataatcataatataaaaaataacatgaaggaaaaaaacctcttagtagatgaaaaaaaagataacactcttattacctttgataaaacatctattacattagacattaacgaaagtaagattataaatattgaaataaaaaatttaaaagaagaaaaagtacatcgtgaatataacatattaacattagaaaatagttactttgttgagaagcctcgaaatattattattatagctgtttttataaatagttctacttctttttattatgataatataaaaacaaaagaaataaatatgaattatatatattatggaaataagaaaaaaatacaaggacaaatcaaaaacgaaaataattataatatatcatatcaatataaaatgaaaaaagtgcatgctttctatacattggacagttttaaaaagtatatacaattaaataatttgtctgctggatctttggaagaggaattatgtgagatggaaaaactattaggacaggaagaaaaagaagataaggaaaaaattatgtccatggcaaaaaaaatgatgaatattgaaaaaaacattcatgttaaaatgaataatgatattgtctgcaatgtaaagaaattaagttatgaatctgtttttttcgaaatagattcaaagcatgatcttttgttcttagaaaaaataaataaaaacaaatcataccttttaaacattccaataataatagatatcacattgtttgttaataaaagtgatgatgaaaaggataagacgaataagataacgacaggggggataagtaaaatgcataatgacaataatatgaaaataataaataaaagaaaaaatataaataataattatgataataattgtggtaataattgtgatgataattatgataataattgtgatgataatcatgataataattgtgataataattgtgataataattgtgataataattgtgataataattatgataataatcatgataataatcatgataataatcatacaaataaccaacacaataataatatttgttgtacaaaccttttacaacttcataagagagaagaaaaagatagagactcttctattcatcatgcctcaaattgttatgaggaagaaataaatttacatttcattttttgtttaacactcccttgcctaataacaaatatatattttttaaattataacaaagtaacgacagaagaaacgaaaagcatgataatagaattactaaataaaaataaatttttaaaaattaattatagcatatctaaaattccttatataacaataaataagaaagaagggaaaataaattcgcaagaaaaagatataattaccattaccctgaaatgtaatgtcataaaaaagattaatgaatatatgtatatttttttttgtaataatttatatttttttgttatttttataaatggagaagtaacacaaatatccaacattaacgatgataatatgcttacaataaaaaatataaaaaatatttatgataacaacatgtgtgatgaaaaaggtattacaaataatgataatacttatatattaaataataaaagtcttttaaaaaaaaaaaaagaaaagagatatacagatgatcataataataataataatgatcatgatgataagatatttgaacgcattcttaaaaatgtagaatgtaataaagtcaatagacatttatatgaacaagatgaacaagtagataaatataaatataatgaacattttattaattatttaaaaaataataaaaaaaaatataacgatgttttgaaatatatgtacaaaaaaagacataattcaaaacataatattctacttaaggataatataaaaaataaggagtctatgataattaaaaaaaattcttcttataatttaaaattttctgaagataataatataaacaaagaatataatatagatcataaaagaacaaatgttttaaaggatatatatatatatgtaaatgaagaatatataaataaaattacaaatatatatatacctcaacatttctatgaagaaaaaagaaaacaatatataaatatgaaaaaaaaaaataataaacaatatttagtgggaacaatgaaaaaagaaaaatatttaaatcaatccttatgtttaaaaaaaaataataaaataaataatattaatattattaataatgatgataattttcaaaatgtacaaattataaaacaatatttaatatatccacaaattattcattttaattatatactattaaataaagaatttgaaacgtttattcatttacataatactaattctatacatccaataaacatatatttttttttccatagtgataatataaaaattacttatcctgaagaaacaaaaaccacatgtcataacaaaattaatgatagcaacatatgtactccttatcaaagtaccaaagttaccataaaacagaatacacaacaaaaaatatgcgtcacacttaatttaaaaaattataatatttttaataatcaaaagaataaaaaatgttctttctctcaatcagaaaatatggataatgaagaagaacaaaaaatatgtaaccatattatttcaaaaagaaatattaaaaataaggagcatcataattatttagatgatacttataaagatgaaccatataatatatatcatcataaaacaacacctttctttgaaatatatatttattatgaatatatctccataaaaactcaagatcattatgatatagacaaaataaaagtccaagctaacttaatattattaaatctttttataaatactaacatattatatttttctattacaaatatggatatagacatgttttatcataactatttagttatatacaatccgtttaatatcacaattcagatgaaattaaaatataatgagcaagtcttaaaaatgaaggaccatataagtatatatcctggtgaatacaaaatggttccagtacaatttattaccagcgaagcaacagcttgtgtagaagaatttatttatgtatacttggatgactttagattatacaaaagcataaaatgtaagtgctttccatccccgtgtagctataaaattaatgtgaatgaaatcaattttgataatgtccttttaaatagaaactatttaaaaagcttttacataacaaacacaagtaactttccacttgtatttatgtgtgtttataaacccccatatgttcatgtccttgcgaaacgtaattatgctaataaaaatgaaaaaataaaaattaccatattaataaatgtaaaagaagaaataaaaataaaagataaacttatattctatgtgaggcaaagggaaaatctaattctacctataagtgtcaagttaacccagtccaatatagtgatcgtgaattgtccagatattattcaagaaagcatggaacgtaaaaggtacaacatcaatattttaaacaaagggatgtacgaagaaagcgttattataaatttcaacgaaattccatttttgaatataaatatagaagatgaggatacaggaaacaaattaaattatataaactataaaaacaaagaatataaacatagtgaagagaaaaaaagaaatacatttgttaatatatataatttagaatatgataataatattataatagatgaatatataaaatattattataatgaatttattttattatataataatttatgtaataataattataaaataaaaggtatacttcatatttcatcagatcaaaaatgtttacaaaattgttataaaattataattccacataatacatttataactttaatgattgaatgttatataaataaaatatatgaaacagatttatatttatatcaacatgtattattaaatttaaacaatccatttgtcaaagatgaatattataaagataaattaaaaattaatataaaaaatgaatatttaaaatttactccttcttatctttactttgaaaatatattattatgtagttcggagaaatatttaatcacttcttttcaaaagactataacaaaaaaaatacaaataataaatgtgtatcatcaaaatttaaaatgtactgtaaagatatgtccttataataaagaacagacaaaaacatcacagcctgtcaaagtatctagtaataataataataataataataataataatagtattataccaagcgatcatgatatgatatgtacacattccacttggtatgaaaataatcatacaaaaaaaaaacaacattatatagacataagcaatgtaccagatattttaaaagttaacgaagagtcttatataaatataaaagttgataatatagaaaaagaaggaagatatgtggaaatgttagaaatatgtgcagaaagtgttaataaaaaagaaaatgaagaaaaaataaaatattctttatatatacttatcaattgtaaagatgttaaattatatttcgatgtttcatatattaatattatacataataaattaaatcaatataattatttctctttccatatatataatgatggttataattatgtaagagccaattatctttttaataatatatttaaagaacactttttcttagatctacaatttttaccaaacaaccaactaaataaaaaaaacaaaaaaatcaaagttcttttgaaatatctatcaaaaattaatatccagcttaaaacatttattacgataactgtaatggaccatgaaaaatatgacatacctttgttcataaatatacacgaggaaattgattatctcatggaaaaggaaaataaacataagcagaattacacacattgtcataattacacacatggtcataattacacacatggtcataattatttacacgaaacggacgagttatacgaaaaacataatgggaatgaaaatagtgatgacataaaacatccttatccttatgatagagaaataaaaagtgaagggtctacaaataaatcccatatattatcatcacacctgtcaggtgatcacaaagatgaaattcctattataaataataataaaaaaaaaaacataagttattttactaaagaagaagaatttgccttatataataatatgtctataccatatagtcttaaatataaacaaaataagaaaatggatgtagacgaaaatcaaaacatgttatccttttatcgaaaaaatatggaagagtcaagtaatatatatactaatatatttaaaaatttacaaattgataatataagaaatccttattataatacaaatgaagaaggaataataaatttaaaagaaattgtacatgattatatgttcatatttcttcaacatattatcatcaataaaaattattttccacaaataattgaaaacactaatgacttatatgtatttttcttaaacattttatatgatctatcttttatatttaatagtaacaagaatatacaaaatataataaatgaactaaaagcatatgagcatatgcaaaatggtagtgttgacgaaaatgaatttataaaaaatatgaaaactctaacagaattgttgaaccaagagaagttactaataaatcatataatgtatgaaaatttactacctcttcatttgtacctacaccatatttttaataacattccagaatacttttatataaaaaaagaaagtgataaaaaggaaaaatgcaaggaaataaacaatttagacaacacaatattatatgataataaaaatgatatagatgaagaagaatataataaggaaaaaaacaaatttctaaatatatctgatatacaatcatttttaaaaaatgacaattataaaggagataataatattttttatttagataatatatttaaaacatatgttaaattatttatatattcttggatcaccatattcttagatattatttttaagaaaatcttttctttcataaatattacatcgttatataattcgagaggtactaaattatgtactcttgaaaataatatatatgaaaatattaatatccataaaataaattatcttatagtattaaatatagaaaacaaaaaaattaacacgtatacatattcctgtacaaatgaaaaaacacaaaaatgcatagttaaaaaaaaatgtatagacgaaaaaaaaaaggaaaatgcaaatataaatgtaaaggagttatcatttaataatcataaaaacaatgaagatttaaatatacaagaaaaagcatactttcatttttatcaaaataattttatcgacataaaggaagtgcttcataaaattggacaatccacagacacagatcactcaaaaaaaaaccaagaaataaaagaaacaaaagaaataaaagtaacaaaagaaataaaagtaacaaaagaaataaaagtaacaaaaaataaggattcttatcatacaaacaatctttataataatactaacaatttcaaaaacacggtgaaggaaaaaaaacaaatcaataaaactttaaaaaaggagacacacaaaatattaaatcaaaaaaaaaaaaaaaaaaataccaatatagatgaagacaaaaagaaaactactgaaagctctacatatacagagaaatatgaacataataagcatctgaacattaaagttcaaacaaatataaattattatagaaagatagaagttattttatatgaatggttatattttcattataataatgtatataacaccaaagtaaaaaaacaaaaatttatatttacccaacaaaaaaaagacatatcaaaacataacaagttatatcttcagtatgatcaaaataaaagaaactctgaaatagaacatacaaatcacaaagaagattattcgatgtgcgacaatgtgataacaagcaaaatgagccacatatctaatatggataatcaatatggtaataatattacatgtgatatacccttttttgttgagaaaaaaacgaacaagaagaagaagacgaagaaaaaaaaaaaaaaaaaaaaaaaaaaaaatatttttaaaaaaaagaaagaattcatcacaagtttatatgaattaaatgattttgttattattatatatactataatttctcatattccttattttttattttttaaaaataaaattaaagacccatgtaaatgtcaaaaagattatttatataacatagacatactattattaattttgaacaccattaaattaaataatttaataagtagaaatgtattaatacaatttaattatgttcatatatttatattcttatcatccttatatatcatattaccaaattatattccaaattattatttaattctagatgatttctcatcatataaaaatgacgtaagggtaataaaaggaaatttacagaaaaacacaaggacaaataaattaaagcaacttcatatgaataataaaaaaaagaataacaataataaaaatacaagtgataaccatgttcataataattataatcaagaaaaacaaacaaaaaaaaatattacatgtgatgaaaaaattaatgaacgaattatttacatatataattctaataaatacatgtgttcatatgatatttttttgataggatcgaacaaatatactattgataaaaatcatataactatacattcaatgaagcatgaagaaataaccatatccattgatccacattatgatgaccatattattaaatatgattgtaataacgagatttatttaaaagatattaataaaataaaatataaaaaaaaggataaaagttctgaacatactaatagtgagaaccgattttccgatagtagtaaaaattattcatcattatcgtcatcattatcgtcatctttatcgtcatcgttatcgtcgtcattatcgtcatcactatcgtcatcactatcgtcatcattatcgtcatcattatcgtcatctttatcatcttccaattcttatattcattctcggtgtgactcatcaaatattcctaaaaacatagacacaaataaaacaaattcaaaacatataaaacacatacattctgataaaagtaacaactattcaattcttgttttgagggaatcaaactataatttattggatttaaaaacggcagaggaaaaattcatttgcataaaaattatagaggggaataatgaagactcggaaaaatcacttttggggaggaacaaaaataatataaaaaataaggataacgaatataattcggtatatgataaaacaaaagagagacaaataaaaggtgatatacataagaataataattcttcagattgtggcgagatgaatattattttaaatgatagtgaagtgaaatatattaattttaaaggtatggtgtatgaaaaaaaaaatatccaaataaatatgataaataatttcgatagaaaagtggaggttacaacaaatgtttatcatatatatttaaataatagaaaaaaggaaattaagaaaaaagaaaattttgataaggatatatataatttgaaacatacaaaagttgaggaaaataacatacataataataataataataataataataatagtagtattccttttaatgctaattatgatatgtactatgaatgtgatgcctgtacaaatataaataatcatctaaaagcatatataaataaagatgatataaattttcagtgtgtttctctagataaagaaaagaatatatatattggtaaagataatggtaataataaaataaattttcatttttctcctttttttcaaggtacgtattattgttttcttttattttatgttttatgtacaaaaacaaaattgcttttatcaacttgtaaaatcaattgtgtcttttatataaattacataaaaccagaagaagttattaaaataaattataaatcttataactttacatatgatcttattttaagaccactgaataaacaattcttaaaaagtatttattacattttatgtaaattaaataatagcagtaataatgtttttttttcaatttatataaagcgatatttagacaatatatatatgaataaagagaacttttttattttatgtgactctattaaaaaagtaaaaattaaagataagaaaatatcattcaacaattttaactatacaaaatattttaaagatgatataataaataaaaatatagacattgatctattatgtgatatgataaaaagggatgtcacgtattcttgtaaattagaaataaatgaagaaatgaatggtgtgtttgaatataacttttatcttttaaaggggaaacaatacaatgtagaaaattgtttattcatgataaaagatggaggtgaaaattatgtaaacgaccaagtgtcactatatataaatgggaacaaaaatgtgaataatatgttttcccctccaaaaaataataatgtcaacagtatgaataataataatgatgatgataatgatgacaataatgatgacaataatgatgataataataataataatcataataataataatcataataataataatcataataataatcataataataataataataatcataataataattattattattataattcctgtgatggtgatgaacttgtaccgttgtgtaaaccaaagggaccagataattaccattattgtgatgttaagctatataaaataatatgcaatgtcaatgatgaaaataatataagaacaattaatataaatattaaagccggtatagtatacaaaaaatatattcctttatataattatactaataataatattacatataaatgtgctttttccgaaattatcaatgataaaaatatgattattaattataatatatttacttgtgatgaacttgttaaattaaaaaaagatatggaaatcttcaattttgaagttacatgtttttgtcctgtaggtggaataaaatacaattcattttttatcttaacaaatgtacttaatgaacaagataaaataaaaataaaaattcaaggatatatatcaaaacctcttcctagagaaaatatacaaataaatgttttaaataaaataaaaaaaaatatacaaatcgaaattttcaatgaattagatattccttgtgaattcaaaatatattctgacttgttcatattatttggagaaaaaaaaaaaattgaaatgatgcctaaacaaaaaaagctatatacattttttataaaaagtgcacatattggtgaatttattgggtgtcttatattcaaattttataaatataaagatacaaataatgaaaataatgcttcctttttttctgattattttttctggtataggttaaatattgtagtacaattaaatgaacctatgaaaattttatatctagaaacttttataggacaagaaatcacaaaagatattattataaaaaataatgcaaatcaaaaagaagaatatttcttgttattatatatcaatgaatatatcgaaaaaaaacaaatacaaatagaaaagaatgatatatatatttataaaattatatatttgccttatatacctaattatttttataatgtcgaaacatcaagttgttcacattctaaaactttaacgggggaagaaaataataattattcaaatgagtttcatgaaagcaagcaattgtttatggataagggagaatatgcatctgatcatttcatacacaatgaagatgaaaaaaatgaaaaaatgaaaaattattcattaaaaaactacataggagatataataaatagtcaaagcaataatcatgtacaacataataatcatattataaacagtctgcacaatttttattattcaaaaggggaatataataatatgccacaatgtttacataaatcaaaaaataaagacacatttacacaagagtacgaagaaaataatgttaatacaagtgaccttctttttgaacataaacaaaaaaagaactttcaatcaaaagaaaaacaaatatttgaagaattaattaactattgtaaaaataagtatattaatataaatgatatcaattatgataatcataatatcggtttcttttttatatataataaaaaacaaggaatcaattattatattttaatattactatccaaatttaaaaataaattgataataaataacttttatacaagtttgtcaaaggcatgttatataaatattcatttccattttcaacatgaacaagaaagacaagtgaaacaattacaagaaaaagataaacaaaaaaaattacatagtttttcatatgaaataatcgaaaaaatggacaaaatggacaaaatggaaaaaatagaaaaaggggaaaataaaaataaaaataaaaataacatatatagtgcatcatatgatatatgttcaaataatgatcaacccttttcttcttctacacatgatgtttataagaatcatttaaaaaattcattattagattcatatggacaaaatcattcttcaaatattaaatgtatatatttgagcaataaaattaattattctattttacaaaatgaagataaatcaagtaataaaattgttattaaatacaaaccaacaatggaaacatctcaagattgttatataatcataagaaataatttatatggagattttatttattttatgaaaggaatgtatgttgtgaaaaaaaaaataaaagaacaaatggttttgtataactcctcttctttgtaccgtttcgctataaatttgttcaacccttttagttgtaatgtatgcgttaaatgtggtctaaaagaaaacaaaataacaaataaaaataataagaaaattataaaaaatataaaatatatgaaaaatataaaaaatcacaacgtacataataataataccaacactgtaaataagaaaaacaaacacaagagctcacgttttaagggaaaccaattattattaaataacaaaaaaaattatttcctaattatcaataatgaaaattttataattaaagacaaaaaggattttcctctacttattatatccttctgtaaatatgaaccatcacatcataaaaccttaatcgtaaagttacaaccaatttataataagaatatgaataacaaacaaattccttatattatatatgaatatagtctagtttttaataaaacaaaccaacaaaaaaatcaaataaaaagtaatacattaaatcattttgtcttatccgatttgtataaaaacgaatcactgacacatgaatatcaaactacagaaaatgaggaaaatgaaaatgatgtggaggaatgtcaagtgaatatacacgagtcaaatgaaaacgaaaatgacatgtgcgagtcaggtgaaaacgaaaatgacatgtgcgagtcaggtgaaaatgaaaataatatttgcgagtcaggtgaaaatgaaaataatatttgcgagacagctgaaaatgaatctattagcatgtacgagtcaggtgacaaatataccacagatagccaaaagagcgatactgcaaatgagttatccattggagagttaagaagagaatatacagataaaatgagtatgagtcaaaatggaaacgacgataagagcaaatatgattattcttatgatgatgattgttatttaataaatggtaatgatataatttattacgataaaaagatatttgttgaaagtatttgtaaaaagaaaacgtgtgtacgaatatacatagacaaaaagaaaatgagagaaagtcaaaattatgatagtttaatggatgatttagaaatatgtaaaaagaaaaagtgcatgttgcacagaaatgttgaaaaggatgaatggaattataaaatatatttaataaaattaacaaatgtgtataataataataataataataataatagtgatgatatagtaaataagcggatgatcacaatgaataatatattatttgaaatgaatgatgaaatgttaaatgattatatttatgtaaatgtaatagaagatacggaagaaggattattaatagatatagagttgttggcctatttaccttttcactgtaatttttttctaatgattaaaaaaaaaatgaaagaaaagataaatggagataagaagggtataaatattttatcagaaacttgtaatgggaaggacattaatatattacctgaatataaagataataataaatatataataaatggtgttgaaaaaaattgtatatttgttgaaatatttaattaccttagaatatgtagtaaatatataaatatttttgttgataagaatttatgttcagaaaagaagttaaatatatattacaaaaataatagtgattcttattttaatgcatatatgataaattacaatgaatatagtacagatgacgttatggatgagatattaaattatgatataaaaccttcttcaggtgtattaaaacataataaatataataaatttataattataaggacaaacaaaaatggcgtggtaaatatgaataacattttccttctcttgataaaaacggaaagaaaaattttttcttacatcatattttctcattacaagcatacaagaaccatatgtacaatggatgagtataataagatatatgatataatgaatgaaaacaaaaaaaaattcagcgaagtttttaatacattcaaggaagaaaaaaaagaaagtgtcgtttttaatgaaaggaaccaaattattattgaagatatacaaaagtcaacaaatgaaattcaaaatatttaa

SEQ ID NO: 24 - DNA, 13302 bp, Artificial Sequence
  Synthetic
atggggttagatgcagagaaaaacaaatttgaccttatagatgtagaacccaaaagtatagaattttattatgaccgcatagaaagcttcgaggaatttgttgaaaaaaaaaaaaaagtgatagaaaaaaaagttataaaaataaaaaatatatgcacaaaaattcaaaacataaaaatattcgaatctgaatcgaaatatttaaaaataaaaggaataaaaaaaaaaaaacttgctccaggtacttatgaaaaaatattagtagaattttcattttgtgatgtaaatataaataaatataaatatgaaaaaataaaaaatattttagatgtaataaataatatagagggaactataaatgtgaaagtacaaagtgaatacacaacattatatattcctatatatataaaaaagaaggttccgatatttaaatttaataatgtatttaatttggggttatgtaaaacaaatttaatttatgatgtaaaattagaagtaaaaaatgttgggaataaaaaaggaactctgtctatatgtctagacaatataaataatgaggcagcaaaaaatgaggcagtaaatgatgataataaaaatagtaataccctttcgaatagtgataataaacatattattgaaaataatttttttatttattttgataaaaatacgatttgtttaaaccctaatgaaagtactataattaatattaaaataaaaaataaaaacgaagaaaagattcaaaaatcttataaaataattacacaagaacatagttatttttctcaatctcctaaaaatttaactatgatcgcaatttttataaatagtcaagtatcatttatatatgagaatatgaaaacgaatcaaatagatttgaattatatttattttggaaattcgaaaagaattaatggacagataaaaaacgaaaatcaatatccagttatttgtaaattaaaaaaaacacaaatcaaaatgttttatgattccaaggagtttgcttcccatgctgttgacagaggcgtagatgtccggactcttttgagggatttggacaattttcccgaatttgatgttaatgatgaggaaaagaaaaaaagtgaagaaaatattttagatgtaggaaataatttaaaactagaagaaaaaataaaaataaagttagagaaacaaaatgatatatatttcatagacaaacaaagttgtactgatattttattatatatcgaaatagaatcatgcatagatattttaaataaaattgataaaaattattcatatatgttaaactatccagtaatggcagaaataacaataaccgcaaataaatgcgatgcaaaatgtaagccttctactaataggaaaaaagcaattaccgatttgcgacaatttgacaaacttagaaatggtgcgaatactgtgaacggtgcgaattctgtgaacggtgcgaattctgtgaacggtgcgaatggtgagagtgtggaatgtagtgaaggttgcgaagaagatttaacattttatgtatttttttgtttaacattcccatcaatcgtttcaaatatatattttttaaactatgatatggtaggacaaaatgaagaaaaaactttgattttagaattaaataatttaaataaatttttaaaaataaattataatttttctaaaatttcgcatatacatctaaataaaaaagaaggtgttattaacccattgtctaaagatattattgctcttaatttaaaatgtaatgtaattaaaaatatagatgattatttatatttatttttttgtaataaattatatttttttgtgttttttgtaaaagcacaaataaaaaaaagaatatcaataggaagatcgatagttatcaaaaatgataaaatgatcaaagctgatggaaaaacaaaacaaattaatagaaacacaattgagttagagaaaattacaatttccaaaaaagaagatgaaaattcaaatgaagatataatttatgaacaaattttaaaaaagctagatttagaaaaaattgacaaaaatttatatacattagatggtaaattagacaaatataaatataacgaaaaatttatgtcttttttaaaggaaaataaaaaaaaatataatgatatattaaaatatatgtataataaaagaaatgagaaaaaagagaaacagggaataaagaacccaatacctgaaaatgatcaaaatgaaaaaattaataatataataaatgataaatttatatatataagtgataaacaagtaataaaaaatactaacatgtttatacataaatctatatatgaagaaaaaaggaaaaaatatattgatatgaaaaagagaaaagagataaaaatatttaatattaaaaaaaatgatgataaaaaagaggatattcaattagaatataatgatatattagaagaaaaccaaattaaaaatcttatttttgataaagtaatttattttaattatgttttatttaatcaagtatatacaaaaatatttaatgtacataataaaaatgatgaatgtaatataaaaattaattttacacatagttctaatttaaatttagacaaaaatttattatttataaaagggaattcaaaagaaatgataaaaactaacttacttttaaacttacctattataaaaacagatgtagaaaattgtaacaaaacaataaacattaatgaaataaaatatagaaatatttttgataacgaaaagacaaattttcaaattttaacagatcatacatttaatcaaaaagagcaaaataatttatcattacatgagaatgagaatctttcaaataacaataaaagttatttatttgatcataaatattatacagaaaatataacattaaagattaatgataattatgaagatatgatagttgttaagggaaatataatacttttaaatatagttattaaaataaatacattatatttttattttaaaaattcagacatatctatgttttgtgaaaataatttaattttatataatccctttaacattcctatccctatcacacttgaattctccaaagaaatttttgaagcaaaaaataaacttgttattaaccctagtgaatataagacagtggcaataaaatttataggaacacaaaatattgagcgcatggataattttattaatttgtatttagacaataatagactttacaaaagggtaaaatgcatatttgatgtaaataaatgttcttgcaaaataaatgtaaatgaaataaagtttgagaatataattttaaacaaaaaatatttcaagcatttttacctgaccagtacaggggatactccggttgtctttaattgtgttgccaaaccagattgtgttaatatttattataaatataattatgttaataagaatgaaaaggttaaaattatagtctcagtaaaattgaaagaaaataaacaaataaaagataaaataattttgtctattcgcgggtctgataatattattatccctattattgttacaaaggtttattcgagtaatttgttatttcttaatgatattaaaatagatcaagaaacaaacgaattaaagaggtatgaaataaaaattgtgaataaaggagggtgtgaagaaaatgtgattttaaatttaagtgaattaaatttaagcgaattaaattttttaaatattgaattaaataaaaaaacagaaaataatagaataatatataataataaagaatataaaaatgtaagaaatatgaaagatgattttatggttttaaaaaaaatatttaagacagaatatgagaatattataaccaataaatgtgtaaaatattattttaataaattaatagatttatataattttttacacaattttagtaaaaataagtttaaaattatatttgtagaaaataaaaataacgatataaaaataaatgagaaaaatatgtacaaaataaaaatatcaccaaaatgttttgtttccttatatattcaatgttattataataatataataaaaaaagaaataaaatttaacaaaatattgtttgaaaataaatgtttacatgaattatcaaatgaagttataaatataaatattaaaaatagccaattaagtatatcaccaaatttgttattgtttcaaaatattataccttataattctgaacgagctttcataacttattataaaaaagaagggattcaaaaaataaaattaaaaaataattctagttaccctattaaattggaaatattattatatgacaaaaaacaaaaagacagtttattggcaacatcatcagttgacaattttgtagaaatggaaagaaaacaaattattaaaaataatgaagagagtaatgaaaaaattgaaataaacaaaacattacctagagcagttccaaaaaaagattctaacctaaatttaaagaaaggaaatattcaagaaaatgtaggaaacaaatcgaatgaacatgttgaaaaaatgtacgaaattgaattaaataaagagagtggaataataaaagcaaatgaagaaattgaagttgaaataaaattaaaaaattgtaaagatataggaaagtttgtagatatattggaaataaaaacgaatgttgtaaaaaatgaacaaacagataaaatctttgatgaaaaaaaatattgtatatatattttatcagtaatcgaaattcctaaattatattttgatattacatatataaatataatatataataaactaaatgatacatttatttttccttttaaaatatataactatggatatagttatgtaagaataaattacaaatttgaaaatttatatacagattttttttttctatcattagattttttacaaggaaatgaaattgacataaaaacaaaaaaaatcaatgtagaattaaaatgtaaatcaattaaaaatttaaaatttaaatcagaaattataattagtatattagattatgatatttataaaataccagtgtttgtaaacattgatgaaaacattgattattttttggaaaaagtacaatatgaaaaatttgatgcattaacaagtcaggcaaataattatagtcttgatcaatatatgtgtaaagaaaattcaagatcaaatttgtctgaaataatagatgaagtagtctggacagataaacagttcaattctggcaaagctgaaaatggcaatgttgaaaatggtaattttgaaaatggcaaagctgaaaatggcaatgttgaaaatggtaattttgaaaatggtaataactacaacatttccaacgttagcgaacaaaataatgagttgctctctttttattgcaaaaatatatgtacagaaaattgtaatatatttaaaaaaagatgtatatataaaaatttaaaaattaataatataagaaatgtatattataataaagaaaatgaatttattgaaataaatgaaataccaaaagattatgtttttgtatttcttcaaaatatattattaaataaaagttgtgctcctacatttttagaaaatactaatgatttgtttttattttttattaatttgttaataaatttttttcatatatatccaaatagaaatatatataatttattacatgaaattaaaatataccaaaatatttcgatgcaagactttgaaaaaaatatatttatccacgaaaatattcttgcaaatataaagcaaattttaaaaagcttaaaagaggaaaaacttttagtgaatcacataatttatgaaaatctacttcccatagatttgtattgttgtcatatactggataaggttcctgacaatttttacataaaaaatgtgaaatgtaaaaaagatagttttgaaataaaagatatatataattttattaataataatgatgataaaatattatattttgatcgaatattaaaaacacatttaaagatatttagctattcatggattacaatttttttcgaattattaagtaaagaaatatttaattgtgttaatatagaatcgttatataaattgagaggaatcaaattatgcaatatagaaaataaaatttatgaaaatataaaaatccaaaaaataaattttttacttattttaaataaagaagacaatgaattaaataaacatacaagcatgttagaaaatatcagaaataaaacaaaaagagaaaaaatatatggggataataatattaatggtggtaataaaaagataaaatattctcaaatcaatgaagaaaaaaaagaaaaaaaagaaaaaaaaattaataatattaaaaatggagttataaagataaggaaacaaccaactacgataaaaaaaatggggaccaaaaaaaaagacctagatttaaattatttcaatttttatgaaaataatttaattgatttaaaagatttggttaataaaataaacaacttgaatttatgcagtggtgaaggagaaggtgaaaaaggaatggaaaataaagcggtgttagaagggtgccaaaataaagcggtgttagaagggtgccaaaataaagcggcgttagaagggtgccaaaataaagcggcgttagaagggtgccaaaataaagcggcgttagaagggtgccaaaataaagcggcgttagaagagtgtcaaaataaaatagctagcaccaatcaaacaaacatttctaagtattacaaaaatattgaatatatacttttcgaatggttatattttcattacaataatgtgtattataataaggtaaaaaatgaacaatatatatttaagaataaagaaaatgaatataataaaaatgaaagcaacttaattaatgatgatactaattctaaacaatcgaattatatgaaaaaaaatgatgacaattcttctggatatataaataatgaacattcatataaaaattttgaaaagtcaaacttttatagagaagaaaagaaaaaaaaaaatattgttagggaaaacaaattgaaaataataaatatatatagtttaaaaaatttagtaattatattatatacaataatttcacatataccatattttttattttttaaaaataaaataaaagtagaatgtaaaaatagaaaggattatatgcataatattgatattttgttaataattttaaatgaaataaaattagataatttaataactagacaggttcttatcgactttaattatattcacatatttttatttttaaattgcttatattttatattaccaaattatttacccaaatattatttagatgtagatgatatatcatcttataaaagtgatcgagttttaataaaagaagaaattgtaaaaaaaaaaaatactataaaaaataaaaaaaatattgatttacataaagacaaggaaagaaaaaggaatcatataaataataatgacaaaagtgtagatcaaaatgaaagaataatattaatatataatttaaatgataataaatgtaaatataccgtttttttaatagggtgtagtaaatatgaaattgataaggaatatatagaaattgattctcataaatcagaagaaattaaattaaaaatagatccaaattacgacaaaaatatattcaaatatgattataatattaatataaaattcccatcttttactaaaaaaataaataatattcacaaaaaagacaacttgttaagtcatgaaaatgttaataatactataccttaccaaaaaaatacttcaaattttggagactattcttctatatcttcttctatatcttcttctatatcctcttcttctattgatagttttcagtcttctttatcttatgatggaattcaaaatgttgaggaaaataactatgaaaataatttttatcataatgaaaattataccatattggtactacgaaaagaaagcaatctcttcccaggagataatgacgaggacaaatttatttgcattaaattggttgaaacaaataagagcaaattaaagtcagaaatagtgaggagtaatagtaatttattggtagaaggaaaggaatggaataaattgaatagcaatttaaaatttgaagaagggaaggttaatatgaaaaatggtagtggaacaaattcatcaaaaataaacataatgttaaaaaatagtgaaataaaatgttttaacatgaggggtaaaatatatgaaaacaaaagtgtagaaataaatttaataaatgatatttcgaaaaaagtgaacataaatatgaatatgtatcatatatatattaaaaatttaaaaagagaagaaataaaaaaagggaaatttgatatagacatattaaataataaagaaaatggtaattataaaaatgatattaatagaaatataaatgaagagaaaggatacaaggaatgtgatgtttgttctttacttaatttgaatttaaaaaaaaatttaaattgtgataaaaattattttaattgtttttatatagaaaatatgaaaccctttgttagtagagaaaatgagaaaagaacgataaaattacatttttgtccatttatagaaggatattatttatgttttttacttttttgtgctaaagatttaaagacaaaatgtgtaatgtcgagttgtaaattaaattgcttattttatattaataatataaaggaagaagaagttataaaaatagattcccaattatcaaaattttcgtatcaaataaaattaattccaataaataaaagatttctaaaatgtatacaatttattttatgttatttaaacaaaatgaataataaagtttttttaagttacataaaaaattatctaaattatataaataaaataagtgaccaattttatataatttgtgataaggttggtaaaaatgggataaaggaaaaattaccatctttccaaaaatataattataatgattttataaatcaaataaatgaaaataatttatttttagacaatttttatgatcaaataaaagaaaaaaataactatttatatgaatttaacataaatgaagaaaatatgggcgtatatgaatataacataattttgatggcaaataataaaaaaaaatataatgaagatataaataaatctatacaattatcaaaatatgatgagttagaagtagatgagaatagtatatataatgcatgtaataaattgtataaaataattgcaaatataaataaaaaagaaaataataatattataaatgtaacatttaatcaaaatgcatatttattatcaaaaaaatgtgtgtctatatataattatactaataaaaatttaatatataaatgctctaatagtgaagtattagatatgaataaaaataaaattgattataaaatatttaattatgatgaatatattgaaatagataaagatgtagaatcatttaattttgaaattagatgttattcggttgtagaagtagaatgttcatgttttatatttttaactaatgtagaaaatgaacaagatgtaataaaaattaatgtaaaagcaaatattgttaaaccatatcctaaagatacaattcatttaaaaatattaaataaaatgaaaaaagcagtaagtattgaaatatataatgaattagattttcattgtgagtataaaatttattcagatttaccaattatatttggagatcaaaaaatcgaaatgcttccaaagcaaaagaaagtttatactttttttgttagaagtatacatataggggaattcattgggtgtattatatttaaatgctttcgatttttagatgtgaataagataaaagattctagagatattattaaaaatgattctaatattacatctaaaaatttttcattggtagattcatttttttggtataaattaaaagttacagtagatttgaacaaaccgatcaatgttttaactcttgaaagcaatgtaggagatgtattaaataaagaaattgtgttaaaaaataatggaaataaaaaagaagaatattttttactttcctatatgaatgaatatatagaaaaaaagaaaattgaaataccagcaaatgatgcatatgtatacacaataaaatatgctccaaaaataccaaattattttgataatattctaaaacataaaaatgaagaagatacaaaagataccaattttgaattagatgaaaaaaaagaaaaaggttcgaaatataataaaacaaaaaaaagtgaatgtatgaaaagaaaagatgaaataaaacaaaatgagaatataaaattttcagacatttcaaatgagttatcaaatttttattttcttcaaaatgaagaagaagaaataaaaaatgaaggagattgtttacaaaaaaatatgacaaaaaaattaaatttggaaaaaaatgaaaaaaaaatatttgaagagttaataaaatattgtgaaaaatatataaatgtagatataaaatatacaagtcataatattggttttttttttatatataataaaaatgaaggggtaagttattatatattaatattattggctagatttcgaacccaattagatattagtaatttctcttcgttattatcgaaatcgtgtttaatacatttgaattttgaattttccaacaaaaataaaagatatgtaaacaaattagaaacagatcaacagaaaaatacaagttattattaccaaataggagaatatattgaggaaaataatataaataaaaataattcatacaattgtattgagaactataaattaaataatgggtttgagttttataatagcaaaattgaaagaggcgcggacaaatcttccaacatcaaagagtttgatggtgatggaaatggaaatgaaaatgatgatggtgatggtgatgaaaatgatgatggtgatgaaaatgatgatggtgatgaaaatgatgatggtgatgaaaatgatgatggtgatgaaaatgatgatggtgatgatgacgatatgagcacggaacaaattgtgagacacaacataaaggggatatatctgagtaataaaataaattatagcatgataaattgtgataataaaaagagtggaaattatattttgattaaatataagcccactgttggaacatcacaagaatgttatgttataataagaagcgatttatttggagattttatttattttttaaagggtggatatatagaagaaaaaaaaataaaagagagaataataatacataattcattttgtttatataactttaatataaatttattcaacccattttcttcaaaaatatatgtaaaatgtattattgaaacagatagaagtaaaaactatgaatataataatatgaataaaataaattgtctaaaattgtacaaatcagaaaaaagtagaaatattgaaaatggaaaagaaaaaatattattaaatacgaaatataaatattttaaattattaagtaatgaacattttaaaataaaaaaaagaaaaaacttttctattttttctacttatttttgtaaatatgcaaaatcgatagaaaaattgataatattattatatcctataaataataaaaaaaaagaaaagaaaattaatcaatatataatatatgaatatgaattattttttaataatacaaaaagcgatttttttcttgaaacagatcgtataatagcagataaagaagaagaagaagaaaaagaagaagaagaagaaaaagaagaagaagaagaaaaagaagaagaagaagaaaaagaagaaaaagaagaaaaagaagaaaaagaagaaaacaacttagacaatatcgaaaatgtttgtactaaacaattaaattatatttcaaatgaaatgtcaaaatcatcatcatttagttttaatggtttagaaaaagatgggtctgctgaagatggtttagaaaaagatgggtctgctgagaatggtgtagaaaaagatagatctgatgaatacagtttagaggagaccgaatcggagagttatagcacaatagaagtgggttctaaaagaaacaaccatatattatatttggataaactaagtatatcggattcatcagatgtgggaatagaagagtgttatgaagaaggaatggaaataaataataaatttgataaaaatatagatggaataaataataaatttgataaaaatatagatggaataaataagaaagaagttgtttattctgatggtaaaatatttatccaaagtatatgtaagattaaaactagtatgaaaatatttataaataaaaaaaaaattggagaaatggaaaaatattatcagacaataaaagagttagaaaaaaaaaaacaagaaaaaaatattatgcatcgaaatattttattggaaaaatcaaattataaaatattttttttaaatctaaacaatttaaaaaattctgataaaatagaaaaaaattgtaaagacaattatggaaatgaagttttaaataacattctttttaatatgaatgaaaatatattaaataattttctttatataaatatagttgaggataaagaaaattatttagttattagtttggaattgttagctaatattccttttcattgtaatttttttataatgataaaaaaagtggaaattagggaaaataataaaaatgatgaattagatgtaagaaatgatgaaatatatcaatatgaaaatgatgataaaatattaaaagttaaatcaataaaaagtgaaaaaataaaagaatttataacagttgaaaaaaattatatttttttagaaatatttaattatattaatatatcaaaaaaatgtataaatatatatatagataataatttatattctgaaacaaatttaaatattttttataaaaataataatgattcatatttcgatgcttatatagttggttataatcaactattagacaaaaacaattcagatgaaatacataactatactattcgaccaaaacaaggaatactgaaatataaccattttaataaatttataattactagaaccaataagataaacattatggcctttaatagttcattcttattgttaattaaaacagagaacaatcttttttcctacatcatttcttcacactttacaccatctcatgaattatacactgaggctgagaaaaacgtgataagagacattttaaaagagaaccgcacaaagtttagcatctttttcaacgaatttaagcaggaaaaaagagaaagcacaatatttaacaaaaaagacgaaataataatctatgacatttcagaatcaaatgaaatacaaagtgggtaa

SEQ ID NO: 25 - protein, 2274 aa, Artificial Sequence
  Synthetic
MDPRQNGRLARWAGLSLCCLLLLLFNALSPCGEGRVRWRSLGEQSGIGTNACSNIIPFYLNSHNFFNPLREINCSNNFSLRWNINYVYFEFLNEEFFINFHLHSLFVKEVQLYEYRERSILLLKKEASHDEVKFLIRVEPDGSVHPGEMRTFYFVIQAGGGPNFSACVNVMLELNVGMVRRYHGGAAVGQLSSGETVDVVKHTNAVKHANVVKGLYSDRPIVEEKKSKLPAHHKNYIIVKDNYYYASSEDYYVRFEWEKEESHADLPSNRAVVVYTVLLFTQFREHGSISFYSSLTEDITKEKNFHMNLKKLSLGKSYLNNMTVNNILRLAHLDKECSRGCVNSVRAKNGLFLHALLENNSVYKFEVKYVRGRGSPGGLAITPDGAHFSMEFSIVRNAAEYTMVDFIREHFTRECMHNTFFPSGIVQTGGGGEESGGSREKPPVQGYPPKGCPNDSTQNCTAANTANPANTANPANPSDQFVIYGRMIELNDNFIFNFDPIKLFEQEKQVSITIAEESLFNLVVTSKADSVYLNLLKRDSPHTEKVCNTYYLNNLNDYTLFNYMAKEMDHFPHVHVNKTFEGDYTDGNYTDGELLRRKKFPLHSVAYINCTLPKGDYHLRITVDGYNLICDSNEVSLTIYPLSLYEEKHKCDSSMNVVTDLFYYHLRAESKKHREEYTNLISPPNGESAATVEGLSKWADHLDTDVKTYQNIYENRWVLNNPIFQDFDFVILYERHITERVDELLVTINNSVFVDLFFVIVEPIMGKNSYYYVYRNSRTVSKYTVQHGDLPFTIYLIASTMHYPGKQLCGFFFVDIDLVTKAKLTWEEEEKQPEEVGLTTAAHNMISRVNYIPDLIIGYDSYSFSNFCFIPKYKKHSLIIYVKEGSIVKINCFSENYVYIYLYDQNKRKLYDGYNQLYIESFTKGEYEIVFEFNAPNDRKDNAFFYLQVYVFHLSLLERCAAGAAGGAAWEAAGTGKGMDTGTGTSIANDTANSTAKGTANGSQTAEAPPAGTSPNAQDVYDLSSYVEASRPDLPTDANETQVKEKNTFWFQAPNAYYLFKRDFLLLHPNKRIVKEVILPHVPSMDPAATFLLKVELIFAKNYLPYKLAIQDTSNSKLQYHDSYTYKNKILMTLPVVNGGPPVGNNSTGETSQRGKLFKLYVDLYEEVNDDLRNNFCTYAYLHIIYTSEMEAFRQWGEQGLSYATVNLPLLLNNLLIKGPPGVGEAAIGEAGVGKGGVIEGDEAGESPTHSNDRSALISLRNADLPVANQSANYTFELVNNNVLLFLASDDLLFDMYMYRENRDIKLSVYKFSDTERLLSGGGITYDEVTKKGGPPGEEIFSFNQRQIYLSVHLTRGLYIFEYERGSGPFFANLSVVWPHGEDMPSGGYLTGEEVGLPKKEVGAILPRGGALLREEVRFLFGKEPKCGGEAKLHHGGEKLHNLTYFWREIIKNENFKILDNSISSVEVKKGEAKNALIYKRFYICLSDEVGKVPQEGGHSNDTHRSSQTVEKNYTLFNLSIEESAQVYVEIKPHYHFFAYPFHLNIVSKRSFFDISSGHKTVITIDLYKGEYEIHLAFPALQREQIKDVIFDLVILIVSPQNGEGEAEKLGNAITHVGNPPGGSPQSDVCKTYPYKPLLNKVNLVDIPENDASVRRNIVSPKYKNKFFHLFGKFFLKGYKEEVFFTIPNGGYVLKIFCLPIDESTGEGAASEEGATNRIGDMGSTPFGNFSDPPTNFVNSFLNVRNVFNVRVVLNHSGEANQDRDGRKDPPQNSPLFVSPLFANEDEEFMLNLYRVDRNFKVVIKTNSYLCNYFQLVVSLYPLDFHNPVHSYAYAQANSVEKCMKNIFDTLSVKAKLYEDIKFEQKKFPSHQYLRIETDVVNLRSAHKEDSFSFPVVVRKGSYFKANVGYDFSLASFQMKLLKKGTTISSSSKMEVSLKENALNTFENISLYLEEGSYTLEIRSYALTANLDINKNFSFCFYFELELFEFSGDNKGDAVLLDVFPHNSVPVDGSGSFGVDLIFWGRVHTKDIHLEDGQKTPVEPTSRRAINYANIEVHKFLLSPEEMAKVEKNFIFKFSPSCNIYVNQEKEKKLKFTLSTDDRSASTGEGTNGVEHSAAVVTSEPNERNGQVHPESVNNSFLLEYSQKEVPPDPGRTSYLQGTSQKESVYDIDRVIQNFRLNEKKRRDEDDSRVSDSPKGEADACIPLTILTIEIYCFEKSYFLIFIFFLCVWFMLCAFLFVLLKLYKNWKYYRNYDVITESEEVVNLFDDDHI

SEQ ID NO: 26 - protein, 2233 aa, Artificial Sequence
  Synthetic
MDRRKNGSLARWAGLTLCCWLLLLLIIMSPWEDGRIRGRSLEEQSGIGKNVCNNIIPFYLNSHNFFNPLREINVSNNFSLRWNINYIYFEYFNDEFFINFNLHSLLVKDVKLYEYRDRSVLLLEKDASHDEVKFFIRVEPDKSVHPGDMRTFYFVIHSGISSNFSSCVNVMLELNVGLVSRYEWASPIYGTAINQLTSGGNTNEVRSLHSDRNIVEEKKSKLPAHHKDYIIVKENYYYASSEDYYVRFERKKEELYANLQGNEEVIVYTVLLFTQFTEHGSISFYSHLTEDITKEKNFHMHLKKLTFGKSYLYNMSVHNILRLAYLDNDCTKGCVHSVRTKNGLLLHALLENNSVYKFEVKYTLGRNSPDWLAKNSDGAHFNVEFSIVRNAAEYTMVDFIREHFTRECMHNTFFPSAIIQGEGKNVAQEYSHKGCKEGQKESCTSSDQVDEFIIYGRMIELNDNFIFNFDPINFFVQNKQISVIISEDSLFNLVVTSKIDNVYINLLKKDLSHGEKVCNTYYLNNLNDYTLFNYMSKEMDHFPHGHVNKAFADDYRDDEEVRRKKFSQHNMAYINCALSKGEYFLRISVDGHNMVCDSNEVSLTIHPVGLYEERHKCDSSMNVVTDLFYYHLRSESKKHREEFSNLISAPQGEGLTNEEGSNSIAGEIKWPKKIEEDGKKRTYQNIYENRWILNNPIFQDFDFVILYERHITERVDELLVTINNSIFVDLFFVIVEPIREQNSYYYIYRNSRNVFKYKIKHGDRPFTIYLLASTVHYPGKQLCGFFFIDIDLVTRKKLTIEEKGPDNAEPTSNAHNMISRINYIPDLIIGYDSYSFSNFCYIPKFKKHSLIIYIKENSIVKINCFSANYVYICLYDQNRRKLYDGYNQLYIESFTKGEYEIVLQFNASNDQKDNAFFYLQFYVFHLSLLDRCAHSAAFITGADTGTDRGIGIVSQMRGESTSIGSEGAATPPVATTPNVQDMYDLTPYVEANRADFTMGANQTQVKDKNTFFFQSSNVYYLFKRDLLLLHPNKRIVKELILPHVTSMDSTVTFLLKIELIFAMNYLPYKLAIQDMSNTRLQYHDSYTYKNKILMTLPLVNSPPLVENRATGEAGQVGKRFKLYVDLYEEVNENLRDNFCTYAYLHIMYTSEMEAFQKWDEQGISYSTVNLPLLLNNILFKGSPGATGKAFSVGEGSNHGGDHSAMISLRNSDLPIANQSVQYTFELLNNNVFLFLVRDNLFFDMYMYRENRDIKLNVYKFSDTEKLLSGGGITYDEVMKKGSSSIGEEIASLNKGQIYLSMYLTRGLYIFVYERGSGPFFANLSVVWHYKEGIPSGSYLTGEEGVGLPKEEVGVILPMGTTPIRRDDAFLREEISFLFGKEVKCNEGTKVHHGGKKLHNLSYFWQEIIKNENFEILENSTTTVEIKKGEQSNALIYKRFYICLSDQVQKISQEGRNSNDSTHKSSETIDKNYTLFNLNIEESAQVYVEINPHYHFFVYPFNLNIVSKRSFFDISSGHKTFITIELYKGEYEIHLAFPGLEREQVKDIIFDLVILIVSSRSGKNETNQRNTLTYIGNSPGLSPQSDVCKAYPYKPLFNKVNLVDIGENDVSVKKNIISSKYKNQYFHLFGKFFLKNYKEEVFFTIPNGYYFLKIFCVPIDESTEENISVEEGEEKNEIREVRKISVGNFSDPSMNFVNSFLNVRNVFNIRVVQEHSGEANEDGDDKKDSSQNSSLSVSPIFANEDEEFMLNLYQVEKNFKVVIRTNSYFCNYFQLVVSLYPLDFYNSVQSYMYSQVNSVEKYMKNIFDTLSVKAKLYEDIKFEQEKFPSHQYVRIETDVVNLRSVQKEDSFSLPVVVHKGSYFKVNLGYDFSLANFQMKLAKNGVAISSSTKMEVNLKDNSLNTFENISLYLEEGNYTLEIHSYHITANLNNINKNFSFFFYFELEIFEFSDDKTGDAILLDVFPHNSMAIDRSYPFGVDIIFWGRVHAKDIHLQDDQKTHIEPTNRTAIKYANIEVQKFVLSPEIMNKMERNFIFKISPNCNIYVNQEKEKRLKFTLSTEDKNASIGESVYGEEKGEAITTNERNEENGEVHPESVNNSFLLRYAEKEIPTDIRGTSYVEGTSQKDSVYDIDRVIQNFRLNEMKRREEEDSTMGNPSKEETEACIPLTILTIEFSCFEKGYFLIFIFFLCFCFMMWVFLFVVLKLYKNWKYYRNYDVITESDEVVNLFDDDHI

SEQ ID NO: 27 - protein, 2322 aa, Artificial Sequence
  Synthetic
MERRKKRKKISLLIDVLLYFILFYSMFPIEKKNERKLSENYSEAGKAFKNILPFYLNTPQYCNLLFDMNVSGTYSIRWGVNYIYFEHLNDSFFVNFNLYSIYVKEVELYEYRHRSFLLLKKESVENEIKFVTSIEYDKSIDAKEQRTFYFTVHSNYVQSFDTYINVTIDVNIVGHVGRGGERGSGQVPSVDEKKNDDITMVDESKNGIKQNRLPLNNNSYIIIKENYYYASNEDYKFNVREADNFYVDSKGNKIIVIYSVLFFVDFNKHCNISFFSLLTQGIEQGKNFSMDLKKIKLGKNYMHNMTTESILKYSFSHKECKEGCIKSSKTKNGVFLHTLMENNNIYKFDIVYTIYSSHSTYGNDSIYGSYSNYDISNSDRGSGHTSTSSDTIRPNEVVHFNLEYSIVNNSKNYTINDYMKEQFYKECVHNTFFPHQIIQVGENDEHFTGLVVTPSGKNKCSQNGGKCIDDDKKKADNSNHVAENVDFIIYGRVIEINDTFILNFDPINLFEEEHKVGLHVSEESLFNFSLISKSESIYVNLLKKGSSQTVCNTYYLNNINDYSLFSYITRDSATFLHKHFNDTFEVNKKDANHKFKNFSLQNILYINCVVPKGEYELKIIVNGYNVICDPNEINLVLYPLLMYEEKHTCDSSMNALTDLFYYHLRAENRSQSGRGSVVGGLGGTTASRVDVSGEATPERTSNSASSYARQNDDLTSEVQKTYSNIYEKKWMMNNPLFEQDDFVILYEKSIEEQVDELVVIVNNSLFVDIFLVIVEFIGEDSYYYIYRNSRSIVKYMLKYKNPFTIYLLASNLHYENRKLCGYFFVDIDFLKGSITVSLDGTEDYKKMIQKINYIPSLIIGYDSYAFSNFCYVPDYKKHILTICVQENSIIKINCFTQNYIYIYLLDQNKNKIYEGYNHLYIESFTRGEYEIIFEFNVPNDKETDASFFYLQIYIYQLSALEKCVFDGQTDSSSGSSSDGNGKDAQFKNYLSSNENGDNSEYDLSSFSATNNSKIELKQKGKNFFFFQNEYAYHLFKRNFLFLFPSKRTTKNLILPQVSHLNNIAFLLKLELVFHKNFLPYKMTIQEGTDDLLMHHSYTYKNKILLTMTILNNSVKVFKLYIDLYEDINEKLKNDYCPYAYLNIIYTNEGGTYKQAAFSGLNFGEISMPLLLNSILLKSFAYARGMGNSERNKDEDMGVVRRNDSGVRNDDWSNKHTSTISFRNSNTVTIGSQSVDYNLKIINNSTTLFLAEHSVFFNMYMYRENNDISLKIYKYLNSDNKLEEGYGVTFDEMNKEHVLSHSEEIMSFSNKHIYISTWLKKGLYIFKYEQSTSPFFINLSVIWNYQDDDKNMGKNYLKRYKYGGQSLTHYNNGKTEEPPADDAISDTILKNEIYFYFDKELKCDGRKDIFYESKKKLHRLTFFLKEVINNNIFETHSDDSMQITVKRDSRNTLIYERYCICIGGGPKVNDMHSDGSTSAVSANEVAQGVVTQPWTGSGSRSSSSSSSSNGSSSSSSSNGSSSSSSNRYKIYHISVEESTKVYIEIKPHFHFFIYPFRVSVTSEDFYFNIFSENRNVINTNLGKGEYEVILSFPQLKENKNTLQNAIFDLIILVILPEKGAIIGDKILASSYMSNRLSGKLLEKELCNISTYKTLFNKVDLVNINENDSYVSKYIISSKFKNNYFQIFDKYYVNNFQQEIFFTIPGESYILKIFCMPIDELGGENMYKEGTYNNTYNGNSNSTYNNNAYNNNITYDSSVHNNSSSETMSNIYNVASNYVNAILNVRNNFSVQVVLADMDNKSNVKNNTSNDNEKDKYDDNNSSNRNNSSNSNAFVSPLHSNENEEYMLILYKVDNNFKLVIKTNNHECNYFKIILSLHPLSFYNSEHYFSYANYLDKYLKNLFNTLSVKTKLYEGINYTHEKSSMYSLTRLTSPIVNLKSVNMKDLFSFPLSINKASYFKINIGYNFSLVSFEIKLMKNSTTISCSNKVEVNLQDDTVNIFENISVYLEKGDYILQIFSFDYMGNSVNINKNFSFPFYFELEIFEFFQETKKSGPILLDIFPHNSVPVDAVFWGEVKGKEMFLEVHNKKYADLRNRKIIKYGNIEIHKSFLLPEDMMNMEESFILKFTPSANIYVSEQNEKKLNFIFSTYSTNIEQNNNSYKVVQGEDKIVMIGSGQRNGQVHEENVNKSFLFEYDQRDITEAGESVNDSQTNIHVDKFFDLDNVIKNYRLNEKKKKREVSYTSLENSNKQNDSCIPLYLFTYKIYCFERSNYLIFIFSFFLFSVSCAFICLLIKLYNNWKYYNNYDIVVESEEVINLFDDDDI

SEQ ID NO: 28 - protein, 2165 aa, Artificial Sequence
  Synthetic
MFVCIIFCVLFLPFGRGIPRNLGDDNIKRRSQCENLVPFYFNAPQYWNMSGDINISDYYSLRWDVNYIYFEKVNHKTFVNFNLYSIYVKNVELYEHRDRNIMILRREVMEGEVKFVHFIDQDVSLHTLEKRIFYFIIRLKKPLHDDTCVKVGLDVNIGKAPITSFEDGMVVHQEKKSKRPLHHKSYIIIRENYYYASNDDFYFTPEDSENDTYIDDNGAKFVIVYSVLLFPQFDKKCNISFFSFLNGEITKKKNFSMQLRKIMLNNNYFHNMNIDSVLKHTYNNMTCSEKCVKSIFTKNGLLIHNLLENNYMYKFEIVYKLTENLNKDQGNYENNTVNGWTYNINSKTYFNMEFSVVMSLKDQSFIDYVKEYFNSECIHNSFFVKKIVQIGIYKGRNDNVISSFSMKKCQEKDKHRCNFTELNNEYVIYGRIIQLNDSYVLNFDPINLFREEEKVHIHISEDSLFNFTLTTTLENIYVNLLKGNSTQNVCNTYYLHHINEYNIFSFQSKDKAPFMHYHFNTTFGDKKKYSKDHRLKKFSLQNVVYINCVLPKGEYDLRIVMNDYISICQPNDVQLTIFPLHIYEERHTCGREMHVLTDLIYYHLRGEGRRGGSSGEGNRPIKNELTEPSSEQSRTISHNVYETMWELNNPLFENFDFVVLHENIIDEEVDEIVVTVNTSAFVDIFLVITESVGEESYYYVYKNSRGVAKYGLKFGGPFHMYLLAPSLHYEDRKICGFFFVDVDLVLLGKKAKGKTPRREDHTVGEETLSSVDEPQGRNVVQRTIHLPELIIGYDTYAFSNFCYVPSYKKHVLTIYVQENSIVKINCFTQNPIYIYLLNSEQKKIHDGYNHLYVESFTKGEYHVVFEFNLQSEKEASSFFYLQIYIYHFNLLERCAFDNPSEALSSSEELPLSSALRIDPLRSDPSQSNDVYDFSIYNEMSTAKDGIKVVDPTSFLFNDEHSYFFFKNMFIVIFSNKIISKKVILPRVATHLEASPFMLKIELIFHNNFLPLKLRVQDITNSLLYHSSYTYKNKIILTLTLSNGRVRVLNLLIDMYEDVSDSLKSNYCPYAYLHFVYTNEVEAYRKIGASGLSHGAIDLPLLLNSLLLRAVWGVDDKGEEIRINTSSDNVDSASTPPIEGGVTFRSPVFPVMVNQSVNYTIEMVNNDTMLFIADNDVFINIYLSKENGIVVLKMYHCLSTGILEKGSGIQYDEVHKNFLSISKEIASLSGKHVYLSTYLERGLYVLKFERDSSPIHVNVSVIWNSNREDPTIIGYVKRQEHANKLVNYASREVLPVMSMHRMNVSAQGTASATDISPAGDPILRKETHLFFGKELHCSGVKQVFRESKKVASVPFFFDEIVKTKLFAVYSGRTTHILVKKNADNMLIYERFYVCLGRETNGYYEQYSGEASRDESDQHNEGKYPLFGITVEDVAQIYVEIKPHYHFFIYPFELNVTSSSSYFSHSSGGKNFVSTNLGKGEYEIDLRFPEWTNNDIKSVMFDFVILLTFPIWEAYDTEVLSKKGTKMLLRDDTCNVSMYRSLFNKVDLINTTENDIIVSKNIISPKFKNKYFHVFDKYFIRDYQQEIFFTIPSGGYILKIFAVVDSEIGEGNDQGEGFPGESHPQGTGRVVDFPANYVNSFLNVRNVFNIRIVESDLNGLGGKNNNVEDKKEEISYVAPVYSNESEDYILVVYKVYTNFKMIIKTNNYDCNYFQLILNLHSVNSYSDVHHFSHAYNFNSYLKNIFDTLTVKAKLHDGINFEQKKLSTYNHVSITTSIVYLKNINPLELFSYPLSIKKGSYFKISVGYNFTQAHFEAKLGKNGEPICSGSKEKVNLKDDTINVFESISLFLDEGEYSLQILSHDLIEDSVNISKKFSFPFYLELDIFEFMHEKIENPILLDIFPHNSVPLDRNYPFFVDLIFWGELRGRAVYAEDGKQKRVQLRSGRMHTHGNVEIHKLILPPEEMDHMENNFVILFDAKESNQMNREKEKRLYFSFSTISTNVLSKNSSYEPIREKQNVMSESKEGNRQIHPESVNTSFLLEYGQRDAKRDDSIQENSNKDNFFDLDTVIRNYRLSQKKDKEYMPVSGEAPPKGESSTCIPLSIFALEIYCFEKNYFLLFLFVVFILCISCAFLFLLIKLWKNWTYYSSYDVITENEEEVTLFDDDI

SEQ ID NO: 29 - protein, 2472 aa, Artificial Sequence
  Synthetic
MRKKKKTCLVLLILSFLCFFYSSLSYDEDYDEEYNYEDKNSDYECNNLIPLFLNNLEYSNISQEISLSKTFSLKYPVHYMYFKYSSDQSIFVNYNIYNSLSFGYVKKIELYEYEIIKNESVLLLQAEKDSDKNIKILYTITKIDNRKNYEGEKDQRLFYFIIYTTIENDNDIIKSCINVNMSIHIRIIKDDTQTNNHNNNNIIINNNNNYYYNYVHNNYGMRGIMPNHLYNHLLPLHNKSYIIIKDNYYYCTNEDYYYNMNNIEDYYYDEKSGDKVVVIYSVLLFIEFKKEANIKFTSFLKHDINKYNNFVIHIKKIDIHNNYLNNMDMEHILKVGNNEEYINGNKKKKSMECEEDCVESYITSQGIYIDTILNNNHLYRLDILYKMGKNEINNKTTEIIKKENNNNNNNNNNNNNNNNNNNNKNNNNNNNNNNNKNNNNNNYYDYDNDKMGDIKNMKDNGKDPFLSSLLFFNFELSIYNITDNYIPMNYINKRLYTECSNNSHAPNKIVQTKEENMLNIFYTNECSKNIEKCSLYDVNEYIIYNNKVIDIHDNFLLNFDIINSFKNEQIIDIYINEDSIFNFSMLIKSDHIFVNLLKNNSSEKICNTYYMNNINDYNIYDYKKNNKSDFFHKHYNDSFLYNQNMNYNNTFEYQNYYQHDHHLKNFPLQNIIYINCILKKGYYDLKIILNGYNNICESNDFNLLIYPMSLYKNQHQCDDKMNQVTNLFNNILKRKYQHNISHVPNQIQTFDQIYQNKWILNNHIFEYFDFVIIYEKELLPQQYKEIMVTINNSPGLDLFFILVENVQGQKYYHIYKDTRKKNKYILKDKHPCTLYVLVSTLLYTTQDVCGFFFVDIEYVDNMKLLNQNDHPNDIMNGTYQHNMIQKINYIPTIIIGYDSYSYSHFCYVPYYKKHKLTILLKPKTIVKINCFTHNYIYIYLLDKSNNNNNNKNNNDNNKKKLYEGYNHLYIESFVEGEYEIIFEFNTQNDKNMNSFFYLQIYIYNLNTLDKCLFFNNNMNYIKESSINKFINTNNYDTFDLSKYEYNDIIINKNVPRVIKKENNMFFIYQTINFYYLYKSFFLYILPTQHKDGNITNEQDKNRIKKKIILPKINYLSNQNFILKIELSFHKNIFPYKMYIDENYDDIIMISTNTYRNKNIMLLKILNNNTNYINIYIDTYEDINDEIRKNYCSYAYFNITYTNQTEEETVDRQQINLYNKITLPLLLNNILSKDYVPMINKNIVENKNDQTYKADHLYYEDDNIGVINSVSNNYFSGENKNMGDIKNKMNNEYGSVHTEQMVHFQNNDNLNNNNNIIFGNVYPYLENHLRGIISLKNSNSLILNQSVNYNFEVVNNNYTLLIIPNDAFLNMYIRNNDDDDDDDNDKNNNNNNNNSNNNNSNSSKKNITLNVYRILDVPKLTNEGISFNEVKKQILSLSHPFLTFTDKYINVYRYLERGLYIFKFDDDSNKENSSYTFRNSSNIYKNNINNYNYHSSDSIKPLSFFMHFNIMPIYTNDIEYKDNIKNNNYNDKTFNYQGHDKNMYPSVIHNYIFKNELLYYFNKEYSCDKENMLFFEHKKIDDVSNFLEHTWNNIPFEIYINDKLDITLNRDNYNTLILKRFFICFEKEKNVNNNDNINNSNNSYVNNTDNINESQDDSYINIFNDTIKKEYLLFNIVLNVKGNIYIEIQPHYHYFIYPFILTIKSKNNPTQLNKSHDKLFLTTDLGVGEYEIYITFLSNNHYKNVKMKKVMFDLFIFIDILKNKNIQLGKNNLYLPYGNHLDDTIDFKIYDNTCKTDNYKRLFHEVKLLENNNSSNVHMNESYIKNKNNIISTNMNTNYFHIYDTYYMKVREHEISFEIPKEAYILKIFCIHIDEQNYDEDITSGDFYNNTNAYNNNNNYGNNNNYYKIEQTDNLSHFSFNYLNTYFDIQNLFHINVITNEDSKFFSDEHTNDQDLYVKPFFSNEENEHILNWYKIDKNFKLVIKKNNYDCTYFKLIISLYPMDYFNSIDYLKYNNYLNKYFTNIFDTLSLKVNQYQDISFEQIKNKNYLYDYLKTNVIFLKSLNSRDLFSYPLTIKMPSYVKINFGYNFTLTSFEIKLLKNNMIIATSNKVQANINNNNINIFENVSIYLEKGNYVVQIYSYDLIEKSSNIINKLSFPFYAEIEIVQFVNDQIDEPILLDVFPHNSVIVNRNYPFFVDLIFWGRANENISVLDTKENNIKLNNTKLIKYGNVEIHKYVLSSEQMRSLENNFTLKLQSNVHISAWMEKRMNFSFTNEGWNYDGQTEHGKLMKNGIKTNNIVDESSKISKGEKLNETVLLEYGKREIKNESEGESKSENKIEGNSNNESDGESEKRSFLQNGVIEKSNLFDLNEVIKNFRYNKKKKKEEEDFLLNGSYMNSNDKCFSLSIFSFEIYCFEKFYFFIFIFILTVLWISCAFIFLIIKIYKNWKGYRNYDIVGEMDEVIGLFHGDDL

SEQ ID NO: 30 - protein, 2196 aa, Artificial Sequence
  Synthetic
MNKRKGKVWLFLFIICLIVINFKISYEQNNERNLSEITKNEIEYERCQNLVPFYLNNPEYYTMLNEINVSNEYSIKFDITYIYFEYINCNFYINLKLYSKDVNKVELYEYVDNKGILIFSENSKDNLIKYLFHVQDDKNSNKNENKNENKNENKRVFYFIIYSNKIETSNECVNIRLDIGIGPVVLEKGDTKIESTKKGDIQKEGRQNEGRQKEDRQIEHHNSYIIIRDNYRYATDGYFNFSLKNNKDFYIYENGKKYGIIYNVIIILEFNEENNINFFSLLSQGKNKWNNFSMQLKKIKLDKNYLHNMNIEYIIKHANADMTCNDMCIKSDESKNGIIFLNALLSNNYIYNFQIRYAENENENNYINNDINYDNENKSNKYLGRNSVESKKEMFNQDNIIYFNFEYNIVNNVDKYTMIDYLKGHFNHENIQNTFFFDKIIQIKDNNINKNNILPIIGFNKECVGENKKECNKYEINNNYIIYGNTIEIDDNFILNFDPNNLFKEETKVNITIEEESFFMFTLESKSENIFVNLLKKNSTENVCNTYYLKHIKDYNLFSYLTKNEKNILKYDKTNFSRHHIKNLSKKIIIYIKCILLEGEYDLKINFEEYNKKFESNNINLIIQPLHIYEKVHSCDRKMNIITDMFYYNLRTENVNKKNDNNVISYKNIYENMWELNNSLFDDFDFIILYENEIFENVDKIIVTINNSIFSDIFIIIFETVKDETHYYIYKNSKSTIEYEIKNKLNNKISIYVLTPSLHYSGRKICGFFFVDIDFIENKKESIGEITLFNNMYKSNRSYIGSINHIPYLIIGYDTYSFSNFCYIPDNKEHVLIIFIEKESIIKINCFMENYVYIELYERKHIGGEIKKIKLFEEYQQVYIESFTKGEYEIVFKFNVQNDREMSNFFYLQIYIYQINLLDKCVFHNEDDFGEIAEGSSSVYDLNEVDRKIKSKNGKKNKEIGYFNDNNGFIFENESSYYLFKRYLLFLFPKKIDEKIEKKIILPETNNSGFVLKAELVFHKNFLPYKLSLEEDGENILAHESNIYKNKIIITFNILNKNVKYVKLYIYLYDNVHEKLIKDYCPYAYLNIVYTHELEKYREHENDEYQYDTMHLPLFLNNILLKRYEYSDDVEEEDVEIGKVETESGNDKNVRVVPFDKKNGMCIGNQKIEYKFITSNNNMMFFLAERDAFLNIYIYKEGKEDIMLNIYKYKNISKFEKNEILPYDEINKDIQSCCEEIFSHSNVNDINISTYFEKGLYIFKFSEKLPYINMIFSVIWVEIPNVNDIIMGSNINNKYEINRYIEENESKFYFSKELKCGDKKGIFYDENKLKDLEKFVIENMFEKNLYTIYFGNKKKEITISNDSKNIVMYERIHICFGNEIFNHQVINNTNYIKENEESHIILTLNVEKECQVYVEVKPHFYYFIYPFKINILSKNSYFKVSNEYKKYVYANIEQGEYNIEISFKMENYINNKIKKIDGVIFDFIIFIYNTSNENKLKNIDFSDEEKKQMVSLKNETSNMNKLYTRKIYKNIFKKVNLKNLNENGIVVNEKITTHKSKYSYFFCYDKYIIKNHQEIFFTLVDNVDYILKVYLAPIYELSGNNNSKNINNDFEHFENFENFENFENSDNNSESSSLYNFSTNYLNYTLNVRNVFNIEVVNESMNNDYIPEKVIKEKRLKNKIIEKKTPDSNILYTNEDDEYILNVYEVNKNFKLVIKNYNNIDSNIELVLSLVPLKYYKMNMNTKYANEYINSYFKSIFNTISVKTNLYSGIIFENREHSTHIYTRVETSVVYLKNIIMEETFSFPLHVKKGSYIKLNIGYDFSRVNFDVKIMRNNKKVSTSNKIKGNMDNGKINIFENISLFLEEGEYTFQIWFYNLTDNYIHINKNMGFPFYFSLEIFEFAENKNNTSVLLDVYPHRSVYVDKAYPFNIDLIFWSEEKREKHGFMEDEMKKIVYLRVGEIIKSGKIEIQKSYLLPEDMSNLKNNMTLKFDLKDNVDMISENNNENNNGNNNENNNKNNRYLFTFLNNDVKENEVAIETNNTNLNNQEKLKQIHNESVNKSILLEYKKEDSEKIEEKDSYISHNVNQDMFYDIDKAIKNYRSRENNESKEITSSIFNKPFSDPDDKSCIYIPIFLIEIYCFEKNNLFYFIFILFIFFMTSAFIFLLIKFYKNWKYYRNYESFKEYDETISLFDDDDI

SEQ ID NO: 31 - DNA, 6825 bp, Artificial Sequence
  Synthetic
atggatccccggcaaaatggtcgccttgcccgctgggcggggttatcgctctgctgcctgttgctcctcctgtttaacgctctgtctccctgcggggagggccgcgtcagatggagaagtctcggagagcaaagcggcatcgggacaaacgcatgcagcaatatcatcccgttttacctgaacagtcataatttttttaaccccctgcgggaaatcaactgctcaaataatttctccctaaggtggaacatcaactacgtgtatttcgaatttctcaacgaagagtttttcataaactttcacctccacagtttgttcgtcaaggaggtgcagctgtacgagtaccgagagaggagcattttacttttgaagaaggaggcctcgcatgatgaagtgaaatttcttattcgtgttgaaccggatggaagtgtccacccgggggaaatgagaactttttattttgtcattcaggcgggggggggcccaaatttcagcgcgtgtgtgaatgtaatgctggagttgaatgtcggcatggtgaggaggtaccacgggggggccgcggttggtcagctgagtagtggcgaaacggtcgatgtggtgaaacacaccaatgcggtgaaacacgccaatgtggtgaaaggcctctactctgatagacccatcgtggaggagaaaaagagcaaactgccggcgcaccacaaaaattacatcatcgtgaaggacaactactactacgcttcgagtgaggactactacgttcggttcgaatgggagaaggaggagtcccatgccgatctgccatcgaaccgagcagtggtcgtctacacagtcctgttgttcacccaatttagggaacacggcagtataagcttctactccagtttaacagaagatataacgaaagagaaaaacttccacatgaatttgaagaagttatcgttggggaagagctatctcaacaacatgactgttaataacattttgaggttggctcatctggataaggagtgctccagggggtgtgtcaacagtgtgagggcaaaaaatggattgttcctgcacgccctgctggagaataactccgtgtacaaatttgaggtcaagtacgttagggggagggggtccccaggggggctagccattacccccgatggggctcatttcagcatggagttttccatcgtgaggaatgcagcggagtacaccatggtggacttcatccgggagcacttcaccagggagtgcatgcacaatacgttcttcccgagtgggatcgtgcaaacggggggaggcggcgaagaaagcggtggaagcagggagaagccccccgtccagggatacccaccaaaagggtgcccaaacgattcgacgcagaattgcaccgccgctaacacggctaaccccgctaacacggctaaccccgctaacccgagtgaccaattcgtcatatacggccggatgatcgagctgaacgataacttcattttcaattttgacccgattaagttgttcgagcaggaaaagcaagtgagcataaccatcgctgaggagagtctcttcaatttggttgttacatccaaggcggatagcgtgtacctcaatttgcttaaaagggattcgccccacacggagaaggtctgcaacacgtattacttgaataacctaaacgattacactctctttaactacatggcgaaggagatggatcacttcccccatgtgcatgtcaacaagacgtttgagggggactacacagatggtaattatacagatggggagctgctgcggaggaagaagttcccgctccacagtgtggcctacataaactgcaccctgcccaagggggactaccatttgaggataaccgtcgatgggtataacctgatctgcgattcgaacgaagtcagtttgaccatttaccccttgagcctgtacgaggaaaaacacaaatgcgatagcagcatgaatgtggtgacggatttgttttactaccacttgagggcggagagtaagaagcaccgggaggagtacaccaatttgattagcccacctaatggggagagtgccgccactgtggaaggactctccaaatgggcagaccacctcgacacagatgtgaagacgtaccagaatatctacgaaaacaggtgggtgctgaacaacccgatatttcaagacttcgattttgtgattctgtacgagaggcacatcacggagagggtggacgagctgctcgtcacgataaacaactccgtcttcgtggacctcttctttgtcattgtggaacctatcatggggaaaaactcctactattacgtctacagaaattctaggaccgtttctaagtatacggttcaacatggggacctccccttcaccatttatctgatcgcctccaccatgcactacccggggaagcagctgtgcggttttttcttcgtcgacattgacctggtgaccaaggcgaagttaacatgggaggaggaggagaaacaaccagaggaggtaggactcacaaccgctgctcacaacatgattagccgggtcaactacatcccagatttgatcatcggctacgactcctactcattcagcaacttttgcttcatcccaaagtataagaagcatagcctcatcatctacgtaaaggaaggctccattgtcaaaataaattgcttcagcgagaattatgtgtatatttacctgtacgatcagaacaagaggaagctctacgatgggtacaaccagctgtacatcgagtccttcacgaagggggagtacgaaattgtcttcgagtttaacgcgcccaacgaccgcaaggacaatgcgtttttctacctccaggtgtacgtcttccacttgagtctgctggagaggtgcgccgcaggggcggcggggggagcggcgtgggaagcggcgggaacaggaaaaggtatggacacaggcaccggcacaagcatcgccaatgacaccgccaatagcaccgccaaaggaaccgccaatggttcccaaactgcggaggcgccccccgcgggaacctcgcccaacgcacaggacgtgtacgacctgagctcctacgtcgaagcgagtcggccggatctccccacggatgctaacgaaacccaagtgaaggaaaagaacactttctggtttcaagccccaaacgcgtactacctattcaagagggacttcctactgctccatccgaacaaaaggattgtaaaggaggtgatcctcccccacgtgcccagcatggaccccgcagctaccttcctcctgaaggtagaactcatttttgcaaagaattatttgccctataagttggccatccaggacacgtccaacagtaaactgcagtaccatgatagttacacctacaagaataaaattctgatgactcttcccgtggtgaatggcggccccccggtgggcaacaactccacgggggagacctcccagcgggggaagctcttcaagctgtatgtggacctgtacgaagaggtgaacgacgatttaaggaacaacttttgcacgtacgcctacctgcatattatctacacgagcgagatggaggcctttcggcagtggggcgagcagggcctgtcctacgccacggtgaacttgcccctcctgctgaataacctgcttataaagggtccccccggcgtgggggaagctgccataggcgaagccggtgtaggcaaaggtggcgtaatcgaaggtgacgaagctggcgaatcgcctacccacagcaacgatcgctccgcgctcatctcgctgcgcaacgccgacctgcccgtcgcgaaccagagcgcaaactacaccttcgaactggtcaacaacaatgtgctcctcttcctagccagcgatgacctcctttttgatatgtacatgtacagggaaaacagagacataaaactgagtgtgtacaaattttcagacacggagaggctactctccgggggaggaatcacttacgatgaagtaacgaagaaagggggtcccccaggagaagaaattttttccttcaaccagaggcagatatacctatcggtgcatctaaccaggggtctgtacatctttgagtatgaacgggggtcgggcccctttttcgccaacctgtctgtggtgtggccgcacggggaggacatgccaagtggggggtacctcaccggggaggaggtgggcttgccgaagaaggaagttggagcgatccttcccaggggaggtgccctattgagggaggaagtccgtttcctctttgggaaggaacccaagtgcggcggggaagcaaaactgcaccatggaggagagaagctgcacaatttgacctacttctggcgagaaattataaaaaatgaaaattttaaaatcctagacaatagcatctccagcgtggaagtgaaaaaaggcgaagcaaaaaatgccctaatttataagcgcttttatatatgcctatcggacgaagtggggaaggtcccccaagaggggggacactcgaatgatacgcacaggagtagccaaacggttgaaaagaattacacgctttttaatttaagcatagaagagagcgcccaagtgtacgtagagataaaaccccactatcacttctttgcctacccattccacctcaacatcgtttcgaagcgatccttctttgacatctccagtgggcataaaaccgtcattacgattgacctgtacaagggggagtacgaaatacatctggcctttccagccctgcagagggagcaaataaaagacgtcatatttgacttggtcatactgattgtgtcgccccaaaacggggaaggagaagcggagaagttggggaatgcaataacccatgtaggtaaccctcctggagggtccccccaaagtgacgtctgcaaaacgtacccatataaaccccttctgaacaaagtcaatctggtggacatccccgaaaatgatgcgtcagtgaggaggaatatagtctccccgaagtataaaaataaattttttcatctttttggaaaatttttcctcaaagggtataaggaagaagtcttctttaccatcccaaatggtggctacgttttgaagattttttgcctccccattgatgagtcaacgggtgaaggtgccgcctcggaggagggggcaacaaatcgaattggcgacatggggagcaccccctttgggaatttctcagaccccccaacgaactttgtaaactcctttttgaacgtgcgaaatgtgtttaatgttagggtcgttctgaaccactctggggaagccaaccaggatagagatggcaggaaagatcccccacagaatagccccctttttgtttcacccctctttgccaacgaagatgaggagttcatgttgaacctgtaccgagttgatagaaacttcaaagtggtgatcaaaacgaatagctatttgtgtaattacttccagctagtggtgagtctgtaccccctggacttccacaaccctgtgcacagctatgcgtatgcccaggcgaactctgtggagaagtgtatgaagaatatttttgacaccctatctgttaaggcgaaactgtatgaggatataaaatttgagcagaagaaattcccctctcatcagtaccttcggatagaaacggatgttgtcaatttgaggagcgcccacaaggaggactccttttcgttccccgtggttgtgcggaaggggagctacttcaaagcgaacgtaggctacgacttttcgctagccagcttccaaatgaagctcctgaaaaagggcacaaccatctcaagcagcagcaagatggaagtcagtctgaaggaaaacgccctgaacacgtttgaaaatattagcctttacctggaggagggaagctacactctggagattcgctcctacgcgctcactgcaaatttggacataaataaaaacttcagcttttgcttctatttcgagctcgagcttttcgagttttccggcgacaacaagggggacgcggttctgctggatgtctttccgcacaactcggtgcccgtcgatgggagcggctcctttggggtggacctcattttctggggaagagtacacacgaaggacatacacctggaggacggccagaagacgcccgtggagccaaccagcagaagagcaatcaattacgcgaacattgaggtccataaattccttttatcccctgaagaaatggccaaagtggaaaagaattttatttttaaattttccccaagttgtaatatctatgttaatcaggaaaaggaaaaaaagctaaagtttaccctctcgactgatgacaggagtgcttccaccggagaaggcactaatggagtggagcacagtgcagcagttgtaacgagcgaaccgaatgagcgaaatggccaggtgcacccagagagtgtaaataattccttcctcttggagtattcgcagaaggaagtcccgcccgaccctggcagaacaagctaccttcagggaacttcccaaaaggagagcgtgtacgacatcgaccgtgtcattcagaacttccgactgaatgaaaagaaaagaagagacgaagacgactcccgtgtgagcgattccccgaagggagaagcagacgcgtgtatcccgctgaccatactgaccatcgaaatttactgcttcgagaaaagctactttctcatttttattttttttttgtgcgtttggtttatgctgtgcgcgttcctgttcgtccttttaaaattgtacaaaaactggaagtactacaggaattacgacgtcattacggagagtgaggaggtggtcaacctgttcgacgacgatcacatatag

SEQ ID NO: 32 - DNA, 6702 bp, Artificial Sequence
  Synthetic
atggatcgcaggaaaaatgggagccttgcccgttgggcggggttaactctctgctgttggttgctcctactgcttatcattatgtccccctgggaggatggccgcatccgagggaggagtcttgaagagcaaagcggcatcgggaaaaacgtatgtaacaatatcatcccgttttacctgaacagccataattttttcaaccctctgagggaaataaatgtctcaaataatttctccctaaggtggaacatcaactatatatacttcgaatacttcaacgatgagttctttataaattttaacctacacagtttattggtaaaggatgtgaaactatatgagtacagagacaggagcgttctccttttggagaaagatgcctcccacgatgaagtgaagttttttattcgtgtggaaccggacaaaagtgtacaccctggggatatgagaaccttttattttgtcatacattcggggattagttcaaatttcagctcatgtgttaatgtaatgttggagttgaatgtcggcttggtgagtaggtacgagtgggcttcgccaatttatgggacagccattaatcaacttactagcggtggaaacaccaatgaggtgagaagcctccattctgataggaatatcgtggaggagaagaaaagtaaactccctgcacatcacaaggattacatcattgtgaaggagaattactactacgcttcgagtgaagattactacgttcgattcgaacgaaagaaggaggagctttatgctaatttgcaagggaacgaagaagtgattgtctacactgtccttttatttactcagttcaccgaacatgggagtataagtttctattcccacttaacagaagatataacaaaagaaaagaatttccacatgcatttgaaaaagttaaccttcggaaagagttacctgtacaacatgagcgtgcataacattttaaggttggcttatctggataacgattgcaccaagggctgtgtccacagtgtgcgaacaaaaaatgggttgttattgcacgccctattggagaataactccgtgtataaatttgaggttaagtacactttgggaaggaattctccagattggttagccaagaattctgatggtgctcatttcaacgtagaattttccatagtgaggaatgcagcggagtacaccatggtggacttcattcgtgaacacttcaccagggagtgtatgcacaacaccttcttcccgagcgcaatcatccaaggggagggaaaaaatgtcgctcaggaatattcacacaaagggtgcaaagaggggcagaaagagagttgtacttcctctgatcaagtcgatgaattcatcatttacgggaggatgatcgaactgaacgataattttattttcaattttgatcctattaatttctttgtgcaaaataaacaaattagcgtaatcatcagtgaggacagtctgttcaatttggttgttacatctaagatagataacgtttacataaatttgcttaaaaaggatttatcccatggagagaaggtttgtaacacgtactacttgaataacctaaacgattacacgctttttaattacatgtcgaaagagatggaccattttccacatggacatgtgaataaagcgtttgctgatgattatagggatgatgaagaggtacggaggaagaagttctctcagcacaacatggcctacataaactgcgccttatccaagggggaatacttcttaagaataagcgttgatgggcataatatggtttgcgactctaacgaagtcagtctaactattcacccggtgggtttatatgaggaaagacacaaatgcgatagctccatgaatgttgtgactgacctgttctactaccatttgagatctgaaagtaagaagcatagagaggagttttccaacctcatcagcgcacctcagggggagggtctaacgaacgaagagggttccaactcgatagcgggtgagataaaatggccaaagaaaatcgaagaagacggaaaaaaaagaacataccagaatatctacgaaaacagatggattttgaacaacccgatatttcaagatttcgactttgtgattctgtatgagaggcacatcactgagagggtggatgaattactcgtgacgataaacaactccatcttcgtggacctcttctttgtaattgtagaacccatcagagagcagaactcctactattacatctatagaaattctaggaacgttttcaagtataagataaaacatggggatcgtccgttcaccatttatctacttgcctccactgtgcactatccaggaaagcagttatgcggttttttcttcattgacattgacttggtgactaggaagaaattaacaatagaagagaaaggaccagataatgcggagcccacaagcaatgcacacaacatgatcagcaggatcaactatatccccgacttgatcatcggttacgactcctactcgtttagcaacttctgctacatcccaaagtttaaaaagcatagcctgatcatctacataaaggaaaactctattgtaaaaataaattgctttagcgccaactatgtgtacatttgcctgtacgaccagaataggagaaaactttacgatggctataatcagctgtacattgaatctttcacaaagggagagtacgaaatcgttctgcagtttaatgcgtcgaacgaccagaaggacaacgcgtttttctacctccagttttatgtcttccacttgagtttgctcgataggtgcgctcactctgcagcgttcatcacaggtgcagacacaggtacagacagaggcattggcatagtcagtcaaatgagaggggaatccacttcgattggatcagagggtgcagcgacgccccccgtagcgaccacaccgaatgtacaggacatgtacgacctgaccccctatgtcgaagccaatcgggcggatttcaccatgggtgctaatcaaactcaagtgaaagacaaaaacaccttcttttttcagagctcaaatgtgtattacctattcaagagggacttactgcttctccatccgaacaaaaggattgtgaaggagctgattctcccccatgtgacgagcatggattccacagttacattcctcctgaagatagaactaatttttgcaatgaattatttgccatataagctagccatccaggatatgtcaaacaccagactacagtaccatgatagttacacgtataagaataaaatcctgatgactcttcccttagtgaatagcccccccttggtggaaaatagggccacgggtgaggccggccaggtagggaaacgcttcaagctgtacgtagacctgtacgaagaagttaacgaaaatttaagggacaacttttgcacgtatgcctacctgcacattatgtacacaagtgaaatggaggcttttcagaaatgggatgagcagggcatttcctactccacggtgaacttacctctccttctgaataatatacttttcaagggttctcctggtgcaacagggaaagcatttagtgtaggtgaagggtctaaccatggtggagatcactccgcgatgatttcgcttcgcaactccgatctgcccatagcgaaccagagcgtgcagtacacctttgaattgctgaacaacaatgtattcctattcctagtcagggataaccttttttttgatatgtacatgtacagggaaaacagagatataaagttgaatgtgtataaattttccgacacggagaaattactttctgggggaggaatcacctacgatgaagtgatgaagaaagggtcgtcatcgataggagaagagattgcttcactcaacaaggggcagatatacctatcgatgtatctaacgaggggtttatacatttttgtgtatgaacgtggatcaggcccttttttcgccaatctgtctgtggtgtggcattacaaggaaggcataccaagtgggtcttacctcacaggagaagaaggagtgggtttgccgaaggaggaagttggagtgatccttcccatgggcacaacaccaattcggagggatgatgcctttctgagggaagaaatcagtttcttatttggtaaggaagttaagtgcaacgaagggacaaaggtacaccatggcggtaagaagctgcacaatttgtcttacttttggcaagaaattataaaaaatgaaaatttcgaaattctcgaaaatagtacgaccaccgtggaaataaaaaaaggtgaacaaagcaatgctcttatttacaagcgtttttatatatgcttatcggatcaagtgcagaaaatttcacaagagggaaggaactcaaatgattctacacacaagagtagcgaaaccattgataaaaattacacgctttttaatttaaatatagaagagagtgcccaagtgtatgttgagataaacccccactatcatttctttgtttacccctttaatctaaatatcgtttcgaaaagatccttcttcgacatttccagtggacataaaaccttcattacgattgagctatacaaaggagagtacgaaatacatttggcctttcccgggctggagagagaacaagtaaaagatattatattcgacttggttatacttattgtgtcttctagaagcgggaaaaacgaaaccaatcagaggaatacactaacatatataggtaactctcctggactatccccccaaagtgatgtatgcaaggcgtacccatacaaaccccttttcaacaaagtcaatctggtggacattggggagaatgacgtatcagtgaagaaaaatattatttcctcaaaatataaaaatcaatattttcaccttttcggaaaatttttcctgaaaaactacaaggaagaagttttctttaccatcccaaatggttactactttttgaaaattttttgtgtccctattgatgagtcaacagaagaaaatatttctgtagaggaaggggaagaaaaaaatgaaattagagaggtgagaaagatctccgttggaaatttctcagacccttcaatgaactttgtaaactcctttttgaatgtacgaaatgtgtttaatattagggttgttcaggaacattcgggagaagccaacgaggatggagatgacaagaaagattcatcacagaacagctccctttccgtgtcacccatatttgctaacgaagatgaagagttcatgttgaatctgtaccaagtggagaaaaattttaaagtggtaatcagaacgaatagctatttctgtaattattttcagctagtggtaagtctctaccccctggatttttacaactctgtgcagagctatatgtattcccaggtgaactctgtggaaaaatatatgaagaatatttttgacaccttgtctgtgaaggcaaaattatatgaagatataaaatttgagcaggagaaatttccttcccatcagtacgtcagaatagaaacggatgttgtgaatttgaggagcgtccaaaaggaggattccttttctcttcccgtggttgtacataaggggagttacttcaaagtcaatctggggtatgatttctcgctagctaacttccaaatgaagctagcgaaaaatggtgtggctatttcaagtagcaccaagatggaagtcaacctgaaggataacagtcttaatacatttgaaaatattagtctatacctggaagagggaaattacacactggagattcattcttatcacatcactgcaaatttgaacaacataaataagaacttcagcttcttcttttatttcgagctcgagatttttgaattttctgacgacaagaccggggacgcaattctgctggatgtgtttccgcacaactcgatggctatcgacaggagctacccctttggcgttgatataatattctggggaagagtacacgcaaaagacatacatctgcaggatgatcaaaagacacacatagagccgaccaacagaacagcaataaagtatgcgaacattgaggttcagaaatttgttctatccccagaaataatgaacaaaatggaaagaaattttatttttaaaatttcaccaaattgtaatatctatgttaatcaggaaaaagagaaaaggttaaagtttaccctttcgactgaagataaaaatgcttccatcggagaaagcgtttatggagaggagaagggtgaagctattacaacgaatgaacggaatgaagaaaatggcgaggtccacccagagagtgtaaataattccttcctcttgaggtatgcggagaaggaaatcccgaccgacattcgcggaacaagctacgtggaaggaacttcccaaaaggacagtgtatacgacatcgaccgtgtcatacagaacttccgactgaacgaaatgaaaagaagggaagaagaggattccaccatggggaatccctcgaaggaagaaacggaagcgtgtattccgctgaccatactgacaatagaattttcctgcttcgagaaaggttactttctcatttttattttttttttgtgtttttgctttatgatgtgggtgttcctatttgtggttttaaaattatacaaaaactggaagtactacaggaattacgatgtcattacggagagtgatgaagtggtcaacctgttcgacgacgaccacatatag

SEQ ID NO: 33 - DNA, 6969 bp, Artificial Sequence
  Synthetic
atggaaagacgaaagaaaaggaaaaaaatttctctcctcattgatgtattactttactttattttgttctattccatgtttcctattgaaaaaaaaaatgaaagaaaacttagtgaaaattacagtgaagcaggaaaagcttttaagaatattctccccttttatttgaatactcctcagtactgtaatctattatttgacatgaacgtatcaggtacttattcaataaggtggggtgttaattatatatacttcgaacacttgaatgatagtttttttgtaaattttaacttgtatagtatatatgtaaaagaggtagaattgtacgagtacagacataggagttttctattattaaagaaagaatcagtagaaaatgaaataaaatttgtaacgtccattgagtatgataagagtatagatgcaaaggaacagaggactttttattttactgtgcattcaaattatgtgcagagctttgacacgtacataaatgtaacaattgatgtaaacatagttggacatgttggtaggggaggagaaagaggtagtggtcaggtgccatctgttgatgaaaagaaaaatgacgatataacaatggttgatgaatcaaaaaatgggataaaacaaaatagacttcctctaaataacaatagttacattataataaaggaaaattattattacgcatctaatgaagactataagttcaacgtaagagaagctgacaatttttatgtggactcgaaggggaacaaaataattgtaatatatagtgttttattttttgtcgattttaacaaacattgtaatataagtttcttctctcttctaacacaagggatagaacagggaaaaaacttttctatggacttaaaaaaaataaaattaggtaaaaattatatgcacaatatgacaacagagagtatattaaaatattcattttcccataaagagtgcaaagaaggttgcattaagagtagcaaaacaaaaaatggcgtgtttttacacacattaatggagaataataacatatataagttcgacatcgtgtacaccatatacagcagtcacagcacctacggcaatgacagcatttacggaagttacagcaattatgacatttcgaattctgatcgaggaagtggccatacgagcacctctagcgatacgatacgccctaacgaggttgtccattttaaccttgaatactctattgtgaacaactcaaaaaattacacaattaatgactacatgaaagaacaattttataaagaatgcgttcataatacttttttcccccatcagattattcaagtaggggagaatgacgaacattttaccggtttagtggttactccgtcgggtaaaaataagtgctcacagaatggcggaaagtgcatcgacgatgataaaaagaaagcagataatagtaaccacgtagcagagaatgttgatttcataatttacggaagagtgatagaaataaacgatacattcattttaaattttgatccaattaatttattcgaagaagaacataaggttggtttacatgtgagtgaggagtctttgtttaatttcagccttatttctaagtcggaaagtatatatgtaaatttgctaaaaaaagggtcatctcaaactgtttgtaatacttattatttgaacaatataaatgattacagtctgtttagttatataacaagagacagtgctacttttttacataaacatttcaatgatacatttgaggtgaacaagaaggatgcgaatcataagtttaaaaatttttctttacaaaatattttatacatcaactgtgtcgtaccaaaaggggaatacgaattgaagattattgttaatggttataatgtaatttgtgatccaaacgaaattaatttagtcctttatcccttgctcatgtatgaggaaaaacatacgtgtgacagtagcatgaatgcacttaccgatttgttttactaccacttgagagcggaaaataggagccaaagtggaagaggaagcgtagttggcggtttaggaggcactactgcatcgagagtcgacgtgtctggtgaagccacccccgagagaacctcaaacagtgcatccagctacgcaaggcagaatgacgatctgacgtcagaagtgcaaaaaacgtatagtaatatttacgagaaaaagtggatgatgaacaatcccctctttgagcaagacgattttgtaatattatacgaaaagagcattgaagaacaagtagacgagttggttgttattgtaaacaactcattgtttgttgatatttttttagtaatagtagaatttataggggaggacagttattattatatatatcgaaactcaagaagcatagtgaaatatatgcttaagtataaaaacccttttactatatatttgttggcatcaaatttacattacgagaatagaaaattatgtggttatttttttgtagatattgattttttaaaagggtctataacagtatcattggatggtacagaggattataaaaagatgattcaaaaaattaattatataccaagtcttataataggatatgactcctacgcctttagtaacttttgctacgttccagattacaaaaaacatattcttacgatatgtgttcaagaaaattcaatcataaaaataaattgctttacacaaaattatatttatatttatttattagatcaaaataaaaataaaatatatgaagggtataaccatttatatattgaatcatttactaggggagaatatgaaatcattttcgagtttaacgtgccaaatgataaagaaacagatgcttcttttttttatttacaaatatatatctaccaactgagtgctttagaaaaatgtgtatttgatggccaaactgatagtagtagtggcagcagtagcgatggaaatggtaaagatgcccaattcaagaactacttatcatcgaacgaaaatggagacaattctgaatatgaccttagctcctttagtgcaactaataacagcaaaatagaattaaagcaaaaggggaaaaacttttttttttttcaaaatgaatacgcatatcatctttttaaaagaaattttctttttctttttccaagtaaaagaacgacaaaaaatttaattttacctcaagtgtcacatttaaataatattgcttttttgctaaaactggaacttgtttttcataaaaatttcttaccttataaaatgaccattcaagaaggcacagatgatttgttaatgcatcatagttatacgtataaaaataaaattttgctaactatgacaattttgaataatagtgtaaaagtttttaagttgtatatagacctatatgaagatataaatgaaaaattaaaaaatgattattgcccctatgcctatttgaatattatctataccaatgagggaggaacatacaagcaagctgctttttcaggtttaaactttggtgaaatcagtatgcccttacttctaaatagcatattgctaaaaagcttcgcgtatgctaggggcatgggcaatagcgaacgtaacaaagacgaagatatgggggtggtcagacgcaacgatagcggtgtcagaaatgatgactggagcaacaaacacacttcgacaatatcatttagaaactcaaacacagttactattggtagccaaagcgtggattataatctaaaaattattaataatagtacaacgcttttcttggctgaacatagtgttttttttaatatgtacatgtacagggaaaataacgatatatcattgaaaatttataaatatttaaatagtgataacaaattagaggaaggatacggagtaacatttgatgaaatgaataaggagcatgttttatcccattcggaggaaattatgtctttctcaaataaacacatatatatatcaacctggttaaaaaaaggcttgtatatatttaaatatgaacaaagtacatctccctttttcattaatttgagtgtaatatggaattatcaagatgatgataaaaacatggggaagaattatcttaaaagatataaatatggaggacagtcattaacacattataataatgggaagactgaagaaccccctgccgatgatgctattagtgatactattttgaaaaatgaaatttatttttattttgataaagaactaaaatgtgatggaagaaaagatatattttatgaaagcaaaaaaaaactacatcggttaactttttttttaaaagaggtcataaataacaatatttttgaaactcattctgatgatagtatgcagataactgtaaagagggactcaaggaatacgttaatttatgagagatattgtatatgcatagggggtggtccgaaagtgaatgacatgcattccgatggttcaaccagtgcggtaagtgctaatgaagtcgcccagggtgtagtaacgcaaccgtggaccggtagtggtagtagaagcagcagtagtagcagtagcagtaacggcagcagtagtagcagtagcagtaacggcagcagtagcagcagcagtaataggtacaaaatttatcacattagtgtagaggagagtaccaaggtgtacatcgaaataaagccgcattttcatttttttatttatccattccgcgtaagtgtaacatcagaggatttttattttaacatttttagtgaaaataggaatgttattaataccaatttgggaaaaggagagtacgaagtaattctgtcttttccacaattaaaagaaaataaaaatacattacaaaatgctatatttgatcttattatattagtaattttgcctgaaaagggtgcaataataggggataaaattttagctagttcatatatgagtaataggttaagtgggaaattgttagaaaaggagttgtgtaatataagcacatataaaactttatttaataaagtagatttagtaaatataaatgaaaatgattcatatgtaagtaaatatattatttcatcaaaatttaaaaataactatttccaaatttttgacaagtattatgttaataattttcaacaagaaatattttttacaattccaggtgagagttacattttgaaaattttctgcatgcctatagatgagctgggaggtgaaaatatgtacaaagagggtacttacaataatacatataatggtaatagtaatagtacgtataacaataatgcatataataataacataacttacgatagtagtgtccacaataatagcagtagtgaaacgatgagcaacatttacaatgtggcttctaactacgtaaatgctattttgaatgtacgtaacaattttagcgttcaagttgtactggctgatatggataataagtctaatgttaagaataacacctcgaatgataatgagaaggataaatatgatgataataacagtagtaatcgtaataacagtagtaatagtaatgccttcgtttcaccgctacattcaaatgagaatgaagagtatatgctaattttatacaaagtggacaacaattttaaactggtaataaaaacaaataatcatgaatgtaattacttcaagataattttaagtcttcatcccctcagtttttataacagtgaacattatttctcatatgctaattacctagataaatatttgaaaaatttattcaataccttgtctgttaagaccaagttgtatgaaggtataaactatacacatgaaaagtcgtccatgtacagcctcacacgtttaacttcacctattgttaatttgaaaagtgtcaatatgaaggaccttttttcctttcccttgtccattaataaggctagctatttcaagataaatattggctacaacttttcgctagtcagttttgaaataaagttaatgaagaacagtacgactatttcatgtagtaataaggtggaagtaaacttacaagatgacactgtgaacatatttgaaaatattagtgtatatttagaaaaaggagattatatattacagatcttttcctttgattatatgggaaattctgttaatataaataaaaattttagtttccctttttattttgagcttgaaatttttgagttttttcaagaaactaagaagagcggccctatcctcttggatattttcccgcacaattcagtccctgttgacgcagttttttggggagaagtaaaaggaaaagaaatgttcttggaggtgcataacaagaaatatgcagatttaagaaataggaaaataattaaatatggaaatattgaaatacataaatcttttttattacctgaagatatgatgaacatggaggaaagttttattttaaaatttaccccaagtgcgaatatatatgtgagtgaacaaaatgaaaaaaaattaaactttatcttttcaacttatagtacaaatattgaacagaacaacaacagttacaaagtggtgcaaggggaagacaagattgtgatgataggatcagggcaaagaaatggacaagtacatgaagaaaatgtgaacaagtctttcttgtttgaatatgatcaaagggacattactgaagcaggagaaagtgttaatgattctcagacgaatattcacgtagataaattttttgaccttgacaatgttattaaaaattaccgacttaacgaaaaaaaaaaaaaaagagaagtatcttacacgtcgctcgagaatagtaataagcagaatgactcgtgtatccctctatacttatttacatataaaatttactgtttcgaaaggagtaattatcttattttcattttttcttttttccttttttccgtgtcttgtgcttttatatgtttactaataaaattgtacaataattggaagtattacaacaattatgatattgttgttgaaagtgaagaagtaataaatctttttgatgacgatgacatataa

SEQ ID NO: 34 - DNA, 6498 bp, Artificial Sequence
  Synthetic
atgtttgtttgtatcatcttctgcgtcttgtttctcccatttgggagaggaatacccagaaatcttggggatgacaacataaaaagaagaagccagtgcgagaacttggtaccattttattttaacgccccacaatattggaacatgtctggggacattaacatttcagattattattcattgagatgggatgttaactacatatattttgaaaaggttaatcataaaacatttgtaaatttcaacctctatagtatttatgtgaagaatgtcgaactttatgaacatagagatagaaacattatgatactaagacgtgaagttatggaaggggaagtaaaatttgtccattttatagatcaggatgtaagtttacatacattggaaaagagaattttttattttattatacgtttgaagaaaccccttcatgatgacacatgtgtaaaagtagggttagatgtaaatataggaaaggctcctattacaagtttcgaagatggtatggttgtccaccaggagaaaaaaagcaaaagaccattgcaccataaaagttatattataattagggagaactattattatgcctctaatgacgatttttactttacaccagaggatagtgaaaatgatacatacattgatgataatggtgccaaatttgtaattgtatacagtgtactattatttcctcaatttgataaaaaatgtaatataagttttttctcctttttaaatggagagataacaaaaaagaaaaatttctcaatgcaattgagaaaaataatgttaaacaataattattttcataatatgaatatagatagtgtattaaaacatacatataataatatgacatgttcagaaaaatgtgtaaaaagtatatttacgaaaaatggattattaatacataatttattggaaaataattacatgtacaaatttgaaatagtttacaagttgacagaaaatttaaataaagatcaagggaattatgaaaataatacagtaaatggttggacatataatataaatagtaagacttatttcaatatggaattttccgttgtaatgagtttaaaagatcaatcgtttatcgattatgtaaaagaatacttcaacagtgaatgtatacataattctttttttgtgaaaaaaattgtacagattggaatatataaaggtagaaatgacaatgttatttcttccttttcaatgaaaaaatgtcaagaaaaagacaaacatagatgcaacttcacagagttaaacaatgaatatgtaatatatggaagaattatacaactgaatgatagttatgtcttaaacttcgacccaattaacttattcagagaagaagaaaaagtgcatatacacattagcgaagactctcttttcaattttaccctaactaccacgttggaaaacatttatgtaaatttgttaaaggggaactctacccaaaatgtatgcaacacttactatctacaccatataaatgagtacaatatttttagctttcaatctaaagacaaagccccttttatgcactatcattttaacacaacatttggagataaaaaaaagtatagtaaggatcacagattaaagaaattttccttacagaatgttgtatacataaactgtgttttgcccaagggggagtatgatctacggattgtcatgaatgattatatctccatttgccaaccgaacgatgtacagttgactattttccctctccatatctacgaggagcggcacacctgtggtagagaaatgcacgtgctcacggacctcatctactaccacctcaggggggaagggcgaagaggtgggagtagcggtgaggggaataggccgatcaaaaatgaacttaccgaaccatcgtccgaacaatcaaggacaatctcccacaacgtgtacgaaactatgtgggagctgaataacccgcttttcgaaaacttcgatttcgtcgtccttcacgaaaatatcatcgatgaggaggtggatgagatcgtcgtgacagttaatacctccgcatttgtagacattttcctcgtgataacagaatcagtgggggaagagagctactattatgtgtataagaactcgagaggcgttgcaaagtatgggctgaagtttggggggccgtttcacatgtacctactggctcccagtttgcactacgaggataggaagatttgcggctttttctttgtcgatgttgaccttgttttgttgggtaagaaggcaaaggggaagacaccacgcagagaagatcacactgtgggggaggagaccctttcctcggtggacgaaccccaaggaaggaacgtagttcagaggactattcacctgccagagttgatcataggatatgacacttatgccttttcaaacttctgttatgtcccatcttacaaaaaacacgttctcacaatttacgtacaggaaaattctattgtaaaaataaattgctttacgcaaaaccctatttacatctatttgttaaacagtgagcagaagaaaatacatgatgggtataaccatctatatgtagagtctttcaccaaaggggaatatcacgttgtttttgagtttaatttgcagagtgagaaagaagcaagttcctttttctacttacaaatatatatttaccactttaatttgttggaaaggtgcgcttttgataatccctctgaggctttgtcaagctcggaggagcttccgcttagcagtgcgctgcgtattgatccgttgcgaagtgacccttcacaaagtaatgatgtatacgattttagcatttacaacgagatgagcacggcaaaggatggaatcaaagtggtagatcccacttcttttttattcaacgatgaacattcctatttcttttttaaaaatatgtttattgtcatattttccaacaaaataatatcaaaaaaggttatattaccccgagtagcaactcacttggaagcctctccatttatgttaaaaattgaattgatttttcacaataactttttacccctcaaattaagagttcaagatattacaaacagtcttctatatcatagtagttacacatacaaaaataaaataatattaacacttaccctttcgaatggtagagtcagagttcttaatctccttattgacatgtatgaagatgtaagcgatagtctcaagagtaactactgcccctacgcttatctacactttgtgtacacaaacgaggtggaggcataccggaagattggagcaagtgggttatcccatggcgcgatagatttgccactgctcttgaacagtctcctcctgagggctgtgtggggagtcgacgacaaaggggaagaaataagaataaatacctcctccgataatgtcgatagtgcttcaacccctccgattgagggaggtgtaaccttcagaagtccagtctttcccgtcatggtaaaccagagtgtgaactataccatcgaaatggttaataacgacacgatgcttttcatcgcggataatgacgtttttattaacatttacttgagtaaagaaaatggaattgttgtattgaaaatgtatcattgcttgagcacaggtatactagagaagggtagtggaatacagtatgacgaggtgcataagaactttctttccatttcgaaagaaattgcatccttatctggtaaacatgtttacctttccacctacttggaaagaggtttgtacgtgttaaagttcgaacgagattcttctcccattcatgttaacgtgagtgttatatggaactctaatagagaggatccaaccataataggttacgtcaaacgacaagaacatgcaaacaaactggtaaattatgccagtcgagaagtcttacctgtgatgagtatgcacagaatgaacgtgtcagcacaaggcacggcaagcgctacagatatatccccagcgggagatcccattttgcgaaaggaaacacatttatttttcggaaaggaactacattgctctggcgtgaagcaagtatttcgtgaaagtaaaaaagtggcaagtgtacctttttttttcgatgaaattgtgaagaccaaattgtttgcagtttactctggacgtacaacacacattttggtgaagaagaatgcagataatatgttaatttacgagaggttttatgtctgtctggggagggaaaccaatggctactatgaacagtacagcggggaggcctctcgcgatgagagtgatcaacacaacgagggaaaatacccccttttcggcatcactgtggaggatgtcgcccagatatacgtggagataaagccacactaccatttttttatatacccttttgaactaaatgttacatcaagcagttcctatttcagccattcgagtggagggaaaaactttgtgagtacgaaccttggaaagggagagtacgagattgatttacgttttcccgagtggacaaataatgatataaaaagtgtcatgttcgattttgtcatattattgacatttccaatttgggaagcatatgatacagaagtgctttccaaaaagggtacaaaaatgcttcttagggatgatacatgtaatgtaagtatgtacagaagtttgtttaacaaagtcgatttaattaacacaacagaaaatgatatcattgtaagtaaaaatataatttcccccaaatttaagaataagtacttccatgtatttgataaatatttcattagggattaccaacaggagatattttttacaataccaagtggtggttatatattaaaaatcttcgctgtggtggattcagagattggagaaggaaatgatcagggagaaggttttccgggagagagtcaccctcagggtacgggacgtgtggtagattttccagcaaattatgtgaactcctttttaaatgtgcgcaacgtttttaacattcgaattgtcgaaagtgatttgaatgggttaggaggaaagaataacaatgttgaagataagaaagaggaaatttcttatgtggcaccagtatactcaaatgaaagcgaagattacatactagttgtgtataaggtttataccaattttaaaatgataattaaaactaataactatgattgtaattatttccaacttattttaaatctccattcagtaaactcttacagtgatgtacatcacttttctcatgcatataattttaacagttacttaaaaaacatttttgataccttaactgttaaggcaaaactgcatgatggcataaattttgagcagaagaaattgtcaacatataatcatgtttccataaccacttctattgtttacctgaagaatatcaatccgctggaattgttctcctacccactgtctatcaaaaagggaagctacttcaagataagcgtagggtataacttcacccaagcgcatttcgaagcaaaactggggaagaatggtgaacctatttgcagcggtagtaaggaaaaagttaacttgaaagatgataccattaacgtttttgagagtattagcctatttttagacgaaggggaatattcattgcaaattttgtcacatgatttaatagaagactctgttaatataagtaaaaaatttagttttcctttctaccttgagcttgacatattcgagtttatgcatgagaagattgaaaatccaatccttctggacatttttcctcacaactctgttcccctggacaggaattatcctttctttgttgaccttatattttggggagaattgagaggaagagcagtttatgcggaggatggaaaacagaaaagggtgcaactaagaagtggaagaatgcatacccatggaaatgtagagatacataagttaattttacccccagaagagatggatcacatggagaataatttcgttatattgtttgatgcaaaagaaagtaatcaaatgaatagagaaaaggagaaacggttgtatttctccttttccactatttccacaaatgtgttgagtaagaatagtagttatgaaccgataagggaaaaacaaaatgtaatgtctgaatcgaaggaagggaatcgacaaatacatccggaaagtgttaacacctcgtttttgctggaatatgggcagagggatgctaagcgtgacgactccatacaggagaactccaacaaagacaattttttcgacctggacacggttattaggaactaccggctgagccagaagaaggataaggaatatatgcctgtttcaggcgaggctccccctaaaggggaaagcagcacttgcatcccactgtctatatttgcccttgagatttactgttttgaaaaaaattattttctccttttccttttcgtggtgttcattttatgcatatcgtgtgccttcctattcctactgataaagttgtggaaaaattggacatactacagcagttatgacgtcattacggaaaacgaagaggaggtaacactcttcgacgatgacatatag

SEQ ID NO: 35 - DNA, 7419 bp, Artificial Sequence
  Synthetic
atgagaaaaaagaagaaaacgtgtttggttcttttaattttaagtttcttatgttttttttattcatctttatcttatgatgaagattacgatgaggaatacaattatgaagataagaacagtgattatgaatgtaataatttaattcctttgtttttaaataatttagaatatagtaatatatctcaggaaatatctttgtctaagactttttctttaaaatatcctgtgcattatatgtattttaaatattcatctgatcaaagcatttttgtaaattataatatatacaattctttatcttttggatatgtaaaaaaaatagaattatatgaatatgaaattattaagaatgaaagtgtgttattattacaagcggaaaaagattctgataaaaatattaaaattttgtataccataacaaaaatagataataggaaaaactatgaaggggagaaggatcaaaggttattttattttattatatatacgacaatagaaaatgataatgatataataaagagttgtataaatgtaaatatgagcatacatataagaattataaaggatgacacacaaacaaataatcataacaataataatattattattaataataataataattattattataattatgtgcataataattatgggatgaggggtatcatgccaaatcatttatataatcatcttctacctttacataataaaagttatattataataaaagataattattattattgtactaacgaagattattattataacatgaataatatagaagattattattatgatgaaaagagtggcgacaaagttgtggttatatattctgttttattatttatagaatttaaaaaagaggcgaatataaaatttacatcattcttaaaacatgatataaataagtacaacaattttgttatacatataaaaaaaatagatatccataataattatttaaacaatatggatatggaacatatcttgaaggttggaaataatgaggaatatataaatggtaataagaagaagaaaagtatggaatgtgaagaagattgtgtagaaagttacataacttcacaaggaatatatatagatacaatattaaataataatcatttgtatagattagatatattatataagatgggaaaaaatgaaataaataataagaccactgaaattattaaaaaggaaaacaacaataataacaataacaataataacaataacaataataataacaataataataacaagaacaataacaataataataacaataataataacaagaacaataataataataattattatgattatgataatgacaaaatgggagatataaaaaatatgaaagacaatggaaaagatcctttcttatcatcattattattttttaattttgaattatccatatataacattacagataattatattcctatgaattatattaataaacgattatacaccgaatgttcaaataattctcatgctccaaataagattgttcaaactaaggaagagaatatgttaaatatattttatacaaatgaatgctcaaagaatatagagaaatgtagtttatatgatgttaatgaatatataatatataataataaagtcattgatatacatgataattttcttttaaactttgatataataaattcttttaaaaatgaacaaattatagatatatatataaatgaagattctatttttaatttttctatgctcattaaatctgatcatatttttgttaacttattaaaaaataactcttcagaaaaaatatgtaatacatattatatgaataatattaatgattataatatatatgattataaaaaaaataacaaatctgatttttttcataaacattataatgatagttttttatataatcaaaatatgaattataataacacctttgaatatcaaaattattatcaacatgatcatcatttaaaaaatttccctcttcaaaatattatttatattaattgtatattaaagaagggatattatgatttaaaaattattcttaatggttataataatatatgtgaaagtaatgatttcaatttattaatatatcccatgtctctatataaaaatcaacaccaatgtgatgataaaatgaaccaagtaactaatttatttaataatattttaaaacgtaaataccaacataatattagtcatgtaccaaatcaaatccaaacctttgatcaaatttatcaaaataaatggattttaaataatcatatatttgaatattttgattttgttataatatatgaaaaagaattactaccacaacaatataaagaaattatggttactataaataattctccaggtcttgatctattctttattcttgtagaaaatgtacaaggtcaaaaatactatcatatatataaagatacacgaaaaaaaaataaatacatattaaaggataaacacccatgtactttgtatgtacttgtatcaacattattatatactacccaagatgtttgtggattcttttttgtcgatatagaatatgtagataatatgaagttattaaatcaaaacgatcatccaaatgatataatgaatggaacatatcaacataatatgatacaaaaaattaattatatacctactataattataggatatgattcgtatagttatagtcatttttgttatgtaccatattacaaaaaacataaacttactatattattaaaaccaaaaacgatagtcaaaataaattgcttcacacataattatatatatatttatttattagataaatcaaataataacaacaacaacaaaaataataatgataataataagaagaagttatatgaaggttataatcatctatatatcgaatcttttgttgaaggggaatatgaaattatatttgaatttaatacgcaaaatgataaaaatatgaattcatttttttatttacaaatatatatatataatttaaacacattagataaatgccttttctttaataataatatgaattatataaaagaaagctccataaataaatttataaatacaaataactatgatacatttgatcttagtaaatatgaatataatgatattattataaataaaaatgtacccagagtaattaaaaaagaaaataatatgttcttcatttatcaaacaattaatttttattatctctataaatcattctttctttatattctaccaacacaacataaagatggaaatataacaaacgaacaagataagaatagaattaaaaaaaaaattatattaccaaaaattaattatttatcaaatcagaatttcatattaaaaattgaattgtcttttcataaaaatatattcccatacaaaatgtatatcgatgaaaattatgatgatattatcatgattagtacaaacacgtatagaaataaaaatattatgttattaaaaatattaaataataatacaaattatataaatatatatatagatacgtatgaagatattaatgatgaaataagaaaaaattattgttcatatgcatatttcaatattacatatacaaatcaaacagaggaagaaactgtggatagacaacagataaatttatataataaaatcactttacctttattattaaataatattttatctaaggactatgttccaatgattaataaaaatatagtagaaaataagaatgaccaaacatataaagctgatcatttatattatgaggatgataatattggtgtaataaatagtgtatccaataattatttctctggtgagaacaaaaatatgggggatatcaaaaataagatgaataatgagtatggtagtgttcatacagaacagatggtacattttcaaaataatgataaccttaacaataataataatatcatatttggtaatgtttatccttatcttgaaaatcatcttagaggtatcatatctctcaaaaatagcaactctttaatattaaatcaaagtgttaattataattttgaagtagttaacaataattatacacttttgataataccaaatgatgcttttctaaacatgtacataagaaataatgatgatgatgatgatgatgataatgataaaaataataataataataataataatagtaataataataatagtaatagtagtaaaaagaacattactttgaatgtgtaccgaattttagatgtacctaaactcacaaatgaaggtatttcctttaatgaagtcaagaaacaaattctgtccttatctcacccgttcttaacgtttaccgataagtatataaatgtatatagatatttagaaagaggattatatatatttaaatttgatgacgattctaataaggaaaattcaagttatacctttagaaatagtagcaatatatataagaacaatataaataattataattatcattctagtgatagtattaagccattatccttttttatgcattttaatatcatgcctatttacacgaatgatatagaatacaaggataatatcaaaaataataattataatgataaaacgttcaattatcaagggcatgataaaaatatgtacccttctgtcattcataactatatatttaagaatgaattattatattatttcaataaagaatacagctgtgataaagagaatatgttattttttgaacataaaaagatagatgatgtatctaattttttagaacatacttggaataatataccatttgaaatttatataaatgacaaactagacattacattaaatcgagataactacaacacattaatattaaaaagattctttatatgttttgaaaaagaaaaaaatgtaaacaataatgacaatattaataatagtaataattcctatgtaaataatactgataatattaatgaatcacaagatgactcttatataaacatttttaatgatacaattaaaaaagaatatctcctttttaatatcgtattaaatgtaaaaggtaatatatatatagaaatacaaccacattatcattattttatatatccatttattttaaccataaaatcgaaaaataatcccacacaacttaataaatctcatgataaattatttttaactaccgatcttggcgtaggtgaatatgaaatctatataacgtttctttcgaataatcattataaaaatgtaaaaatgaaaaaggtcatgtttgatttatttatttttattgatattttaaaaaataaaaatattcaacttggaaaaaataatttatacttaccatatggaaaccatcttgatgacactatagattttaaaatatatgataatacatgtaaaacggataattataaaagattatttcatgaagtaaaattattagaaaataataatagttccaatgttcatatgaatgaatcatatataaaaaacaaaaataatattatcagtacaaatatgaatacgaattattttcatatttatgatacatattatatgaaagtacgtgagcacgaaatttcttttgagataccaaaagaggcgtatattcttaaaattttttgtatacacatagatgaacaaaattatgatgaagacataacaagtggagatttttacaataatacaaacgcttataataataataataattatggaaataataataattattataaaattgaacaaactgataatttatctcatttttcttttaattatttaaacacctattttgatatccaaaatttattccatattaatgtaataacaaatgaagacagtaaatttttttctgatgaacatacaaatgatcaagatttatatgttaaaccgtttttttccaatgaagaaaatgaacatatattaaactggtataaaatagataagaattttaaattagttataaaaaaaaataattatgattgtacatatttcaagctaattataagtttatatcctatggattattttaatagtattgattatttaaaatataataattatcttaataaatattttacaaatatatttgacaccttatcacttaaggtcaaccaatatcaagacatatcctttgagcaaattaaaaataagaattatttatatgactatttaaaaacaaatgtgatttttttaaaaagtttgaattctagggatcttttttcttatccgcttacgattaaaatgccgagctatgtaaaaatcaattttgggtacaacttcactttaaccagctttgaaatcaaactcttgaaaaacaatatgataatcgctacaagcaacaaagtacaagccaatataaataataacaatatcaacatatttgaaaatgtgagcatttatttggaaaagggaaattatgttgtccaaatttattcttatgatttaattgaaaagtcttctaatataataaacaaactcagttttcctttttatgcggaaattgaaattgtgcagtttgtaaacgaccaaattgacgaacctatattgttggatgtgttccctcataattcggtcattgtgaatagaaactaccccttctttgttgatttaattttttggggaagagcaaatgaaaacatatccgtgttggatacaaaagaaaataatatcaaattaaataataccaagttaataaaatatggaaatgttgagatacacaaatatgttttgtcatctgaacaaatgaggtctctggaaaataattttactttaaagcttcagtccaatgttcatataagtgcatggatggagaaaagaatgaattttagctttacaaatgagggttggaattacgatggacaaacagaacatgggaaattaatgaaaaatggaataaaaacaaataatattgttgatgaatcttctaaaatatccaagggagaaaaattaaatgagactgtgttattggaatatggcaagagggaaataaaaaatgagagtgagggtgaaagtaagagtgagaataagattgaaggtaatagtaataatgagagtgatggtgagagtgagaaaaggagtttcttacagaacggtgtaatagaaaaaagcaacctttttgatctgaatgaagttattaaaaattttcgatataacaagaaaaagaaaaaagaagaagaagatttccttttgaacggatcatatatgaattcaaatgataaatgtttctctttgtctatattcagttttgagatatattgttttgaaaaattttatttttttatttttatctttatcttaaccgtattatggatatcttgtgcctttatatttttaattataaaaatatataaaaactggaaaggttatagaaattatgacatcgttggtgaaatggatgaagtgataggtctttttcatggagatgatttataa

SEQ ID NO: 36 - DNA, 6591 bp, Artificial Sequence
  Synthetic
atgaataagagaaaagggaaagtgtggcttttcctttttataatctgtttgatagttattaattttaagatttcttatgaacaaaataatgaaaggaatcttagtgaaataacaaaaaatgaaatcgaatatgaaagatgtcaaaatttagtacctttttatttaaataacccagaatattatactatgttaaatgaaattaatgtatcaaatgaatattccataaaatttgatataacatatatatattttgaatatataaattgtaatttttatataaatctaaaattatatagtaaagatgttaataaagttgaattatatgaatatgtagataataaaggtattttgatattttctgaaaatagtaaagacaatttaataaaatatttatttcatgtgcaagatgataagaatagcaacaaaaatgaaaataaaaatgaaaataaaaatgaaaataagcgcgttttttattttattatatattcaaataaaatcgaaacatctaatgaatgtgttaatataagattagatataggaatagggccggttgttttggaaaagggggatacaaaaatagagagtacaaaaaagggggatatacaaaaagaggggagacaaaatgagggtagacaaaaagaggatagacagatagaacatcataatagctacataataattagagataattatagatatgcaacagatggttattttaatttttctttaaaaaataataaagatttttatatttatgaaaatggtaaaaaatatggaatcatatataatgttataattattctcgaatttaatgaagaaaataatataaattttttttcacttttatcacaaggaaaaaataaatggaataatttttctatgcaattaaaaaaaataaaattagataaaaattatttacataatatgaatatagaatatataataaaacatgcgaatgctgatatgacttgtaatgatatgtgtataaaaagtgatgaatcgaaaaatgggataatatttttaaatgctttattaagtaataattatatttataattttcaaataagatatgctgaaaatgaaaatgaaaataattacataaataatgatattaactatgataatgaaaataaaagtaataaatatttgggtagaaatagtgtagagagtaaaaaagaaatgtttaatcaagataatataatttattttaattttgaatataatatagttaacaatgttgataaatatacaatgatagactatttaaaaggccattttaatcatgaaaatattcagaatacatttttttttgataaaataattcaaataaaagataataatataaataaaaataatatattgccaataattggatttaataaagaatgtgtaggagaaaataaaaaagaatgtaataaatatgagataaataataattatataatttatggaaatactattgaaatagatgataattttattttaaattttgatcctaataatttatttaaagaagaaacaaaagtgaacataacaatcgaagaagaatctttttttatgtttacactagaatcaaaatcagaaaatatttttgtaaatttattaaaaaaaaattcaacagaaaatgtttgtaatacatattatttaaagcatataaaagattataatttatttagctatttaactaaaaatgaaaaaaatattttgaaatatgataaaactaattttagtagacatcatataaaaaatctatcaaagaaaattataatatatataaaatgtattcttttggaaggagaatatgaccttaagataaattttgaagaatataataaaaaatttgaatcaaataatattaatttaataattcagccattacatatatatgaaaaggtacattcatgtgatcgtaaaatgaatataataacagatatgttttattataatcttcgaactgaaaatgtgaataaaaaaaatgataataatgttatatcttataaaaatatatatgaaaatatgtgggaattaaataattcattatttgatgattttgattttataatattatatgaaaatgagatatttgaaaatgttgataaaataatagttactataaataattcaatattctctgatatatttattataatttttgaaacagtaaaagatgaaacacattattatatatacaaaaattctaaatcaacaattgaatatgaaataaaaaataaattaaataataaaataagtatatatgtattaacaccaagtttacattatagtggtagaaaaatatgtggttttttttttgttgatatagattttatagaaaataaaaaggaatctattggagaaataacattatttaataatatgtataaatcaaatagaagttatattggaagtattaatcatattccatatttaattataggatatgatacatattcatttagtaatttctgttacataccagataataaggaacatgttcttataatttttattgaaaaggaatctataataaaaataaattgttttatggaaaattatgtatatatagaactatatgaaagaaaacatattggtggtgaaataaaaaaaattaaattatttgaagagtatcaacaagtatatattgaatcatttactaagggagagtatgagattgtgtttaaatttaatgtacaaaatgatagagaaatgagcaattttttttatttacaaatatatatatatcaaataaatttattggataaatgtgtttttcataatgaagatgattttggcgaaatagcggagggttcgagttctgtatatgatctaaatgaagttgatagaaaaataaaaagtaaaaatggtaaaaaaaataaagaaattggatattttaatgataataatggctttatatttgaaaatgaatcttcttactatttgtttaaacgatatttactatttctttttccaaaaaaaattgatgaaaaaattgaaaaaaaaataatattaccagaaactaataattctgggtttgtattaaaagctgaattagtgtttcataaaaattttttaccttataaattatctttagaagaagatggggaaaatatattagcccatgaaagtaatatatataaaaataaaattataataacttttaatattttaaataaaaatgtaaaatatgtaaaattatatatatatctgtatgataatgtacatgaaaaattaattaaagattattgcccatatgcttatttaaatattgtttatacacatgagttggaaaaatatagagaacatgaaaacgatgaatatcaatatgacactatgcatttgcccctttttttgaataacatattgttgaaaaggtacgaatattctgatgatgtggaagaggaagatgtagaaattggtaaagtagaaacggaaagtgggaatgataaaaatgtaagagtagttccttttgacaaaaaaaatggaatgtgtattgggaaccaaaagatagaatataaatttatcacaagtaataataatatgatgttttttttagcagaaagagatgcttttttaaatatatacatttataaagaaggaaaagaagatattatgttaaatatttataaatataaaaatatatcaaaatttgaaaaaaatgaaatattaccatatgatgaaataaataaagatatacaatcttgttgtgaagaaatattttcacattctaatgttaatgatataaatatatcaacttattttgaaaaagggttatatatatttaaattttctgaaaaattgccatatataaatatgatatttagtgtaatatgggtggaaataccaaatgttaatgatataattatgggttcaaatataaataataaatatgaaattaatagatatattgaagaaaatgaatctaaattttattttagtaaagaattaaagtgtggtgataaaaagggaatattttatgatgaaaacaaattgaaagatttagaaaaatttgttatagaaaatatgtttgaaaaaaatttatatacaatttattttggaaataaaaaaaaagaaataacaatttcaaatgattcaaaaaatatagtaatgtatgaaagaatacatatatgttttggaaatgaaatatttaatcatcaagttattaataatacaaattatattaaagaaaatgaagagtctcatattattttgactttaaatgttgaaaaagaatgtcaagtatatgtagaagtaaaacctcatttttattattttatatatccatttaaaataaatatattatcaaaaaattcatattttaaagtatcaaatgaatataaaaaatatgtgtatgcaaatattgaacaaggggaatataatatagagatttcttttaaaatggaaaattatataaataataaaataaagaaaattgatggtgttatatttgattttataatatttatatataatacatcaaatgaaaataaattaaaaaatattgatttttcagatgaagaaaaaaaacagatggtatcattaaaaaatgagacatctaatatgaataaattatatactcgaaaaatttataaaaatattttcaaaaaagttaatcttaaaaatttaaatgaaaatggaattgtagtaaatgaaaaaataactacacataaatcgaaatatagctattttttttgttatgataaatatattattaaaaatcatcaggaaatattttttacattagtagataatgtggattatattttgaaagtttatttagcaccaatctatgaactaagtggaaataataattcgaaaaatataaataatgattttgaacattttgaaaattttgaaaattttgaaaatttcgaaaatagtgacaataatagcgaatcttcatctctttataatttttctacaaattatttaaactatacattaaacgttcgaaatgtttttaatattgaggtggtaaatgagagtatgaataatgattatattcctgaaaaggtaattaaagagaaaagattaaaaaataaaataattgaaaaaaaaacaccggattcaaatatattatatacaaacgaggatgatgaatatatattaaatgtatatgaggttaataaaaatttcaagttggttattaaaaactacaataatatagatagtaatattgaattagttttaagtttagttcctttaaaatattataaaatgaatatgaacacaaaatatgccaatgaatatattaacagctattttaaaagtatttttaatacgatatctgttaaaacaaatttatatagtggtataatttttgagaatagagaacattctactcacatatatacacgtgtagaaacttcggtggtgtatttaaaaaatattatcatggaagaaacattttctttccctttgcatgttaagaagggtagctacatcaagctaaacattggatatgacttttcaagggttaactttgatgtcaaaattatgagaaataataagaaggtctcaactagtaataaaattaaaggaaatatggataatggaaaaataaatatttttgaaaacattagtttgtttttagaagaaggagagtatacatttcaaatatggttttacaacttaactgacaattatattcatataaataagaatatgggttttcctttttatttttcacttgaaatttttgagtttgctgaaaataaaaataatacttcagtgttattagatgtgtatcctcaccgttcagtatatgtagacaaagcttacccctttaatattgatcttattttttggtccgaggaaaagagagagaaacatggctttatggaagatgaaatgaaaaaaatagtatatttaagagttggtgaaataataaaatcaggaaaaatagaaatccaaaaaagttatttattaccagaagatatgagcaatcttaaaaataacatgactttaaaatttgatttaaaagataatgttgatatgataagtgaaaataacaatgaaaataacaatggaaataacaatgaaaataacaataaaaataatagatatcttttcacttttttaaataacgatgtaaaagaaaatgaagtagctatagaaacgaataatacaaatcttaataatcaagaaaaacttaaacaaattcataatgaaagtgtaaataaatcgattttacttgaatataagaaagaagattctgaaaagatagaagaaaaggatagttacatttctcataatgtgaatcaagacatgttttatgatatagataaagctatcaaaaattatcgttctagagaaaacaacgaatcgaaagaaataacatcttctatttttaataaaccattctcagacccagatgataaatcctgtatatacatacctatatttttaatagaaatatactgtttcgaaaaaaataatttgttttattttatattcattttgtttattttttttatgactagtgcatttatatttttattaataaaattttacaaaaattggaaatattacagaaattatgaaagtttcaaagaatatgatgaaacaattagtctttttgatgatgatgatatataa

SEQ ID NO: 37 - protein, 572 aa, Artificial Sequence
  Synthetic
MNTSNSDLKKENLWDGHDVSLHKGDTSRNTYSNNSGSNGKKTKKKKNEKSNFLANEYKDTFHVEGLPTRNNNVNIYYGNEEDVNHSAINDDDDIFGSGGNTFLNKNLTTVNLSDMKRGGGPPHGTSNSNGIANFDGSFGVAAYSGYNLSNNTGSPLNLSIAQGVANQMGSAANESGSRGYHSNKSGMYHQRGPLEQEFNAGDVHETPLGNVPSGKMNIINLSDNENDDDWGVETASRSSLEGGGNNSFDLFPFFKSLNRIRTKLLCYYDVDSDVIIYRCMCALLPYLKVDKSYDSMDSLDDIEKNAGEASAGRSRRDQRNGRSGVKGGSKSGGNNYGSNGGSNDGVASADENEADDENANIRKISNVNDAFDYYDNKLSIEGNPDLYGFVWVNIFISFTIFFLFNWKNIFFGDSPDGATSSSEETNNQAYVTQNKLNILYTTLIFLYLFNTQTPLLIYVTNFFVTKRVFPIRLSFLISLMSYNNVILFPLILLYKFTLINTSLSFVLFLCSALRFFIFAYYMMSSLFYIHKYTIRTFRNNFSDNVIYMYYGIFCLSYLLLYLQLRNYIFSYL

SEQ ID NO: 38 - protein, 565 aa, Artificial Sequence
  Synthetic
MNKTNRDLEEENLWDDHDVNLHKGNTSRNTYSNNSGSNGKKTKKNGRSNFMANEYKDTFHLKDLPRSNNKVNIYYGNEEDIIDCAINEDDDIFGSGGNTSLNKNLTTVNLSEMKRGGGPPQGISNSNGITNSDGSVSVAAYYGYNLNNNTGNPPNFSTGQGVTNQIIIDANDSGNRRYHSNNKDMYHQRAPLEQKFNAGDVHDMNFRNAPSGKMNIFNLSDNENNDDWGVETASRSSPEGGENNSFDLFPFFKGLNQIRTKLLCYYDVDSDVIIYRCMCALLPYLNVDKSYDSMDSLDDLEKHACQPRSRRSNKDKRNVKSGDKNYGSSHDVASADENEVDDENAHIRKISNVNDAFDYYDNKLSIERNPDLYGFVWVNIFISFTIFFLFNWKNIFFGDSSGIDFTPEDDITWSSEEINNQAYITQNKLNILYTTLIFLYLFNTFTPLSIYMTNFIVTKRTFPIRLSFLISLMSYNNIILFPLILFYKFTLIKTSLSFILFFCSSFRFFIFAHYMLSSLFYIHKYTIRTFRNNFSDNIIYAYYGIFSLSYLLLYLLLRNYIFSYL

SEQ ID NO: 39 - protein, 556 aa, Artificial Sequence
  Synthetic
MANEEYNRKISWDEDGFIGINEDNLSSIRHNNSVNEKSSLLITNECKDSFLIDDFSRRPKNMMSVYYENDDEDNIFGGGENSSLNKNLKTLNLSDIKKKGVKSDSNNLNYGSNNSNGRSSSNNNTRSITNGGSSSSPFQGLMSNVNDALNFVPNSSTITSNKLEDPANTDCSSSNNYNSNNNSNVYHHNSFFEKEYSNTNNVGENKNNYENYEHSLSGKMNIINLSENINVDNKDSNNNNSFDLFPFFKSLNSIRTKLLSYYDLDNDVVIYRCMCALLPYFNVYKTYDFMNNFIDIEKNEHEMDDKNRDNANINETENDDYDDENENITKINNMNNNFNNYNNKLSIEKNPDIYAFVWLNIFISFVIFFLFNIKNMFFSNSIYVDSTSTGTTSNGGAIYDDTQDSDVIINMKNYMKENKLNILYNSLLFIYSFNIFIPVLVHVTNYFVTKKVYPIKLSFLISLMSYNNIILLPVIFIYKYLIIETTSTFFFWFFTLLRFILFVFYMTTSIFYIYKYVNKMLRIHFESNIVYLNYAIFLFSYMSFYLILKSYILNYL

SEQ ID NO: 40 - protein, 511 aa, Artificial Sequence
  Synthetic
MGGGNDGVYGHDYPGRGGSNSRNGHANVHMNTQSTSRNSSSNRELERDNERANLLASQYKDSFHIDDLSRRNNYAVGDTGGGGNVEGIFGKGSDGGFSNENLKTLNLNDIKRGVHGNNAFLSSSNNMGYSLDFAVEGTVVGDKINTVSAFPSATTVTSGVTASQQVNGIFEKAYSMSDMDKQHGNNISGKMNIIDLSANGNEEKRGDIHSGNNSFELFPFFKSLSAIRNKVLCYYDVDNDVIIYRCMCALVPYLQVDKTFECANNFNDIENNVNQTGNRNMNTLNSLEKETYDENEHIRNISNIHDAFDSYDNKLSVLNNPDVYGFVWLNMLITCIVFFLFNLKNIFYSKVSFIDVDTYTKDTQLMQDYIETNKLTILYSTILFVYLFTVLVPTVVHVTNYLLTKKIMPIKLSFLISLMSYNNITLIPVIFMHKLTSIETNTPFLLFVCSTLRFLVFLFYIITSVFYIYKYTIRVLRNNFADNITHFNYAIFAISYMSFYFLLRSYIFNYL

SEQ ID NO: 41 - protein, 536 aa, Artificial Sequence
  Synthetic
MVNQDDKFKKPKNEIWENDEIYKKKNKCVNSTNDNSFMNMNGKNNFPLEDEYKDIFQINNFSKNTDHNKNNVHLINNHNMKHNNNFITNEESEKNSLLSNKDLIIFNLQDIKNDGNMKRFDHTNNTFQTKSNTTTNNNNHRNSLDVILSNSNMNPIETNQLNNVLKNDNTLNMYENNSYYEKNIQGKMNIINLSDNDINQDDDKRNSFDIFPFFKKFKGIRTKLLSYYDIDTDVVIYRCMCALFPYLNVDKNYDVINNIYDIEKNCVDTNENGFDNNTSTKEYTSQEKNMNTNNSKTNVRNNDKMNKEKTNLFDDETYDEENVRKSSNVNDALDYYDNKLGLEKNPDIYSFVWLNLFISFLVFFLFNIKNVFFNDINNNISTNHISNNKNNHILDNQSKLNILYNTIFFIYSFNIFIPIIIYLTIYFKTKKIPPFKLIYLISLLSYNNIMLLPIIFIYKIIIINTSINLVLYLYAILRFLIFIFYINTSIFYIYKYTNNIFFNHFTTDLIYVLYAIFFLSYVSFYILLKYYIFNNL

SEQ ID NO: 42 - protein, 527 aa, Artificial Sequence
  Synthetic
MGNERNNQNYNDNDEKSRLVQNEFNNDFLINSNSRVDNNNSNIYYTNKNDKYDQNDKYDQTDKYDNEIIFGSNIRNIQNGNLKTFNLNDINKESSNNSRNKNSNFTLNNSNKNLPSHFLNFSEDIITDNINKNERKNGIQNESQNESQNNATTILDAQNNPPFLLNEIHKNSNFYQTEGFEKEHNFFSEKQNNTQTNIMGKMNIINLSDKTSEDNFDENNENTPFGFFPLFKKLNNIRTKLLRFYDIDNDIIIYRCMCALFPYCDVDKKSYFINNFDDIEQGHINSKIQNDCKTTEYSTDINNYTSNISDNENYDENENIRNVTCINDDAFDYYDNKLNIIQNPDLYGFIWLNIFISFIYFFIFNLNNTIFNTTININNDYINSYIYQNKLNVLYNTLFFIYLFNILLPIFILLANYFITKKKFAIKLLSLISLASYNNIILLPLILIYKFTIIDTTILIIQYISSFIRLLLFIFYVATSLMYIYKFTIKIYRNNFSNEIIYFNYFIFTISYISLYFILKSYVFNYL

SEQ ID NO: 43 - DNA, 1719 bp, Artificial Sequence
  Synthetic
atgaacacatcaaatagcgatttgaaaaaggaaaatttatgggacggccacgatgtgagcttacacaaaggcgatacgagccgcaacacgtatagcaacaatagtggcagtaatgggaagaagacgaaaaagaagaagaatgaaaagtcgaatttcttggccaatgaatacaaggacaccttccacgtggagggtctacctacgcgtaacaataacgtgaatatatattatggcaatgaggaagacgtcaatcacagtgccatcaacgacgacgatgacatttttgggagtggaggaaacactttccttaataagaatttaacaacggtcaatttgagtgacatgaaaaggggaggagggcccccccatggtacaagcaactcaaatggaatcgccaattttgatggcagtttcggtgtagcggcatacagcggatataacctaagcaacaacactggtagccccctcaacctttcgattgcccagggtgtagctaatcaaatgggaagcgccgcgaacgagagcggcagtcgaggatatcacagcaataagagcggcatgtatcaccagaggggccccctcgaacaagaattcaatgccggagatgtgcacgaaacgcccttgggaaacgtcccgagcggaaaaatgaacataattaatctgagcgacaacgaaaatgacgacgactggggggtcgaaaccgcctcgagaagcagccttgagggcggaggaaacaactcgtttgatctgttccccttttttaaaagtctgaaccgaatcagaacaaagctcttgtgctactacgacgtggacagcgacgtgatcatatacaggtgcatgtgcgcgctgctgccttacctgaaggtggacaagtcgtacgattccatggacagcttggacgacattgagaagaacgcgggagaggcgagcgccgggcgcagccgcagggaccagcgcaatgggagaagtggcgtcaaaggtggcagcaaaagtggtggcaacaattacggtagcaatggcggcagcaatgacggcgtcgccagcgcggacgaaaacgaagccgacgacgagaacgcgaacataagaaaaatcagcaacgtgaatgacgccttcgactactatgacaacaagctgagcatagaggggaacccagacctgtatggatttgtgtgggtgaacatcttcatttccttcaccattttttttcttttcaactggaaaaatatctttttcggggactcccccgatggtgccacctcgagtagcgaagaaacaaacaaccaagcgtacgtcacgcagaacaaattaaacattctgtacaccactttaatttttttatatctcttcaatacgcaaacgcctttattaatatacgtcacaaatttctttgtcacgaaaagggtgtttcccatcaggttatcctttctcatttcgctaatgagttataacaacgtaattttgttccccctcattttgttatacaaatttaccttaataaatacgagcctttcgtttgttctcttcctttgcagtgccctgcgtttttttatttttgcctactacatgatgtcctccctattttatatacacaaatacaccatccgcacttttcgcaacaatttttctgataacgtcatttatatgtactatggaattttttgcctgtcctaccttttgttataccttcagttgaggaactacatattcagttatttgtga

SEQ ID NO: 44 - DNA, 1698 bp, Artificial Sequence
  Synthetic
atgaacaaaacaaatagggatttggaagaggaaaatttatgggatgatcacgatgtgaacttacacaaaggcaatacgagccgcaacacgtatagcaacaatagtggcagtaatgggaagaaaacgaagaagaatgggaggtccaatttcatggccaatgaatacaaagacaccttccacctgaaggaccttcccaggagtaacaataaagtgaatatatattatggcaatgaggaagatatcattgactgcgccatcaacgaagacgatgacatttttgggagtggaggaaacacttctctgaataagaatttaacaacggttaatttgagtgaaatgaaaaggggaggaggccccccccaaggtataagcaattcaaatggaatcaccaattctgatggcagtgtcagtgtagcggcatattacggatataacctgaacaacaacacgggtaacccccctaacttttcgactggccagggtgtaactaatcaaataataatcgacgcgaatgatagcggaaatcgaagataccacagcaataataaggacatgtaccaccagagggcccccctcgaacaaaaattcaatgccggagatgtgcacgatatgaacttcagaaacgccccgagcgggaaaatgaacatatttaatctaagcgacaacgaaaataacgacgactggggggttgaaacagcatcgagaagcagccctgagggcggagaaaacaattcgttcgacctgtttcctttttttaaaggtctaaaccaaatcagaacaaagctgttgtgttactacgacgtggacagcgatgtgataatatacagatgcatgtgtgctcttctgccttacctgaacgtggacaagtcgtacgattccatggacagcttggatgaccttgaaaagcacgcgtgtcaaccgagaagccgacgcagcaacaaggacaagcgcaatgtaaaaagtggcgacaaaaattatggcagtagtcacgatgttgccagtgcagatgaaaacgaagtagacgacgagaacgcgcacataagaaaaataagcaacgtgaatgacgccttcgattactatgacaacaaactgagcatagaaagaaacccagacctgtatggatttgtatgggtgaatatcttcatttccttcaccattttttttctttttaattggaaaaacatattttttggtgattcctccggtatcgactttacacccgaagatgacatcacctggagtagcgaagaaataaacaaccaagcatacatcacccagaataaattaaacattctctacaccactttaatttttttataccttttcaatacgttcactcctttatcaatatacatgacaaatttcattgtcacaaaaagaacgttcccaatcaggttatcctttctcatttcattaatgagttataataatataattttattccctctcatattgttttacaaatttaccttgataaaaacaagtctgtcattcattctcttcttttgtagttccttccgtttttttatttttgcccactatatgctgtcttccttattttacatacacaaatataccatccgcacttttcgcaataatttttctgataacatcatttatgcctactatggaattttttccctgtcctaccttctgttataccttctgttgaggaactacatattcagttatttgtga

SEQ ID NO: 45 - DNA, 1671 bp, Artificial Sequence
  Synthetic
atggcgaatgaagaatacaacagaaagattagctgggatgaagatggtttcattggaataaacgaagataaccttagtagcattaggcacaataatagtgtcaatgaaaagtccagcttgttaattacaaatgaatgtaaagatagttttcttattgatgacttttcaagaagaccaaaaaatatgatgagtgtatattatgaaaatgatgacgaggataatatttttggtgggggggaaaacagctcgttaaataaaaatttaaaaacgcttaatttgagtgatataaaaaaaaagggggtaaaaagtgatagcaataatttaaattatggtagtaataacagtaacggtagaagtagcagtaataacaatactagaagtatcactaatggtggtagtagtagcagtccatttcaagggctcatgagtaacgtgaatgacgctcttaactttgtcccaaatagtagtactataacaagtaataaactggaagaccctgcaaatacagattgttcgtctagtaataattacaacagtaataacaacagtaatgtatatcatcacaattccttttttgaaaaggaatatagtaacacaaataatgtaggagaaaataaaaacaattatgaaaattatgaacatagtctaagcggaaaaatgaacataatcaatttaagcgaaaatataaatgtagataataaggatagcaataataacaactcttttgatctatttcccttttttaaaagcttaaatagtattagaacaaaattattatcatattatgatttagataatgatgtagtaatatataggtgtatgtgtgcactactaccttattttaatgtatataaaacatatgactttatgaacaattttattgatatagaaaagaatgaacatgaaatggatgataaaaatagggataatgcaaatattaacgaaacggaaaatgatgattatgatgatgaaaatgaaaatataacaaaaataaataacatgaataataactttaataattataacaacaaattaagtattgaaaaaaacccagatatatatgcctttgtttggttgaatatattcatctcctttgtaattttttttcttttcaacataaaaaatatgttttttagtaacagcatatatgttgatagtacttccacaggtactactagtaatggtggtgcaatatacgatgatacgcaggatagtgatgtgataattaatatgaaaaattacatgaaggaaaacaaattgaacattctctacaattcattactttttatctattcatttaacatttttatacctgtacttgtgcatgtaactaactattttgttacaaaaaaagtatatccaattaaattgtcatttcttatttctttaatgagttataataacataattttattaccagtgatttttatttacaagtatttaattatagaaactacttcaacattttttttttggtttttcacacttttacgatttattctttttgttttttatatgactacatccatattttatatatataaatatgtcaacaaaatgttgcgcattcattttgaaagtaatattgtatatctcaattacgctatttttttattttcgtacatgtccttttatctcatattaaagagttacatactcaattatttataa

SEQ ID NO: 46 - DNA, 1536 bp, Artificial Sequence
  Synthetic
atgggtggtggtaacgacggtgtttatggtcatgattatcctggtaggggtggaagtaactcgcgcaacgggcatgcaaatgtgcacatgaacacacaaagtacatcccgcaattcgtccagtaacagagaattagaaagagataatgaaagagctaacctactggcgagtcagtataaagatagttttcacattgatgacttgtcacggaggaataattacgccgttggtgacactggtggaggtggcaatgtggaaggaatttttggaaagggctccgatggtggtttctcaaatgagaacttgaaaacactaaatttaaatgatataaagaggggagtgcatggaaataacgcgtttttaagcagttctaacaacatggggtattccctcgactttgcggttgagggaactgtagttggggacaaaataaataccgtttcagctttccctagtgctactactgtaacaagcggagttactgcttcccagcaggtgaatggcatttttgaaaaggcatatagcatgagtgatatggacaaacaacatggaaataacataagtggaaaaatgaacataattgatttaagtgcgaatggaaatgaggaaaaaagaggagatatacatagtggaaataattctttcgagttatttcccttttttaaaagtctcagtgctattagaaacaaagtattatgttattatgacgtggacaatgatgttattatttacagatgcatgtgtgcattagttccgtacttacaggtagacaaaacgttcgaatgtgctaacaattttaatgatatagaaaataatgtaaaccaaactggtaatagaaatatgaacacactgaacagtttagaaaaagagacttatgacgaaaacgaacatatacgaaatataagtaatatacacgatgcgtttgattcttatgataacaaattgagtgttctaaataacccagatgtatacggttttgtctggttaaatatgcttataacgtgtatagttttttttctattcaatttgaaaaacattttttatagtaaggtatctttcattgatgtagatacttacacaaaagacacccaacttatgcaagattacattgaaacaaacaaactgacaatactatacagtacaattcttttcgtatatctctttaccgttttggtcccaacagtggtacatgttactaattaccttctaacaaaaaaaataatgccaataaaattatcgtttcttatatccctaatgagttacaacaatataaccttaatacccgttatctttatgcacaaacttacaagcatagaaacaaatacaccttttcttcttttcgtttgttctactctacgttttcttgtctttcttttttacataattacatctgttttttatatatataaatatactataagagtattacgtaacaattttgccgacaatattacccatttcaattacgctatttttgcaatttcttacatgtctttttattttctcctaagaagttacatattcaactatctatga

SEQ ID NO: 47 - DNA, 1611 bp, Artificial Sequence
  Synthetic
atggttaatcaagatgataaatttaagaaacctaaaaatgaaatttgggaaaatgatgagatatataaaaaaaaaaacaaatgtgttaattcaaccaatgataattcttttatgaatatgaatggaaagaataattttcctttagaagatgaatataaggatatatttcagattaataatttttctaagaatactgatcataataaaaataatgtgcatcttataaataatcataatatgaaacataataataattttataacaaatgaggaatcagaaaaaaactctctcttgtcaaataaagatttaattatttttaatttacaagatattaaaaatgatggtaatatgaaaaggtttgatcatactaataatacatttcaaacaaaatctaatactactacaaataataataatcacagaaatagtttagatgttattttgtctaattctaatatgaatccaatcgaaacgaaccaattaaataatgtattaaaaaatgataatacattaaatatgtatgaaaataattcatattatgaaaaaaatatacaaggaaaaatgaatattataaatttaagtgataatgatataaatcaggatgatgataaaagaaattcttttgatatcttccccttctttaaaaaatttaaaggtataagaacaaaattattaagttattatgatatagatacagacgttgtcatatatagatgtatgtgtgccttatttccatatttaaatgttgacaaaaattatgatgttataaataatatatatgatattgaaaaaaattgtgtagatacaaatgaaaatggtttcgataataatacaagtacaaaagaatatacatcacaagaaaagaacatgaatacaaacaacagcaaaaccaatgttagaaataatgataaaatgaataaagagaaaactaacctttttgatgacgaaacatatgatgaagaaaatgtaagaaaatcaagcaatgtaaatgatgctttagattattatgataataaactagggttagaaaaaaatccagatatttattccttcgtatggttaaatttatttatatcctttctagtgttttttctttttaatataaaaaatgtatttttcaatgatattaataataatatatcaacaaatcacatatcaaataataagaacaatcatattttagataatcaaagcaaattaaatattttatataatactatatttttcatatactcatttaatatatttattcccattataatatatctaacaatttatttcaaaaccaaaaaaataccacctttcaaattaatatatcttatatctttattaagttataataatattatgttattaccaatcatctttatttataaaattattattataaatacctccataaatttagtactctatctttatgcaattctacgtttccttatcttcattttttatattaatacatctattttttatatttataaatatacaaacaatatattttttaatcattttactacagatttgatatatgtactttatgcaatctttttcctttcatatgtatctttttatattcttctaaaatattacatatttaataatttataa

SEQ ID NO: 48 - DNA, 1584 bp, Artificial Sequence
  Synthetic
atgggtaatgaacgtaataatcaaaattataatgataatgatgaaaaatcaagattagtacaaaatgaatttaataacgattttcttataaattcaaattcaagggtagacaacaataatagtaatatttattatacaaataaaaatgataaatatgatcaaaatgataaatatgatcaaactgataaatatgataatgaaattatttttggatcaaatattagaaatatccaaaatggtaatttaaaaacatttaatttaaatgatataaataaagaatcaagtaataacagtcgaaataaaaattcaaactttactttaaataattcaaataaaaatctcccttctcatttcctcaatttttctgaggatattataactgacaatatcaataaaaacgaaagaaaaaatggaatccaaaatgaaagccaaaacgaaagccagaataacgctacaaccattttagacgcacaaaataaccctccttttttattaaatgaaattcataagaattcaaatttttatcaaacggaaggtttcgaaaaagaacacaattttttttctgaaaaacaaaataatacccaaacaaatataatgggaaaaatgaatataataaatctaagtgacaaaacaagtgaagataattttgatgaaaataatgaaaacacaccttttggtttttttcctttatttaaaaagttaaataatataagaactaaattgttacgtttttatgatatagataacgatataattatatatcgatgtatgtgtgcattatttccatattgtgatgtagacaaaaaatcttattttattaataattttgatgatattgaacaaggccatatcaactcaaaaatacaaaatgattgtaaaactacagaatattccacagatataaataattatacatcaaatatttcagacaatgaaaattatgatgaaaatgaaaatatcagaaatgtaacatgtattaatgatgatgcttttgattattatgataataaattaaacattatacaaaatccagacttatatggatttatatggttaaatatatttatttcttttatatatttttttatttttaatttaaataatactatatttaataccacaataaatattaacaatgattatataaatagctatatttatcaaaataaattaaatgttttatataatacattattttttatttatttgtttaatatattacttcctatttttattttactagctaattattttattacaaaaaaaaaattcgctattaaattattatctttaatttctttagctagctataataatattattttattgcctttaattcttatttataaatttacaattatagacacaactattcttattatacaatatatatcatcatttatacgtttattactttttattttttatgttgctacatcattaatgtatatatataaatttactataaaaatatatcgtaacaatttttcaaatgaaattatttattttaattatttcatttttactatttcttatatctctttatattttatcctcaaatcttatgtatttaattatttataa

SEQ ID NO: 49 - protein, 60 aa, Artificial Sequence
  Synthetic
FSVQYFKEANKINNKCIKKSNIFTESICTDNNCKTACNAYETWINVNKSKWNTLLKSFAN

SEQ ID NO: 50 - protein, 60 aa, Artificial Sequence
  Synthetic
FGVQYFKEAIKINNKCTKKSIISTESICINNACKDECNTYETWISVNKSKWDALLQRFSE

SEQ ID NO: 51 - protein, 60 aa, Artificial Sequence
  Synthetic
FSVQYFKEANKINNKCTKKSNISTESICINNECKNAYNTYEAWISVNKSKWDTLLQRFSQ

SEQ ID NO: 52 - protein, 60 aa, Artificial Sequence
  Synthetic
FSVQYFKEANKINNKCTKKSNISTESICINNECKNACNTYEAWISVNKSKWDTLLQRFSQ

SEQ ID NO: 53 - protein, 60 aa, Artificial Sequence; identical to SEQ ID NO: 52
  Synthetic

SEQ ID NO: 54 - protein, 60 aa, Artificial Sequence; identical to SEQ ID NO: 52
  Synthetic

SEQ ID NO: 55 - protein, 60 aa, Artificial Sequence
  Synthetic
YMSELPKEQGKLNEKCASKLYYNNMAICMLPLCHDACKSYDQWITRKKKQWDVLSTKFSS

SEQ ID NO: 56 - protein, 60 aa, Artificial Sequence
  Synthetic
YVSELPTEVQKLKEKCDGKINYTDKKVCKVPPCQNACKSYDQWITRKKNQWDVLSNKFIS

SEQ ID NO: 57 - protein, 60 aa, Artificial Sequence
  Synthetic
YITELPTERQKVKEKCDGNIIFTKKKVCTVSQCKNACNLYDQWITRKKEQWDVLSNKFKS

SEQ ID NO: 58 - protein, 60 aa, Artificial Sequence
  Synthetic
YILELPKELQKLKEKCDRKFYYTDINVCNVFPCKNACILYDQWITRKKKQWDLLSNKFIS

SEQ ID NO: 59 - protein, 56 aa, Artificial Sequence
  Synthetic
NCRLMKEKEKLLTGECSVNRKKSDCSTGCNNECYTYRSLINRQRYEVSILGKKYIK

SEQ ID NO: 60 - protein, 56 aa, Artificial Sequence
  Synthetic
FLLERDNRSKLPKSKCKNNTLYEACEKECIDPCMKYRDWIIRSKFEWHTLSKEYET

SEQ ID NO: 61 - protein, 56 aa, Artificial Sequence
  Synthetic
FLMEKKRLFLNIKDKCVENKKYEACFGGCRLPCSSYTSFMKKSKTQMEVLTNLYKK

SEQ ID NO: 62 - DNA, 67 bp, Artificial Sequence
  Synthetic
ggggacaagtttgtacaaaaaagcaggctaggcctagggccccatatgaatctttccaatctttccc

SEQ ID NO: 63 - DNA, 55 bp, Artificial Sequence
  Synthetic
ggggaccactttgtacaagaaagctgggtttaataaaatctacaggtatatattc

SEQ ID NO: 64 - DNA, 51 bp, Artificial Sequence
  Synthetic
ggggacaactttgtatagaaaagttgcaaaatattgaattgaagccttatg

SEQ ID NO: 65 - DNA, 63 bp, Artificial Sequence
  Synthetic
ggggactgcttttttgtacaaacttgggccctaggcctaaatatgactatgcataccatcatg

SEQ ID NO: 66 - DNA, 52 bp, Artificial Sequence
  Synthetic
gcgaagttcctattcttcaaatagtataggaacttcaggaaacagctatgac

SEQ ID NO: 67 - DNA, 55 bp, Artificial Sequence
  Synthetic
gcgaagttcctattcttcaaatagtataggaacttccctgaagaagaaaagtccg

SEQ ID NO: 68 - DNA, 22 bp, Artificial Sequence
  Synthetic
gtataatacatatgaggcatgg

SEQ ID NO: 69 - DNA, 25 bp, Artificial Sequence
  Synthetic
gcatttttacattcattattaatac

SEQ ID NO: 70 - DNA, 22 bp, Artificial Sequence
  Synthetic
gtgtaatacatatgaggcatgg

SEQ ID NO: 71 - DNA, 25 bp, Artificial Sequence
  Synthetic
gcatttttacattcattattaatac

SEQ ID NO: 72 - DNA, 27 bp, Artificial Sequence
  Synthetic
cgatagtgaaaatgccaataaattggg

SEQ ID NO: 73 - DNA, 22 bp, Artificial Sequence
  Synthetic
gatatgattaatagttttggag

SEQ ID NO: 74 - DNA, 23 bp, Artificial Sequence
  Synthetic
gaagaaacttgtagagataaagc

SEQ ID NO: 75 - DNA, 21 bp, Artificial Sequence
  Synthetic
caacaggaggtataatatttg

SEQ ID NO: 76 - DNA, 22 bp, Artificial Sequence
  Synthetic
aaccgatgataatgctaaaagg

SEQ ID NO: 77 - DNA, 23 bp, Artificial Sequence
  Synthetic
tttgtttaaagaagatgacgatg

SEQ ID NO: 78 - DNA, 26 bp, Artificial Sequence
  Synthetic
tcaacctcaacctcaatttccaattc

SEQ ID NO: 79 - DNA, 21 bp, Artificial Sequence
  Synthetic
tgaatctttccaatctttccc

SEQ ID NO: 80 - DNA, 22 bp, Artificial Sequence
  Synthetic
ccatgtctctccgttttcaatg

SEQ ID NO: 81 - DNA, 29 bp, Artificial Sequence
  Synthetic
ccaaatgcatagagttttatattttatta

SEQ ID NO: 82 - DNA, 20 bp, Artificial Sequence
  Synthetic
tacaattaaaatgctacaca

SEQ ID NO: 83 - DNA, 24 bp, Artificial Sequence
  Synthetic
ccgatggacgcacaaatgaatatg

SEQ ID NO: 84 - DNA, 24 bp, Artificial Sequence
  Synthetic
cgttggctagcattattatatttt

SEQ ID NO: 85 - DNA, 22 bp, Artificial Sequence
  Synthetic
cattatcacaaatccaagcatg

SEQ ID NO: 86 - DNA, 25 bp, Artificial Sequence
  Synthetic
gttgaagttggattgatagttacag

SEQ ID NO: 87 - DNA, 26 bp, Artificial Sequence
  Synthetic
cttcatatgcttctttaaattcgatc

SEQ ID NO: 88 - DNA, 27 bp, Artificial Sequence
  Synthetic
caatatgttcatgatttttccaccaag